(19) United States
(12) Patent Application Publication
(10) Pub. No.: US 2011/0179520 A1
(43) Pub. Date: Jul. 21, 2011

(54) TISSUE-ENHANCED PROMOTERS

(75) Inventors: Suqin CAI, Union City, CA (US); Hans E. Holtan, Emeryville, CA (US); Peter P. Repetti, Emeryville, CA (US); T. Lynne Reuber, San Mateo, CA (US)

(73) Assignee: Mendel Biotechnology, Inc., Hayward, CA (US)

(21) Appl. No.: 13/009,039

(22) Filed: Jan. 19, 2011

Related U.S. Application Data
(60) Provisional application No. 61/296,776, filed on Jan. 20, 2010.

(51) Int. Cl. A01H 1/00; C07H 21/04; C12N 5/10; A01H 5/00

(52) U.S. Cl. 800/279; 536/24.1; 435/419; 800/298; 800/301; 800/278; 800/282; 800/289

(57) ABSTRACT

Tissue-enhanced promoter sequences were identified that enhance expression of a polypeptide in one or more plant tissues. These promoters may be used to produce transgenic plants that have an altered trait relative to control plants. In preferred embodiments, the transgenic plants with the improved traits are morphologically and/or developmentally similar to control plants (examples of the latter include wild-type or non-transformed plants of the same species). Any of these tissue-enhanced promoters may be incorporated into a nucleic acid construct that comprises a polynucleotide regulated by one such promoter and that encodes a polypeptide or RNA molecule that, when ectopically expressed, confers an improved trait in plants.

### CROSS-REFERENCES TO RELATED APPLICATIONS

This application claims the benefit of U.S. Provisional Application No. 61/296,776, filed Jan. 20, 2010 (pending). The entire contents of Application No. 61/296,776 are hereby incorporated by reference.

### FIELD OF THE INVENTION

The present invention relates to plant genomics, and more specifically pertains to promoters that mediate gene expression.

### BACKGROUND

To expand the knowledge and use of optimization strategies for genes and proteins that improve a plant's traits when the gene or protein is overexpressed in a plant, an effort was made to identify tissue-enhanced promoters. A number of these promoter candidates may be found that control gene expression such that expression is present or enhanced only in particular cell types or tissues. Thus, this project may identify and characterize candidate promoters that can regulate gene expression in specific cell-types, groups of cell-types (tissues) or in specific whole organs.

Numerous transgenic plants using these promoter sequences to regulate polypeptides were developed and the plants were analyzed for improved traits. Many of these promoter sequences can be used to produce commercially valuable plants and crops as well as the methods for making them and using them.

The present description thus relates to methods and compositions for producing transgenic plants, where tissue-enhanced expression of polypeptides of interest confers improved traits with reduced or no impact on yield, appearance, quality or fitness, as compared to plants constitutively overexpressing the same polypeptides. Other aspects and embodiments are described below and can be derived from the teachings of this disclosure as a whole.

### SUMMARY

The present description and claims are directed to promoter sequences that may be used to transform a plant. The promoter sequences are active only in specific cell-types, groups of cell-types (tissues) or in specific whole organs, and can be used to drive the expression of a polynucleotide sequence that encodes a polypeptide or RNA molecule that can confer an improved trait when expressed in some specific subset of cells. Thus, the polypeptide may be expressed in a specific tissue-regulated manner.

The description also provides an isolated nucleic acid comprising a tissue-enhanced promoter that includes any of the promoter sequences provided by SEQ ID NOs: 1-66. A tissue-enhanced promoter may comprise a functional part thereof, provided the functional part also includes a tissue-regulated promoter function. The functional part of the promoter may have about 25, 50, 75, 100, 125, 150, 175, 200, 225, 250, 275, 300, 325, 350, 375, 400, 425, 450, 475, 500, 525, 550, 575, 600, 625, 650, 675, 700, 724, 725, 750, 775, 800, 825, 850, 875, 900, 925, 950, 975, 1000, 1025, 1050, 1075, 1100, 1125, 1150, 1175, 1200, 1204, 1300, 1400, 1500, 1600, 1700, 1800, 1900, 2000, 2100, 2200, 2300, 2400, 2500, 2600, 2700, 2800, 2900, 2999, or 3000 contiguous nucleotides of the nucleic acid sequences of SEQ ID NOs: 1-66, as well as all lengths of contiguous nucleotides within such sizes.

The description also pertains to expression vectors that can comprise a tissue-enhanced promoter sequence. The tissue-enhanced promoter may comprise any of SEQ ID NOs: 1 to 66, or a functional part thereof, provided the functional part also includes a tissue-enhanced promoter function. The promoter comprises a transcription initiation domain having an RNA polymerase binding site. The promoter is located 5′ relative to and is operably linked to a coding sequence encoding a polypeptide that confers to a plant gene and/or protein regulation when expressed in a specific cell-type. Nucleic acid constructs or recombinant polynucleotides that comprise a promoter of any of SEQ ID NOs: 1-66 may be introduced into plants, and the plants may have an improved or desirable trait relative to a control plant. In some cases, the nucleic acid constructs or recombinant polynucleotides are non-native to the transformed plants. In some cases, the transformed plants are of wild-type or near-wild type morphology and development. This may be of significant utility in that many polypeptides that confer improved traits upon their expression can also cause undesirable morphological and/or developmental traits when the polypeptides are constitutively overexpressed. Non-constitutive regulation of expression, such as that found in some cell types, but not (or to a lesser extent) others, may be used to confer the improved traits while mitigating the undesirable morphological and/or developmental effects.

In a preferred embodiment, there is a strong and specific gene expression only in shoot apical meristems, or general meristematic tissue, such that the operably linked DNA sequences that encode useful polypeptides are expressed in a strong and specific manner. In another embodiment, there is strong and specific up-regulation by the promoter in the vascular tissue, with little or no expression elsewhere, such that the operably linked DNA sequences that encode useful polypeptides are expressed only, or much more strongly, in the fluid conducting tissues of a plant. In another embodiment, there is strong and specific gene expression only in nascent leaf primordia, such that the operably linked DNA sequences that encode useful polypeptides are expressed in a strong and specific manner in emerging leaves, but not (or to a lesser degree) elsewhere.

The description encompasses a host plant cell comprising a tissue-enhanced promoter, comprising any of SEQ ID NOs: 1-66 or a functional part thereof, wherein the functional part includes a promoter function.

The description also encompasses a transgenic plant comprising a tissue-enhanced promoter, comprising any of SEQ ID NOs: 1-66 or a functional part thereof, wherein the functional part includes a promoter function, and transgenic seed produced by the transgenic plant.

Methods for producing a transgenic plant having tissue-enhanced gene expression, relative to a control plant, are provided. The method steps include the generation of a nucleic acid construct (e.g., an expression vector or cassette) that comprises a promoter sequence of any of SEQ ID NOs: 1-66 or a functional part thereof, wherein the functional part includes a tissue-enhanced promoter function. The promoter sequence is operably linked to a nucleotide sequence that encodes a RNA molecule or polypeptide that improves a trait in a plant, and the promoter sequence drives expression of the nucleotide sequence in a tissue-enhanced manner. A target plant can be transformed with the nucleic acid construct to produce a transgenic plant. When the polypeptide is overexpressed in the transformed plant in response to different cellular or tissue environments, the transformed plant will express the improved trait relative to the control plant. A transgenic plant that is produced by this method may be crossed with itself, a plant from the same line as the transgenic plant, a non-transgenic plant, a wild-type plant, or another transgenic plant from a different transgenic line of plants, to produce a transgenic seed that comprises the expression vector.

### BRIEF DESCRIPTION OF THE SEQUENCE LISTING

The Sequence Listing provides exemplary polynucleotide and polypeptide sequences. The traits associated with the use of the sequences are included in the Examples.

Incorporation of the Sequence Listing. The copy of the Sequence Listing, being submitted electronically with this patent application, provided under 37 CFR §1.821-1.825, is a computer-readable file in ASCII text format. The Sequence Listing is named “MBI-0097P.txt”, the electronic file of the Sequence Listing was created on Jan. 14, 2010, and is 280,493 bytes in size (or 274 kilobytes in size as measured in MS-WINDOWS). The Sequence Listing is herein incorporated by reference in its entirety.

### DETAILED DESCRIPTION

The present description relates to polynucleotides and polypeptides for modifying phenotypes of plants, particularly promoter sequences associated with tissue-enhanced gene regulation, and which may inducibly regulate an improved trait with respect to a control plant. Examples of control plants include, for example, genetically unaltered or non-transgenic plants such as wild-type plants of the same species, or non-transformed plants, or plants that have mutations in one or more loci, or transgenic plant lines that comprise an empty expression vector. Throughout this disclosure, various information sources are referred to and/or are specifically incorporated. The information sources include scientific journal articles, patent documents, textbooks, and World Wide Web page addresses. While the reference to these information sources clearly indicates that they can be used by one of skill in the art, each and every one of the information sources cited herein are specifically incorporated in their entirety, whether or not a specific mention of “incorporation by reference” is noted. The contents and teachings of each and every one of the information sources can be relied on and used to make and use embodiments of the claims.

As used herein and in the appended claims, the singular forms “a”, “an”, and “the” include the plural reference unless the context clearly dictates otherwise. Thus, for example, a reference to “a host cell” includes a plurality of such host cells, and a reference to “a stress” is a reference to one or more stresses and equivalents thereof known to those skilled in the art, and so forth.

### DEFINITIONS

“Nucleic acid molecule” refers to an oligonucleotide, polynucleotide or any fragment thereof. It may be DNA or RNA of genomic or synthetic origin, double-stranded or single-stranded, and combined with carbohydrate, lipids, protein, or other materials to perform a particular activity such as transformation or form a useful composition such as a peptide nucleic acid (PNA).

“Polynucleotide” is a nucleic acid molecule comprising a plurality of polymerized nucleotides, e.g., at least about 15 consecutive polymerized nucleotides. A polynucleotide may be a nucleic acid, oligonucleotide, nucleotide, or any fragment thereof. In many instances, a polynucleotide comprises a nucleotide sequence encoding a polypeptide (or protein) or a domain or fragment thereof. Additionally, the polynucleotide may comprise a promoter, an intron, an enhancer region, a polyadenylation site, a translation initiation site, 5′ or 3′ untranslated regions, a reporter gene, a selectable marker, a transcriptional activation or repression domain, or the like. The polynucleotide can be single-stranded or double-stranded DNA or RNA. The polynucleotide optionally comprises modified bases or a modified backbone. The polynucleotide can be, e.g., genomic DNA or RNA, a transcript (such as an mRNA), a cDNA, a PCR product, a cloned DNA, a synthetic DNA or RNA, or the like. The polynucleotide can be combined with carbohydrate, lipids, protein, or other materials to perform a particular activity such as transformation or form a useful composition such as a peptide nucleic acid (PNA). The polynucleotide can comprise a sequence in either sense or antisense orientations. “Oligonucleotide” is substantially equivalent to the terms amplimer, primer, oligomer, element, target, and probe and is preferably single-stranded.

A “recombinant polynucleotide” is a polynucleotide that is not in its native state, e.g., the polynucleotide comprises a nucleotide sequence not found in nature, or the polynucleotide is in a context other than that in which it is naturally found, e.g., separated from nucleotide sequences with which it typically is in proximity in nature, or adjacent (or contiguous with) nucleotide sequences with which it typically is not in proximity. For example, the sequence at issue can be cloned into a vector, or otherwise recombined with one or more additional nucleic acids.

An “isolated polynucleotide” is a polynucleotide, whether naturally occurring or recombinant, that is present outside the cell in which it is typically found in nature, whether purified or not. Optionally, an isolated polynucleotide is subject to one or more enrichment or purification procedures, e.g., cell lysis, extraction, centrifugation, precipitation, or the like.

“Gene” or “gene sequence” refers to the partial or complete coding sequence of a gene, its complement, and its 5′ or 3′ untranslated regions. A gene is also a functional unit of inheritance, and in physical terms is a particular segment or sequence of nucleotides along a molecule of DNA (or RNA, in the case of RNA viruses) involved in producing a polypeptide chain. The latter may be subjected to subsequent processing such as chemical modification or folding to obtain a functional protein or polypeptide. A gene may be isolated, partially isolated, or found within an organism's genome. By way of example, a transcription factor gene encodes a transcription factor polypeptide, which may be functional or require processing to function as an initiator of transcription.

Operationally, genes may be defined by the cis-trans test, a genetic test that determines whether two mutations occur in the same gene and that may be used to determine the limits of the genetically active unit (Rieger et al. (1976)). A gene generally includes regions preceding (“leaders”; upstream) and following (“trailers”; downstream) the coding region. A gene may also include intervening, non-coding sequences, referred to as “introns”, located between individual coding segments, referred to as “exons”. Most genes have an associated promoter region, a regulatory sequence 5′ of the transcription initiation codon (there are some genes that do not have an identifiable promoter). The function of a gene may also be regulated by enhancers, operators, and other regulatory elements.

A “promoter” or “promoter region” refers to an RNA polymerase binding site on a segment of DNA, generally found upstream or 5′ relative to a coding sequence under the regulatory control of the promoter. The promoter will generally comprise response elements that are recognized by transcription factors. Transcription factors bind to the promoter sequences, recruiting RNA polymerase, which synthesizes RNA from the coding region. Dissimilarities in promoter sequences account for different efficiencies of transcription initiation and hence different relative expression levels of different genes.

“Promoter function” includes regulating expression of the coding sequences under a promoter's control by providing a recognition site for RNA polymerase and/or other factors, such as transcription factors, all of which are necessary for the start of transcription at a transcription initiation site. A “promoter function” may also include affecting the activity or level to which a gene coding sequence is transcribed to an extent determined by a promoter sequence.

A promoter or promoter region may include variations of promoters found in the present Sequence Listing, which may be derived by ligation to other regulatory sequences, random mutagenesis, controlled mutagenesis, and/or by the addition or duplication of enhancer sequences. Promoters disclosed in the Sequence Listing and biologically functional equivalents or variations thereof may drive the transcription of operably-linked coding sequences when comprised within an expression vector and introduced into a host plant. Promoters such as those found in the Sequence Listing (i.e., SEQ ID NOs: 1-66) may be used to generate similarly functional promoters containing essential promoter elements. Functional promoters may also include a functional part of any of SEQ ID NO: 1-66, provided the functional part also includes a tissue-enhanced promoter function.

A “polypeptide” is an amino acid sequence comprising a plurality of consecutive polymerized amino acid residues e.g., at least about 15 consecutive polymerized amino acid residues. In some of the instances referred to in this application, a polypeptide comprises a polymerized amino acid residue sequence that is a transcription factor or a domain or portion or fragment thereof. Additionally, the transcription factor may comprise: (i) a localization domain; (ii) an activation domain; (iii) a repression domain; (iv) an oligomerization domain; (v) a DNA-binding domain; or the like. The polypeptide optionally comprises modified amino acid residues, naturally occurring amino acid residues not encoded by a codon, non-naturally occurring amino acid residues.

“Protein” refers to an amino acid sequence, oligopeptide, peptide, polypeptide or portions thereof whether naturally occurring or synthetic.

A “recombinant polypeptide” is a polypeptide produced by translation of a recombinant polynucleotide. A “synthetic polypeptide” is a polypeptide created by consecutive polymerization of isolated amino acid residues using methods well known in the art. An “isolated polypeptide,” whether a naturally occurring or a recombinant polypeptide, is more enriched in (or out of) a cell than the polypeptide in its natural state in a wild-type cell, e.g., more than about 5% enriched, more than about 10% enriched, or more than about 20%, or more than about 50%, or more, enriched, i.e., alternatively denoted: 105%, 110%, 120%, 150% or more, enriched relative to wild type standardized at 100%. Such an enrichment is not the result of a natural response of a wild-type plant. Alternatively, or additionally, the isolated polypeptide is separated from other cellular components with which it is typically associated, e.g., by any of the various protein purification methods herein.

“Homology” refers to sequence similarity between a reference sequence and at least a fragment of a newly sequenced clone insert or its encoded amino acid sequence.

“Identity” or “similarity” refers to sequence similarity between two polynucleotide sequences or between two polypeptide sequences, with identity being a more strict comparison. The phrases “percent identity” and “% identity” refer to the percentage of sequence similarity found in a comparison of two or more polynucleotide sequences or two or more polypeptide sequences. “Sequence similarity” refers to the percent similarity in base pair sequence (as determined by any suitable method) between two or more polynucleotide sequences. Two or more sequences can be anywhere from 0-100% similar, or any integer value therebetween. Identity or similarity can be determined by comparing a position in each sequence that may be aligned for purposes of comparison. When a position in the compared sequence is occupied by the same nucleotide base or amino acid, then the molecules are identical at that position. A degree of similarity or identity between polynucleotide sequences is a function of the number of identical, matching or corresponding nucleotides at positions shared by the polynucleotide sequences. A degree of identity of polypeptide sequences is a function of the number of identical amino acids at corresponding positions shared by the polypeptide sequences. A degree of homology or similarity of polypeptide sequences is a function of the number of amino acids at corresponding positions shared by the polypeptide sequences.

“Complementary” refers to the natural hydrogen bonding by base pairing between purines and pyrimidines. For example, the sequence A-C-G-T (5′->3′) forms hydrogen bonds with its complements A-C-G-T (5′->3′) or A-C-G-U (5′->3′). Two single-stranded molecules may be considered partially complementary, if only some of the nucleotides bond, or “completely complementary” if all of the nucleotides bond. The degree of complementarity between nucleic acid strands affects the efficiency and strength of hybridization and amplification reactions. “Fully complementary” refers to the case where bonding occurs between every base pair and its complement in a pair of sequences, and the two sequences have the same number of nucleotides.

The terms “paralog” and “ortholog” are defined below in the section entitled “Orthologs and Paralogs”. In brief, orthologs and paralogs are evolutionarily related genes that have similar sequences and functions. Orthologs are structurally related genes in different species that are derived by a speciation event. Paralogs are structurally related genes within a single species that are derived by a duplication event.

In general, the term “variant” refers to molecules with some differences, generated synthetically or naturally, in their base or amino acid sequences as compared to a reference (native) polynucleotide or polypeptide, respectively. These differences include substitutions, insertions, deletions or any desired combinations of such changes in a native polynucleotide of amino acid sequence.

With regard to polynucleotide variants, differences between presently disclosed polynucleotides and polynucleotide variants are limited so that the nucleotide sequences of the former and the latter are closely similar overall and, in many regions, identical. Due to the degeneracy of the genetic code, differences between the former and latter nucleotide sequences may be silent (i.e., the amino acids encoded by the polynucleotide are the same, and the variant polynucleotide sequence encodes the same amino acid sequence as the presently disclosed polynucleotide. Variant nucleotide sequences may encode different amino acid sequences, in which case such nucleotide differences will result in amino acid substitutions, additions, deletions, insertions, truncations or fusions with respect to the similar disclosed polynucleotide sequences. These variations may result in polynucleotide variants encoding polypeptides that share at least one functional characteristic. The degeneracy of the genetic code also dictates that many different variant polynucleotides can encode identical and/or substantially similar polypeptides in addition to those sequences illustrated in the Sequence Listing.

Also within the claimed scope is a variant of a gene promoter listed in the Sequence Listing, that is, one having a sequence that differs from one of the polynucleotide sequences in the Sequence Listing, or a complementary sequence.

The term “plant” includes whole plants, shoot vegetative organs/structures (for example, leaves, stems and tubers), roots, flowers and floral organs/structures (for example, bracts, sepals, petals, stamens, carpels, anthers and ovules), seed (including embryo, endosperm, and seed coat) and fruit (the mature ovary), plant tissue (for example, vascular tissue, ground tissue, and the like) and cells (for example, guard cells, egg cells, and the like), and progeny of same. The class of plants that can be used in the instant method is generally as broad as the class of higher and lower plants amenable to transformation techniques, including angiosperms (monocotyledonous and dicotyledonous plants), gymnosperms, ferns, horsetails, psilophytes, lycophytes, bryophytes, and multicellular algae (see, for example, Daly et al., 2001, Ku et al., 2000; and see also Tudge, 2000).

A “control plant” as used in the present description refers to a plant cell, seed, plant component, plant tissue, plant organ or whole plant used to compare against transgenic or genetically modified plant for the purpose of identifying an enhanced phenotype in the transgenic or genetically modified plant. A control plant may in some cases be a transgenic plant line that comprises an empty vector or marker gene, but does not contain the recombinant polynucleotide of the present description that is expressed in the transgenic or genetically modified plant being evaluated. In general, a control plant is a plant of the same line or variety as the transgenic or genetically modified plant being tested. A suitable control plant would include a genetically unaltered or non-transgenic plant of the parental line used to generate a transgenic plant herein.

A “transgenic plant” refers to a plant that contains genetic material not found in a wild-type plant of the same species, variety or cultivar. The genetic material may include a transgene, an insertional mutagenesis event (such as by transposon or T-DNA insertional mutagenesis), an activation tagging sequence, a mutated sequence, a homologous recombination event or a sequence modified by chimeraplasty. Typically, the foreign genetic material has been introduced into the plant by human manipulation, but any method can be used as one of skill in the art recognizes.

A transgenic plant may contain a nucleic acid construct (e.g., an expression vector or cassette). The nucleic acid construct typically comprises a polypeptide-encoding sequence operably linked (i.e., under regulatory control of) to an inducible regulatory sequence, such as a promoter, that allows for the controlled expression of polypeptide. The nucleic acid construct can be introduced into a plant by transformation or by breeding after transformation of a parent plant. A plant refers to a whole plant as well as to a plant part, such as seed, fruit, leaf, or root, plant tissue, plant cells or any other plant material, e.g., a plant explant, as well as to progeny thereof, and to in vitro systems that mimic biochemical or cellular components or processes in a cell.

“Wild type” or “wild-type”, as used herein, refers to a plant cell, seed, plant component, plant tissue, plant organ or whole plant that has not been genetically modified or treated in an experimental sense. Wild-type cells, seed, components, tissue, organs or whole plants may be used as controls to compare levels of expression and the extent and nature of trait modification with cells, tissue or plants of the same species in which expression of a polypeptide, such as a transcription factor polypeptide, is altered, e.g., in that it has been overexpressed or ectopically expressed.

A “trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g., by measuring tolerance to a form of stress, such as water deficit or water deprivation, or particular salt or sugar concentrations, or by the observation of the expression level of a gene or genes, e.g., by employing Northern analysis, RT-PCR, microarray gene expression assays, or reporter gene expression systems, or by agricultural observations such as extent of wilting, turgor, hyperosmotic stress tolerance or in a preferred embodiment, yield. Any technique can be used to measure the amount of, comparative level of, or difference in any selected chemical compound or macromolecule in the transgenic plants, however.

“Trait modification” refers to a detectable difference in a characteristic in a plant ectopically expressing a polynucleotide or polypeptide of the present description relative to a plant not doing so, such as a wild-type plant. In some cases, the trait modification can be evaluated quantitatively. For example, the trait modification can entail at least about a 2% increase or decrease, or an even greater difference, in an observed trait as compared with a control or wild-type plant. It is known that there can be a natural variation in the modified trait. Therefore, the trait modification observed entails a change of the normal distribution and magnitude of the trait in the plants as compared to control or wild-type plants.

When two or more plants are “morphologically similar” they have comparable forms or appearances, including analogous features such as dimension, height, width, mass, root mass, shape, glossiness, color, stem diameter, leaf size, leaf dimension, leaf density, internode distance, branching, root branching, number and form of inflorescences, and other macroscopic characteristics at a particular stage of growth. If the plants are morphologically similar at all stages of growth, they are also “developmentally similar”. It may be difficult to distinguish two plants that are genotypically distinct but morphologically similar based on morphological characteristics alone.

The term “transcript profile” refers to the expression levels of a set of genes in a cell in a particular state, particularly by comparison with the expression levels of that same set of genes in a cell of the same type in a reference state. The transcript profile can be presented as a list of those genes whose expression level is significantly different between the two treatments, and the difference ratios. Differences and similarities between expression levels may also be evaluated and calculated using statistical and clustering methods.

“Ectopic expression or altered expression” in reference to a polynucleotide indicates that the pattern of expression in, e.g., a transgenic plant or plant tissue, is different from the expression pattern in a wild-type plant or a reference plant of the same species. The pattern of expression may also be compared with a reference expression pattern in a wild-type plant of the same species. For example, the polynucleotide or polypeptide is expressed in a cell or tissue type other than a cell or tissue type in which the sequence is expressed in the wild-type plant, or by expression at a time other than at the time the sequence is expressed in the wild-type plant, or by a response to different inducible agents, such as hormones or environmental signals, or at different expression levels (either higher or lower) compared with those found in a wild-type plant. The term also refers to altered expression patterns that are produced by lowering the levels of expression to below the detection level or completely abolishing expression. The resulting expression pattern can be transient or stable, constitutive or inducible. In reference to a polypeptide, the term “ectopic expression or altered expression” further may relate to altered activity levels resulting from the interactions of the polypeptides with exogenous or endogenous modulators or from interactions with factors or as a result of the chemical modification of the polypeptides.

The term “overexpression” as used herein refers to a greater expression level of a gene in a plant, plant cell or plant tissue, compared to expression in a wild-type plant, cell or tissue, at any developmental or temporal stage for the gene. Overexpression can occur when, for example, the genes encoding one or more proteins are under the control of a strong promoter (e.g., the cauliflower mosaic virus 35S transcription initiation region). Overexpression may also occur when expression in a particular cell-type, groups of cell-types (tissues) or in specific whole organs is increased relative to the level normally found in those cells (e.g., in non-transgenic plants of the same species), or in comparison to the average expression level in all other tissues in that plant. Thus, overexpression may occur throughout a plant or in a specific sub-group of cells or in a specific tissue or organ, depending on the promoter used. See, for example, U.S. Pat. No. 7,365,186, or U.S. Pat. No. 7,619,133.

Overexpression may take place in plant cells normally lacking expression of polypeptides functionally equivalent or identical to a polypeptide that can confer an improved trait, for example, increased stress tolerance or improved yield. Overexpression may also occur in plant cells where endogenous expression of the present proteins that confer an improved trait, for example, improved stress tolerance, or functionally equivalent molecules, normally occurs, but such normal expression is at a lower level. Overexpression thus results in a greater than normal production, or “overproduction” of the protein that confers the improved trait in the plant, cell or tissue.

The term “transcription regulating region” refers to a DNA regulatory sequence that regulates expression of one or more genes in a plant when a polypeptide having one or more specific binding domains binds to the DNA regulatory sequence. Polypeptides, for example, transcription factors, may possess a conserved domain. Transcription factors may also comprise an amino acid subsequence that forms a transcription activation domain that regulates expression of one or more target genes (for examples, genes that confer stress resistance in a plant when the transcription factor binds to the regulating region.

A “nucleic acid construct” may comprise a polypeptide-encoding sequence operably linked (that is, under regulatory control of) to appropriate inducible, cell-specific, tissue-specific, cell-enhanced, tissue-enhanced, condition-enhanced, developmental, or constitutive regulatory sequences that allow for the controlled expression of the polypeptide. The expression vector or cassette can be introduced into a plant by transformation or by breeding after transformation of a parent plant. A plant refers to a whole plant as well as to a plant part, such as seed, fruit, leaf, or root, plant tissue, plant cells or any other plant material, for example, a plant explant, to produce a recombinant plant (for example, a recombinant plant cell comprising the nucleic acid construct) as well as to progeny thereof, and to in vitro systems that mimic biochemical or cellular components or processes in a cell.

A constitutive promoter is active under most environmental conditions, and in most plant parts.

Tissue-specific, tissue-enhanced (that is, tissue-preferred), cell type-specific, and inducible promoters constitute non-constitutive promoters. Promoters under developmental control include promoters that preferentially initiate transcription in certain tissues, such as xylem, leaves, roots, or seeds. Such promoters are examples of tissue-enhanced or tissue-preferred promoters (see U.S. Pat. No. 7,365,186). Tissue-enhanced promoters can be found upstream and operatively linked to DNA sequences normally transcribed in higher levels in certain plant tissues or specifically in certain plant tissues, respectively. “Cell-enhanced”, “tissue-enhanced”, or “tissue-specific” regulation thus refer to the control of gene or protein expression, for example, by a promoter, which drives expression that is not necessarily totally restricted to a single type of cell or tissue, but where expression is elevated in particular cells or tissues to a greater extent than in other cells or tissues within the organism, and in the case of tissue-specific regulation, in a manner that is primarily elevated in a specific tissue. Tissue-enhanced or preferred promoters have been described in, for example, U.S. Pat. No. 7,365,186, or U.S. Pat. No. 7,619,133.

A “condition-enhanced” promoter refers to a promoter that activates a gene in response to a particular environmental stimulus, for example, an abiotic stress, infection caused by a pathogen, light treatment, etc., and that drives expression in a unique pattern which may include expression in specific cell and/or tissue types within the organism (as opposed to a constitutive expression pattern in all cell types of an organism at all times).

### DESCRIPTION OF THE SPECIFIC EMBODIMENTS

Tissue-enhanced promoters that regulate expression of useful proteins may be of significant value for a number of reasons, including, but not limited to, the following:

1. Tissue-enhanced promoters are capable of causing, in response to a particular cellular or tissue or organ identity, sufficient expression of a transgene so that the protein encoded by the transgene will be produced at a level sufficient to confer an improved trait in a transformed plant, or result in the suppression or inactivity of one or more endogenous proteins in a plant through a repression approach.

2. Tissue-enhanced gene expression is fundamental to multicellular organisms, and is the underlying mechanism for how cells and tissues with different structures and functions arise from progenitor cells that all contain identical complements of genetic information. Recent advances in our ability to isolate and identify mRNA transcripts from specific cells, tissues and organs (such as laser capture microscopy) have enabled us to harness the DNA elements that control expression (i.e., promoters) and to use them to alter one or more genetic pathways to obtain highly-desirable traits. The use of tissue-enhanced promoters in a heterologous construct, driving the expression of a gene encoding a protein involved in signaling variety of processes, will provide a targeted approach for altering tissue-enhanced pathways in response to the cellular context or environment. Some of the traits that can be controlled by such a system include, for example, seedling vigor, plant height, photosynthesis, and photosynthetic pigment synthesis and photoprotective pigment synthesis, root area, flowering time, senescence, biomass and yield.

3. Fine-tuning the ectopic expression of useful polypeptides in transgenic plants to obtain effective expression without significant adverse morphological or physiological effects is often required as an optimization step in order to generate a commercially-applicable technology for improved traits such as, for example, improved water use efficiency, improved low nutrient availability, improved cold tolerance, improved yield, and the like. One such means of optimization is through the use of tissue-enhanced promoters that can confer improved traits while mitigating undesirable effects that might come about during high-level constitutive overexpression of proteins of interest.

4. Tissue-enhanced promoters driving the expression of visible markers are valuable in studying phenotypes that rely on, or are caused by alterations of specific gene expression patterns. The expression of such markers for specific cell types and tissue boundaries may be altered in plants that, for example, are expressing a polypeptide that broadens or narrows the expression of a gene or genes that wholly or in-part define a certain tissue type. Thus, plants transformed with tissue-enhanced-promoter::marker constructs can be used to understand and analyze important spatial changes in tissue boundaries or tissue types that are caused by ectopic expression of polypeptides that confer improved traits. Additionally, such plants could be used to screen for genetic mutations which may lead to changes in the expression pattern or in amplitude of a quantifiable marker signal, for example, LUCIFERASE. Such an approach can be used to identify “target” genes which can then be overexpressed in either crop or model plants and confirmed for their ability to confer beneficial traits such as improved yield or stress tolerance.

The selection strategy for identifying commercially valuable tissue-enhanced promoters considered the following criteria. Promoters of interest would be identified from genes that were:
expressed at a low basal level in non-target tissues; and
strongly enriched in specific cell, tissue or organ types under normal growth conditions. Transcript profiling (TxP) is a powerful tool for promoter discovery, providing a global insight into gene expression, regulation and induction levels across any and all tissues that can be specifically isolated away from the whole organism. As outlined below, tissue-enhanced promoters have been identified using microarrays by transcript profiling of plant parts that have been specifically isolated away from the whole plant. When a polynucleotide sequence that encodes a polypeptide (for example, a transcription factor) known to confer an improved trait but which also causes significant adverse morphological consequences when highly or ectopically overexpressed, and the polynucleotide expression is under the regulatory control of tissue-enhanced promoters, the result is often the production of plants of normal (i.e., wild type) or near-normal stature and development.

Promoters showing tissue-enhanced expression with little or no background expression in non-target cells (i.e., “tissue-enhanced promoters”) can be used to drive expression of polypeptides without significant side effects that reduce yield (also referred to as “yield drag”), or to enhance phenotypes by concentrating proteins into efficacious cell types. Such promoters can be used to regulate traits conferred by proteins that are influenced by specific cellular environments, or partner proteins, processes, or chemical compounds that are restricted to or from particular tissues or cells. For example, a regulatory polypeptide may interact positively to produce a beneficial trait when in contact with some other proteins that are present only in the vascular tissue of a plant. However, the same example polypeptide may cause deleterious or unwanted effects when it interacts with proteins that are found in other, non-vascular cell types. Therefore, if expression of this polypeptide were enhanced in vascular tissue, but restricted from other tissue types, thorough the use of a vascular-enriched promoter, the beneficial effects would be maximized. Another such example would be a case where a regulatory protein causes a beneficial effect when expressed ectopically from a constitutive promoter, but an enhanced effect is obtained when the protein is specifically concentrated into, e.g., shoot apical meristem tissue. These examples could apply to virtually any specific tissue or cell type.

Promoters are provided as SEQ ID NO: 1-66, and expression vectors or cassettes that may be constructed using these promoters may be introduced into plants for the purpose of regulating expression of polypeptides of interest to confer improved traits. The instant claims also encompass a tissue-enhanced promoter that comprises a functional part of any of SEQ ID NOs: 1-66, provided that the functional part of the promoter also includes a tissue-enhanced promoter function. The functional part of the promoter may have about 25, 50, 75, 100, 125, 150, 175, 200, 225, 250, 275, 300, 325, 350, 375, 400, 425, 450, 475, 500, 525, 550, 575, 600, 625, 650, 675, 700, 724, 725, 750, 775, 800, 825, 850, 875, 900, 925, 950, 975, 1000, 1025, 1050, 1075, 1100, 1125, 1150, 1175, 1200, 1204, 1300, 1400, 1500, 1600, 1700, 1800, 1900, 2000, 2100, 2200, 2300, 2400, 2500, 2600, 2700, 2800, 2900, or 3000 contiguous nucleotides of the nucleic acid sequences of SEQ ID NOs: 1-66, as well as all lengths of contiguous nucleotides within such sizes, provided that the functional part of the promoter includes a tissue-enhanced promoter function.

Promoters that are similar to those listed in the Sequence Listing may be made that have some alterations in the nucleotide sequence and yet retain the function of the listed sequences. At the nucleotide level, the promoter sequences will typically share at least about at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity with any of SEQ ID NOs: 1-66.

Percent identity can be determined electronically, e.g., by using the MEGALIGN program (DNASTAR, Inc. Madison, Wis.). The MEGALIGN program can create alignments between two or more sequences according to different methods, for example, the clustal method (see, for example, Higgins and Sharp (1988)). The clustal algorithm groups sequences into clusters by examining the distances between all pairs. The clusters are aligned pairwise and then in groups. Other alignment algorithms or programs may be used, including FASTA, BLAST, or ENTREZ, and which may be used to calculate percent similarity. These are available as a part of the GCG sequence analysis package (University of Wisconsin, Madison, Wis.), and can be used with or without default settings. ENTREZ is available through the National Center for Biotechnology Information. In one embodiment, the percent identity of two sequences can be determined by the GCG program with a gap weight of 1 (see U.S. Pat. No. 6,262,333).

Software for performing BLAST analyses is publicly available, e.g., through the National Center for Biotechnology Information (see internet website at www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul (1990); Altschul (1993)). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989)). Unless otherwise indicated for comparisons of predicted polynucleotides, “sequence identity” refers to the % sequence identity generated from a tblastx using the NCBI version of the algorithm at the default settings using gapped alignments with the filter “off” (see, for example, internet website at www.ncbi.nlm.nih.gov/).

### EXAMPLES

It is to be understood that this description is not limited to the particular devices, machines, materials and methods described. Although particular embodiments are described, equivalent embodiments may be used to practice the instant claims.

The description and claims will be more readily understood by reference to the following examples, which are included merely for purposes of illustration of certain aspects and embodiments of the present description and claims and are not intended to limit the description and claims. It will be recognized by one of skill in the art that a promoter that regulates expression of a particular gene may also be used to regulate expression of other genes. The function of a listed polypeptide that is associated with a particular first trait may also be associated with at least one other, unrelated and inherent second trait which was not predicted by the first trait.

### Example I

### Identification of Tissue-Enhanced Transcripts in Arabidopsis

To identify promoters that control gene expression in specific cell-types, tissues and organs, several specific plant tissue types (vascular tissue; hypocotyl tissue; shoot apical meristems, “SAM;” leaf primordia) were dissected using laser capture microdissection (LMD) and transcriptionally profiled via Affymetrix GeneChip microarrays. For vascular tissues, an additional method was used in combination with LMD to identify transcripts that were specifically-expressed or enriched in that particular tissue-type (see method described in “Polysome-Mediated Cell Type-, Tissue Type- or Condition-Enhanced Transcript Profiling;” U.S. patent application Ser. No. 12/557,449). Data obtained from tissue-enhanced TxP experiments were then cross-compared with other expression data sets that measured expression in other non-target tissues to confirm cell-type specificity. Several different tissue-enriched or -specific genes, the promoters of which represent tissue-enhanced promoter candidates, are shown in Tables 1, 2, 3, and 4 below.

TABLE 1
Expression profiles from vascular tissue-enriched Arabidopsis
microarray TxP experiments

| SEQ | | | | | | | |
|---|---|---|---|---|---|---|---|
| ID | Gene is | | | Ribotag | Ribotag | LMD | LMD |
| NO. | enhanced in: | Name | AGI ID | Ctrl Expr | Expr | Ctrl Expr | Expr |
| 1 | Vascular | | AT2G39850 | 1.04 | 12.81 | 0.11 | 10.78 |
| 2 | Vascular | G1554 | AT2G03500 | 0.54 | 7.11 | 0.13 | 7.75 |
| 3 | Vascular | G2041 | AT3G42670 | 0.12 | 3.08 | 0.05 | 1.64 |
| 4 | Vascular | | AT1G24735 | 0.26 | 1.50 | 0.06 | 1.75 |
| 5 | Vascular | | AT5G56530 | 0.38 | 3.45 | 0.07 | 1.64 |
| 6 | Vascular | ZIP1 | AT3G12750 | 0.75 | 6.88 | 0.17 | 9.39 |
| 7 | Vascular | | AT3G16340 | 0.26 | 1.39 | 0.07 | 3.58 |
| 8 | Vascular | | AT1G65150 | 0.78 | 11.61 | 0.07 | 1.45 |
| 9 | Vascular | | AT5G27690 | 0.48 | 5.33 | 0.04 | 0.78 |
| 10 | Vascular | | AT1G10155 | 0.67 | 4.20 | 0.08 | 2.00 |

Legend for Table 1. Expression profiles from vascular tissue-enhanced Arabidopsis microarray TxP experiments. Column header descriptions: Tissue=specific tissue that the indicated gene is specifically expressed/enriched in; Name=gene common name from public literature, or from Mendel Biotechnology, Inc.'s internal naming system; AGI Identifier=Arabidopsis Genome Initiative locus identifier; Ribotag Ctrl Expr=the baseline expression level of a given gene, as determined by a 35S::ribotag pull-down (i.e., measurement of the selected transcript from all the cell types specified by the constitutive CaMV 35S promoter); Ribotag Expr=vascular expression level of a given gene, as determined by a SUC2::ribotag pull-down (i.e., measurement of the selected transcript from the vascular cell types specified by the SUCROSE2 (AT1G2271) promoter); LMD Ctrl Expr=the baseline expression level of a given gene, as determined by laser capture microdissection of Arabidopsis leaf mesophyll tissue; LMD Expr=the vascular expression level of a given gene, as determined by laser capture microdissection of Arabidopsis leaf vascular bundle tissue.

TABLE 2
Expression profiles from hypocotyl tissue-enhanced Arabidopsis microarray TxP experiment

| | Gene is | | | | | | | | | | |
|---|---|---|---|---|---|---|---|---|---|---|---|
| SEQ ID | enhanced | | | | Leaf | | | Leaf | Leaf | Inflor. | Inflor. |
| NO. | in: | Name | AGI ID | Hypocotyl | Primordia | SAM | Root | AM | PM | AM | PM |
| 11 | Hypocotyl | | AT3G30340 | 2556.24 | 62.36 | 70.22 | 52.23 | 61.75 | 85.22 | 65.26 | 74.41 |
| 12 | Hypocotyl | | AT3G44970 | 6532.28 | 42.46 | 56.21 | 19.6 | 31.98 | 24.53 | 25.11 | 24.58 |
| 13 | Hypocotyl | MIR156c | AT4G31877 | 1233.79 | 68.35 | 83.06 | 19.99 | 64.13 | 36.23 | 24.93 | 23.92 |
| 14 | Hypocotyl | | AT1G49320 | 1297.39 | 67.87 | 74.67 | 121.79 | 48.24 | 39.55 | 46.05 | 41.32 |
| 15 | Hypocotyl | | AT4G14819 | 784.76 | 18.77 | 20.3 | 13.56 | 142.74 | 22.46 | 68.8 | 28.37 |
| 16 | Hypocotyl | | AT1G24130 | 765.77 | 43.01 | 46.46 | 145.86 | 29.63 | 27.22 | 25.88 | 22.16 |
| 17 | Hypocotyl | | AT5G16410 | 724.64 | 60.23 | 104.58 | 58.04 | 130.11 | 59.5 | 69.14 | 51.07 |
| 18 | Hypocotyl | | AT5G42655 | 549.97 | 30.06 | 26.16 | 188.87 | 31.08 | 29.62 | 25.09 | 23.81 |
| 19 | Hypocotyl | G777 | AT4G36060 | 455.87 | 58.86 | 72.6 | 179.2 | 45.45 | 37.34 | 39.46 | 39.58 |
| 20 | Hypocotyl | | AT1G14190 | 1944.22 | 28.83 | 27.5 | 197.88 | 61.21 | 43.25 | 146.05 | 127.81 |
| 21 | Hypocotyl | | AT4G37970 | 613.94 | 24.9 | 25.14 | 117.51 | 112.54 | 73.56 | 30.41 | 27.96 |
| 22 | Hypocotyl | | AT5G58780 | 1546.58 | 29.8 | 31.13 | 145.66 | 20.07 | 19.34 | 20.37 | 20.33 |
| 23 | Hypocotyl | | AT4G36470 | 996.11 | 62.81 | 55.15 | 112 | 74.35 | 56 | 124.09 | 57.07 |
| 24 | Hypocotyl | | AT3G02500 | 2328.21 | 211.65 | 93.64 | 45.11 | 36.36 | 32.49 | 79.89 | 60.19 |
| 25 | Hypocotyl | | AT1G52100 | 3310.15 | 385.87 | 53.39 | 86.7 | 21.81 | 20.25 | 19.07 | 18.93 |
| 26 | Hypocotyl | G971 | AT3G54990 | 814.89 | 150.07 | 100.32 | 170.21 | 23.39 | 19.44 | 28.24 | 27.1 |
| 27 | Hypocotyl | | AT5G14070 | 1185.36 | 165.91 | 223.12 | 66.76 | 20.77 | 20.63 | 31.63 | 27.82 |
| 28 | Hypocotyl | | AT4G36850 | 2071.32 | 171.44 | 103.17 | 46.34 | 85.45 | 88.42 | 142.75 | 91.16 |
| 29 | Hypocotyl | G2554 | AT1G64625 | 1355.41 | 124.97 | 156.32 | 60.17 | 43.81 | 28.85 | 189.05 | 95.61 |
| 30 | Hypocotyl | | AT4G12450 | 427.27 | 66.06 | 86.36 | 98.49 | 44.41 | 38.4 | 41.46 | 38.79 |

TABLE 3
Expression profiles from shoot apical meristem tissue-enhanced Arabidopsis microarray TxP experiment

| SEQ | Gene is | | | | | | | | | | |
|---|---|---|---|---|---|---|---|---|---|---|---|
| ID | enhanced | | | | Leaf | | | Leaf | Leaf | Inflor. | Inflor. |
| NO. | in: | Name | AGI ID | SAM | Primordia | Hypocotyl | Root | AM | PM | AM | PM |
| 31 | SAM | CLV3 | AT2G27250 | 4623.15 | 87.72 | 24.77 | 16.03 | 21.19 | 21.86 | 18.14 | 19.8 |
| 32 | SAM | G3581 | AT4G31610 | 1086.72 | 219.85 | 36.85 | 22.96 | 35.89 | 32.99 | 95.15 | 73.23 |
| 33 | SAM | | AT1G37140 | 1608.79 | 52 | 21.05 | 18.03 | 21.8 | 22.71 | 19.42 | 20.37 |
| 34 | SAM | | AT1G60540 | 508.58 | 28.11 | 22.78 | 15.7 | 25.62 | 25.02 | 23 | 21.68 |
| 35 | SAM | | AT3G59270 | 2142.54 | 65.01 | 36.46 | 22.76 | 27.16 | 28.94 | 27.3 | 25.64 |
| 36 | SAM | G2291 | AT1G80580 | 511.03 | 54.53 | 42.76 | 25.39 | 38.49 | 35.27 | 31.13 | 29.43 |
| 37 | SAM | G3583 | AT4G31615 | 351.56 | 79.1 | 23.93 | 16.59 | 21.73 | 20.91 | 24.75 | 26.65 |
| 38 | SAM | | AT5G39330 | 441.31 | 99.51 | 29.74 | 43.19 | 36.48 | 36.16 | 42.46 | 45.42 |
| 39 | SAM | | AT5G64910 | 1345.28 | 199.07 | 28.78 | 13.97 | 30.48 | 28.48 | 59.48 | 49.62 |
| 40 | SAM | UFO | AT1G30950 | 7735.55 | 193.45 | 56.7 | 43.99 | 45.37 | 43.21 | 38.61 | 39.59 |
| 41 | SAM | G1917 | AT3G50870 | 1158.8 | 221.32 | 28.83 | 51.67 | 21.41 | 22.1 | 57.37 | 42.5 |
| 42 | SAM | G3582 | AT4G31620 | 3260.4 | 324.3 | 34.14 | 15.93 | 24.72 | 30 | 36.72 | 179.02 |
| 43 | SAM | | AT1G49475 | 1453.62 | 242.02 | 53.66 | 109.8 | 61.75 | 84 | 41.42 | 40.04 |
| 44 | SAM | | AT1G77145 | 1367.86 | 56.08 | 49.35 | 125.95 | 50.33 | 48.82 | 28.65 | 30.19 |
| 45 | SAM | | AT2G19910 | 1170.11 | 234.08 | 27.29 | 21.46 | 19.82 | 20.5 | 160.36 | 140.18 |
| 46 | SAM | | AT5G61070 | 1609.25 | 269.16 | 41.53 | 14.63 | 36.05 | 36.61 | 45.47 | 36.6 |
| 47 | SAM | G2636 | AT3G15170 | 985.32 | 175.2 | 95.04 | 119.24 | 86.5 | 79.81 | 77.49 | 74.57 |
| 48 | SAM | G2694 | AT5G35770 | 1510.19 | 305.94 | 48.1 | 40.82 | 31 | 27.7 | 25.01 | 25.43 |
| 49 | SAM | G1540 | AT2G17950 | 1194.06 | 38.01 | 32.41 | 20.36 | 29.16 | 25.66 | 274.24 | 216.05 |
| 50 | SAM | TFL1 | AT5G03840 | 4943.56 | 182.48 | 169.57 | 328.49 | 29.44 | 31.19 | 175.82 | 252.67 |
| 51 | SAM | G1584 | AT2G33880 | 528.26 | 75.99 | 46.55 | 22.33 | 40.44 | 36.93 | 405.2 | 340.34 |
| 52 | SAM | G2649 | AT5G12330 | 1337.65 | 309.84 | 61.04 | 80.41 | 26.8 | 26.23 | 31.2 | 35.65 |

TABLE 4
Expression profiles from leaf primordia tissue-enhanced Arabidopsis microarray TxP experiment

| SEQ | | | | | | | | | | | |
|---|---|---|---|---|---|---|---|---|---|---|---|
| ID | Gene is | | | Leaf | | | | Leaf | Leaf | Inflor. | Inflor. |
| NO. | enhanced in: | Name | AGI ID | Primordia | SAM | Hypocotyl | Root | AM | PM | AM | PM |
| 53 | Leaf primordia | G2456 | AT4G00180 | 1635.57 | 375.18 | 53.33 | 19.46 | 605.03 | 359.86 | 535.22 | 437.45 |
| 54 | Leaf primordia | YAB2 | AT1G08465 | 1051.28 | 110.71 | 153.82 | 24.99 | 111.82 | 88.93 | 240.06 | 220.26 |
| 55 | Leaf primordia | | AT4G31805 | 565.76 | 36.86 | 26.85 | 23.22 | 41.34 | 44.29 | 85.9 | 88.08 |
| 56 | Leaf primordia | JAG | AT1G68480 | 845.93 | 169.92 | 28.67 | 18.07 | 21.47 | 18.88 | 140.07 | 56.83 |
| 57 | Leaf primordia | G212 | AT3G27920 | 685.89 | 78.4 | 23.28 | 16.33 | 23.25 | 24.38 | 23.39 | 23.63 |
| 58 | Leaf primordia | G1912 | AT1G31310 | 661.26 | 119.47 | 36.06 | 20.74 | 22.33 | 24.25 | 66.85 | 53.03 |
| 59 | Leaf primordia | G2699 | AT3G49950 | 655.46 | 143.83 | 66.94 | 45.21 | 47.4 | 51.15 | 40.1 | 39.54 |
| 60 | Leaf primordia | G1229 | AT5G53210 | 632.75 | 47.71 | 35.37 | 14.8 | 23.69 | 21.62 | 110 | 108.76 |
| 61 | Leaf primordia | | AT1G60630 | 520.92 | 99.42 | 60.55 | 85.73 | 65.24 | 67.87 | 182.78 | 150.59 |
| 62 | Leaf primordia | G581 | AT4G00480 | 1025.65 | 239.81 | 229.58 | 280.74 | 52 | 45.8 | 142.31 | 150.83 |
| 63 | Leaf primordia | | AT1G68780 | 2103.42 | 260.43 | 33.56 | 15.63 | 196.15 | 37.32 | 219.86 | 183.58 |
| 64 | Leaf primordia | | AT1G29270 | 1616.92 | 135.93 | 28.1 | 369.63 | 32.35 | 33.42 | 30.39 | 27.96 |
| 65 | Leaf primordia | | AT5G20740 | 847.07 | 78.64 | 73.1 | 16.33 | 58.05 | 69.36 | 3526.3 | 5413.43 |
| 66 | Leaf primordia | G2457 | AT1G69180 | 921.32 | 75.26 | 52.84 | 34 | 39.64 | 36.03 | 1216.83 | 1099.63 |

Legend for Tables 2, 3 and 4. Expression profiles from hypocotyl (Table 2), shoot apical meristem (Table 3), or leaf primordium (Table 4) tissue-enhanced Arabidopsis microarray TxP experiments. Column header descriptions:
Tissue = specific tissue that the indicated gene is specifically expressed/enriched in;
Name = gene common name from public literature, or from the Mendel Biotechnology, Inc. internal naming system;
AGI Identifier = Arabidopsis Genome Initiative locus identifier; Hypocotyl, Leaf Primordia, SAM (shoot apical meristem), Root, Leaf AM (morning), Leaf PM (evening), INFLOR. AM (inflorescence tip morning) and Inflor. PM (inflorescence tip, evening) each = the baseline expression of a givengene in the indicated tissue.

### Example II

### Regulating Expression of Polynucleotides Encoding RNA Species which Act at a Non-Protein Level

In addition to use of the tissue-enhanced promoters to regulate the expression of a polynucleotide encoding a polypeptide, the promoters can also be used to regulate the expression of a polynucleotide encoding a non-coding RNA species (that is, one which acts at a non-protein level), such as a microRNA, a microRNA precursor, or a sequence designed to act through RNA interference (RNAi). For example, a substantial number of microRNA (miRNA) species have been implicated in stress responses and these molecules have been shown to be involved in the control of many aspects of plant growth and development (Bartel and Bartel (2003); Aukerman and Sakai (2003).; Bartel (2004); Juarez et al. (2004); Bowman (2004); Sunkar and Zhu (2004)).

It should be noted that, for particular families of highly related plant polypeptides such as transcription factors, overexpression of one or more of the family members produces a comparable phenotype to that obtained from reducing expression (for example, by mutation or knockdown approaches such as antisense or RNA interference) of one or more of the family members. For instance, overexpression of the CBF family proteins has been widely demonstrated to confer tolerance to drought and low temperature stress (e.g., Jaglo et al. (2001). Nonetheless, Novillo et al. (2004) showed that homozygous cbJ2 mutant Arabidopsis plants carrying a disruption in the CBF2 gene also exhibit enhanced freezing tolerance. Such results can be accounted for by cross regulation between the genes encoding different transcription factor family members. In the study by Novillo et al, (2004) supra, CBF2 was shown to be a negative transcriptional regulator of the CBF1 and CBF3 genes. Comparable mechanisms likely account for the fact that we have observed stress tolerance from both overexpression and from knockdown approaches with certain NF-Y family genes.

### Example III

### Preparation of Transgenic Arabidopsis Plants

The above-identified promoters may be used to regulate expression of genes of interest in specific cell types. Transformed plants may be prepared using the following methods, although these examples are not intended to limit the description or claims.

Promoter cloning. For genes showing appropriate patterns of regulation, typically approximately 1.2 kb of upstream sequence are cloned by polymerase chain reaction (unless this region contains another gene, in which case the upstream sequence up to the next gene is cloned). Each promoter is cloned into a nucleic acid construct (e.g., an expression vector or cassette) in front of either a polynucleotide encoding green fluorescent protein (GFP) or another marker of gene expression, or in front of a polynucleotide encoding a polypeptide or a regulatory molecule of interest, for example, a polypeptide found in the Sequence Listing, such as SEQ ID NOs: 68, 70 and 72, among others. In some instances the promoter may be used to regulate the expression of a polynucleotide that is expected to cause beneficial traits by reducing or eliminating the activity of a target gene or group of genes through antisense or RNAi based approaches. P21103 is an example base vector that is used for the creation of RNAi constructs; the polylinker and PDK intron sequences in this vector are provided as SEQ ID NO: 73. The promoter may also be incorporated into antisense or RNAi constructs which target genes encoding homologs of the transcription factors.

In some of these cases, the polypeptide may produce deleterious morphological effects in the plants when they are constitutively overexpressed at moderately, but which negative effects can be mitigated to some extent, or entirely, when expression of the polypeptide is regulated in a tissue-enhanced manner.

Transformation. Transformation of Arabidopsis is typically performed by an Agrobacterium-mediated protocol based on the method of Bechtold and Pelletier (1998).

Plant preparation. Arabidopsis seeds are sown on mesh covered pots. The seedlings are thinned so that 6-10 evenly spaced plants remain on each pot 10 days after planting. The primary bolts are cut off a week before transformation to break apical dominance and encourage axillary shoots to form. Transformation is typically performed at 4-5 weeks after sowing.

Bacterial culture preparation. Agrobacterium stocks are inoculated from single colony plates or from glycerol stocks and grown with the appropriate antibiotics and grown until saturation. On the morning of transformation, the saturated cultures are centrifuged and bacterial pellets are re-suspended in Infiltration Media (0.5×MS, 1×B5 Vitamins, 5% sucrose, 1 mg/ml benzylaminopurine riboside, 200 μl/L Silwet L77) until an A600 reading of 0.8 is reached.

Transformation and seed harvest. The Agrobacterium solution is poured into dipping containers. All flower buds and rosette leaves of the plants are immersed in this solution for 30 seconds. The plants are laid on their side and wrapped to keep the humidity high. The plants are kept this way overnight at 22° C. and then the pots are unwrapped, turned upright, and moved to the growth racks.

The plants are maintained on the growth rack under 24-hour light until seeds are ready to be harvested. Seeds are harvested when 80% of the siliques of the transformed plants are ripe (approximately 5 weeks after the initial transformation). This seed is deemed T0 seed, since it is obtained from the T0 generation, and is later plated on selection plates (kanamycin, sulfonamide or glyphosate). Resistant plants that are identified on such selection plates comprise the T1 generation.

For polynucleotides (e.g., SEQ ID NOs: 67, 69 and 71) encoding polypeptides (e.g., SEQ ID NOs: 68, 70 and 72) used in these experiments, RT-PCR may be performed to confirm the ability of cloned promoter fragments to drive expression of the polypeptide transgene in plants transformed with the vectors.

T1 plants transformed with promoter-TF combinations comprised within a nucleic acid construct are subjected to morphological analysis. Promoters that produce a substantial amelioration of the negative effects of TF overexpression are subjected to further analysis by propagation into the T2 generation, where the plants are analyzed for an altered trait relative to a control plant.

### Example IV

### Use of Tissue-Enhanced Promoters to Drive Expression of Transcription Factors for the Production and/or Enhancement of Traits

Directed expression of plant transcription factors in specific cell or tissue types can produce or enhance beneficial agronomic traits such as greater yield, greater biomass, greater plant size, greater plant volume, greater disease resistance, greater resistance to fungal pathogens, greater resistance to biotrophic pathogens, greater resistance to necrotrophic pathogens, greater resistance to diseases caused by ascomycetes fungi, greater resistance to Fusarium, greater resistance to Botrytis, greater resistance to Erysiphe, greater resistance to Sclerotinia, constitutive photomorphogenesis, greater photosynthetic capacity, dark green color, more chlorophyll A, more chlorophyll B, more carotenoids, more anthocyanin, altered light response, reduced sensitivity to light, greater early season growth, greater height, greater stem diameter, greater resistance to lodging, greater internode length, greater secondary rooting, greater cold tolerance, greater water use efficiency, greater tolerance to water deprivation, greater tolerance to heat, greater tolerance to salt, reduced stomatal conductance, altered C/N sensing, greater tolerance to low nutrient conditions, greater low nitrogen tolerance, greater low phosphorus tolerance, greater tolerance to hyperosmotic stress, greater late season growth and vigor, greater number of primary nodes, and greater canopy coverage relative to a control plant. In some cases, enhancing a native expression pattern by increasing the amount of target protein observed in tissues where a transcription factor is normally active can produce positive effects. In other cases, the positive effect may be obtained when the pattern of expression is extended beyond the normal range of tissues for that target protein. For example, the NF-YB transcription factor G481 (SEQ ID NO: 68) can be used to produce beneficial traits such as increased drought tolerance and dark green coloration (related to increased photosynthetic capacity) when expressed under control of vascular-enhanced promoters such as those in SEQ ID NO: 1-10. Likewise, the shoot apical meristem or leaf primordia promoters listed in SEQ ID NO: 31-66 could be used with an HD-ZIP transcription factor such as G1543 (SEQ ID NO: 70) to produce increased photosynthetic capacity and beneficial effects on yield. Additionally, promoters that enhance expression in hypocotyl tissue, such as those in SEQ ID NO: 11-30, can be used with the B-BOX transcription factor G1988 (SEQ ID NO: 72) to alter light response. Altered light response, including reduced sensitivity to light, has been shown to affect a wide range of potentially useful traits (see, for example, U.S. Pat. No. 7,692,067). In addition to these three examples, other transcription factors, regulatory proteins, or other proteins of interest could be targeted in the manner presented in this example to produce the aforementioned improved traits.

### Example V

### Transformation of Eudicots to Produce Improved Traits

Crop species including, but not limited to, crops such as soybean, potato, cotton, rape, oilseed rape (including canola), sunflower, alfalfa, fruits and vegetables, such as banana, blackberry, blueberry, strawberry, and raspberry, cantaloupe, carrot, cauliflower, coffee, cucumber, eggplant, grapes, honeydew, lettuce, mango, melon, onion, papaya, peas, peppers, pineapple, pumpkin, spinach, squash, tobacco, tomato, watermelon, rosaceous fruits (such as apple, peach, pear, cherry and plum) and vegetable brassicas (such as broccoli, cabbage, cauliflower, Brussels sprouts, and kohlrabi), fruits and vegetables whose phenotype can be changed include currant, avocado, citrus fruits such as oranges, lemons, grapefruit and tangerines, artichoke, cherries, nuts such as the walnut and peanut, endive, leek, root, such as arrowroot, beet, cassava, turnip, radish, yam, and sweet potato, beans, woody species such pine, poplar and eucalyptus, or mint or other labiates that overexpress polypeptides of interest may produce plants with improved or desirable traits when a sequence encoding a polypeptide of interest is placed under the regulatory control of tissue-enhanced promoters SEQ ID NO: 1-66, or related sequences from other plant species with similar sequence structure and regulatory function. These observations indicate that these genes, when overexpressed, will result in improved quality and larger yields than non-transformed plants in non-stressed or stressed conditions; the latter may occur in the field to even a low, imperceptible degree at any time in the growing season.

Thus, promoter sequences listed in the Sequence Listing recombined into, for example, a nucleic acid construct, or another suitable expression vector, may be transformed into a plant for the purpose of regulating tissue-enhanced expression and modifying plant traits for the purpose of improving yield and/or quality. The cloning vector may be introduced into a variety of plants by means well known in the art such as, for example, direct DNA transfer or Agrobacterium tumefaciens-mediated transformation. It is now routine to produce transgenic plants using most dicot plants (see Weissbach and Weissbach, (1989); Gelvin et al. (1990); Herrera-Estrella et al. (1983); Bevan (1984); and Klee (1985). Methods for analysis of traits are routine in the art and examples are disclosed above.

Numerous protocols for the transformation of tomato and soy plants have been previously described, and are well known in the art. Gruber et al. (1993), and Glick and Thompson (1993) describe several expression vectors and culture methods that may be used for cell or tissue transformation and subsequent regeneration. For soybean transformation, methods are described by Miki et al. (1993); and U.S. Pat. No. 5,563,055, (Townsend and Thomas), issued Oct. 8, 1996.

There are a substantial number of alternatives to Agrobacterium-mediated transformation protocols, other methods for the purpose of transferring transgenes or exogenous genes into soybeans or tomatoes. One such method is microprojectile-mediated transformation, in which DNA on the surface of microprojectile particles is driven into plant tissues with a biolistic device (see, for example, Sanford et al. (1987); Christou et al. (1992); Sanford (1993); Klein et al. (1987); U.S. Pat. No. 5,015,580 (Christou et al), issued May 14, 1991; and U.S. Pat. No. 5,322,783 (Tomes et al.), issued Jun. 21, 1994).

Alternatively, sonication methods (see, for example, Zhang et al. (1991); direct uptake of DNA into protoplasts using CaCl2 precipitation, polyvinyl alcohol or poly-L-ornithine (Hain et al. (1985); Draper et al. (1982); liposome or spheroplast fusion (see, for example, Deshayes et al. (1985); Christou et al. (1987); and electroporation of protoplasts and whole cells and tissues (see, for example, Donn et al. (1990); D'Halluin et al. (1992); and Spencer et al. (1994), have been used to introduce foreign DNA and expression vectors into plants.

After a plant or plant cell is transformed (and the latter regenerated into a plant), the transformed plant may be crossed with itself or a plant from the same line, a non-transformed or wild-type plant, or another transformed plant from a different transgenic line of plants. Crossing provides the advantages of producing new and often stable transgenic varieties. Genes and the traits they confer that have been introduced into a tomato or soybean line may be moved into distinct lines of plants using traditional backcrossing techniques well known in the art. Transformation of tomato plants may be conducted using the protocols of Koornneef et al (1986), and in U.S. Pat. No. 6,613,962, the latter method described in brief here. Eight day old cotyledon explants are precultured for 24 hours in Petri dishes containing a feeder layer of Petunia hybrida suspension cells plated on MS medium with 2% (w/v) sucrose and 0.8% agar supplemented with 10 μM α-naphthalene acetic acid and 4.4 μM 6-benzylaminopurine. The explants are then infected with a diluted overnight culture of Agrobacterium tumefaciens containing an expression vector comprising a polynucleotide for 5-10 minutes, blotted dry on sterile filter paper and cocultured for 48 hours on the original feeder layer plates. Culture conditions are as described above. Overnight cultures of Agrobacterium tumefaciens are diluted in liquid MS medium with 2% (w/v/) sucrose, pH 5.7) to an OD600 of 0.8.

Following cocultivation, the cotyledon explants are transferred to Petri dishes with selective medium comprising MS medium with 4.56 μM zeatin, 67.3 μM vancomycin, 418.9 μM cefotaxime and 171.6 μM kanamycin sulfate, and cultured under the culture conditions described above. The explants are subcultured every three weeks onto fresh medium. Emerging shoots are dissected from the underlying callus and transferred to glass jars with selective medium without zeatin to form roots. The formation of roots in a kanamycin sulfate-containing medium is a positive indication of a successful transformation.

Transformation of soybean plants may be conducted using the methods found in, for example, U.S. Pat. No. 5,563,055 (Townsend et al., issued Oct. 8, 1996), described in brief here. In this method soybean seed is surface sterilized by exposure to chlorine gas evolved in a glass bell jar. Seeds are germinated by plating on 1/10 strength agar solidified medium without plant growth regulators and culturing at 28° C. with a 16 hour day length. After three or four days, seed may be prepared for cocultivation. The seedcoat is removed and the elongating radicle removed 3-4 mm below the cotyledons.

Overnight cultures of Agrobacterium tumefaciens harboring the expression vector comprising a polynucleotide are grown to log phase, pooled, and concentrated by centrifugation. Inoculations are conducted in batches such that each plate of seed was treated with a newly resuspended pellet of Agrobacterium. The pellets are resuspended in 20 ml inoculation medium. The inoculum is poured into a Petri dish containing prepared seed and the cotyledonary nodes are macerated with a surgical blade. After 30 minutes the explants are transferred to plates of the same medium that has been solidified. Explants are embedded with the adaxial side up and level with the surface of the medium and cultured at 22° C. for three days under white fluorescent light. These plants may then be regenerated according to methods well established in the art, such as by moving the explants after three days to a liquid counter-selection medium (see U.S. Pat. No. 5,563,055).

The explants may then be picked, embedded and cultured in solidified selection medium. After one month on selective media, transformed tissue becomes visible as green sectors of regenerating tissue against a background of bleached, less healthy tissue. Explants with green sectors are transferred to an elongation medium. Culture is continued on this medium with transfers to fresh plates every two weeks. When shoots are 0.5 cm in length they may be excised at the base and placed in a rooting medium.

Protocols for the transformation of canola plants have also been previously described. See, for example, Pua et al. (1987); Charest et al. (1988); Radke et al. (1988); De Block et al. (1989); or Stewart et al. (1996) who teach Agrobacterium-mediated transformation of canola, or Cardoza et al. (2003), who teach a method of Agrobacterium-mediated transformation of canola using hypocotyls as explant tissue.

### Example VI

### Transformation of Monocots to Produce Improved Traits

Cereal plants and other grasses such as, but not limited to, corn, sweet corn, wheat, rice, sugarcane, turfgrass; sorghum, barley, rye, millet, Miscanthus, “miscane” (Miscanthus×sugarcane hybrids), and switchgrass, may be transformed with the present promoter sequences such as those presented in the present Sequence Listing, cloned into a vector such as pGA643 and containing a kanamycin-resistance marker, and inducibly express a polypeptide, for example, a transcription factor, that confers an improved or desirable trait. The expression vectors may be one found in the Sequence Listing, or any other suitable expression vector that incorporates a tissue-enhanced promoter sequence, may be similarly used. For example, pMEN020 may be modified to replace the NptII coding region with the BAR gene of Streptomyces hygroscopicus that confers resistance to phosphinothricin. The KpnI and BglII sites of the Bar gene are removed by site-directed mutagenesis with silent codon changes.

The cloning vector may be introduced into a variety of cereal plants by means well known in the art including direct DNA transfer or Agrobacterium tumefaciens-mediated transformation. The latter approach may be accomplished by a variety of means, including, for example, that of U.S. Pat. No. 5,591,616, in which monocotyledon callus is transformed by contacting dedifferentiating tissue with the Agrobacterium containing the cloning vector.

The sample tissues are immersed in a suspension of 3×10⁻⁹ cells of Agrobacterium containing the cloning vector for 3-10 minutes. The callus material is cultured on solid medium at 25° C. in the dark for several days. The calli grown on this medium are transferred to Regeneration medium. Transfers are continued every 2-3 weeks (2 or 3 times) until shoots develop. Shoots are then transferred to Shoot-Elongation medium every 2-3 weeks. Healthy looking shoots are transferred to rooting medium and after roots have developed, the plants are placed into moist potting soil.

The transformed plants are then analyzed for the presence of the NPTII gene/kanamycin resistance by ELISA, using the ELISA NPTII kit from SPrime-3Prime Inc. (Boulder, Colo.).

It is also routine to use other methods to produce transgenic plants of most cereal crops (Vasil (1994), such as corn, wheat, rice, sorghum (Cassas et al. (1993), and barley (Wan and Lemeaux (1994). DNA transfer methods such as the microprojectile method can be used for corn (Fromm et al. (1990); Gordon-Kamm et al. (1990); Ishida (1990); wheat (Vasil et al. (1992); Vasil et al. (1993); Weeks et al. (1993); and rice (Christou (1991); Hiei et al. (1994); Aldemita and Hodges (1996); and Hiei et al. (1997). For most cereal plants, embryogenic cells derived from immature scutellum tissues are the preferred cellular targets for transformation (Hiei et al. (1997) supra; Vasil (1994) supra). For transforming corn embryogenic cells derived from immature scutellar tissue using microprojectile bombardment, the A188XB73 genotype is the preferred genotype (Fromm et al. (1990) supra; Gordon-Kamm et al. (1990) supra). After microprojectile bombardment the tissues are selected on phosphinothricin to identify the transgenic embryogenic cells (Gordon-Kamm et al. (1990) supra). Transgenic plants are regenerated by standard corn regeneration techniques (Fromm et al. (1990) supra; Gordon-Kamm et al. (1990) supra). Agrobacterium-mediated transformation of switchgrass has also been reported by Somleva et al. (2002).

### Example VII

### Confirmation of Improved or Desirable Traits in Plants

Northern blot analysis, RT-PCR or microarray, or protein-blot analysis of the regenerated, transformed plants may be used to demonstrate expression of a transgene or its encoded polypeptide or other active molecule (e.g. a microRNA) that is capable of inducing an improved trait as compared to a control plant.

To verify the ability to confer an improved or desirable trait, mature plants overexpressing a polypeptide under the regulatory control of a tissue-enhanced promoter, or alternatively, seedling progeny of these plants will be created. By comparing control plants (for example, wild type or parental line untransformed plants, or plants transformed with an empty vector or one lacking the polypeptide) and transgenic plants, the transgenic plants may be shown to have an improved trait, for example, with one of the physiological assays provided below, or by the observation of, for example, increased yield, increased biomass, increased plant size or plant volume, increased disease resistance, increased resistance to fungal pathogens including, for example, biotrophs, necrotrophs, Fusarium, Botrytis, Erysiphe, or Sclerotinia, constitutive photomorphogenesis, increased photosynthetic capacity, dark green color, more chlorophyll A, more chlorophyll B, more carotenoids, more anthocyanin, reduced sensitivity to light, greater early season growth, greater height, greater stem diameter, increased resistance to lodging, increased internode length, increased secondary rooting, greater cold tolerance, greater tolerance to water deprivation, greater tolerance to heat, greater tolerance to salt, greater water use efficiency, reduced stomatal conductance, altered C/N sensing, increased low nitrogen tolerance, increased low phosphorus tolerance, increased tolerance to hyperosmotic stress, greater late season growth and vigor, increased number of primary nodes, and/or greater canopy coverage.

After a eudicot plant, monocot plant or plant cell has been transformed (and the latter regenerated into a plant) and shown to have an improved or desirable trait, for example, by producing greater yield, stress tolerance, greater biomass, or plant quality relative to a control plant grown under the same conditions, the transformed plant may be crossed with itself or a plant from the same line, a non-transformed or wild-type plant, or another transformed plant from a different transgenic line of plants.

These experiments would demonstrate that polypeptides can be identified and shown to confer an improved or desirable trait such as, but not limited to, greater yield, greater stress tolerance, or greater quality in eudicots or monocots.

### Example VIII

### Physiological Assays

There are a number of assays one can perform to identify useful traits. In these Examples, unless otherwise indicated, morphological and physiological traits are disclosed in comparison to control plants, including, for example, wild-type plants, plants that have not been transformed, or plants transformed with an “empty” expression vector (lacking a polynucleotide that has been introduced into an experimental plant). That is, a transformed plant that is described as large and/or drought tolerant is large and more tolerant to drought with respect to a control plant, the latter including wild-type plants, parental lines and lines transformed with a vector that does not contain a sequence of interest. When a plant is said to have a better performance than controls, it generally is larger, had greater yield, and/or showed less stress symptoms than control plants. The better performing lines may, for example, have produced less anthocyanin, or are larger, greener, or more vigorous in response to a particular stress, as noted below. Better performance generally implies greater size or yield, or tolerance to a particular biotic or abiotic stress, less sensitivity to ABA, or better recovery from a stress (as in the case of a soil-based drought treatment) than controls. Plate Assays. Different plate-based physiological assays (shown below), representing a variety of abiotic and water-deprivation-stress related conditions, are used as a pre-screen to identify top performing lines (i.e. lines from transformation with a particular construct), that are generally then tested in subsequent soil based assays. Typically, up to ten lines are subjected to plate assays, from which up to the best three lines are selected for subsequent soil based assays.

In addition, some transgenic plant lines are subjected to nutrient limitation studies. A nutrient limitation assay is intended to find genes that allow more plant growth upon deprivation of nitrogen. Nitrogen is a major nutrient affecting plant growth and development that ultimately impacts yield and stress tolerance. These assays monitor primarily root but also rosette growth on nitrogen deficient media. In all higher plants, inorganic nitrogen is first assimilated into glutamate, glutamine, aspartate and asparagine, the four amino acids used to transport assimilated nitrogen from sources (e.g. leaves) to sinks (e.g. developing seeds). This process may be regulated by light, as well as by C/N metabolic status of the plant. A C/N sensing assay is thus used to look for alterations in the mechanisms plants use to sense internal levels of carbon and nitrogen metabolites which could activate signal transduction cascades that regulate the transcription of N-assimilatory genes. To determine whether these mechanisms are altered, we exploit the observation that wild-type plants grown on media containing high levels of sucrose (3%) without a nitrogen source accumulate high levels of anthocyanins. This sucrose-induced anthocyanin accumulation can be relieved by the addition of either inorganic or organic nitrogen. We use glutamine as a nitrogen source since it also serves as a compound used to transport N in plants.

Germination assays. The following germination assays may be conducted with plants expressing sequences regulated by tissue-enhanced promoters: NaCl (150 mM), mannitol (300 mM), sucrose (9.4%), ABA (0.3 μM), cold (8° C.), polyethylene glycol (10%, with Phytogel as gelling agent), or C/N sensing or low nitrogen medium. In the text below, −N refers to basal media minus nitrogen plus 3% sucrose and −N/+Gln is basal media minus nitrogen plus 3% sucrose and 1 mM glutamine.

All germination assays are performed in tissue culture. Growing the plants under controlled temperature and humidity on sterile medium produces uniform plant material that has not been exposed to additional stresses (such as water stress) which could cause variability in the results obtained. All assays are designed to detect plants that are more tolerant or less tolerant to the particular stress condition and are developed with reference to the following publications: Jang et al. (1997), Smeekens (1998), Liu and Zhu (1997), Saleki et al. (1993), Wu et al. (1996), Zhu et al. (1998), Alia et al. (1998), Xin and Browse, (1998), Leon-Kloosterziel et al. (1996). Where possible, assay conditions are originally tested in a blind experiment with controls that had phenotypes related to the condition tested.

Prior to plating, seed for all experiments are surface sterilized in the following manner: (1) 5 minute incubation with mixing in 70% ethanol, (2) 20 minute incubation with mixing in 30% bleach, 0.01% triton-X 100, (3) 5× rinses with sterile water, (4) Seeds are re-suspended in 0.1% sterile agarose and stratified at 4° C. for 3-4 days.

All germination assays follow modifications of the same basic protocol. Sterile seeds are sown on the conditional media that has a basal composition of 80% MS+Vitamins. Plates are incubated at 22° C. under 24-hour light (120-130 μE m⁻² s⁻¹) in a growth chamber. Evaluation of germination and seedling vigor is performed five days after planting.

Growth assays. The following growth assays may be conducted with plants expressing sequences regulated by tissue-enhanced promoters: severe desiccation (a type of water deprivation assay), growth in cold conditions at 8° C., root development (visual assessment of lateral and primary roots, root hairs and overall growth), and phosphate limitation. For the nitrogen limitation assay, plants are grown in 80% Murashige and Skoog (MS) medium in which the nitrogen source is reduced to 20 mg/L of NH₄NO₃. Note that 80% MS normally has 1.32 g/L NH₄NO₃ and 1.52 g/L KNO₃. For phosphate limitation assays, seven day old seedlings are germinated on phosphate-free medium in MS medium in which KH₂PO₄ is replaced by K₂SO₄.

Transformation experiments may be performed with Arabidopsis thaliana plants such as ecotype Columbia (Col-0), soybean, maize, canola, cotton or Miscanthus plants, and many other plant species. Assays performed on Arabidopsis are usually conducted on non-selected segregating T2 populations (in order to avoid the extra stress of selection). Control plants for assays on lines containing direct promoter-fusion constructs are Col-0 plants transformed an empty transformation vector (pMEN65). Controls for 2-component lines (generated by supertransformation) are the background promoter-driver lines (i.e. promoter::LexA-GAL4TA lines), into which the supertransformations of opLexA::Gene constructs are initially performed (where the gene is a transgene of interest, the regulated expression of which is desired under control of the tissue-enhanced promoter included in the background promoter-driver line).

Procedures

For chilling growth assays, seeds are germinated and grown for seven days on MS+Vitamins+1% sucrose at 22° C. and then transferred to chilling conditions at 8° C. and evaluated after another 10 days and 17 days.

For severe desiccation (plate-based water deprivation) assays, seedlings are grown for 14 days on MS+Vitamins+1% Sucrose at 22° C. Plates are opened in the sterile hood for 3 hr for hardening and then seedlings are removed from the media and let dry for two hours in the hood. After this time the plants are transferred back to plates and incubated at 22° C. for recovery. The plants are then evaluated after five days.

Wilt screen assay. Transgenic and wild-type soybean plants are grown in 5″ pots in growth chambers. After the seedlings reach the V1 stage (the V1 stage occurs when the plants have one trifoliolate, and the unifoliolate and first trifoliolate leaves are unrolled), water is withheld and the drought treatment thus started. A drought injury phenotype score is recorded, in increasing severity of effect, as 1 to 4, with 1 designated no obvious effect and 4 indicating a dead plant. Drought scoring is initiated as soon as one plant in one growth chamber had a drought score of 1.5. Scoring continues every day until at least 90% of the wild type plants achieve scores of 3.5 or more. At the end of the experiment the scores for both transgenic and wild type soybean seedlings are statistically analyzed using Risk Score and Survival analysis methods (Glantz, 2001); Hosmer and Lemeshow, 1999).
Water use efficiency (WUE). WUE is estimated by exploiting the observation that elements can exist in both stable and unstable (radioactive) forms. Most elements of biological interest (including C, H, O, N, and S) have two or more stable isotopes, with the lightest of these present in much greater abundance than the others. For example, ¹²C is more abundant than ¹³C in nature (¹²C=98.89%, ¹³C=1.11%, ¹⁴C=<10⁻¹⁰%). Because ¹³C is slightly larger than ¹²C, fractionation of CO₂ during photosynthesis occurs at two steps:

1. ¹²CO₂ diffuses through air and into the leaf more easily;

2. ¹²CO₂ is preferred by the enzyme in the first step of photosynthesis, ribulose bisphosphate carboxylase/oxygenase.

WUE has been shown to be negatively correlated with carbon isotope discrimination during photosynthesis in several C3 crop species. Carbon isotope discrimination has also been linked to drought tolerance and yield stability in drought-prone environments and has been successfully used to identify genotypes with better drought tolerance. ¹³C/¹²C content is measured after combustion of plant material and conversion to CO₂, and analysis by mass spectroscopy. With comparison to a known standard, ¹³C content is altered in such a way as to suggest that overexpression of a transgene of interest, such as G1988 or its related sequences, improves water use efficiency.

Another potential indicator of WUE is stomatal conductance, that is, the extent to which stomata are open.

Data Interpretation

At the time of evaluation, plants are typically given one of the following scores:
(++) Substantially enhanced performance compared to controls. The phenotype is very consistent and growth is significantly above the normal levels of variability observed for that assay.
(+) Enhanced performance compared to controls. The response is consistent but is only moderately above the normal levels of variability observed for that assay.
(wt) No detectable difference from wild-type controls.
(−) Impaired performance compared to controls. The response is consistent but is only moderately above the normal levels of variability observed for that assay.
(−−) Substantially impaired performance compared to controls. The phenotype is consistent and growth is significantly above the normal levels of variability observed for that assay.
(n/d) Experiment failed, data not obtained, or assay not performed.

### Soil Drought (Clay Pot)

The soil drought assay (performed in clay pots) is based on that described by Haake et al. (2002).

Procedures. Previously, we have performed clay-pot assays on segregating T2 populations, sown directly to soil. However, in the current procedure, seedlings are first germinated on selection plates containing either kanamycin or sulfonamide.

Seeds are sterilized by a 2 minute ethanol treatment followed by 20 minutes in 30% bleach/0.01% Tween and five washes in distilled water. Seeds are sown to MS agar in 0.1% agarose and stratified for three days at 4° C., before transfer to growth cabinets with a temperature of 22° C. After seven days of growth on selection plates, seedlings are transplanted to 3.5 inch diameter clay pots containing 80 grams of a 50:50 mix of vermiculite:perlite topped with 80 grams of ProMix. Typically, each pot contains 14 seedlings, and plants of the transgenic line being tested are in separate pots to the wild-type controls. Pots containing the transgenic line versus control pots are interspersed in the growth room, maintained under 24-hour light conditions (18-23° C., and 90-100 μE m⁻² s⁻¹) and watered for a period of 14 days. Water is then withheld and pots are placed on absorbent paper for a period of 8-10 days to apply a drought treatment. After this period, a visual qualitative “drought score” from 0-6 is assigned to record the extent of visible drought stress symptoms. A score of “6” corresponds to no visible symptoms whereas a score of “0” corresponds to extreme wilting and the leaves having a “crispy” texture. At the end of the drought period, pots are re-watered and scored after 5-6 days; the number of surviving plants in each pot is counted, and the proportion of the total plants in the pot that survive is calculated.

Analysis of results. In a given experiment, we typically compare 6 or more pots of a transgenic line with 6 or more pots of the appropriate control. The mean drought score and mean proportion of plants surviving (survival rate) are calculated for both the transgenic line and the wild-type pots. In each case a p-value* is calculated, which indicates the significance of the difference between the two mean values.

Calculation of p-values. For the assays where control and experimental plants are in separate pots, survival is analyzed with a logistic regression to account for the fact that the random variable is a proportion between 0 and 1. The reported p-value is the significance of the experimental proportion contrasted to the control, based upon regressing the logit-transformed data.

Drought score, being an ordered factor with no real numeric meaning, is analyzed with a non-parametric test between the experimental and control groups. The p-value is calculated with a Mann-Whitney rank-sum test.

### Example IX

### Field Plot Designs, Harvesting and Yield Measurements of Soybean and Maize

A field plot of soybeans with any of various configurations and/or planting densities may be used to measure crop yield. For example, 30-inch-row trial plots consisting of multiple rows, for example, four to six rows, may be used for determining yield measurements. The rows may be approximately 20 feet long or less, or 20 meters in length or longer. The plots may be seeded at a measured rate of seeds per acre, for example, at a rate of about 100,000, 200,000, or 250,000 seeds/acre, or about 100,000-250,000 seeds per acre (the latter range is about 250,000 to 620,000 seeds/hectare).

Harvesting may be performed with a small plot combine or by hand harvesting. Harvest yield data are generally collected from inside rows of each plot of soy plants to measure yield, for example, the innermost inside two rows. Soybean yield may be reported in bushels (60 pounds) per acre. Grain moisture and test weight are determined; an electronic moisture monitor may be used to determine the moisture content, and yield is then adjusted for a moisture content of 13 percent (130 g/kg) moisture. Yield is typically expressed in bushels per acre or tonnes per hectare. Seed may be subsequently processed to yield component parts such as oil or carbohydrate, and this may also be expressed as the yield of that component per unit area.

For determining yield of maize, varieties are commonly planted at a rate of 15,000 to 40,000 seeds per acre (about 37,000 to 100,000 seeds per hectare), often in 30 inch rows. A common sampling area for each maize variety tested is with rows of 30 in. per row by 50 or 100 or more feet. At physiological maturity, maize grain yield may also be measured from each of number of defined area grids, for example, in each of 100 grids of, for example, 4.5 m² or larger. Yield measurements may be determined using a combine equipped with an electronic weigh bucket, or a combine harvester fitted with a grain-flow sensor. Generally, center rows of each test area (for example, center rows of a test plot or center rows of a grid) are used for yield measurements. Yield is typically expressed in bushels per acre or tonnes per hectare. Seed may be subsequently processed to yield component parts such as oil or carbohydrate, and this may also be expressed as the yield of that component per unit area.

### Example X

### Polypeptide Sequences that Confer Significant Improvements to Crops

It is envisioned that the disclosed tissue-enhanced promoter sequences (e.g., SEQ ID NOs: 1-66, or a functional part thereof having a promoter or gene-regulatory function) may be used to improve the yield that may be derived from a non-Arabidopsis plant species, or from a crop plant species. Said yield improvement may result from, but is not limited to, the plant having greater biomass, greater plant size, greater plant volume, greater disease resistance, greater resistance to fungal pathogens, greater resistance to biotrophic pathogens, greater resistance to necrotrophic pathogens, greater resistance to Fusarium, greater resistance to Botrytis, greater resistance to Erysiphe, greater resistance to Sclerotinia, constitutive photomorphogenesis, greater photosynthetic capacity, dark green color, more chlorophyll A, more chlorophyll B, more carotenoids, more anthocyanin, reduced sensitivity to light, greater early season growth, greater height, greater stem diameter, greater resistance to lodging, greater internode length, greater secondary rooting, greater cold tolerance, greater tolerance to water deprivation, greater tolerance to heat, greater tolerance to salt, greater water use efficiency, reduced stomatal conductance, altered C/N sensing, greater low nitrogen tolerance, greater low phosphorus tolerance, greater tolerance to hyperosmotic stress, greater late season growth and vigor, greater number of primary nodes, and greater canopy coverage relative to a control plant, when one or more of the disclosed tissue-enhanced promoter sequences is used to regulate transcription in the non-Arabidopsis or crop plant.

Tissue-enhanced promoter sequences may be used to regulate the expression of genes of interest in crop or other valuable plants. The ectopic overexpression of protein sequences, or any other sequence that may confer an improved or desirable trait, may be regulated using tissue-enhanced regulatory elements found in the Sequence Listing. In addition to these sequences, it is expected that newly discovered polynucleotide sequences from, for example, other species having similar sequences (e.g. the promoters from genes that represent homologs of tissue-enhanced genes listed in the Tables 1-4), may be closely related to polynucleotide sequences found in the Sequence Listing and can also be used confer improved traits in a similar manner to the sequences found in the Sequence Listing, when transformed into any of a considerable variety of plants of different species, and including dicots and monocots. The polynucleotide sequences derived from monocots (e.g., the rice sequences) may be used to transform both monocot and dicot plants, and those derived from dicots may be used to transform either group, although a preferred embodiment may include a sequence transformed into a plant from the same major clades of angiosperm as that from which the sequence is derived.

The examples above show that polypeptides that confer an improved or desirable trait may do so when they are expressed under the regulatory control of a tissue-enhanced promoter sequence, or have their expression repressed under the regulatory control of a tissue-enhanced promoter sequence, without having a significant adverse impact on plant morphology and/or development. After identifying as plant lines that display useful traits, such as the traits provided above, said lines may be selected for further study or commercial development.

Dicotyledonous or monocotyledonous plants, including those listed in Examples V and VI, or other plants, may be transformed with a plasmid containing a polynucleotide of interest. The polynucleotide sequence may include dicot or monocot-derived sequences such as those presented herein. These polynucleotide sequences may be cloned into an expression vector containing a kanamycin-resistance marker, and then expressed under the regulatory control of a tissue-enhanced promoter sequence.

It is expected that closely related and structurally similar promoter sequences, may also regulate gene expression in a tissue-enhanced expression pattern similar to the sequences provided herein. It is thus expected that the same methods may be applied to identify other useful and valuable promoter sequences, and the sequences may be derived from a diverse range of species.

All publications and patent applications mentioned in this specification are herein incorporated by reference to the same extent as if each individual publication or patent application was specifically and individually indicated to be incorporated by reference.

### REFERENCES

Alia et al. (1998) Plant J. 16: 155-161
Aldemita and Hodges (1996) Planta 199: 612-617
Altschul (1990) J. Mol. Biol. 215: 403-410
Altschul (1993) J. Mol. Evol. 36: 290-300
Aukerman and Sakai (2003). Plant Cell 15:, 2730-2741
Bartel (2004) Cell 116: 281-297
Bartel and Bartel (2003) Plant Physiol. 132: 709-717
Bechtold and Pelletier (1998) Methods Mol. Biol. 82: 259-266
Bevan (1984) Nucleic Acids Res. 12: 8711-8721
Bowman (2004) Bioessays 26: 938-942
Cardoza et al. (2003) Plant Cell Rep. 21: 599-604
Cassas et al. (1993) Proc. Natl. Acad. Sci. USA 90: 11212-11216
Charest et al. (1988) Theor. Appl. Genet. 75: 438-445
Christou et al. (1987) Proc. Natl. Acad. Sci. USA 84: 3962-3966
Christou (1991) Bio/Technol. 9:957-962
Christou et al. (1992) Plant. J. 2: 275-281
D'Halluin et al. (1992) Plant Cell 4: 1495-1505
Daly et al. (2001) Plant Physiol. 127: 1328-1333
De Block et al. (1987) Plant Physiol. 91: 694-701
Deshayes et al. (1985) EMBO J.: 4: 2731-2737
Donn et al. (1990) in Abstracts of VIIth International Congress on Plant Cell and Tissue Culture IAPTC, A2-38: 53
Draper et al. (1982) Plant Cell Physiol. 23: 451-458
Fromm et al. (1990) Bio/Technol. 8: 833-839
Gelvin et al. (1990) Plant Molecular Biology Manual, Kluwer Academic Publishers
Glantz (2001) Relative risk and risk score, in Primer of Biostatistics. 5^{th} ed., McGraw Hill/Appleton and Lange, pub.
Glick and Thompson (1993) Methods in Plant Molecular Biology and Biotechnology. CRC Press., Boca Raton, Fla.
Gordon-Kamm et al. (1990) Plant Cell 2: 603-618
Gruber et al. (1993) in Methods in Plant Molecular Biology and Biotechnology, p. 89-119
Haake et al. (2002) Plant Physiol. 130: 639-648
Hain et al. (1985) Mol. Gen. Genet. 199: 161-168
Herrera-Estrella et al. (1983) Nature 303: 209
Hiei et al. (1994) Plant J. 6:271-282
Hiei et al. (1997) Plant Mol. Biol. 35:205-218
Hosmer and Lemeshow (1999) Applied Survival Analysis: Regression Modeling of Time to Event Data. John Wiley & Sons, Inc., Publisher.
Ishida (1990)) Nature Biotechnol. 14:745-750
Jaglo et al. (2001) Plant Physiol. 127: 910-917
Jang et al. (1997) Plant Cell 9: 5-19
Juarez et al. (2004) Nature 428: 84-88
Klee (1985) Bio/Technology 3: 637-642).
Klein et al. (1987) Nature 327: 70-73
Koornneef et al (1986) in Tomato Biotechnology: Alan R. Liss, Inc., 169-178
Ku et al. (2000) Proc. Natl. Acad. Sci. USA 97: 9121-9126;
Leon-Kloosterziel et al. (1996) Plant Physiol. 110: 233-240
Liu and Zhu (1997) Proc. Natl. Acad. Sci. USA 94: 14960-14964
Miki et al. (1993) in Methods in Plant Molecular Biology and Biotechnology, p. 67-88, Glick and Thompson, eds., CRC Press, Inc., Boca Raton;
Novillo et al. (2004) Proc. Natl. Acad. Sci. USA 101:, 3985-3990
Pua et al. (1987) Biotechnol. 5: 815-817
Radke et al. (1988) Theor. Appl. Genet. 75: 685-694
Rieger et al. (1976) Glossary of Genetics and Cytogenetics: Classical and Molecular, 4th ed., Springer Verlag, Berlin
Saleki et al. (1993) Plant Physiol. 101: 839-845
Sanford et al. (1987) Part. Sci. Technol. 5:27-37
Sanford (1993) Methods Enzymol. 217: 483-509
Smeekens (1998) Curr. Opin. Plant Biol. 1: 230-234
Somleva et al. (2002) Crop Sci. 42: 2080-2087
Spencer et al. (1994) Plant Mol. Biol. 24: 51-61
Stewart et al. (1996) Plant Physiol. 112: 115-120
Sunkar and Zhu (2004) Plant Cell 16: 2001-2019
Tudge (2000) in The Variety of Life, Oxford University Press, New York, N.Y. pp. 547-606
Vasil et al. (1992) Bio/Technol. 10:667-674
Vasil et al. (1993) Bio/Technol. 11:1553-1558
Vasil (1994) Plant Mol. Biol. 25: 925-937
Wan and Lemeaux (1994) Plant Physiol. 104: 37-48
Weeks et al. (1993) Plant Physiol. 102:1077-1084
Weissbach and Weissbach, (1989) Methods for Plant Molecular Biology, Academic Press
Wu et al. (1996) Plant Cell 8: 617-627
Xin and Browse (1998) Proc. Natl. Acad. Sci. USA 95: 7799-7804
Zhang et al. (1991) Bio/Technology 9: 996-997
Zhu et al. (1998) Plant Cell 10: 1181-1191

All publications and patent applications mentioned in this specification are herein incorporated by reference to the same extent as if each individual publication or patent application was specifically and individually indicated to be incorporated by reference.

The present claims are not limited by the specific embodiments described herein. It will be apparent to one of ordinary skill in the art that many changes and modifications can be made thereto without departing from the spirit or scope of the appended claims. Modifications that become apparent from the foregoing description fall within the scope of the claims.

What is claimed is:

1. A recombinant polynucleotide comprising a nucleic acid sequence having a promoter function capable of modulating transcription of an operably-linked heterologous transcribable nucleotide molecule;
wherein the nucleic acid sequence has a percentage identity with any of SEQ ID NOs: 1-66, or a functional part thereof having the promoter function, or a complement thereof, or the nucleic acid sequence comprises at least 25 contiguous bases of any of SEQ ID NOs: 1-66, wherein the nucleic acid sequence, the functional part thereof, the complement thereof, or the at least 25 contiguous bases, enhance expression of a polypeptide in one or more plant tissues;
wherein the percentage identity is selected from the group consisting of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% and 100%.

2. The recombinant polynucleotide of claim 1, wherein the nucleic acid sequence enhances expression of the polypeptide in vascular, shoot apical meristem, hypocotyl or leaf primordia tissue.

3. The recombinant polynucleotide of claim 1, wherein the functional part thereof having the promoter function comprises a continuous region of at least 25 base pairs, 50 base pairs, 75 base pairs, 100 base pairs, 125 base pairs, 150 base pairs, 175 base pairs, 200 base pairs, 225 base pairs, 250 base pairs, 275 base pairs, 300 base pairs, 325 base pairs, 350 base pairs, 375 base pairs, 400 base pairs, 425 base pairs, 450 base pairs, 475 base pairs, 500 base pairs, 525 base pairs, 550 base pairs, 575 base pairs, 600 base pairs, 625 base pairs, 650 base pairs, 675 base pairs, 700 base pairs, 724 base pairs, 725 base pairs, 750 base pairs, 775 base pairs, 800 base pairs, 825 base pairs, 850 base pairs, 875 base pairs, 900 base pairs, 925 base pairs, 950 base pairs, 975 base pairs, 1000 base pairs, 1100 base pairs, 1200 base pairs, 1204 base pairs, 1300 base pairs, 1400 base pairs, 1500 base pairs, 1600 base pairs, 1700 base pairs, 1800 base pairs, 1900 base pairs, 2000 base pairs, 2100 base pairs, 2200 base pairs, 2300 base pairs, 2400 base pairs, 2500 base pairs, 2600 base pairs, 2700 base pairs, 2800 base pairs, 2900 base pairs, 2999 base pairs, or 3000 base pairs of any of SEQ ID NOs: 1-66.

4. The recombinant polynucleotide of claim 1, wherein the recombinant polynucleotide comprises an RNA polymerase binding site located 5′ relative to and operably linked to a coding sequence encoding the polypeptide, and the polypeptide confers an altered trait relative to a trait in a control plant.

5. The recombinant polynucleotide of claim 1, wherein the recombinant polynucleotide encodes a polypeptide that regulates transcription in a plant cell.

6. The recombinant polynucleotide of claim 5, wherein the polypeptide is selected from the group consisting of SEQ ID NO: 68, SEQ ID NO: 70 and SEQ ID NO: 72.

7. A host plant cell transformed with the recombinant polynucleotide of claim 1.

8. A transgenic plant transformed with a nucleic acid construct comprising a tissue-enhanced promoter sequence and a polynucleotide;
wherein the tissue-enhanced promoter sequence regulates expression of the polynucleotide and its encoded polypeptide in one or more plant tissues;
wherein the tissue-enhanced promoter sequence has a percentage identity with any of SEQ ID NOs: 1-66, or a functional part thereof, or a complement thereof, or the promoter comprises at least 25 contiguous bases of any of SEQ ID NOs: 1-66, wherein the promoter, the functional part thereof, the complement thereof, or the 25 contiguous bases;
wherein the percentage identity is selected from the group consisting of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% and 100%.

9. The transgenic plant of claim 8, wherein the nucleic acid construct comprises an RNA polymerase binding site located 5′ relative to and operably linked to a coding sequence encoding the polypeptide, and the polypeptide confers to the transgenic plant an altered trait relative to a trait in a control plant that does not contain the nucleic acid construct and is of the same species as the transgenic plant.

10. The transgenic plant of claim 8, wherein the tissue-enhanced promoter sequence regulates expression of the polypeptide in vascular, shoot apical meristem, hypocotyl or leaf primordia tissue.

11. A transgenic seed produced by the transgenic plant of claim 8, wherein the transgenic seed comprises the nucleic acid construct.

12. The transgenic plant of claim 8, wherein the polypeptide regulates transcription in the transgenic plant.

13. The transgenic plant of claim 12, wherein the polypeptide is selected from the group consisting of SEQ ID NO: 68, SEQ ID NO: 70 and SEQ ID NO: 72.

14. The transgenic plant of claim 8, wherein as a result of the expression of the polypeptide in the transgenic plant, the transgenic plant has greater yield, greater biomass, greater plant size, greater plant volume, greater disease resistance, greater resistance to fungal pathogens, greater resistance to biotrophic pathogens, greater resistance to necrotrophic pathogens, greater resistance to Fusarium, greater resistance to Botrytis, greater resistance to Erysiphe, greater resistance to Sclerotinia, constitutive photomorphogenesis, greater photosynthetic capacity, dark green color, more chlorophyll A, more chlorophyll B, more carotenoids, more anthocyanin, reduced sensitivity to light, greater early season growth, greater height, greater stem diameter, greater resistance to lodging, greater internode length, greater secondary rooting, greater cold tolerance, greater tolerance to water deprivation, greater tolerance to heat, greater tolerance to salt, greater water use efficiency, reduced stomatal conductance, altered C/N sensing, greater low nitrogen tolerance, greater low phosphorus tolerance, greater tolerance to hyperosmotic stress, greater late season growth and vigor, greater number of primary nodes, and greater canopy coverage, relative to a control plant that does not contain the nucleic acid construct and is of the same species as the transgenic plant.

15. The transgenic plant of claim 14, wherein the transgenic plant is morphologically similar and/or developmentally similar to the control plant

16. A method for producing a transgenic plant having an altered trait relative to a control plant, the method steps including:
(a) generating a nucleic acid construct comprising:
(i) a tissue-enhanced promoter sequence having a percentage identity with any of SEQ ID NOs: 1-66, or a functional part thereof, or a complement thereof, or the promoter comprises at least 25 contiguous bases of any of SEQ ID NOs: 1-66, wherein the promoter, the functional part thereof, the complement thereof, or the 25 contiguous bases enhance expression of a polypeptide in one or more plant tissues, and the percentage identity is selected from the group consisting of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% and 100%; and
(ii) a nucleotide sequence that encodes a polypeptide or RNA molecule that alters the trait in the transgenic plant;
wherein the tissue-enhanced promoter sequence is operably linked to the nucleotide sequence that encodes the polypeptide, and the promoter sequence drives the expression of the nucleotide sequence that encodes the polypeptide; and
(b) transforming a target plant with the nucleic acid construct to produce the transgenic plant;
wherein when the polypeptide is expressed in the transgenic plant, the transgenic plant alters the trait relative to the control plant, and the control plant does not contain the nucleic acid construct and is of the same species as the transgenic plant.

17. The method of claim 16, wherein the promoter sequence comprises an RNA polymerase binding site located 5′ relative to and operably linked to a coding sequence encoding the polypeptide.

18. The method of claim 16, wherein the polypeptide regulates transcription in the transgenic plant.

19. The method of claim 18, wherein the polypeptide is selected from the group consisting of SEQ ID NO: 68, SEQ ID NO: 70 and SEQ ID NO: 72.

20. The method of claim 16, wherein the transgenic plant is morphologically similar and/or developmentally similar to the control plant.


SEQUENCE LISTING (73 sequences)

SEQ ID NO: 1 - DNA, 3000 bp, Arabidopsis thaliana
  AT1G08465 chr12672813-2675812 FORWARD (source_file=TAIR9_upstream_3000_20090619)
aaaacctgtttctgaatttggtagttttgacaaatcttaaccttaaaaaattcgactttgatttgtgattattgctttctcatgtaatccattgtgatccttgtacagagtacattgagaagctagtggaggcagataaatcaggagaaaggtgtgaaattgccgcaggtactttcatgagcccaggctcgtgggaagctgctcttcttgcagctggaacaactctttcagcaatgcaacatattcttgattgccatgggaaaattgcatatgctctggtacgcccaccaggtcaccactcacagcctactcaggctgatgggtattgcttcctcaacaacgctgctcttgctgtcaagctggcattgaattcgggttcttgttcccgagttgcggttattgacatcgatgtacattatggaaatggcacagctgaagggttttatacatctgataaggttcttacggtgtcactccatatgaaccatggatcatggggatcgtcccacccgcagaaagggtcgattgatgaacttggtgaagacgtgggattgggttacaatctgaatgtccctttaccgaatggcacgggtgaccggggatatgaatatgccatgaatgagctggtggttcctgcagttagaaggtttggacccgatatggttgttctcgttgttggtcaagactctagcgctgtaagttctttctttgcccaaatacgtgtagactatctgactgagcacttactcagggttttgttcgcaattattgttttggttacaagtaaatcgcttatgtatgctgctccaatatggctctgctgtgataagtctgcataagtagttagaagagtgcagctgttgcattagtcatgtcatctctctaatattcagtcagcaaaagataaacgaatcagtataaatggaagatgaccaaaggaggaatgggttgtctagggatttgggtattggttttgcattggaagagtctagggtgctcgagaattcaccataagattatatctttggattctttgcttcagtttgcattcttattcagagaggaataaaggaagatatggtttttatggttcttattacgctacctagtggtccccttgtcgagcttagtgaatttatattcagcacagatgttactaacagagctgttcactttttggcccatggaagttaatgaattttctggttcacaggaaactctctcttaaagttctccggcttaaatccattagtttggtttcaaacatttgctagggatgctgattgtctagtttgtgaggggatttgaatgtcatggactgacggataaccactatttactgtcggatctgcagtttgatccaaacgggagacaaagcctgacaatgaatggatacagaaggattgggcagataatgaggggagttgcagaggagcacagccatggaaggttgcttatggtgcaagaaggcgggtatcatgtaacatatgcagcttactgcctccacgccatgctcgaaggggtgctgaagattccagaaccgcatctatctgatccgatcgcttattatcccgaagaagaagctaatgctgttgctgctgtggagtcaatcaaaacatatcacaccgaatttgttccatttctcagaggaacttgaaaatgggacgtatgaatgagtttatgttattcctcgttgtcttgtgaccttttggaacacagatccaaatcaatcaggtcaaaccgtatatatcaaaatacttgttgatcaaactatcttttgtatgaaaacttattcacacgatccgactttgttttttgagtttttttttaacaactagttaggtttagactagaagaacccaaattttttgtgacagaataataattttgcagaaagagattttataaatatattccagatacaatcaaatcaagcttggaaataagattggcattgtcgtgagtatcactgtagagagaatagctcgcgggtccgtaaaaaatgcatgtcaacctcgatcgtggaaatatgtcaggataagatctgtattctcgtctctatgatttgttcgaaagaaacttgtttatatgaatcattttgtaagaaatgtttgttgcattgattccaaatactttagatgtgttcaattagtttattcgctttattttttattttatttttggtaaaaggttaataaagacaaaataggaaattgcattgcaaatacaaatacaaatacaaaacaatgaaacatctatattatgaaataaattattaaaaattaaaccagtaataaatgtcgttttgttgatccacactcaatctaaaatgtacatctatgttcacttgcagaatgaatgggattttaatgaactttaaaaactcaatttcaagtaattacaccaatccaatgaacatgtttaagtttgcagaaaatgccaaaatgaattcatctttcgttttatactgttaatcacttattcacaaaatagaccatcattgtcacttgcaatatgttttatttgattctgaagaggattatgtatcgttttcaattgtccctgaattcacaaactgtatcgttttactaaatttactgttgctgctaacaaagtccatgtatcgttttcagatatgtgtttgaaaaaacaaaatttaatcgaactgttaatctcatttcttatctgctagacaaatacaatacacattctcatgatctagtactattcattgtaaaatgactgtaacaataagattatatattgagctactagcctactacttatcctataaaaatgtaattgaaattaacttatttggtgataggactatatatcttaactaaagatgaaaaaacaataaaaataacaaagggaaatcttcactttattatggagtaatagaactctctaattatctaaggaaaaaaaaatgcaagtaccgaaaaataaaaaagaggaacattgtagtatagtccccacttgtctctcttcgtctcc

SEQ ID NO: 2 - DNA, 3000 bp, Arabidopsis thaliana
  AT1G10155 chr13328449-3331448 REVERSE (source_file=TAIR9_upstream_3000_20090619)
taggaggctgaggatatacagacccaaatccaatggataaagactaaaggaagatcaagagccttcaaaatattgatgagaaactcccagttgacattatcataggctttagatatatccacctgaagacagcctctagtagtctctccatcagcctcgaaattatccaccaactcagatgcaagaagaacattctcacatagaagccgacccttaatgaacccaacctgatttgcctgaaccgcttggtcaatgaaaagctttagccttctggaaatgatgcgagtaatgaccttataaatagtagtgcaacaagccacagggcgaaattgagttagacgatctgcccctgttacctttggaatcagagtgatggctgtagcattaaagccccttggaagattcccagagataaagaattccctgatcgcagccacaacactagacttcacaatcgcccaagcttcgatgaagaattcaactggaaacccatctggacccggtgctttgttacggggcatagagaagagcacctgagttatttcctcttccgaaggaatagtagtcaactgagaagccaggaaggaatcacaacgaaaggggagaagacctttgattttttcaacagagaatggggtgacattctctgaaggaatgccaagaagatgcgaataataagcaatcaacatcccttttatctgatccacattctcgaccctaaaaccatcatctcctctaagaaacttgataagattagtcgcttggtgagcaatcacagctctgtgaaaaaatctagtattagcatcaccttcatgaagccatcttattctggatttttgtcgaaagaatgactctaaagcagcagcaaagaagatccattgcttgcgggcaacatgttcccgacgaaaaagagtatcagaaggtgacgtgaggagttccacttgaatgtcctcaagccgagttagagattgggctgtcctttgttgaatattgctgaaacggagcctgttaagagtcctgcaacacagcttagcaacttttaaatgctgcctcaaagaaaacatgtgagacccaaccaaagtattctcctcccaagcagtagacaaagccgccaaataagaaggatgagaagacaagaaggagaaatacttaaaggacttcttagaaggaggaggctgattatcgattaagatgatacaaggggcatgatcagagtcacctggaggatcaaagaccgcaagagcagagggaaagaccgcgaaccactcaccattagctaaggccctgtcaagcttccgaaggatgggattgtcctgttgatgatttgaccaagtgaaaaacacacctcgagaaggcaggtctgaaagctgactgtctctcaaacaacattgaagatcctccataccacgcaggttcaataaggattgattgatagaataatgttccgaagcagcggctatctgattaaaatctcccaacaaaagccagggagtaacagaaagaggagaagtcctggacagcacgagaatgtcttcccacagagatcttctatctaactcagaattcctgccataaacaaaagcaaccgcaaaagattgaagaagagaaggaatcttaatgctgcaaaacataatctgatccgttctcttaaacaccaaaactgaaatggaaggatcccagactatccatatcctcccaagttcagagcaacaatagttgctatccatcctccaacccggaagggttgaggccaaaaccgagtttgcattctcttgtgccacatgagtttccaaaaaacatcccacaagaagattattagaagcaatccagcttcgcacaactcgttgacgattacggctatttaagcctctaatattccaacaaaagaccttcatattcacaacaaaaggtgaaggtgaaagcaaagctttcaatgagaagaaggaccgtcaccggccctgccatcgacgaaaccttgaagctctgtcacggggggacgaatacggttgtgccacttcttaagttctttcttttttgccttagtagacatcactgcccagcccaggggaagagggaagtttggatccggatctttgatagactctctcatagattttttagggagagtaggaggttgagaaattccagaatctgcaccaatgacaacagtaggaactggcgaattcaaacctggtctcacaacccatttcttactagccgaaggcttgggcaatagagggacctgctgagaaatgggaatcaacgggtcaacaatccgaggtggagaactgagagagcgcttctttgatctcgaacgccttctttttggtttggaccaagggagctcaggtggattagctgaagctgaaccaatgacactagtttctgaaatcccagcacctgaaggaaggttaacagcagagagagccacttcattagccccggagggatagtcttttttaaagggaagtttcttctgcaaggggatgagacaacggcttattaaatgaccatacttgccacaattcaagcacttcccatatgtgtcatattctgcccactagcaaaaattgttatcgagaataaaatctagaacatcattaggacataggaatctaaaatgacaaaggccaactagttcaaaacttcattacagtctcaaaaatttcactctttggcaaattgaaatcaaatatgtgatcatttgattctcatacaaaagcagagatagaagaagagccacaaactgagaatatttgctttagcttttgccatccaattgaattataccttttacatggttatggcactaaagtaaaacttacaaggagaatattgagaagaaaagattgggtccaacttcatttattcttcttctgtcacaatactaatttttttctacagaaagtttccatatagtgaaaagaaacaaatctctatataaagccaacttaagagaagtgttcttttataaacaaacagcaaagaagaaagagagaa

SEQ ID NO: 3 - DNA, 3000 bp, Arabidopsis thaliana
  AT1G14190 chr14849802-4852801 FORWARD (source_file=TAIR9_upstream_3000_20090619)
ccataaacaaagtagcccatttcatgatttagaagaaagaggtttttgttacttgcataaacttattgtttgatttgctctcgtgataagtattttgttcgatctctaacaacacttggaaattaaatatattcaagataggataagaatagctattcttattacattatggggaagaataaaagaaaaagaaaaaagagaggtggagccaagtcaactagatcgacaagatagtgatttgattttagaaaaattcaaataattatccaacaaagagagcatgaatcatgatctccaaccatatatgcgttcattattgtaacaatttttttttggaattatttattcattgaccaccttctcagaccatgcgttccacgacacaaaatactcacttagacccacaacctcttccatcaaaccaaacaatcccttttcctcagacaaaactttgtctctgaatctgaaaatcaagattagacatgtctcattttgtgtttctctttattatttccatgttcattaatctgtcacaaggtattaataatgtagactatctccttgtttgaaaatactatagtttatatgcagtgaaggttttcattaactgttgtttgacgttttcaggagcgcaaatgccttacatgactactgatccgaaagaagtttccggcaagtcattcgattacatcgttgttggaggcggaactgcggggtgttccttagctgctactctgtctgagaaatactctgttctggtcattgaacgcggtggctcgccttttggagatccattagtagaagataaaaagtactatggatactcattgataaatacagatgaatactcatccgttgcgcaaagttttacctccgtagatggaatcaaaaaccacagaggacgtgttcttggaggatcgagcgctatcaatggcggattttacagccgagctagcgatgagtttgtgaagaaagctggttgggacaaggatctggttcaagaatcttataaatgggttgagtctaaggttgtcttcatgccggagttgactcgatggcaatccattgtgcagttcggatttcttgaagcagggttttatccgtataacggatatagtttggagcacacgcaagggacaaagatcggtggaagcatatttgatcagtgtgggaaaagacatacatctgcagatcttttgggatatggtaaaccaaattgcatcactgttcttctaaacgcaacggtaaagagcataatctttgatgctaacaagactcgcgcagttggagttaggtttatggagagtgatgggaactcaagtaagagctacaaagttcatgttgagcagcatagaggcgaggttatacttgcggctggggctttaggtagtccgcagattctcctcttaagcggtattggacccgagaatcatctcaatgatttcgacattcctgtgattgtcaatctcaaagaagtcggaaaacaaatgtcagataatccagcgatctctcttctcgttgatagattctcgcaaaacctcacagtcgatccacctcaagttgtagcgataacagaaggttttaaattcatactccaatccctggttcttccaactaacattaccacaacaagaaccgctatatcagccaaaatcgcattccctaaatccaaaggaagactcaagcttaacaacactaaccctagggagaatccgtcagtgacgttcaactacttggaaaacaaggcagaccttgacgcatgccaagagatggttttgcatcttcaacacgtggctaggtcaaagactgtgacgtttttcttggggacacaagctcaggataagctagtggcaggtgacgaagagcttaagaagttctgcataaagaatgtgagaacttattatcattaccatgggggttgcgttgtgggatctgtcgtgaatgaggaatataaagtgaatggtgtgaagcgtttaagagtcgttgatggttcaacgtttgaagaatcaccaggaacaaaccctatggcaacagtattaatgctgggaagatatcaaggaatcaagatactcaaagaacattaggaaaggttgtttagcccttaaaatattttgaaccaaaacgatgtaaatgcaaaaatgttcatgaaaaagattttaaaccaaaacgatgtaaaaatgttcattatagtttaaatccaaatgagaactgaaatccaaatgctcgtatcttgtaatatagcttttgtttcaaggattagatgtttggttttaaatcttctccatcgaccattggtattaatgtctcttgaatgtgtatatttcgttttgatacagtccactacacattgctctagatttttcagacaatggtggtcgtctcttttcgtactctcttatcattatgaatttcaggttgttagttttggatcttttggttaaggttaatttttttttttaaccattatcaaaaaaggttaattgtccatttttctaactaattttgttgcctcgtcgtccactcagatttttagttggaacaagaaaaggtaaaaatcataattatccagggccatgacttaaaggcatgaatatatgattatgcaaggaaaatttcataattatccaacaaagagagcatcattgtctctaaccatgcgttccttattgtaacaaactttttcaatattatttatttgttgactaccttctcagatcattatgtctacactctcattgctttaagagacacaaaatcatcacctagactcacaacctcttccattaaaccaaacaatcccttttaatcagacaaaactctgtctctcaatctcaaaatcaagattgacatgtctcgttttgtgcttctctttatcatttccacattcattaatctgtcacaaggtattaatgtagagtatcttcttgtttgaaacaaatatattatatatgtgcaataaggtttttcattagatgttgtttgatgttttcaggagcgcaa

SEQ ID NO: 4 - DNA, 3000 bp, Arabidopsis thaliana
  AT1G24130 chr18535431-8538430 REVERSE (source_file=TAIR9_upstream_3000_20090619)
ttgggggcaaacgaaattttcttaatattttataatagagaaagacacccaattaaattgtccattgaggatgctctaaagagtcctctagtgaaggtgctcttatgactccattagtcatacactaaccgtatgcaaagaaaacctatcacttggtggtagaatataattaagtattattaacccaaagcacaccaaaaaaaaaatgtaattaaaatctgtaactaaaatgaattagtcagtcaaaaatatcataactacgaaataaatgtacactaaacactatcaaacgtcccttagtttagattttggttaataaagctatgcgtttacaggcttctaagttttaataatgttggtgcaaacgtggttgaactaattctgaaaacaatttttagcctaaatggatctaacatgaaaatctacatttttctgtttatatgtatcctaattctgtttagaaagggcttcaaacttttggcccaaaactttatatgcatgtatgatatatcaattaattttaccgatggagacttgcatgttatacccattacaggaaaaaattagatagcctaacaaacaagtcatggcgttactcgtgtaaggtggagattttacgttgattttttcaaccaataacaacaacctccggaaaaattatcacaaaaagaaaaaagaccaattcaaacattcaacgacccaaaaaaaacaaaaacactttcaaccaatttagttgattctgccgccttcaaccatttttgttgcatggttctttttccaactttgcatttaaccggtcctttaccaccggtcaatcacaatttctagtcatccaagactaaatcgggcctaatctattgcaccaagccaccaatcaactttaattatccataaaacgacactatctattggcagaagcttcattagtctttttcttcgtgagaccctttgaaacgaggacggaggaagtttcctttgaataatgcagcattttcaacatagaaaattctccaaacgcaacgcagaaatgacctctgctttctccaacgtcaaacttgtttagttttatctatgtcatctctctaattctcttgtaaatctcaataatacctcttcacttaatctttttaatatttctcttttggatggtgaggatttgtgttttcatggttttcttgttgttcttcgctccttctccagtctccgctggtttctatacaaactcctcagccattccgccgcagcttctccgaaacgccaccgggaacccctggaactcattcttgaatttcactggatgtcacgccggcaagaagtacgatggtctctatatgctcaaacaatattttcaacattttggctacatcaccgaaactaatctctccggaaacttcactgacgacttcgacgatattctcaagaacgccgtcgaaatgtaccagagaaacttccagctaaacgtcaccggagtactcgacgagctcactctcaaacacgttgtgatcccacgttgcggcaaccccgacgtagtcaacggcacctcaactatgcatagcggtaggaaaaccttcgaggtttccttcgccgggcggggacaacgcttccacgccgttaaacactactctttcttccccggagagccacggtggccgagaaatcgtcgtgatctaacctacgctttcgacccgcgaaacgcgttgaccgaagaggtcaagagcgtgttctcacgcgctttcactcgttgggaggaggtgactccactaacgttcacacgcgtggagaggttctcgacctctgacatcagcatcggattctactccggagagcacggagacggagaaccgttcgacggcccaatgagaacgctagcacatgcgttctcgcccccgacgggacacttccatctagacggagaagagaactggatcgtctccggcgaaggaggcgacggttttatctccgtgtctgaggctgttgatctagaatcggtggctgttcacgagattggacatcttctagggttaggccattcctcggtagaaggttcgatcatgtatccaaccatcaggacgggtaggcgtaaagttgatttaacgaccgatgacgtggaaggagtccaatatttgtacggtgcgaatcctaacttcaatggttctagatctccaccaccgtccactcaacaacgagacactggcgattctggtgctcctggtagaagcgacggttctagatcggttttgactaatctacttcagtattatttttggattatatttggattatttttgtatttagtgtagatgattggattagttcattaatttacagattattaagacacggtttggattatttttcctagattggttcttcattcatgcatagtttctctttttgaacataatagtatacttttttttttgttactaattaattttgagatgaaatttatgagttcaaagtataaagttcaaactaactttttaggagtaaagttcaaattattttatttaaactcaaattcaataacaattttaaatggaaatcactattgatggggaccattataactagaaataatgattacaatttgaccctggtaaagggaatttaacataactttggaagcatcgggtttgatctctcaactcaaaacacacataactttataataagccaaaactgattataagggaaatataataattacaaaaaggtaagttaggaacacaaagatatgttaattaattacaaacacatcatcacactatcacagtaacgacacatgggtcaatcagttcccccaccctacactaccctaaactttaattttcttttaattatcttttacctttctatataattctccattaaactccccagatcttccctacctcgggatacgaaaactcatcttataaagcaaagcatatccacgattattcccgacaacaacaaaacaaacaaaacataaaactctctctagcttggtcaagaaca

SEQ ID NO: 5 - DNA, 3000 bp, Arabidopsis thaliana
  AT1G24735 chr18754977-8757976 FORWARD (source_file=TAIR9_upstream_3000_20090619)
cttcttggaaaagaagcttccagcaaaatggctcgtcgtgatcctgacccgtatccttaaataaaagagccaacttataaaatctctctgcttgagtattgcatcatgattcaaatccttatatgatgcatcattgaatggtatcttcagaattgcaaaatccaaatccaagaatagcaatttccttgacattgctctagaatctatgaccgtgaatgggaagacgacaagaggagagcagagcggaagcggagagatcggaagtagagagtgatgatttgcagatcctttggtttgttcaacgaagagagagacaaatactggtattgaacactgcttatgttgtacacgtactattcaatgaccgtgcgggtctactttgtcatttggctccgccgagtttgataaatgacttgccagacttcagatatattcatagcaattgacttattcgaaatttttttcagatgatttatgactgagcttgtaaactgtagctagccagggaaattgatagtgtaacaaatatcgaattttgtttggggattttctatgtgagaaaaaaaagcatctgaagacaataataacaaatggttcatcaaattatatagctttgacctgccccggttttattaatcatgtcactttgctcagctaaacactgaaatagttacaacttttggtttgcaatggggatatgtggagttgtaatgttgattatgcccattgatttccattaacttcggcgaaatcaagagtaatatgtctttagttaaagtcacgcttttggtatataagctgatggcaaataattccttatgctgtaattttcctcggttgttatcagtgtgctaaggtagttatcttatccaaacctatggcttaatctcatcacaatccaatgatgtaaaggtagactaatgcagattgattctgttcttcttgtggaatagtcatgagtcagatttctgaattcttttaatgatttcaacttattgaattagtcagagaataatgtgaccatacaatctgtttgtttcaagttaaactaagcaagaaataatcatcaattacaagttttaattcccaaaattatcagttattcctattttcttaacaccttgttgcctgtgataatccaaaatcaagctcattattttgggtcattaatttattttttatgaaaccataagaaaatagtcacgaaaacaaaaacaatagagcaaaaaagggttttcaaagaaataagtactctttcagttcaggtctccaatttttttcagcatattgaaatctcaggagaacgaaagttttgacatcataagagcttaccccagataaaaaagacaccataatttgtaaaaacaaaaagaaaagagatgatgaaattgcctcagctcagttcaggtctccataatttctccgcatgataaaatctcggaagaacgaaagttatgatttgatcatcatcggaggcaatttctacacaaattgaaaaacatcgctagatttgaaagaattttatcaaaacccaaaacaacgataatagaatcaatacgcgtgaaaaaatcaataaagagagaacagaagtatcaaaacgactaaaagtgattggtaatgtgataatttcatcactgcgtcatttatatggctctggaagagggaggctgagctggggcgagtaaaccctagctctatctttgagtaaatgggcttttaatgggcctttgttattgggtctttcaaaatcagtccataatatttctttgtagattctttatttcttttactttttaaacaacttaattagaagtatgcatttaggatttttattattattatcgaaaatcatattatactcaaacgatatcaaagattattacacgttctaccatcttagagattacaacaatacgattaaaagcatgtagggatgcaacataagaaaaacaaaaatagaccgacgatgaatccatgagatttcggagacaaatattgagaaaacataacacgagaaagcactaaaatacactatttttttcctcttaatttgatgacattggtttgtgaatgataatggatcggagcacaggagaatccaaaaccagagtttgattttgcatttgttgatgctgataaaccaaactatgccaacatgcacgagagactaatgaagctggtgaaggtaggtaggaggaatcatagcgtttgataacactttgtggtacgggtttgtggcggagaaagaggaaacgtgccagagcacatgagggtgaacagaaaagtctttttggagttgaacaagcggctagctgctgatcatcgcatcgaggtctctctagtctccgtaggtgatggtgtcactctatgcagacgccttgtatgattaagagactcatcaaaatattaaatgtttctttagttttgtttggtttgggatttgaattgtcgaatgcttgttgcgatacctttatttttttctcaagttctgctccaaccatgccatatatatgtcacgctagcaactgtcatacagtccatattttcaatcaccgatttcaattttctagtgtaaatctcattatttgtttttggaaaattagtggtgtatgaaaagacttcttattttaaaaggaaaaataataatttgattacctaaatcataaaaatacacttattttcataactcatcatgaaaaaactttacaattagagtttgagttgaagcggttgtagaaaatgtaatatactacaacgtcatgattcttaatatcatacgacacatttttttaatagtaaaccgatcgtcggcggtggtttaggcttccatcataaaaaaaaaaaaacgaaagatgtatacacttgtcgagctgttaatatcattactaggccactacttttacagattctctttaaaaagagtaatagagacatgtaagttcgtgtgttacaaagcttttaaagttgaaggcgtcgagagagagagagagagagagagct

SEQ ID NO: 6 - DNA, 3000 bp, Arabidopsis thaliana
  AT1G29270 chr110227319-10230318 FORWARD (source_file=TAIR9_upstream_3000_20090619)
aaaacactttttttttttttctgagtttggtttaattaaattttgtttagatgttgaggatttagattggttcatatatagtttggtttagttcaatccggaccaaaatgataaccaaattcaattagtgattttaacaaaaaaaactacaacttaacattggtgttgcttccacatatacacctttttctttttttttctttttgggtttaacatacacatatacttgtaagtccatggttaacatgaagctgattatcaaattttgaaataccatatacaacaagaacggttaaaatggaggagatatagaaacagacaaggaaaaatcacaccaacgaaacacttatcactggaaaagatcaggaggtaaggaaccatgcagcaaaaaaaaattaaaaaaatttaagggtttgtatttttgtgtatatatactgttttcaatataatctctttatttaagatgcattattcagtttaacatagtgttttccttaattagtcatattgtataattaatcaaaaacatttgataaatatactgaaaaattaatagtgattaattatttttttctccatttcccaaagatttaacattctgatttgaaatattagtcttaacctattagggcatctccaaccataacaccattttggtattgaaataacactatatttagtgtatagtttttacaataaaatttggtgaagaaactattaaccctctattctttcatctgatagtggttatttagttgtttattttactttttagactgtccttttaactgagaaagatacgtggatataaatatggtaaattctctatgtatctattatccgatctcaagtaatccattttgtaccagatctaaaagttcacggatatacataaatgtggatatcgagaaacttaaatgtatatacttggatatttacatgtggtgtattaccaatatatatttttaattttcatttaatttgttttttcgacactttgtacaaaacatacgtgtattttcgattgattcggttttcgggatattaggatatatatatttcatatcatgatgacaaatattctgagtttatggatttataaacacgagtactaagtaagtcaagttaaattagcatcgattgtttatttgatgtacgagttcatattataatttttaatttttaatcatcacaaatgaaaatgaaaacgtaaacagaccattactaatttaaaaaaaaagtaattagactccatttatcttgaatatatgtgatactcatattctccaaccaaattccatcaaaaataaaatatttatctgatattatgaaggttaaaggagtacaaaacaaatagcagatcgtactagataaactgtgatgacctaaaaagaaacaaatgtcgagtgatgggttgagacaagactgtcatcttaatgttaacatctccatgtttttttgttgttttgtatccaaaaaagtgctttagtctattggtcacctcttcagctttcacattacacccaacgcttatcatatctctatctatttgtctacatttcttaccaaatgtctcgtgatagatcttcgtcgtgtactatccatttttggggttgatcatttgtcttttcctttttttctcttttgtctaggtccatccgtgagcaatgtttatgtgtatttttttaaattagtattttacaaatgattctaacgaataattttattcgccggccagttgtatatgtattgtgatacgccctcaaccaaaatttgtcaaacaattatatcaagaaatttggaaacaaaaatttcatacaaaaagccaatgtagcactacattatccaaaaggataaatttcatacatttctatatatatatatatataaaaaaaacatatacgtagtagtgtgatcatttatacgttacgacttacgatattgcgtttgctaataataggaaagaaaaggatagagaataaaatagaggaaatggtactaacgaagcatgtgaaacaaagcagcctcatgcttacaacttagtctgctttcttacgtacctttcgaagcgagacaaacgtaagaaaacaacccgtgactcctcttatctctcttccctcgacagatcttctcttataatatacatttattaatatttagctaacacacacattttgtattcgttgcttatgctatatatgtgtcggtaggattcttttggctaattcataggtatctatctgtctctgacaactggaattttgccattgttttatcctcctttcgcgttcgtcgatgtatgcttccactctctttatgtatttttcatttattttatgttcaaattatgtaagtctattgattttcgagttttaatttgtgatttgtttacaagcaaatgtttcttaagtacccgtgcttttaaagtgggcaagttttaaattatgattcaacttataagtgagggaggtaccatgtaaagagatatatataatctcagacttcacctttcaacttcaagataatggcctggtcggtgcattttgaaaccattgcatttgtaacttttcacaggcccaatctacaatatgaagaaaaatatatatatcttcaaccaccatacatattctaattatactttattatttaaaatctgattttgtttatgatgtttcaataagctaccagtttatcaccaacatgtatgtttggtacacgacaagaaatgtgtgtatatatatagagagatcccgagatatatcctttaaagttcaaacaatattaatcgttgtctcaaagagttcaaaaaattgagaaagtgatggtggctagtgaacaaaagaagagggcaagccaagggaaacctcattttcttaaaaatctcacccactttaaatttgtatgtacaacataaatatatgttcatatcacttgatgattctactattttaaaatgtttaaagccaatgagttattatgcagagtatacatgaacagagc

SEQ ID NO: 7 - DNA, 3000 bp, Arabidopsis thaliana
  AT1G30950 chr111033180-11036179 FORWARD (source_file=TAIR9_upstream_3000_20090619)
gcacatatgtactagacgcaaataatgtcacaaaaacggtttaaaatatctggttttcagtttgcatgtcgtacttataaccgaacgtgtgtaaagttctcatgtcatataccagttctgttaaactaatgaaatatgttcagaggtttaaccactataactattagacccgatttcaattaatcaagaaatatccaaactttttaatatatgcattatgctttacaaaattgtttgatcttctaatgatgtaaagacatttacaaaatataattatttaattgattctaactaatttgtccgcgcataacgctggccgtaaactagttcttcttacaaattacaatatcaatttccactttgggccggaaatttatatccataactctactacgtaccaattaacacatgtgtgtaaataaaactacataatggcatgaatggagtttcgaatcagttaatgtgggcgacaaaataaaatcagattatgttttattattaagtttagaaagagatgcttcattaaaagtttatgctttaatgttaaatcagactatttattatgggtacgacttgtaagcgtaattcaaagttaaacaaaacctagttgggaaatgggaagatagatgatttgataaaaacaagacactgtttggtttcagagacttcctttagcatcaaaacacaaacaaaagcgaagcctcttgagttactgcaaatagagaatattattacccttttgcgacttgtcagcttcagatattctcacttgtattattattttcacggtaaacaatgccttaaataagaaaccctgattggacttttgatctgactctactcttcactctttcttcttctttattttcagtcatgatgtctctctaaccctaatctcaaaaaatccaaactccttttatttatttctaaaccttgattatagctagcaatgattaatataagaattttttttctggataaagaattaattagaaattgagttgtaaatgttttgtgatgtctaaaattcttttgtttgcaaattagatgtaaattgatatatttgagatttgtatgaaagctagttttattttccctaaacaagaagttcattatcttggacttggaggttttagagtttgaaagagtttacaatttataagaaaaaataatcactatatatatatatatagtgtatataatgaattgtttcacattaaattgcaacaacatcaaataagggtaacatactaacatatagtttgtttgtttactctttaaaaaaaggggataaactaagagctatttctgctataatttaggaacaaactggatcacatgacaaaaatgcatcataattcatattaaattttgtgtatatctatttctcatgtttagaaataacattcttgtgtgttatacatgttatcagtttttcttcctagatggaagttttattgttggagtcttttaaaaccatactcactatgttcctctttatttgatgttttggattttagatagaaattaataaaaaatatgttttctatttttataaaattatttttttgttagtttagtaattttatttttctttttttattattagtcacaagcaaaaataataacaatatttttataaaacattaattttggtcgaacaagtaaaaataactcaaaacatcaaataattagaaactaaaaaagtagatattgtcaaattttgtgttgagttcgaataagataatgtggtctcctccaacaaaattatttagaataaatgcacttctatgacattagagaaccaactaatttatttagaataaaacacacatatatattaacatataaagtaattctaattggcttgatataatatataagtaaaaaaatgatcttaataatctctagttttcttgggttgatctccacgagtacaatttgactgaccattatagaagttgagaagcgtgcatgtaataaaagttgtatattacaaattagagaggaaaaagaaagaaagaaaaaagatttgaagatgtgatcaagtgtgaaaagtattggagtagtctccaaattaataatttcgatgctgggcattgacaagataactctgaagctctcaactttaagaccatcacttcctctgccaccattttcacgtttacccaaacacacacatatacaaacaaaattttgttagtcaataattatcaccaaactggggttataacaaggcttttggatacttgtgcttgttgatgttctaggttcgtatgataacaaagtacatccgttatatatattcgaaacacactttaatattaaaaatatatatccaattttcttgtgaaatttagattatttggaattaaacctatttctcttgtcttggccacttgaccggtttagttttttagacgtattttattatttctgtttagaaaataacaacatatatcaactgattttttacttccaatctctttttgtcagcacacaaatagaaaaacgtctgtaagctaagctatcaactaaaacattaacatatataatcttttacgttgatagaaaataaacataaatttctgagttattttttttttggttggtgtgtcactacttacttactactatacctttttaacaataaagaaacactatttctttttctattcaatataatatatgttttctatttgtataaatccattacctttgtttgttttataccaaatgttctttatatataagtatatgccaccttactctattgaagtcaagaacatatcaaaaacccatgcaagaagctcacagagaaagaccaaacacttttgtctctttcttcaaaacttttacatatgatctttgcctcttttcctacaatgggttttgcataactttcaccaaaaccctcctcaaaagcccttcacatattcccaacacaagaaaataaactctaaatccactttcaccaaatcttttcatttttcagctaaa

SEQ ID NO: 8 - DNA, 3000 bp, Arabidopsis thaliana
  AT1G31310 chr111200142-11203141 REVERSE (source_file=TAIR9_upstream_3000_20090619)
tcattgttttgacgtgactaattatagtggcatgcatgtcaaccatcaaacaaacataaataagacgagagagagaaatagagacatagcattttaatgattgttaaggttgtatgtaacgtcggtcttgttattgattcatgtatgcttttgatactagatttgatttatttatttaaagcaaataaactttgaaggtctaagagatatgatctttacgaaaagcatacacgatccatgcatgcagaactacatatgattcaatccactcctccatgcaaatttcgatagatcctttacttgtttaattttatttctttatttattttattgatatatcatcgactttgaagcaaatcatacttataaatatgtaggtataggtatagaatagacacacacattctcatgaaagacgtcttaaattatttaaaatttaataaaaacaaagtaaagatatttgaaagctttttgaaaattttttatttcgaacatatgtgatgttgtaaagtagagttgcaatatatcaacaagttatttgaaatcaaacatttttttcggatgtataaattacatgtagacaaggctcttcccatgacaacatgagcaataccactagtattagttcaatgttcttagtacagaactgataggaattactcagggccggctcaaactttttgaataccttgtgctgatttataaaaactgactttagttaatattggtttttaatttttgaaaattttggaccctaaaaccttaaagaaaattgaaaaaaaatttgaaaaaataattgaggactctgtgcttttgctcctctagcactcccctagagccggcactggaattactgatcgacttatcttatttgatacacacgtttgtttgttcggacttcggagtaatgtatatatatatatatatatatatatatatgtagatgaaaaaatacaattttgattttcgcgtataaatcatccaaaaaaaatttggatataaatatatatatttcaaaaagttggtcgtaattttcttatacattacatttcctaagaaatcatttgtttttagactttcatgaaagacttatatctctgtatcgtagacttgtaacgtgggaacttgattgtttacgtattattgtgcagacaatgatgaacggtctgtgtttttcatatgggatgtaataatatccctcgccatgcaatgcaacttacgtagtgtacgcaaattagtatccatatattattttatctattttgattagtgagaaagttctttagagcaaatgatactgtttaagaaaaaagaagaagttatgagtgcaccatgcaacgcattgatacatctaattactgtgagaaattatgaaaaaaaaatatttgtatgcgtttaaactttaaggtcacattgcagtagattttctaaaatatttacataatataaaattttaaccaacaaataattgtttggtgctagaaattgtagttgaaatgaaaaatcgatttgcattaaaccttttttacttttcacaaaaatttatatattgattacagatatgacaaaaattcaaatttgatataagatcttataattaccaaaaatgatatataaaatgaaatttttttaactaaaaattaatttcaaattttgaaggatatatatatgtagttatgtacaactttgtttgatgtcctttgaaaatttgcatgatgttatattcattctatgttggatctagctatgagactttccagtccgaggaatgtttcatgtgtgtatatatttctaaacatgttttgtgtaagaaacctaataagaaatccctatacaggagtccaattgtcatacaattatattaataaggaaactaagcattcacatattctacaaacatacatgcgcataaataaggttcaaggcaaagacaattaaaaaagtaactatactcttctacttatagctcgtgcacatgaattagagattcgtaaatcaaatcctttttaaaaagaaagtatgagaagcaatcatcatgcaaagccaatagcttttcgaagtttgaaaaaagtatatacatcactttatttgttttattaaaatggaaacgagatctacataggatgcctccaaaatcttccaatttcttataagtttgaaaacagatacatcactcatgcaattgccttgttgaataagaccgcttatgaactatttgtggtcctaaactaataaaatttatcgaaatcgcattgttctcatttgtggttttcttgatattttgtcgtgtatatatacacgtctagctctgcctacctttcataacatagaaaacttatggtatttaaatatgtagcaaatggtgattgaaaaacctaacaacaagaaaaaaaatcaatagatatgttcttctcgattttcatttactaatttctcatctgattttcataaatatagtcacaataacgttgctaaatagtcacaaatttgtgaccaataatgttggtctctaaatttatacgtaccacatttacttcgtttttgataaatctacaaatcacaaatgttcaccattgtgattatttaagaaacagtggggaagtagattttatgaaacattgaaacattaactccatctatttgaaattgtatacataattagaaatgctagattcacctaagatttcctgtccccaaagaaattaagagagacatgaatgaatgaacattttataagaaaatgaaagaaaagaaaatggtcagtttaataaactaatgaatgaatgttgccaaaacaaacaaacaaaatttaatgaatgaatgggttacacattacatagttttcttttcacttggctcccaatattgcaatacttgtcctttctcattgcacttggtgacaatcttagtactacaacaaaaagattttgaccagtcttctcaatcacacacccacaaaataaaataataatcagaactatccccaaaacttaaacacac

SEQ ID NO: 9 - DNA, 3000 bp, Arabidopsis thaliana
  AT1G37140 chr114172042-14175041 REVERSE (source_file=TAIR9_upstream_3000_20090619)
aagaaaactatgaaccagagactcttgaagcatcttttggtgaacgcagatgttatttagcaattagcaccattaacactaagataaacaaactgcctttatatatactgtggatattgtcctttgttgcaacttttttgtgtatgttttgactcattaatgttccttgagggttcgttgaagaggaattggaagcgttgatcatctaattatctagttctgtaattagaaataatgatttgggtccaaactgtttattgaaaatattctcaaactctcttcttattaatatgacaaaacttaagcaaattgtacgtatattatacattcatcatacctaagctattataaacaactgacaagaaagttatttttttgtaatattcacaattttatatcacctaatctgtaggtagtcgtcacgtactacaaatacatggtgatattatttttaattatgatacaactaataataatgaggaacaaacaagtgtttttttcctggggaacatgaaattcactgcgggaataagacaaaaactgtatatttaccttactcgttttcttgaaaacctcgtttaataattttctgaaaattactgtaaaatcatcattcaaccgccagtaaagtgtattcagaaaatcactttcaaatttttttcaatcggtgcaacttatacttctccagtgtcatgcaatttttcttatagttatctaaaatgtgaaacagagcaaagaagccttacattgtcgtttggtttaataccaaaattaataataacattgtatgattattttgtgtttccaatagctgcatgtaaaggtaatgatgcccaaaccaagggaagacttttcacgtcactaatcttctatcaaatctatcaaacccatacttgtactcagacttctcacgtttgtcttcataggttatcatcacctctttttgcctactcaatttttatttatttttacttattcacctaatatctatttgagtacacgttttatgtttcatgatgatactcatattaataattacaacttacgtattttagaagatcgactaacgtattttgtgacacgttttatgtttcatgatggattcatttcatgccacttgtttaaaactcagttaattaagaatcatttgaagacttatcataaattgctatgttttaccaaatatatagtataacttttttgcttacgttgtatgttttataatcaaatcaggagtcagattttggtataagaatatatactcaaataaacaaaaagaaagactcaattactgtttagttacgtcaatacgagtgatcagaacttaaaagagaagtcaaatatgtgaaattgaccttagctaagaacttccaaaaaaagaattcaaaccccagtggcaaaatagaagcaaaaattagctttaaaatcaaagcttttctgatcaattttttcttgggactctgcctctctatctctctgcctccgaattttcttttacctttcacatgagtaaagcgactcacaaagacaagtttcaatgaaacaaagaaagttatcataaaataagacaaaacagaacctaaagagacaagacagaaggtacatactacatctctgcgtctgagaaacaaattgcttttgttttcttgtcttcttctagaaaatgctactcttatttttccttggatgttatccttttgctttttttttctttgttatttacttatttatatacacttggtccttccattttttcttttttggtaattgtccttcgaattatttattttataaaacaaaaacataatttaatttactggtatatttcactctgtcactcttgttacattgactgaaattatttattttataaaacaaaaacataatttaatttaaacaaaaagaactctatgaacacttgtagtttcctctaaactcctctcaactcgagtacccccatcattcgtcacacgaactgatgaacagaagatcattataagagacagtctaaatttcttttaaacgctattgataattttctttacgcaaatataatttgctttatttaaaactacttaatccataatacatattcctgactacacatttatggcataacaatagattgaccaccaaacggacacattcacttccactgtttcacctgaacaactagtgttttaaaatagtatagattcaccaactctttaacactataaaattacaatctttagtttgatacagttttaaaaaaaggttggatattgtaacgtaaatatgatcagtagtttgcatggaaagaaatgaacatggaacaattgcatgttcataagcataccatttcatcttaactatcctaatattcttttgattttattctatttcgatttggattttattaatatagctaaaaaataaaagaatctaaaccaaattaatcaatattttcataaataatagcataacaaagggaatgatagaaaaaaagtaactacacaaatttgaaaaaattcaaacgagattccaacagagaaaaagtgtctaaacatttgtcctacgattgcaatttgaccaattagatagtacaaatcagaagatgtctcgtcgacaatacgattgatagcccagtggataaactctgcttctccctctctcaatcatgccgccagatcaaaaatattcaatagaccataaaaagcctaccatttcttagtattgggacaagagacattaaatgcaaaaacattaaaaacaaaataagaaagaaaaaaaacctaaaaacttgtctgaaaaaagcttctactctgattcttcttttctcttttctactgacacgttttcaatcaccttaaacgacaaatctctccacaacaagacacgacacaacaaagcttatcttctcaaaacccttcactcacactctctcatctctagggtttcactaacacaagcttcctttatccctctctctctctttctcttcctagtttctttagcttc

SEQ ID NO: 10 - DNA, 3000 bp, Arabidopsis thaliana
  AT1G49320 chr118243305-18246304 FORWARD (source_file=TAIR9_upstream_3000_20090619)
tatatatatatatatagcatccgactttacagtaccatggtaaaataatacactgaaatcttataaggataatttttaaataaaaaatgttagcataaattgacattatttgtaaaatgaaacgagaatgtattatactcgttcatatatatgttatcatttcatgtcctcaaagatgtagcaaatgggaaacaaccaaatattgtcacaataaatcaaaaatatttgagaatggagataaatatcagtattcaaatattgtcgtttatatttttttgtcctttcattcattttcctttaatatatctaatcacttgcttggcagttaaatccgaaagcaaaactacaatatttttggggtggtttagctcataaacaaaatgaggtaaatatttagctaagatataatagggttctttatcaaggaaaaaaattaaagaaacgttacaaaaaggtctaaagttcgaaataataataagaaaaccggcatttgcttttgttttgcggttgtgataccttcgtcagttgagtttttgggcgagagaaggaatgagttgctagatatttaatgcttcaagaaatacgagctcacatgtatttatataatctccaataattgtctctatttttaaaaaaaaatataaatgaaaaatagagatttctatttttactctgatggtttctcagatagatatttatttgttagaaaaaatattaaaaaaaatagaaatctctattttgttttttttatctatagagaaaaaaataaagatgtgttggagtaaaatataggtaaaaatagagtaaaaaaataaaaatagagatgggttaaaaaactcttatttgatatgtttgtctcatgtattatgtcggaattgattcaaaactgaaaaatcgtgtgagattaatttaggcaaaatatttacattgttttggactttgtattgatttaccgtaaaactaattttttctgattcatcttctcccaaccaatttactcatatttttgtttgcactctgcaatttcctattaatgtcacacaatttattttcttaaaagtatatgaatgttagacataagaaaagcagatttcaaacaaatcttgacaatggattaaaacccttattagtttgctgtgattttcggcacattaaaattctcttaaattcgttattctttttcttttgtaaaataaaaccgcacatccatatgctaaaaacagattccacgaacatgaatattcattctgattttcttcttcttatttttttgcaattcggatgataaaacataatggatgatgtattaaagtcaaagattttatatagtgggttggttgtcgccttaggattagagtgactatgtaaaaaaaataaaaaaaataagtcaatgatactgcgactaagttcaatgaaagatcaatgagctttttattcagttattttttcttattattatttgtggtcctaattaaattttaaaatatactttttattgttttaatccttttcaattcaccaaaggtccatcattcacattgtcaacaaaagatttattgaaaagttataccacaagacttcgtaaaaatgaccaactttcctatatataaaaaaatagaatcagaatcattaatacaaattaagtacacacgcatatatatatgacatatacatatatataagagacaaatttgagctacacaaaatatctacaaatttataatatggaagccgtgattttttttttaatttataaaaatattaatttatacagcgatttgataaattttttaatttttaaaacttttctaaccaaaatcgttataatcaatatatttttaagacttattaaaagtaaaaaagaataattctgtctaaaaacataatattgttttgatgaaataaaaatattagaaatgaactctaacaataactacgacaaatttaagaaaaaccaacaataatatatagtcatatttcactaaagttaacacaattgttaatttataatatcgatgaaagtatgtatttatataacaatattctaaaaaaagtattatcttattaatttatcgaatcgtattaaatgttaaaatagcataactcagactagaattttttttttatttacagtaaaatgaatattaatttatcaaatatcaatctataaaagttatactgtataaatcatccatcacaaataaatattagtctcaacttcatttaacaggtttctaacctctccatcatattttttttttaataattcacactcacaatatatcaacacatacttataataattttgagcttctttttttctttttaaatatacaaattataatttttacttcatctactgagatctgaacctattatatatttgacgcaagtatcgaaaagttttttagaaaacactcattgtttcttatggctgattgttttttgtttacgtatatccatatatataggccttagcgttgaaccccgatagatcgtagagactacaagtaaatgttgatacccaattttttgcacgtataatataagaattattttccaattaaaccttactatttacagatgagtctagaattctctatctatttttaaaaaaatgattacgaccacaactaatatacaaacaagattcatttctttctttattttatttttatttttcaattacgacgtaaaggatgatccatgaactatattgtttgtattagagaatccaatcaaaccaatctctttggattaatatcaaattattttatccccgtatcttttacagtttcaatttagatgacgatttaagaagctgcaggcgtagcaattattttaataattacaaataaaattgagtgaaaactgtcaacagtctacttatgtgtaaaactaaataaatgaataaatcctcgaaatttaaaaataatgaagaagtcagcatcttcataatggattattataaatatacacatagatggatgttcagacttc

SEQ ID NO: 11 - DNA, 3000 bp, Arabidopsis thaliana
  AT1G49475 chr118309920-18312919 FORWARD (source_file=TAIR9_upstream_3000_20090619)
tcatatacatataaattatacattgttgtttgttgaaacaatccttccaaccaattgcactctccatatatatttttctaactctacattcaaagaagtcacactcgactttgcatctctaaacttcgtgctaataattaagtgcatgcacaaaccttttgcatcactatctataaaatattaaagtttatatcttttcaaaaatgaaacaaaactatagagaaaatgagaaaattaaagcctttaaatgaataaaaattacacacgacgttcctctctgcttttcaaatgctttatgtttctttatgataatggatattcagtttccgtttctgtttctttttgaaaaagtttttcttttttctttttgaagaaagaattctgtttctgtttctttaaccaagttttcgataacgtctttaatttcttcgccagaattttaaagtggtacatattggtgaagccatcttttcttcttctttttcctcatcttttttagtggtattaaccgtcacgcaaacacatatgatttatcatccatatatataaacacttcttcaccaaaacctcatacaacaccacaattaacagatttattgtttttggaaataatcaaaacttaaaaactatcagaatgggaacgttcttaggacactttgtgcctggtctctcacttgctcttcttggtctttggcatctcttcaacaccataagatcttactgtcttaaaggccctgaagctttctctgcaaaattctggttccctttccctaaactcaaacatcttgagcttatcttgattctcttcttctccttcctctccatcactctcctcaccctagatttccctaatttcaacttctcctctctcaaaccagacaacctagaacatgcttccatgttcctccacctcattatctttgcgtgtttcgctctcttctgcgagctaaccctttgctcagatctcttctccggtctaatcggagttctttctgcctctgtcttcgctcaagaactcttccttcttcattttcattccactgatcattcaggtcttgaaggtcactaccatttccttctccaactcattgctttcgtctccttctcctcggcgctagcttctgcttcgttccctaaatccttctctgcggctctattcctcccgatatcggttatgtttcaaggatgctggttcttgaacatggggttcatgctttggatcccagagtatgttcctagaggttgcgtgagtaacatgtcaacatctacagacaataatagaaggagtgtttatcatagtggtgcggtggcttgtgagtcccctggtgctgagattagggcaaaagcattggcgaatctgcagtttagttggatgttgtctgctatacttatcattacatgtgccttgtgcctcaagtattccggtaaagttgtgttgcctaagattaggtcatcgttggagtacgagcgtctttgccggcaaggaagtgatcgttctgcaaccgtaacggtggaagcaccggccagttccgatcaacaatagattttccttactttaatttatcatctgaatatgatgtaatatgtacatatagtacactaataagtagccctaataatagttacataatttataagagggggcagagtgtaacgcactaatgatagtggaccctttcgcatgctttgtgaactaataaaaatttgcatatagcaacaacttggttacgagtttgaaaattaaatttaataacttactaatatatccctaattcttgctaaacagaaataaaataaataaattaacactaaaacttggttgtagatgataataacaaatttatatgttttatgtcaaaatataaccctcacttaaaatgatatacctatgattgaaaggttatttatcaagtacatttaaaagaggctgtacaaaaagtacatttatataagagtatacgttgtgttgatgaaatattgactgctaaataattgatcatgtataaccatttattattttcgcttggccgatatcagaaatgatggaaagctttagtgcaataacaaattttgatgagcgtattatcgatgataacactcaccagaatatattttattttttttactatatagttgttaagtacatgtaatagttttggtatcttctgcaacgtagttttgcggtgaaaacttttgttttgtttatgtctagcaagaattagtgttgtgtgttgaatgttgatcaataagatatttaaatttaattttcaagcccaaactatcctttgttaacaaaataaaataaaataaaacttttgattgagcagccaagtttttatcattgcatatatgatatatccaccttacctccataaattaaaatacttcatatatatatatttagttgtaatatgtagggtgaaagaaagctaacagagttaaacattagggtcaaattattaatttaggagataatatattgcaaattaaactatcagtttaatgcaaatctacgttttacgtacaaaaaatgcaagattacatccattcaactcattaaaactcatgcaatatagtaatatacaatagtttttgacggtaactaactgaatttcagttactcaaacattttatgcagaaagatttttatgataaattagaaacaaacatttctatatagggaattaacatcacgagtcacgaaaaaataatcatatctccacagtttatatggttttttacttctttttttgtctgatcaacaaaaattttagtttatttaacaaatctagtatttacttcatttgtaccgtttttctagtatttacttcattttcagtataatcttctttactttctatcaactttaatatttctgattttagttgaaaaatttattacaaactaaataaaggattcttgggaatctaaaagacttttaactttttccaatgtgtaataaactaaataaaggattcttgggaatctaaagtgcttcct

SEQ ID NO: 12 - DNA, 3000 bp, Arabidopsis thaliana
  AT1G52100 chr119385534-19388533 REVERSE (source_file=TAIR9_upstream_3000_20090619)
acgaagtggtccgaaaggcattcagtacatcaagtttctgtatgttgacaagtatggacatctgaaagatgggccaatccatggttctatctcaggaaaaggttccatacacttggtatgtattcatgtcatggaataatattagttagaaatatgtttttttttcaaaactgaaccgcagaaactaaaccgaatccaactgctgtgtggtttggttgcaccgaataagggtattttggtccgaaaacgattaaaaaaacaaacagtgaaaaccatatctcactttgacaaactatgagagtaattcttttttcatccacttatgtgaaagtttcttataataatattttacgttaattgtattttttttagtttgagatctaacatgtttacaaggaatatctggtgtcagttgagggttactacaatgatcatgacgaatctggagtcattcaggcacttcgattcaagactaatatcaagacctctgaactaatgggatccaacacgggtaagaagtttagacttgcagccagtgaaatgaagattgttgggtttcatggatacgctgggaagaatctaaggtctcttggtgcatatttcactccgattactcctacaaaattggcatggcaaggtactgctctaggcacactttgggatcatggtgctttccaggctgttagaaagctttccgttttcgagattggtggttatataacgtgcctcggtataacctatgacaacgatggcaaagtagaaaagcgtgaccacggaatgcaagatggttaccctggagaagaagtagaggtcagaattcctataactatatattcgttgtttaacagaaaataaaataaaatacatccctcgaccatatatatacatatattgttggatagcttatctgaagctccttttattgcagtttgtggttgactatccaaatgaatttatcacgtctgtggtcgggaccatgagtactgatagggtagcatcattgacttttaaaacatcaaaagggagaacctctcaaagatttggagacagaactgctaatttagttgaatttgtccttgagaacaaagattgtgctattgtcggatttcatggatggtggactcctagttatcttacggctcttggtgcatattcttttccgatgcctctttctcctgcttcggaaaagctagaagcacaaggtggtgatggaggagcttcgtgggacgatggcggtaatttcgaaggtgttagaaagatatgcattgggacaggtgagattggtatcgtttctgtcaagtttctgtatgaaaatgacactcacgagatagtagtgggagatcatcatgggaacaagaacctgattaaacatgaagaggtgattactacttcacttttcttaattaattgttcaattacgtaccctaacttagttatataattgagcttgatattattatacttgattttctaatttttatttgtagttcgagctggattatccgggtgaatacctcacattagtggaaggtagttatgacatagtacctggaagtgaggaaactgaagttataatcatgctcatgtttacgaccaacatgcgagcctctccatgttatggactcgatgataacccaagctttgtgctccaaaagagaggtcacaagatcgttggattccatgggaaatcaagtaaaatgcttcatcaacttgggatccacgtactccccatcacccacccttgatcatgtttatatgacttagaacgaaacattctcctcctatgttttcattaactttgattcgtttctcctctttctttttttccctctgttttgattttgtttcttttttctttgtttgaacttttgaataattcatgttctaagaaagtgtatggagttggagtgaagctcattgtgacttatatatctgttttcaataagttgtttccaaaagcaagaaccattttgaagattccacgaaaaaaggatataagctaaatttgttttttatgaagttcatttttttggtatataatcgtcggtaacacaatttcatcaacaatttgttggtagagtactgataacaaatgcgtcggtaattgttgttaggttactacatgccaggaattttttggcaatttgtcgacgtgagtttccaacaactaattagtaaaacaactttcatactactattaaccatctctatgaaaatcctaaagattatgtagtgaacttagttaacttaaatttaaattatttagttgcaatctagattttttttttttttttttttatggttctaataatatttttattaaaagatttataattcaaatcaatttaatttttctttttaaaatagttatttaattttataaattttatatattatatatattctaaacgtttttcgtcaaatattctaaatattttataaattttagataattataatatttatttttaataaaatataatattaataaaattttaagactttcttttctaaaaatagttagaattgatataaatatattaaataaagaaatattaataattataaatattaattaattgataataattaatgcgaatgacattctttttaaataagttacagatttatttgccaaaacctgttgttaaataataatgttagatcgtacgttcaccacgttacgatgttgttccacgttacgatgtactattgttccacgttacggtaagactataaacatgtttagatgttggaagtttaaattgtatggcctccctcacgacacacgagcaaataagaagatggtaacttaattaagattattatatagtctcgttggaccacggatgatataaattcaaaaaccttatccagacagtaagaacattaaaactttatataatattggtctctcgtggatcacgattcatatttaaaaaatcatttctaacaatgaaactgtgtataaaaaggcaccttcactagtacctgcacatc

SEQ ID NO: 13 - DNA, 3000 bp, Arabidopsis thaliana
  AT1G60540 chr122304986-22307985 REVERSE (source_file=TAIR9_upstream_3000_20090619)
ctgtagtgcttcgtcgatggatgatagattccataaggttcacggtgaagttccgacccacgaagtcgtttggaaaaaaactgatttctttggtgaaggagataacaaggagtttgttgatattatttatgagaaagcccttgatgaaggcattgcaaaggtttgtgcttgtaaagtgtttgatgatattcctgagagagttatgtatttggtgaagtgggttttgaattgcatagtttttggtttctggggtttatgtagattactataaaccggccagagagaagaaatgcgttccggcctcagactgtgaaggagcttatgcgtgcgtttaatgatgctagagatgatagctctgtaggagtcatcatactcaccggcaaggtaaagctcttggtatttgaattgatatctgctagttaaatgctatgaagcattaaaatgtaatagaagaactatatcagagttaatttgcgtatgtgtatgaacattgaagatggagtctaggaagaaaaatatggatttaggttattttacttaagtcagtgtgtctagctatgagctttgtgttgagtctcataagtgccaagatcaaatcctacatttgagttgatgactgttgacaaagagttcgattgtattcctatttttttgtttttaatttgtttgtgaaacgattgttctgaatagtgatattgatggtttagggaacaaaagcattttgcagtggtggtgatcaggctttgagaacacaagacgggtatgctgatcccaatgacgttggccgccttaatgttcttgatcttcaggtatgtccttgatggacaagacaataaaaagtttgggtttcctcatccttatcgggctcatgtattttccaccttccttcttctgtttctactcaggttcaaatccgcagattgccaaagccagttattgcaatggtaagtaccttcaaacaactgctcgtttactgtatgcatctttgtactaatcctggattagctagttcctgaggttgatctgtttgattttggactgagtcaggtagctggttatgctgttggaggaggacacattttgcacatggtctgcgatctaacaattgcagctgataatgcgatttttggtcaaacgggtcctaaggttttccctctagcttctgtttcagccttgaattgactgtgtaagtgaagtaagtactagagttattactaatccccactgattatgtggattgacttgatgtctgcaggttggaagttttgatgctggttatggaagttccatcatgtctcgtctggtaattgaactcactattactgcaatgcaatatccaaaatctaacaaacataggctaatcaaagtgtatgaatgtatacaggttggtcctaaaaaggcacgagaaatgtggtttatgacgaggttctacacagcttctgaagcagagaaaatgggacttatcaatacagttgtaccggtaaattttaccagtaaaccccatagtgttgtcacagatacacaaaaaaatcagtctatttgttggtaattgttttgttccttcactgagacctatttttctgttccagctagaggatttggagaaagaaactgtgaaatggtgcagagaaatcttacggaacagtccaactgcaatccgagtacttaaggcagcacttaatgcagttgatgatggccatgccggccttcaggtacctccttcaatcaacttgcttagtctttttccgttctaaaggaacaaaatcctattagtcaaatttgcaatctaagtcacaaaatcacaatttggataggttcagttcttatgcaacccactagcacacagttttttaacggatttacaaacttttcttgctgcagggactcggtggtgatgcgacactcttattctatggaaccgaagaagctactgaagggagaactgcttatatgcaccgccgaccaccggacttctctaaatttcaccggcgaccttaaacacatttctagctctgttcaggtgttttcattagagcatttgttctcattccaatccaagtctcttttttttttctttttttttttccattttttttatttgtttaccaaactatgaattaacgattttatgaaaataaaacgggaaacgttcaacattaaaaaaaactcagcattaccaaacagaaggaaaaatccagagttcaagagatcacaaaagccttgcctcccattgaaaattcattccaggacaagaagttactttaatctctaaaccaaaacgataattatcaatcaatcacatacatttgcagtggtgacatttgcatgtggtgaagagtgattgaaatgacggaacgaaatgagcagagcaactcgttgggaagtagagtgctgctgagtgctgacagatactcatgatacagataaacaaatttacatttacttataatatagatttatacaaaatatgaattgttgtaaactgaagttttttaccaaacaaaaaactgaaaaaaaaagaattgaatgtaatcaaaattcaggctgatgagagggagtttgtttagttctaattatgaagcgaatatgtatgtcagtaacatctatgagattccttctgtgagctacaacttaggttataattccaaatgtcttgtctacctgaaaatagtaaaatattgatatctcacacattcattatattaaaggatttgagagaggcttttttatattcaactatgagctaatatctataggtaataggtgtaatcatgaacttaattaattagctttgttcagttccaatatgctttttacttacgtcatttcatgtcaaacgtctgcttctgtagtatagtgctcataaacacaaagcccttcaaatatctcagtatatatatttgcatatctctttgataaatctcaactacgcttatctcacttacttctattctctatctcattctattctactcttgtattgtctcctcaagcaacaaa

SEQ ID NO: 14 - DNA, 3000 bp, Arabidopsis thaliana
  AT1G60630 chr122336909-22339908 REVERSE (source_file=TAIR9_upstream_3000_20090619)
tcgatatgataaaccctaattactcaccgctaatccaaaggtcagacctttctgcggttttggcgattggtgatggacaatgaaataactgcagaggattaacttcaactcaataagtatatttattgggataaaatgttgtccactcaatgactaaaacacccttataaaagttacttaatttacaaaaataacctaaaaaccctaaaacccttctctttagcaaagctttccccttttcagtctccaagctctcttctgcggaactcacggtacgaaagtctcctcttttctatttactttaatccgaagtttgctcctttacttttgatttgttggagaatttcgaattctaaatttgggttttgtctctgtagagtaaacacgaagtaaaaatgataaagagacgattttacaagcttgagcatggtgataaagacagtggatccgattcatcgtgcttctcttctgactctgatcctgagacggaagaatcagaacaatctgaagaagaagactctgttgcagaagtttccgaggacggtgatgattcaggagatgatgaatccccaggtttgcttcttcaattgagcaattatctcttttagttctcttgtgattgtactctcagagaacctttggtaaaatctttgcttattctctgttatagctgctggcgaagacgctgatgttgatgatggtgatgataactctgatgctgatgactatggtgggacattggagaagatgtcaatgaatcgttttttagaagaaccacccgaagaagaggaagagaactatattctcggttgtatgatacaatccaaatcggtttataaatgcagatattgtccaaccgttgtttgcttgaatgagaacacaatgcaagcacatgtttcatcaaaggtaaattaggtagctttgaattagcatttgagtgtttggtcctttttggaatcaagtagtagatgataagcacgggattagttttgagcttttcctcatcagtcttgtagttgatgagatagtattttttgtcgtttgactcatttcagaagcatgctcgtatggagaaattagtgaaagaaggaaagattagaactgatgacgaagaagttgacgatctagagacagcatctcaggttacattagagaatttgccaacgagtgctttttgttttgtcaatgagtcctaaccaaattcttgcacaaatcttaaatgtaggagaaggagaaaaaaggaaacagaagatcacaaagacagggaaagagatctcaaaaacaggaaaaggtaaatgtagcttgctcaatactgaagcattgtgaccatggttggtttgttggaaacctaattgattcgtgttttcaatataaacgtctaggattcattgaccaaaaatggagaaaacgaagaagttgaagatccagagacgccatctcaggtgagatttaagacaatgccatcaaatgcttgtgttagtccaatgagtcctaaccaaagtcttgcaaatatcctaaatgtaggagaagcagataaaaggaaacagaagagctcggagagagcttaggagatcacaaaaacaggaaaaggtaaaatttgcctgctctataaccctgtgaccatggttgacctgtcaaaaaccttatctattcgtttcttaatatgaacatccaggattcatcgaccaaacatggagaaaacgaagaggttgacaattcagggacgccatctcaggtttttctttttgacgttcgagcaaatgtcaacaagtgctttgtgttcttcccatgagtcatttactcaaccaaattcttgcaaaactactaaatgtaggggaagcagataaaagaaaacagtagagcacgaagacagcgtaagagattagaaaaacagggaaaggtaaaagtagcctgctcattactgtaaccttgtgacgatggatggtttgtctgaaaccttattgattcgtctttttaatatgaacatccagggttcattaaccaaacatggagaaaacgaagaagttgacaatccagagacaccatcacaggttttctttttgaattctagcaaatgcaaacaagtgctttgttattcttccgctgagacctaacaaattgtttcaaaaatcttaattttaggagaagcagataaaaggaaacagtagctcacgaagacagcttaagagatcagaaaaacaggaaaaggtaaatagcctgctcataattgtatccttgtaaccattgttggtctgtcaaaacccctaatcgattcattctttcttaccaatatccagattccatcgactaaggaaggagaaaacgaagatgcatcacaaaaatctagaaagaaaaggcgtcaaactaaggattgaaactgctgtattgcccaatatggttagccagaaagattcatctttttttacttggatgtcgatgaatagatgaagaaacataggttgagagtgagttaattatgtgctttttaaacttcaagttttgtctaccaaatacggtgcttgttataactgatatgattttgataattgagttatacaattcttttttacaagttgtatccttttttcatttcgaaccatccccctcttactcaacgagagagattgggaaataattgcttgaagcagaaaatcgaataacagattagttgaattaaaaatatgtttacttgaaactatgttcctccaataccatcaatgatctctggcatttccctccaatttctgtcgttttctcttcccaagtcagcactcagcacttgtcttgagaaagttcaaagacttgagtgaaaaagacaaaaactttaatattttcttctcttcttttaatcttggtctccaccataaacaattattgatctacatatctgtgtaaaacagtggaagtgtacacacgaaataatatttttatgaatgcattgaaaaaggaaacaagtgagacaagtgagatttgattaacttctcctactgctcttaaagt

SEQ ID NO: 15 - DNA, 3000 bp, Arabidopsis thaliana
  AT1G64625 chr124013656-24016655 FORWARD (source_file=TAIR9_upstream_3000_20090619)
gagggaactgtgggagtatataaagaaaagtacatatgtaatggtggtttgattaaaactaaaaggttgttttactttgaagacatcagtacgttttattttcccagtttgtaagaaatgacacaataattgaaattgatgttccatcaataaaatgtgattcactatcttggtttccaatggaacacaaaaagaccaagaaaaaaggtgaatcattatatacgataacaaaaataagaaagattaaaataagttaagccttaaatattagaattaagataaattacgaaaacacggatattacatgaagtcttaaagatgttgacctcgaggggatgcatgttgtgtaaactagaaaattgatttttggagtttttcttcttcttattcgtatcaagttcattattactttcatttgtcaaccaagaaaatagttttaatatcactaaacttataataaaaataaaaaatattcttttttaaattgggtgaaaaatccaaaacatcattctttttaaaacatggaaagtataaattaactctgaaactcatttctattttatgtagactagggtatagggctaacaaaaattggtctcataatagatatcgaacaaaagacacagaacctaaagcaataacaaaggagactaataaccacaaatttttctcatatagaaaaagtttacatattctccatagctattttttcgaacaaattaagagtcaaatgcaaaagattttaaaaattttgtgtgctttcaaagatgaagtttttagcggttaaccatgacatataaattcgtaatattttgtagttgtttttaaaaatatgattggattatataataagttttaccttgttttccacaactcattgaataaagaatttgtcacataaacaatacatatttgcaactgtcatagtaaaaatatattatcaaatctaacaaaaattatgtatattatgaataaaatactctaaatttaaagaaaagtatgaatccaacactatctctatcttagtacatatatatgtggactaatggtagttagttagagttactcttaagttttaagcgataaaggaattttgttttgtttttgacagaaaaatatgttctctagtcacatattttaacgaaaagttttaaaatagcttatctaacaaaaagcaagtgtacaacataaaatccaacttttggatttatttcaccactttcggacacaattgtaataagaacggaaagtttcgaaaagggacaataaatcaactaaagacgaaaaaggattaaggtgaaaatcttgatgccgttgacttttgttctcaacggccatgattgagcgtgtgatgagtataagagaccaaattatggctatttctaataaccccaaaaaataaattgattgatgagtttttcttcatttgatatgtaattataatcacttattccatttcaagatttgtggatatctattgatattctctaccactatcaagaagctaaaagtgtattaagtgcatgtacattagcttaggccgagaagttttttttttttttttttaaaggtgatgtatttgtggcacttaggcttaggcatgtaatgtgtggtgtgagatgttgcgcgacttgcgagtaacaaaaatgggtctcaaatattgaggcatgtatttctaacgtttgctatatatgcctttgatgttctctaaattctattttatatcgtatttccatagtaatattcagtaaaatcatgaaattcgaaggttgatatcagagtttatatagtataaagttttaagatataaataatccattgaaaactcaaaacataggtacaaaatagcaccatgtacaaataaatagctaaaataaagtaataaagtaacaaattaaaacacgtttaatactattcatgaactaatgttcaagtctatttaacattcacatttttccttgcttgtgatcttctatataaacttaaaattaaacatgcatggtttcctgataataataagatgtttagttatattaaagaaaaaagatagtagctctcaagacttaaggcggaagcataaccttattttttttaggttggtaccacaagagaccacaactaaacatttggatgaatataggtataacaaatctcttatgatatgtatatatctggcttatatatgaatgcatactctacgaatgtatgtgactgctgacgacattggtgttagactgttgatataattatatgaggcagtggatcgagatttgagaactacttaagtcaagattatcataccgagaattcaagatttaggtgacacatattttgggaacctcattggcccatacactaaagaggcggtcattctttctttataactctaaaccatattttccaaccaaaaacggtcgatgttaagctttaatatgatgttcaagatataggatacgagggagaagatgatcgctagaaactccacagcaccatttatcttcctctgttgtggggatcacattctcctcatttttatttttccattgcaccagtttccactatagaaattttcatcaaatgttagttaattaatcatatctaaatatattgaatactattaaagtatatcataaattcgtatcgagtatttaatgaatcgatgataaaaagtaaaatcaaaataaaatctccctttatggaaattataccctcgaagatcctctcccgtcctaaatttttcaaaagacaacaaataaatatatagtaatcaaagcaaactttttcactaaaccggcaatagccggtgtaattcacgtcacttccgggacaaaagcgtgactggacaccggcagacaaatcacatcaaataaataatagtaatatatttaattcctcagacaaaaaggctcttccacttcttatcgaattacaaacatttcatgttattgtttggttatatatatagagagacaaagctcttaactcctttttaattgcttg

SEQ ID NO: 16 - DNA, 3000 bp, Arabidopsis thaliana
  AT1G65150 chr124205776-24208775 REVERSE (source_file=TAIR9_upstream_3000_20090619)
aaagaaaagactttgaacaaggaagataaaaactagacataaccttggctagggttcttgacaaacataaaagcctgaaactacatagatctacaaggattaaataactcattttgattatgtacaaggcaaaccaaattttgattctgaaagtacaagcagtacattctcttcgaagcagatattctcttcaataaactgaaatttttaacttgttgtggaaacagacttgaagttgtcttcttttgaaacttagatgcaaagtgtaacaagactctaaggaccagagtaaacaaggacaagtgcaaaagagaatctcaaagcacaaattttaacaaaatagataagttggtaaatactctgttgcagctgttttgtgttttgtcattttgttttcttttactactaaacacaaggaaatttgagtagaatgcaatggataccgataactcacagctatagcagaggcggtaagaccagatctttcccgtggatctctacggtcatagtttgtcaaatacaaaaacgtacaacgcaaatgagaatgcaaatccaagaagaaacctaacagaaacatgtaaaagaatctgcttaagcaagaatctattgttgcgtactctcaataattagactaaatgatgatataaacaaggccaaacttgcatgtattgtttgtgttaaggagcattacaaaatggtctattacttctggaacttaagagacagagctatgttttatctacacacaagtgttcaatgagcatcacaaaatggtctattacttctgaaacttaagagacagagctgtgttttatctacacacaagtgttcactgagcataacaatccggatagtgaatttgggctaaaacatatacaaaatctgtccacaatagtgtaacaccaacaaggcatcaatctgattcctataaagtttttgacttcagactcaagcacttttatgtctatacatcaatctgattcctatgccattgaaaaagcttttctatttattgctccctttgcataacctgcaattcaaagtaggtggaattgagagaaaacacacataataaaagcaaaacatgagcaagtacgcagtcgaaagggccttacgttttcgaaaaacagaatctgtggacggatattctttaaacaaatgctgacaacattctcccagtctccaataacctggagaagcgacatagcctcactcaggtaaagaagtcacaattccacatacaaaaatgtctaaaagactaaaagctttatacctctatctcagaacgaagatatctgacccatctattgttttcataagctacacagttgtatcgatttccatgcaagcaaacctgaatgttaacattggagttgtttctggtaacatagtaaagttaaatggaactgaaacaaattgtaggtacttggcccttaccattgagacaagacggtacttggtatatcgaatgtcaccttcgcattggtatatcacattaacatcaatctcagtcgccagaacagaaactgtatcgaatatttctcctgcagtctcattctttgtccactcaagtgctaaacaatacgagagaaaaaaaatatgctcaaaactactgcattttgttgtgatctctactaacaaattaacttaatagcttataccataggcttaccaattgtgaaaacagatggaagtttggttatcatacggtgaacatagctttgtttcccacatccttccttatcacaaggcatctttaagttcatcttgatgaccttaatgatattctcaaatgtaaaatccttgaatgcagactgcaaataaccaaaacactttcacgagactgaattctactttcaatgtataagggaaaaaaagagacttgccttgacttctctaagtgaactagcactgatgataagacgaaaagaacgttcagcaggatattccaaatccagtttgcatctattgcaaatctttttagccacttcgtatacttcaaagtacaaaccccagttaggcagagcctccaagatgtttagaaacaggtcaaaaacaccatttgcttcctaaagcgtagagaacaagaaaacatcaagtcttgagaaagaagtaattggtaacatagattttttaaagatcagaggttagaccagaggatgcttgagacttagatcgttgagaaggaactcatgaaaaacagattcatcttctctggagcttgcgaaaacgttaatgttccatatagactgcacaaatccacattcttgaaaacttaaataaacaaaatcgagaaaaacaaattctttaggtttgtgaagtttgcgaaaccttgagtatcatagcaagagctgaaacacaggtggcgtttagcttccctataggttcctctgcatcgtcttgcgttagtgagagatctctaaacaaaagataagcaccaagtcgaaaatgtatgagaaaagcataaatatatactcggaggatgagatttaaactacttacggatctatttcttcgagcttctccacttcttgaacgtttgatggatccatctcaatgatcgaaggcaaaaccttctttctttggcagattttcccaagaaaaacaacaatggagattttctttctgagttttctcgagaaaataaacggcagagaaagaaaatgggaaaagttgaagagaagaccaaaggcgattaggcgaggacttaatagggttagagtctgttagtttgtttacgtgatcctcacgtgatatatattaagaattgtgcgccaaaaattgttatgaagctttaatcaacattacatagctcttcgagtacatattatcaaaatcttaaaacacttttttggagattgaaacgtcaacgttttgaagcagaagaagctaaaataaaaaacgagccgggccagtcttgtagctttaggcccgttatatgaaaataatatctaaaccctagaaaatctcttcttacttgttagtgacgaagaatccctt

SEQ ID NO: 17 - DNA, 3000 bp, Arabidopsis thaliana
  AT1G68480 chr125686166-25689165 REVERSE (source_file=TAIR9_upstream_3000_20090619)
ccaacggaatagagctgatgtagtagccgtgtaacaaaatgtgcttatgaacaaagcactacgtagtacataaatagatttccacaattagtcaattttcaccgttttaattgaacgctcaacattttaatcgtattgtatagttcgcttattttgcacttattcaacttgtttacatcaatcattggttgttaatttgttattattattattttggcggtgataagatagacaaaaccatttatggattggattggtttttatttgctatttatcaattatagtctaccaatataaaaaaattaaaataaatttattggagaccaacaacaaactgattttacttataaataatctattggttttgctaattcgtaaagtgttcactacgagattttcataagagttatgacaaggaggatgcagaacaatataattgttatgctccaactatttgaagtatcaatatacaaatgtgtgtattctgtcatctcaacaattacaaaatataggaaacaaaaataaatattgtaaaatgaaagaaaacaaagggaaggaccacgtaagagagaaaggagtaaaaagtatgattgtcccaaagagagttgcagatgttacatgaagagggagagattagaagtcgtagcttactagccgtgacagtcttggagtagtagtgtggtccatctctcacctgatcccgtctctctttcctactcataacttatgcaaatcattaacctccccaaaagaaaagaaaataaactatggtttttggtgcctcatcattaacattttaattaaaatcagtccaatttgtaccctatattcatgtatttttttatatcgatgatgcatgtgttcttggatattgaaacaagtaattgttcatcttaccaaaaagatagaagttattgatcatgtcaaaattatttatatacgaatatcaacttatatatgtctattttatacacaaatatttcgcttacaatttacattaatagtttaatactaactcattaacatcttaccaaaaaagaaaaattaattaaagaatttgttttattgatttctcttattatttttatttttggtaaaatgatttcgcttatacttacttacaagtattttctgaaaaatttttgttgagaatttttattccaaattatgaaaaagcatacaaaaatgataagagaactataaacgatggtgtgcaatttaattggcgagattgtaaaatattggtcttagtgtatttacaattatccacatgcaaaattcttattatggttacatatcttatgattattacaaaattaagaatatctaagtctaactaatgaccaaaaaacacgacaaagaaaagatgttcctagctagtgtcgactatgattgggctttggctttgcatttgtgttgatttgatgaactcgaaattatcaatctccgtctcgtcaccattcatactctgcttatcatataaacatgacgccgtgaaataacggtgttgggcttgactttttaaacaaatactattcctttttatcttgtaggtcggcacgagcatacttttttttttctttgagttttgaagttttgaccttgaagccacttgacacgtgattcgcacgcttccgtgagagtgaattgtgaaatgtaaataatctcacagtatctacttaagagacactaatctatctcattcttttatggatgaggatagtgttatagatcttacgttagttctgcaaacatgtctacagtataaatagcatgtggtatggagggaacttgatagcattataatgttttacttttgattgaattacattgttttggattatttacaagttacaactagattatattaaccatgtaataatgaataaaaacaagtaatgtgaaggtagggataatacacgacatgcatatctaacttaagatcacaaaggaccctaataattagttatgtgatagcaacacttgcatgacccatttgcaaaaccctagggtacaaaaaacaattttgaaagaaatttgtttttgatgtggatgccccaggagaggcctagcaatagcataacattacatctacatatgggggaccattactagctaactattttcttacatttatatacaaatatttattgtaaaacttctcaaaacgtaatgttcaaagcagcaggaccatagaactagcaatgtctcggattgtccttaaaagtataaaatctattctcttttagttacttggttctttcctctgagtgtttaagtgaggatcacaagttcatcaaggaccatcttggagctgcaaactattcatgaaagattctttatcaagagacaatgattcgctagatgtgtctttccacatttaattcattgttgataaagtactactcttgtttcttttaagtcgccttatattaatcaaacctaactaaaatacaacgtttagcatattctttcaagtttctgtatcaagtgcagcttacacttaacgtttccttctattatgtgtgaaattttgaggttgatttcatgagaacaacccataacttttcttttattcatgtatcccctaaaagtctacaataacagtcatttcttcaaaactgggaatttgtttttactatctacagtttgattttttaactagttgaaaggaattttttttactatttctttttcctatgatatgtatctcacaactaaaagatccaaaactcattgattttttttgagaaaaagagttcttaaaatattaagggtttgtcttttttaacatgaattaattgcacagtaatagggttctagggtagtagccctagcatctcctttcactcaggaaatgtccctctcatctctctctttcatcacacacacacatacatgcacacacacacaaacactttctctctggtttaattaaacccccttaagcccacatgaccacctctttctttttaaggtcacaagacccactttgctaat

SEQ ID NO: 18 - DNA, 3000 bp, Arabidopsis thaliana
  AT1G68780 chr125833845-25836844 REVERSE (source_file=TAIR9_upstream_3000_20090619)
aggaacgtgaagaactgaaacaagataaggaaagatttgagaaagagtgggaagctttggataagaagagggctaacataactagagagcaaaatgaagttgctgaagaaaacgagaaactgagaaatttgcaaatatctgaaaagcataggctgaagagagaagagatgacctcaagggataatttgaagagggaattggatggtgtcaagatgcagaaagaatcatttgaagccgacatggaagatcttgagatgcaaaaaaggaatcttgacatggaatttcagaggcaagaagaagcgggtgaaagagattttaatgaaagggcgagaacatatgagaagagaagccaagaagagcttgacaacattaattacacaaagaagctggctcaaagagagatggaagaaatgcaatacgagaagcttgctctggagagggaaagagaacaaatttcggtccgaaagaagttgttgaaagaacaagaagctgagatgcataaggacatcactgagctagatgtactcagaagctctttgaaggagaaacgaaaagaatttatctgtgaaagggagcgtttcctagtatttctggaaaaacttaagagctgcagttcctgtggagaaataaccgaaaattttgttctgtctgatcttcgactacctgatgttgaagatggggacaaacgtttcgggaaacaaaaattaaaagcagaggaggctttgaacatttctcccagtgcagagaatagcaagagaacatctttgcttgggaaaatcgcttcaaagctactgagcatatcaccaattgggaagacagataaagtcactgatctgggtataacagttaagcttccagaatcttcgcaacctgatgattctctggatagagtgtcaggcgaggatcatgaaccgtctgctactgaacagagtttcacagatagtagaatacaagaaggtcctgaaggttccctccaatcagagatgaagagtgataaacctagacgaggtagaggtagaggtagaggtaggggtaagtctgttagaggaagatcgcaagcaacaaaagctgtgtccagagattcaaaaccatctgatggggaaactccaaggaagcggcaacgcgaacagacttctagaatcacagaaagtgaacaggcagctggtgatagtgatgagggtgtagacagtattacaactggtggacgtaggaaaaaaagacagatagctgttccagtctctcaaacgcctggacaaacccgatatcagctcaggagacatagaaagtaagcccctactactttttaactgaaaaatgagttggagggtgttgcataagaaatttgaaattcaatacgcgtgaaacttatatatgtatgcaaaatcctttcagtgtaggaacagaagaagataaagcacaagcatcaaagggtgccactgaaaaacaagaacgtgtcaatgatgatatcagaaaagtgccaagtccgaaggaaactcgtactccaccggaaggtgagaatagagaaaatggcaaagctgaagtattagtggagacggtaacacatgaggagattgttacggtatggttttgtttatttctttattagttcttaatttctgtctgagttgaacatatgggcatggtaatcttaggatataattttgaaccatggtttttttatataggttgaaacagaaactgtgttcaaggttaacaacacaggaaagaacccagtcgaagatccgcagttggaagtaggaggcagtggtgaaataagagagcatggtgaagaagatgatgagaatatcagcatgatcgaagaggagaacgaaggagaagaagaagaagaaacagagcgccaaggaaatgatgcttccataggaaaaaagatttgggtctttttcacaacatgatcattttcttcatttctctctaaaagacaaaattatcatcccataagtaggattttgcaacatcttgttatgtgttggttcgttttttaactatcacctttctttatctaagcaactgaaaaacaaaaaggaagtggaatcttgggaaggcatgggcattgtagaaaatgtaacatttgatttttactctgctactccataaggtaatgctggtattcaaggaaccaaaccagactcgaagggtaagtaacaaagttacataagtcattgtcaaggacctgacatctttttttggttatgtttgcctgaaaatcgccaaaccataaaaaagttccaaagcacaaaacggtaaaaagaagaaaaggactacacaatggcttatatgtaggatcttaccaagatttcaaacttaaattggttagatttatagggggtagatagtagatacgcctcacctttcaatgttaagacaaaaatgagtgtggtccaaaacaaaggacatgcaatatagcatcaaactcactcttcattacactctcttagaaacatcctcattaatttctcaatggcaaacttctattaatgctctaattaactaaatctaaatttacaaaacattagttccaaccaattaagcttcttccttttgagttttttctttttttttttctttttcttcaggaaataaaataactgtttttgaagaaagggacagttgatagaatttcagaaacctataaagaaaaacattataataaaataaaataaaaatctggaagttgcagaattaacagagtccttataaaagacttgatcttctctgtctttcgctttcacttgcattatcaacacaagatgttggacctctctcttcttatcattttcattaccattattgtttctttgctttggtaactgtgtcttgcttttaaagatttggcttctttttttttttttggttcataagttttaattgctattgctaagtcgttgtctaaatctctggctatgtaccctataacttcacaacttagttcaatttgcttctactctatgtataaaaccagctttgagcatgttaaacatt

SEQ ID NO: 19 - DNA, 3000 bp, Arabidopsis thaliana
  AT1G69180 chr126009060-26012059 REVERSE (source_file=TAIR9_upstream_3000_20090619)
aacataaaaaatataaacgctcgtctcctcataaccggtaagctaattgattagttatcattcaatattaagtcgactaagccatgataattaggcacaagaaaaatttagtacacgacaagaaaaagagggcatattttgagatatacatgacaaatagaaaaattattatatattaagtgtacgtaaagtagtggatgcatgaataatgggtagtattaaattgctagatagctagaggagagggtaaaggcaacgctttaaagagtcggagtaatgaagttatacgagcatggaaacctgactaaaccctaatttcttattagctctcttcccttttttggcaatcgtcccatctctcacagtcatagttgatagtccccaaccttttttaaaataaacataatttaatttcggtcacctttttaagatgcagatatagatatcgtctcattatttctagtccacgattaccctattacgtacaactaattaatcttctattttcaactgatcatattctaaatttttcagttacacctctggtctaggatgtttcaagtcaaaatttgaaaatattcacatttgtctcttaatgccaatgatgagatttggtttagttttttttttttttttatgatgcaattttctattgagaaaaatggatacataaataaaacttgaggataatttctcattgtccatagttaaagtttcttagatgcaaattaaagtatcaataaaattttaaaattagatactatatcgatcagagactttgttattgttgattttggaattagactaccgtacagtggtacaaaatatatgtttaaaagcatattaaataaaaatagttagttattcatgttttcttcttgttttttttgtttttgtttttgttgttattaactaaagaaatccatgacttttattaatctatttatgtattagttctaactttgagagcaaacttcaatgtatgtatatattggttctaacattgagaggaaaactctaatcaaataaaattgtaagtaagctaaatatgtgtgtcgaagtaggaaatctaaggaaagtgaagaatgcaaaaaagttagtcactgaaagagacgtgtgattgatcacttgtctccttccaatttccctgtgggggcagtgcatgcacaaaagatggaatccatcctatagctcctcctttcttcactatcttttcaagtttctctttttgaatatattttttttaaattgattttgatttaactcaaagcccacacttcttaatccatcttctctataattagtatgccaacaaatatatatgcttgttctgtctcaaatttcaacaataaagcgtgtatagctcccaaaaagctatgttgatgagttatcactctttttatggattggactaatagtttgtctcatgtcaaaatgattatacataactcagaatccagcagtatagagaacttaacttttatatatatgttaaccctcataacctcaaacagaaaatttgtagtgcagtgtagaaaacatccttaaacataaagtaaatcctttaacaaaaaaaataaatataagtggcaaatcaagagattctgcttttgagtccaatgtatgtatagttggatgttctgaagaagatccattgttttggttgtttctctacagttaaacgatttttatatttaacttattaatgcaattgagtgaaaatttcacgatgaagaaaaaagttgtgaatcatgacaagttttgtcctcagttttgcagtgaaatcattttgcgtgaaaattttatgagagaaacaaaaaaaaaaaaaactgatcaaaagatcaggaaaggaccattcttaatttataacatgtgggcaagtcaaggcttgctggcatcaaacttgaataaatgtaatcatattcacaccatgtattgtttaatatataattttaatatcttaattggtcatctatttttgtctttttgcatcgcatatgtgtcataattaatatctgtacgatgtagaggctagtgaatataaagtgaaaacatagatatatcgatcttaaaccaatggtatcttacaccatatgtatatgcttgtttgaaaatatttattctacgaaagtattaattggattgattgttttcactctcttgagtaattttggataagttaatagttattatttttattacacatgattaaggtgtattgttaattataaatcatatttatacgaagacatggttgaataatgtccaacacagcttattttagcaaaaactaatgtacgaggagtgacatttagggtcttgatatttttgaagcaaaattaaatacgtctgcaagattttgcagagggaggcatgtactatatcatctgcaatagtagcaacatagataaaagtcataaacccctagatccggtccctttaatttaattcatatatatgatttgacataaaattacttttgttaaaaagtttgtttttacattaaattcttctatcttttttctaaatatcagtttaatatacatttgtaatttttcttgcaatccctagccagcttaatgatatcattttttttccttctttatatccagagaaattttatgcggaaacgtagattaatatcatcgaacttgttgatccaatttgttttatcttttaaaacatttacagtgattacaaaatagaatatatagcaagtaaatatccaacgaaaacacataatgtctttaacaaaaaaaaagaacgaaaaaaaaaagaaaacacataatgtcaccattagaaactcgtgtctacaccagaagtcattagtttaagatattcattttttttttttttgataagatgatattaaatgttgttaaaataaacaataaaaatgcaattatggactgcatatacacgtccaacactttgacaatctgtatcgctctctctctctctctatatatatatatgtcatggtctcactatgattgttct

SEQ ID NO: 20 - DNA, 3000 bp, Arabidopsis thaliana
  AT1G77145 chr128991347-28994346 REVERSE (source_file=TAIR9_upstream_3000_20090619)
atactatatttctgttttacatgtgattttctaatatgccatgctattgtttaatttgacatgctattgttcaaaaatacatttctttcgttatttttactcgattcatgttattttaaaaaagtacatgattttttttctttttttctttaattttttgacgtgagatgactctctttctggactatttactttataataatattacgatgattattctttcgttatattaatttgattcatgttatttttaaaaagtacatgatttcttttttttctttaattttttgtcgtcagatgacactctttctgggctacttactctgtaataatatcacgatgattatgacttccacctctgaatctcttttctttttctccctgaatcttatgagtctcttttctttttctcactgaaccttatgaagtctccaccaccacaaacaaatgtatcatcgtaaaaactataagtttacaaggttaattggtcttttcttcacccctaccccaccataaaaaaaagtcatttaccaaaaaaatctgacacatagttaatttctttattaagatgtcaaatttggctattgcctaatattacccatattagattcattataattagatcagttaaaaacattcagtattttcttaaaaaaatattcggtggaaattttgtaattaaactttcctttcaaaatcaaaagaatttgatttgaatgctcaacgtctaaaaccctccagcactgaattttaatgatgacaaattttccaatttagcatgaattgcatatttgcattcattgttgcacatgtaaccaattggtagttttcttacgaaatatctaaaatcagaatcatatgataagaacgatgatgtgtatttatataagacctcaacaaaatacctaaagtttaaactgtaattaaattataaccaaaccatcactttcaatcatatgtactttgataattataagtaatctctcttttttgtttgttttgtcttaatctatttcgtcacgctttattctttcgagctaaatccggtggtagtttgctagaaacttcggctaagcacacaataatcaaaattcaaaaagtcagttaactaattataactaagaagttgcttgcgtccatggaggtcttgtagaggaaggaaaaacgtatttcgtgatgatgaagtcagagttcgaactagaaccaggattatcgcattacggatgcatagttgatctgttgagccgtgacgggcagcttaaggaggccaataaagttgttaaagagatgccgatgaagcctaacgtgatggtttggggttgcttgatggtaggggtttatgttgtcttaattggctaatgtgtatgcactcagagggatgtggaaagatgttgagagagttaggaagttgatgaagaagaagaaggttgctaagattcctgcctatagctatgcttcaacgacaatttgaaaatatatgctttcgatacttgtctctcttcatgttcgaacaatgtaacatcaggataatttttctaattagatacttaatccttctattttattcactcattgagaaacagcacctgcagatataaccaaatctacaaaaacaaacatacaatgcaaacaaagtaatctaattattagatgatctgtataacatttaattttagtaaattaagtacctaaataacgaatttgggaaagttttctagtcattttcgacatcatgatcatgtcagaggggtttaaaactcgatttaactccgctaatttcaggaatccggtttaatgtgttcaatttccggtttactgatttcatttcggaaaataagaaggtaggaccgaatttcaaagcaaaaattatacaaataaagaagatacggtccacacgcttttagcccgtcttaattccaacaattgcaagtttaaacgagttattatagcccaatacgaaaattttaaattccatgtgactagagagcatccaaatcaaaacgctgtaaatcatcggattattagctaccgtcactcggattattattataattatcttagagattcaatatcgtcatgctaatattttatcttgataagtatatatatatgcgtgtggtatcgtcgtataagacaaaatctattaaaaggttatataagatggtttagccatcaatgaacagacaaaattgattacgagataaccttgcttccaaatatgtgacagtgagtttggatcgatggtgagcccatttcgatcaacattatagtatatgctatgacgtgcatatctatctactttgatcgataattcaatagacacagataaagtattgaagaaacattagtctcgtgagggttttgcattttggttttctgttgagtgttgacattgtgattttaccaaagactgagggatcggtccaaatcaacgaaaaataatattaattttaatttctttgaataatatgaatatatcttctatatagactcctgatcaattaatatttcatttaaaaaaaaaaaaaactagtactattttttgtgtgattagacatagtgaaattaccactttattagatataacattctaaaacaaattaaaatatcaaaaaatattaaataaaaatcaagacttacgaattgagtttaatctaaaatatccacgtatacataattataatctcaagcatcgactatgtggaatatacgacaagtggaaaatgacatgaaacgactttttatacagtcgcgtatgacatcaaacgcaaagtagtttgtatttcgaagaaaagggcaaaaaagaaagaaaagtcacgacgcaaactcatgttatctaaaacatataagctaatacaacaaaacattagaaaacgaataataaaacagttcatttaataatgcaaatgacaatggataagattatgacacacgtcactcagttatattcataaagagactcaactgcataataacttttgccctcacaacctttttctc

SEQ ID NO: 21 - DNA, 3000 bp, Arabidopsis thaliana
  AT1G80580 chr130290558-30293557 FORWARD (source_file=TAIR9_upstream_3000_20090619)
cttgggaactaattcacaatttagacatctctgatgagtttttcctctgttttttatttctatcttatcaattttgtttctggaaattttcatggctttttgaatgttcattgggattgcgtatcttggtggtggtaagtaaaaagtcaaacgccttcgtttttctgcataatctctgagttccatcaaagctctgttttgtgttttcaaggtggacttttcacttgacatgttaggctttgatccttttttgttcacacagatagacaaatggatgaattgccagaccatttggtttgggacattctcagtaagcttcatactaccgatgacagaaactcgctttcactctcatgcaaacgtttcttctcgctggacaacgagcaaagatactctctgcggattggttgcggtttagttcctgcatccgatgcattgctttctctctgcaggaggtttccaaatctttccaaagtggagattatatactcaggctggatgtcaaaacttggcaaacaagttgacgatcaaggccttcttgtcctgaccacaaactgtcattctctcactgatctcactctgagcttctgcaccttcatcaccgatgttggcataggccatttgtcgtcgtgtccagagctttcttcgttgaagttaaatttcgctcccaggatcactggttgtggtgtgttgtcacttgctgtaggatgcaagaaactcaggagacttcatctcattcgatgcctaaacgttgcaagtgtagagtggttggagtatttcggaaagcttgagactttagaagaactctgtatcaagaactgcagagccattggggaaggtgatttgatcaagctgaggaatagctggcgaaagctgacaagtctgcagtttgaggtagatgccaattacaggtacatgaaggtttacgaccagttagatgtggaacggtggcccaaacagttggttccctgtgacagtctggtggagctcagcctcggtaactgcatcatagctcctggtagagggctcgcttgtgtgctgagaaattgcaagaacctggagaagcttcatctggacatgtgtaccggtgtcagcgactcagacatcatagcattggttcagaaagctagtcacctaagaagcatctcgctccgtgtcccgtccgattttactctgcctctcttaaacaacatcacgttaagattaaccgatgaaagcctcagcgccatagctcaacactgctcaaagctcgaatctttcaagatatctttctcagacggtgaattcccttccctcttctctttcacactccaggggataatcactctgatccagaaatgcccagtacgcgagctttctcttgaccatgtgtgtgtattcaacgacatggggatggaggctctatgttcagctcagaaacttgagatcttagagcttgtccattgccaagaagtgagcgatgaggggttgattctggtgagccagtttccttctctcaatgtgttgaagttatcaaaatgtctgggagtgacagacgatggaatgagaccacttgttggatctcacaagttggagttgcttgttgttgaagattgtcctcaggtttcaagaagaggtgttcatggtgctgcaacgtctgtctcgtttaagcaagatttgtcatggatgtattgagatcgtatgatttgcttcctatgtacaattttactcatgtgtaagtaagtgtaaatgttcgatctttgggacagaaaaattactaatgcctcagtgatgatcaagtttaaaatgacgtcatagcagttgtatataaacagtcatggatgatgtaatgttgttactctttaatttaatggtgcgttttataaatatagcgtcataataattaattgactgatgtctttctaatccatgagctaatcttatcaagtcgtcattctaaagatttttaaatattgatattgccacgtcaaaacgcaagtagtagtacttgtttgatcagttagagttcaactgggctgacatttttagatcttgtgtcggtaatatataactttcaccatctttcttaataccaaacgtattttgttctatagaaatttctgtcaaatgttatgattgtctgtctactaaaaccacatatttacgtttgaaacctttaattttaaatcctttattttagcagataaacatactcctttaatagtagcttttatatgattcctttaatatttggtaattagttgaagaagtgtgtaccctaagcgaaattgtaatcagacaaactgtgtgtctattgacgccagcataagtccatgaagagatcacgcgcgcgccttacgtaatcaaaacggatgtcaattcgaccaagtcaaatcgtttggagtctacagaacacgatttgccgtttgatttaatgggcctatgtctttgtcaaggcccaacttataccgtattatgcaagttgtcagatacacgagaaaatttgttactgtatggatttatgtaaactcatgacatttgcgacagcaataatcgtggctttgcataagatgaaaatattctcagatgcatagtattattaggatcaaagttctataattatttccttgttcttccatgaccatgtcacttattttattcaaaggttgataataccaatcagatgtctgcaaaataaagggaagattataaaacttttaaatcaagatttaagagtgtttgtctacctctctctattgaaaacaaaaggtacaaactttgtctttgcactgtgaagcacagatgtgcataataaatgtgaaagattttactgtctcatcgaaattgacagcagtatcttttaattattccactaaacaaaaatacttttaccataatatttttaattaggaaagaaaactttcaaatgaaatcatctttatttacccccaattataaataaatggtaacgaaccaatcatttttttgcatagaagctttctcacctttataaaatagaaa

SEQ ID NO: 22 - DNA, 3000 bp, Arabidopsis thaliana
  AT2G03500 chr21056710-1059709 FORWARD (source_file=TAIR9_upstream_3000_20090619)
agaaatcctgtttatatcccctataatactaatctgactttaactgaaccaggtcggttcagtctgcaaagtttttgacactacaacatctctatttgttattgagatcaccattgattttgtaagaaaggagatatataattccactcttatttcgaatcatagtttagaggtcgtgatgacaaggatgttcaaataattgttatttctgaatttatagaaagtaactaaaaatttcaaatttttggttaattaacaagagttaaagagaataaggtacaatgatatttgtacttaagaaaatattagcatatagaataatacaccatcacttgaattgaaagaactttgaggaaaatatgaaatatatatgaattggatatccaaaattttaggatatgcatgtcggagggtctgtccgaaaatttgatgtgtggcattagatgccaattggttgcttgaaactatatattataaaaaaaattataaaagaaaagacaagagtaacaaacttttaaaatgcgatatcatacaaaataagtgagttcattacttatacagtgatgaaaaactgattttatttgataagattttaaaattaacttgatctttttcatcataattttctcagaattaatatacacatgtaacacagagttacagatattgacctcactggaacaactaatagtagaatattagaaataagaaacattttttgtaggatcactcttactggagtataatttattgattgaatgcgatgttccgattaattttagaaatttaattggtcaatggtataattgatagtaattagaatatatatatttatttcagcttttttatctatacgtctagactataatatcgttgcaagcaggttctaacgtcatgtgtagatcctttaagcagttcttgtagcatcacttgcattttcacgatcacgtagccttattgcttagttcaatttttattttctttgactgtgcctttagaaaggtctgagggtgatcctacaaaaaactagataactcgatatataagaccacatgtcagaaacttggaagggtttagtgattagacgactaaaacatcataagctatccaattaggaaaattaagtactaataagaggacgagattctcacatctcaaatacactttacatatgtaaagcttatttttgcttcattagtttttagagattaatgttctagaaatgttattatatgaaagcatagagcaactaaacaattacaacttctaaatgctctcttatttgccttgaatataaatttgattgggtagaaaaaatcttgtatcatcaaatcaaattgatatatccgccccgaatttgttttaatatgcatatatctaataattaatttgtattacatatgtgcatcaaatattaaacagaagcattttagagaattaattttatcaggtccacaattagttcatcggttttcaatgaaacctagatacgaaaagaaattttgataagactttgaattgcaaaaactctttaaaaaagtttaaaatatttctaagtaagaagaatcttctaccttcctggccacttcactaaagtccggtgggaagattctgcgactctcaaacttctgtcaatattatttacttaagtgaatatctgtcctagtttatggtaataaattaaatcagatattccctttctttctcttttttgcatccactaaaatttagaataattattcaacttttttaagaaattcaatttacaaaaaaatattgatcaatcgttatctctgtctctctctctcgccgtcgtgcaactataaatataaaaatcttgtggcctaatatgtgcttctttgatttatattgaatctcaattccttttgaaaatttaaaataattaactatataaattaagaaggtggtagaagaatatttgtttttctggcaacgactataaatttcccaaaatgtactaaaacgttcacaatttattcctttcacataattgcgttggtgattccataattttattttaactttgtcgaatatataagataattaagaaccaaatacctgaaatgttagcttagatctatttttcagtcaacatgagctaagatttataggagaattaggaaacatattttatcttaagaattatgagactgaatttacgaaagtataagctatgtgaagtggtgaaccaacaaaagcagaatctgagattaaaaacaatatatcgctttattagaaataatttaaaagtttgtattcttacccatttgttctttataacaagtttccatatattcccttttgctctgctgctttatatcttcctcttttctcctttagcctctcttttctctttctttcatatggaattatacatagattaattttctgaagaagtgattaaaaactaagcattataagagaagataaaaaaacaaatcaaatggttactaatttaatgattttactaatatttaccaagccatagttttttatatattaactattttgtttaagattcatgaagccatatatggtagcatcaaaagccttgattataagctttttgagaaatgagagtcacacatgatacacctttagtaatattgactcattagaaaataaaaataatttatctttgttatattttctcaaattttgctcaaacacaagcagcacttgatgtaaaacagtttttttccgaataattttacataatattaaaaaaaataaaaaaatattgactcattagagaagaggaacgacttctaacagtaagaagcctctcactctctctctctaaaaaatacacctccttagtctctccctctctcgatagatatatgtgtgtgtgtgtgtgtggtgacagatcacattattccgttcctttgaattgagccctctaaaaaggacaggaacctccttgtgatcctccttgtgtccacaacctaacattttattccccttttcctctatctt

SEQ ID NO: 23 - DNA, 3000 bp, Arabidopsis thaliana
  AT2G17950 chr27810796-7813795 REVERSE (source_file=TAIR9_upstream_3000_20090619)
taaaatataaatgtattagagaatgataaaattttcaaaacttatatatataataaataattcttaaattttaaaaattactactttaaaaaaaatcaagggacgggtaaagaaattacagaacgggttttattttggaattgggttatatggtagatgtattttaatcaatatttagaatatttataaaattttattaatatgttgttttgataagagttaaaaacttcagttttttaacaattgtctaatgtattcgtggtatagtgttacttaataacaattataaactgtaaaatataaatattttataaaaataaaattagcaagttttaatatatattacctttaaaaaataaatcgtcccgcgttatatcgcgggttaaaatctagtcgatttatatttacaagatcttaggttgaaaataacatacaaactaccttcgtctataaattaaatataaattggcataatatctggaaaatttcttacaatcaagtcatgacgattgacgaatattgaaagctcaaacactaatgttgtacgttgatgaactaattaaaaattcaataaatgctctaaacatataaacagtgtaacgtaaggtgtaaatagcgaataacatatgacagactgaatataaggctcggatcgtagaggggaccatgttccaaacccaattttcccccattaatttccgggaaaccaaaataataaaagctaattataaacgttttcatgaaattagggcaaaagtacaaactgaaactaatgatgccctaaatgaaaatgaaatctctgcaataaagaagagtggctttaggggccacgtagacattagttacaaaagctctgcgtcttttagccctctgcctcttcaacagtactttacgacaatgaaaaagatcgtgatgcgtaaattaagatcccctgtttctcactgcatgctcgttaccgtacgtactcataggagtttataaatcaaagggcctatagagagtattctaagtagcaaccactacgtaaagttttaccaaaaaaaaaaaccagtatgtaaatgtgaccatatgttcatgtagttgcttgtgtacatctttttttttttttttaacttcaaactttatgctttattatagtgaagaagtataatcctcatggagtgtacatctatatatacactgcatacgcatgtagagtaacaagacattctttgtttcttgtcaaaagacattcacaatccaactagagatatgattttatcatttgttagttagtttatagtttggtgacttggtgtaatctcctagttggataaaaatattggaccgttgattcgatagaaatgccaaacgacattacaaatcggatatatcgattggatggaatccaaaagtttatccaatataatcgactaaagttaaaaaatgagtaatccaccagtctaactaaatactttctttaccaacaaaaatttgggatacaactgagtttatttaggtgataaaagattataagacttaaccttttaacaaaaaaaattttttaagacttcaatccttagattgttttccaaatattaatagttacaatagttagtttgtaaaacctatttttaattttccgatactctttgcacgttgtgttgaatccgagaaatcatgggggctatataaacaaaactatactatgaaatagccaagcaacttcagtgatcggatctttggttttttattttaaaaaaaataaatattttatcagccgtatcttcagggccattccccataaaaactataatagcttctatgaaatttgtagattcctaaaaaaatctttatatatcttttatatacaatatgtggctgttacgtagaacaataagactatacaaaagttgggtattgttccttttcaattacaaaagtcatattcactaaaacaaagcccttcaaactttttaaaaaaaatgaataaatcacgcatttatacaaaaagatccatccatgctcctataaattcgagcatataattaactaaagaatactcaacatgttcataagtacacctgtcttcacactcgtttcacacattgtaattgacataaattttaatatcttttaaagcaaagcataagtaaaattatatagagtatttcttcaacacatttccggacgatatcaccacgaaaaatatcctatatcacctatgtgtcatgactcatgaccataaggaaggaaaaatgtatttttatgatcaaacggttttcactttttttattattcattctagaaaaaaattataatcgttttctttcattcctacgatacaataattcataacgatcttttagaatttttgttttgttttctgtgtgtatgggtaaatatctttcatacgtagacagatacataacaattaaagctggtgaagtaataattgtgaagagacagagaaattgaaaaaagagagtttgtttttcgtctaagtaaataccgatgatgccctcacctttctaattccctcctttgtccctttccattttcatgtttatttctaacttctctgtccctttcaatccttctccaccagatatcctctgattcgatcccccacaaattttttcataaaaattattattattctcactaaaactctcaagagtagtttcgatttttcctctgaataaaaaaaaatcaaataatatatttgacaagagaatttccttcctaaacataacattttatttgaatgttaattcaattgattatttttctttcttatatccttttctttggcaaaatttatttcttatatccaagcacatttttcaatagggttttgtatgtatgaaatcatatctacatgcataaaagaataatatagtgaataactgattttctagaatattgtaaaaccttctttttgggtccccatttatatatataactcaataccttactatctctctatatatatataatcttctctctcacacaaaacct

SEQ ID NO: 24 - DNA, 3000 bp, Arabidopsis thaliana
  AT2G19910 chr28600758-8603757 REVERSE (source_file=TAIR9_upstream_3000_20090619)
gaaaacttcctattgacgaatcatactatctcatggggacagtggatcccaccggagagctaaaggaagatgaagtctgtgtcatcctgtatgctaccaaaattttatttatttttttgtaatataaagtgtggatggaagaataaacaaaccgtgcaacttgcttcattggagttgtgagttagctcttcatttcctcactactttctttttcgttgttttgcagcgggtctggccaaatctcaggatgtgctagtttacaggaatcctggactacattttggagacatacatattctaaaggctacatatgttaaatccttggaacagtatgttggaaattccaagtatggtgtgttcttccctcaaaaaggtccaagatctttgggagacgaaattgcaggtggtgactttgatggcgatatgtatttcatatccagaaatcccaaggtacactgctatatccatcacagttatagtaaaatttcattgctgagacctaacttataaaccgcaacttcctctgtttagctacttgagcactacaagccaagtgaaccgtgggtgagttcttctccacgtagtaaaatttacactggtagacaaccaagtgaactttcacccgaacagttggaagaagagcttttcaaaatcttcttaaagacaggatttagtcccaggtacattctttaattctcgggttactttcaaggtcacatagtcctttgggttaaatatgtcttgttttctgggtttacagctcagttataggacaggctgcagatagctggctagcaataatggaccggttcctcaccttaggagatgagaatgttaaggaaaaagctgaaatgaagaagaaaatgctaaagttgactgatatatactatgatgctattgatgcaccaaaaacggggactgaggtcaatctcccgttagacgtaaaggtcgatctttttccacattatatggagaggaataaaacattcaagtccacttctattcttggattaatctttgacactgtggattttcataatgcagaggacaccacaccatctggtacgggtcttcttgttactattgataatcaaagtacacaataactcatcacgttgaatatgttttaacggtttagctctgggttacctttcaggaatcagtaaacttcagtgttttgaagatgagccggtctctgagtttgacatggagaaatgtaaactctggcacaaggattacagaaaggagatgtgtcaagcgatgaactcagatgatgatgattcctgtaatgaagttatccagaaatacaagcaggtaagctcttcaatttagttaattctaagagaaaagctttaaccaatagccttcatggatcactggttttttgttgattgtaatgctttataaacaaatcattggttgaacggtttcttctctctgtaacaggagttctacagtgctgcagggtttaaagaaagcaagaaaattctagaagaactctacccaaaggcccttgcactttacaacgtcacttaggactatgccatcaagtggaataacgttaaaagttgcgggtttgtctggaaggttgcaggtccggtcctgtgcagattctacgagaagaatttagtaatgtgttcgatttccatgctgaaagagattttcggctgaacactcttactgttctgttctggtgataacaataagtatttgctgtcttcatgtctcataatatgcgtgttaggcaagtaacctacttataggccaaaccaagatgtgtggtgtagtactggctttgatttaggtctaatcttagatttgtcctctagttaacatataattccgtgtgtatgtatatttgcttatggaatagaaaaaaatgatgacctttatagtgtggtggtgtacaaagctatcttctccagtgcgactcctctctgcgtcctcatgcttccatttggattttccacaaccttcttccttccattactatatttttcaagaaaagtagcaaaagtttcttcctttgtgaatagtagcaaatatagtataatatattagcaaatatagtaatggaagtagcaagactaaattcttcagaagttaatgaatagtgcacttgcagagcccaccttcctatatcttggttctggttaagtaagaatcaatttttggaatcacacagtctgatcctttatgtccacatatttttattatgtcttcttgtttggttattgactggaacagaaatagcgtaactcataaacctccattggaattttcgagttctgaggtgtaggggaataataacctctaataatgaactctctccgtcttgcttgtagcaaagtttcttcttcactgaaccgctgatatacctcagtatccatttcactgcatgccaatgcttctttatgattttgctcatgaatctacagactaagtaacttagctaataggtaccaagatgttgtagcgactttggtttggttctaaacttctaatatagatttgttgcctctgctgagtattttttgtgaataaatgcttatgacaatgaataaaattacctctgcaggtgtgtttttgtaaccttgaaacttgtaatatgaatgtgcatatataaataattcgcgattatgcaatgagacaaaatagtccgaaaacgcgattttacttgcgcgtttgcagcgccggcgagaaagtagtggcagatttgtaaatatagtagtgatggttgggcattttgataaaaattaaaaaattaataaacggctagaatagcgtactgcatataaatctccgtgtagtcaaatcaaagtatcaaacagatttacacaaaacttctgtttttttctttttttgaaacctaaaggacgaaactttgagaaaatggcagcaaaagcaattccagagttaaacaagtttctctcttcccactagggctatatattatgaaccctagattatagacactgactccgattttggggagaaaaa

SEQ ID NO: 25 - DNA, 3000 bp, Arabidopsis thaliana
  AT2G27250 chr211665770-11668769 REVERSE (source_file=TAIR9_upstream_3000_20090619)
tttagcttaatttttctaatatatgcttgctctttttaggatgccacatgaagttgatctatggtgagggtggcgattaaacttctcataaggagaagtaaggaaaataaactcagctaaaatcttatactatcatacaaattttagatttcaaataaaatatttaagtagattagtttgagaagaaagtcaaatggcttatttataagaacaaaaaaaatagctcgaaaaaatgattaaatttttgctttgaattgtgacgcttgctccatcatatgtttgttagctcattgcatttggctatgcattgataaaagttcatgaagatgtgagatactaaattctcgctcggtaatacataagcttcaactctagctagggttcgtttaccatacaattatgataatccttcattggtatatctttttcttttatttacttttttcatccacaaactacattaacactctatattctcctccaaatatcaaaactattgatgccagttttttcgaacacactcatatgttaggtgcttcagttttaaagtttctaataacaaaactcttagatgtgcatgtacatgtgggagaaatgaaagtgtgcaagtaatggtgctcattattgacgtgaaacttgtccttaaatttcaaagaattctacgatttttttttgtatttgttgatcaataagatttttttggttccaactctataaaattcatttactattacactcgcataaattacgatgatattatgaaatgtattaagactctaacagtttaacagttaacgacaaaaatatctactattgtaaatttaaatctttctttccaactaaattgcatattttcggttccggtataatgaaggcctaaaatcataagctatattaatcacatggaagaattaaataaagaaacctgatcgatatggcagtagaaagaagtcaaaagatttggattttggattgcaacttttttcttattggtttatataattaacaaactagttgatatatattatatgctgcagaatcttacctccttttttacatagtttacactttttgtgtgcagctctcaactcaagtaggttaattagacatataaccaatgaacgtaataatatttaaaacttagaattgacctttatgaattcatcaatcagtctcttgtcgcttaacgttacactcgtcttttctggttactactctcttccctctcattgcattttccaggctgtttcatctttctttgacatcactgtatcggtaacgattcgacgacatcaataaatatattgttggccaacgaaaaatagacatgagtaataatttaatataatacttaccagtaagtttacaataattttgattgtcttctgatctaataaattgttggccaaaaaaagacctaataaattcgtggacttggagttgatgcagatctttagcagtatggtggtgttgaattcaatctttttggtccgaatgtctttaaaattgttcttgatattcatatccataaaattaatcgaattccggattatccataataaaaacaaaactagatacattttctaagtacattaacacataagaatatcatttttgtactaccaaaaaaaaagtaaaaacttatgtccatggtattattataattggtggtaaatcagtaaattagttgttagcaaaaaaataaaatttagttaaaaagtagtggcaccttattggcccaaaagagtaacgaagcaaaacggaatctgaaaactgttaaaacttaaaagaatagataattgagatataatatcgtatgatcggacggctgtgatctgatgccatcggatgggcacgtgtcagaagtgtactccaggtatcattctctctgcctcatgacatcagccgatggtaccctcgatacggcgccggcaaggctcatataatccattcaatttatgttttttctgctagcaaatagatttgccattgatttgatactctcaataatattatccgattaaggtatcatccatattgttcaatataatttaagcatataactgtttccagattaaacaatataatttataagagcaactgtaatactttactttaaaagttttcaaatcagaatctcttttctttttctacaaatctggaaacaatttaatccatattgtttttgttttttttaccttctcatatttagatgctatttattattgtgacacaagcttagtttaatttcttgttggttacatttttttttctaaatacacaaatattatatgtttaatattatttcacacatttcttagaagaaatctaaatacatgaaattatatttattaaaaagaagtctaaactatatatgtatttaaatggaattttaaatatgggtagtaatatagaaacaccattgatatattagagtatgtgccggtgccgtatacatactgttgtattatagtaacctatatatggtatacctagtcaatgtgtacggtatttttcatatgtattagttgtgaacctccacagcatgttagacttaggaattaattataactgaaccagaactttgagtctaattatacccctgccgttttatacaactttgaatattgattccagtggctaatttaaataagttaaaggaaacaaaaactgcaagtaagtgaaaaatacacaattgtaagtaaatgttagatatttaatttatttatagctaaatcatgaacaagttcgtataagatctagatatatgtaccatataccttttctatgcccactatatatacttactacacataatatataaaccaaccaaaatatttgaatagttaataactatgatacacgtttaggacaaataaaattaaaaaataggagatcccatttctcgcccttgtaggcttacgctataaattggactgtccccttctcatttcattaccaaagtaaagaacagtttctatatttctctctttatctctc

SEQ ID NO: 26 - DNA, 3000 bp, Arabidopsis thaliana
  AT2G33880 chr214343779-14346778 REVERSE (source_file=TAIR9_upstream_3000_20090619)
taaaattgaaagaaaatatattgtatacctaagcaaacgtataaatgtaccacagcatatgaagaattcagagactgagcctctgttatttatgaaaagatggttaaagtacaattccaatccaatagcttatgaaatgatagattttgaaactattttattataacaagataaacctagatactcccaaaaacatataggaaaaattcaggctgtatgttaatcacttcgacgtttcttataaccaaaaattaaacgcaggatatgttaattagtaacttttttttggtatgtagattgtcatggaaaaatcttcttaattagtaaattaacttgaaaaacttatagtacatcgaattgtttgttgttgttttttttttcaatcaaaacgaattgtttattgtttcataagaatagatggaaatatagatatttaaatggttgcattgatcgccattttctgattcagccgaagttaagctgcaaaggcccgagcaccaaaacagcctaaacatatattttatattttaattttaagaacaaataaaatactactatatccacataactaaaatatctttatttttttgcttaaaaataaggccataaattgttttgctttggaatccttaataatcttggaccagtccttggaacgagactttgcaatgttacttacagatctagttaacactgataaccgcttgtgttaaatggagatgccacattttgttttgatttatttgcatcctatcacaaagttatgatttataaactaagactgtttagcattgtcccatagtcccattcaatagttagtcttttaaataaagctcacatttagttttttttttttgtctgataaaaaaggctccatttagtttatatttgaaataaatgcagatgaaaagaatcttaatatttcaaaagaaactgaagaattattaatacgttattaaagggaacgatcatcaacaaagaccctcgagtcaatccaaaaaaaaatgaccttaatttctttgcaattactatacaccaccacccatcctgacctcttaaaatttcgtattttccccattaatacatgtttcttttttccgtactattacatcacaaaactcctaaaatcgtgtttgctaatttgattatgactattcctacgtccaaacaaaccatttgctccccatccgaatttaaggccgatgccccgaaggtgaccgagagaatcctcttctgcacagactgaaaatccagaaactacatgattttatttttttgtaatacaagcaaataataaaaacccatcttgttatagttaataaataaacataaaatgtgagaaaatttacaatctttctgtcttgttttaattaatgtttttttttgagatgtcagaggagaaaataataaaaaggagaaaaaatcctagttcagaaccaaacatggatcactttcaaagaatctgttgtttcaatttctgaagaaatttgtgttcgtacgtttgaactgcagaagcagtttggagtttaatgcaagaagtgtaaggaagtgaggcatggggttgcattaatgatcactttgagtttcttcctgtctcttactgttttctatctcagaaattgatattaggaatgtgaataacaaaacaaacgaatgaaataccatgcaaactctaatatggatgtagtaacagaaaatctgtattgctctactaattaaattttttaatttagtaatatgaattcagagtctattagtataaacctaaagtaaacagaaatttttggagtgcgcttcaaatacccttgtaaaattacagatgactttgtatggcaactttttaaaattgctaaaataatttcttataataaatattgaatatttttaaataattcagtacccactctataaatctatataactagtctctctctttttttttttttttgggcacatataactagtctctctatcagttattttcatatggattttggatttttttgtacatatatgaaaaacacaaatgataaaattatttaatggtattaagcattcgccacagggtatgctatcaatttggtcatttgtgagtgagattgatatggtttggggctaattcagtgaagctccttcggactctcaaagtggtttattagacagtgtgaatgcatgcggtcttctatttgtttgcgaggctaaaagaaacgctcatgatacacaactaatcaaatgactgttgaagaaatttaagatttccaaaattgtcttcttctatttgttattgaagtattggtcaatccatcagtgatttaacgttatagcaatataggctactcgtcgactagcacattttgacgtacttaaatactaaccttgtcactctttttttaatactgtaataatgccttttgaatttccattggtgatatacacattttcccactagatgacactaaagggaactaacccctaaaacagtttttattttatcataattgaaccatgacaagcaaaactggattcaaatctaaaatgtcaaagtgctcctcataatctatatataagcgatcaaagaccttctatagaattaataaatttactttttaatgcaattttttttgttcaaaactatttaatgcaagtatgcaactaaatcatactgtttgactgaagtagttaataataaaacgtaatgaaaacagtgttcctattttcttttataacatttctgttaataggatttagcctacttctctaattcatttgattccctaaacaagagaagtagtgatagttttttttgtctttactaaacatatacacataatgatatagatacacatctatacatcatagatagatacatagatatagagagaaagagaaatggtgtatagaaatatgggtgagactttaaattaaatttatgtaaaagagatgaatgaatatatgaaaacaggttgggtaggtgaagagaggctataagagggagagaaa

SEQ ID NO: 27 - DNA, 3000 bp, Arabidopsis thaliana
  AT2G39850 chr216627473-16630472 FORWARD (source_file=TAIR9_upstream_3000_20090619)
actcggcggttctcgatgatataatcaggcgactcacggaggtccggttagctagaccaggaaagcaagtccagctctctgaagctgagatcaaacagctctgtactacagctagagatattttccttcagcaacccaatttacttgagcttgaggctcccatcaaaatctgtggtatcttcccaatttgattcgttgattttaataatttcgtcggaattgattttagggttcttattgtgtttcttagattttccaaaagtgtgaacttggttaggttttggggataaagttgttagaccattgtgagacctttttgtttgatgttgtgaagtgactagaatctgagtgagaaatgttttggctttttgtggataatgattgagtggagaggcttatcttgttgcttatgatactgtgtgtgttcatgaaaaatcctatcccgttctgtgatttccttatggtttgtggtgtacatgtcttaaaccaggtgacatacatgggcaatatagtgatttgttgaggctatttgagtatggtggcttccctcctagtgccaattatctatttcttggggactatgtggacagaggcaaacagagtcttgaaacaatatgtctcttactagcttacaagatcaaataccctggcaacttttttctactaaggggaaaccacgaatgtgcttctatcaataggatatatggattttatgatgagtgtaaaaggaggttcaatgtgagagtatggaaggtttttactgattgttttaactgccttcctgttgcggcgcttatagatgacaaaatattgtgtatgcacggtggactttcgccggatcttgaccatttggatgagattagaaacttgccgagaccaactatgattccggataccgggcttctctgtgatttgctctggtctgatcctgggaaagatgttaaaggatggggaatgaatgatagaggtgtttcatacacctttggtccagataaagtttccgagtttcttacgaaacatgatttagatcttgtgtgtcgtgcccatcaggtagatatttttcaatcatgtctctttgatactactgcttttgtgatgttaacaagggagttggattttttatggcaggtcgtggaggatgggtatgagttctttgctgatagacaacttgtgacggtgttttcagctcctaactactgtggtgaatttgataatgctggtgcgatgatgagcgtggacgagaaccttatgtgctcgtttcagattttgaagcccgctgagaagaagaccaagttcatgatgtccacaaagatttgatactttcattgaggttagtcaatagtctgtccttggcggaaaccaaaacgaaactagtgaatgaaaggattctcttgcactgatttgttcatgttattcattacaaagtcaagttcacatttctgttgaacatgatttcttttggattcttccaggctaaactttttacttaaatgggtcttgatttcggcttttccttctttcacagatggatttggtctttcttgagaaattgattgaaaccattgaagatttggctgtgaagtgtgtacttatggtagtgagaactcttggaaaggagcaagaccttcttagtcttctaatctctttcttcccccaaattcactttcagcattttttttctatcttcttttatgggttttacatgattttcatctgcctgttgggatcacatagttggtgtgtacatgcatatggtcgtttcttggattttaggacaccaaatgaaaatgatgttataaaaaaggtttagtatgatgagtttcgtctcttaagcggcatgtgaaagtgaatgagacataagtaacgcatatgaacatcttcaattaaccatcataaatatcgaatgagcatctaaattggacaacaagtttcacacatgttcatcatgtttaagtaaaagaagaatttgagaagctattgaaagtaaagtccactggttttggtatgatcgtgagtgaagtgatatatgatccgtttggtggactcgggaaatagaatagaatataataattggagggactgaaattttatgttgttctttgttgtggatgagaagtaaacacggaagcgataagttcgtaaaagtaactttttttttgggttgcaaacgggaaccaaaaccacatgtatccaattctttagacacatatcatgtcattatcctatctttgaatatgttcatattcttagtccatttcctcaccagaagatgttggacaaaacgtatcctttgatcatagtatcctcaaacatttcctacatagtattgtgaccatgtcatattcttctagacttcctagttcctacatagattatgaccatttcatactattctcaaaactcaaacaaacaaaaaaatcaattttgaaatataattatttctaattttcatatttttgtaaattgaatacacaagcgttcgtcgtatacgaccattgttttcagcgtgtaagtttagcaaaaaaaaaatcttaaaacttgtggttcatttggaaaagtaagaaaatatacacgaacgtaataaacacaaacaaacaattgaaacatgaatgtacaggtttagtgttattatctgaccacattgtgcgcgagtgtggaccatgtggacgttataagtttcatcaaaccaagcatacatatatatgtgcgtcaacgtagaccctcaagttttcttgattttcactcgaatcaataccgtatactcatgttttatcgtaacatagaaaaaaaaaaaatcatggactttctcaagaatcaccaaaaaaagcatgcagcttcataatcgaagaataagattctttaaattgattgatgagcgtctgcactatgaaatgtttcgatttcaatcttctcctccaattttcttcttcattagtcctttttcgctcccaatatccttcatgaaatttctctataaatagagacataaagcattaacat

SEQ ID NO: 28 - DNA, 3000 bp, Arabidopsis thaliana
  AT3G02500 chr3516364-519363 FORWARD (source_file=TAIR9_upstream_3000_20090619)
taagagaactttggaagataagtgatttaatggagaagatacttgttcgagtgagtttttatagggagaagttattgtctgcattctacagtaacttaataaacaattaataataggtatacattggacccttaaatttatctctaatcaaccatataaatgattatattgttcgaggaaattattatcatttgtatgtggtttagtcacgtcaccaaactctatataacaatgtgaaaatccaccaacaaagatacatttgtcggccatttggttgcataacatattcttttatccactatcctaataagtgataaaagtaatcattttaattaataaaaatggtattatatatcgaactaaaagcaagtcttgtaaagttcttcaaatttaattacggcctcttaagatttgagtaactttttcgttcgaaaagactaatattttgggattgatagtatactatatttattttctttgttggtgaacacattgaccaaattagtacaactagagccattttgcttcaaatgaaatgaacatatcccataaaatgtggatacattgagtcatttttactcgcaaataaaaacatgaccattttataacatttgatagtttatctatgctaaaattagctggcaaaacttatataaataaattaacttgttatataacgttctggtatatacaaaaaactaagtaacttataaaagtaaacttcttagagtgcggatgcatataaaattgaaaaccgaagttatcagagatcaatatatagtttcgtatgaatctcatagtcgccaaaagtgcataaatgcgcaagtggatttgatttcctcaccgtaaacacaaaatgccaaagatgatatgatatgtatataggacttcatcgaagctaacataactggaacaagattatatttttaattacttgaaatttgcgattatgagcttacataaaagttaatattatggccaaaacataatcccacatgtcttttctcgtgttccatcaccaaattagccgtaacttgaacctggcccttgcttcttaaaaattatttttgcctaactactgtattatattagcgcatcaattcttggaaattgtttttgtctccccgaatatattatatcccgattttccctatgccatgcttcaagagtagaacattttgaacttatcattgttatatgcatgtcaatatttttttggttatatgttcaactacacaaacacatactacgtacttgtacatttttatattaccaaaaatagatatatgatatgcgtattagcatagaggacaccactcttgaactcttgttccctcactttccatttagttatttataaatttagaggtaagataacaaactaaaattgaaaggaagaaaaatatatacaaaaaaaagcaatcagaagaaccaatgtacagtagtatgctgcacgtgaaattcgcatgggtatgggttactgtttggcataacctagacaaccttcgttctcttgtcccctaagtactgatacagactatacagtagtattatttcattattagaaaccattcatgataaaaatttaccatatgatatagcttaattaacaatgaacttaaacacaattaagaatcattaacccgtatcttggtcttggatgactcaaaagatagtgttcgtaattaaaaactgttccggtgcaacgcatcgagaaccagtttcgacttcgtaatcttggtttactaatcatttgaattaaggttgattggtttcttgctaatgaagcatccaatatcttggagctagtaatctaatcaagatccttacacatacaaacatgaaacattaatgtattatatatgttcacaattgaaacttttgaagctgtggatttgggggaaaacttataacactggaggtaattgtgaacactgaacagtctttagttatgggcctattcttttagtccatcaagggggttatgggctatggattgggtccttgaaagtctaagtgtaaccaactgctatttaaccaacgcgcatcagttgcgcgtgtcgctaaaatgtatggactaaaagcgctgtttcttaccaaaacactttttctttaatttctcagcgcgtgaaacagggatgaacagtttggttttgtattttcttgtacctttttttctaatctattttcgcattatcaaaaagggtgaattgcaaaactaaccattttggataaaataaaaaaaaaacagagaagagatagcaacagccattaaatgaaccaaaatctccatgttacaccactgtcacaagtgatgcctacatttgcatgcactctttgggccactgtaatcatgagttctctaaatgcaggtgaatcattcttgtgaaatcaaaataagcaatacaatctgtcttccttaccgtcaaattttataaaatattaataaatattattttgaaattttcacatatgagagcttttaattttctgatctgaaaatccttttttcagtcaatttaaaatacagtgagaaatagccaaacatgttaaatcaatcattcttgtcaagataatcaatttcattattattaactgtcactatacaaaattaaaaacatgaaaaagaaaccagaactaatcgatcataacctttcgagaaaacaatggaacatgccaagtgaatttgtttcctatatatggattacacccaaaaaaacaaaagacaaaaaactaaaactttattgtgaggtgtctctaatatatatgtcagtcatccaattaagattcaacatatactatcttacaaactttcgagataacttatttgaaaatacccaatataatgaaacaaatttaaagatgacaaaagtactacaaaaagtcaatattttaaagaataaaaaaaaactcaaccatcccttttaaattcatatatgtacatttcttccctctctccatcatccttagctttaccttggctctttattttaatttcca

SEQ ID NO: 29 - DNA, 3000 bp, Arabidopsis thaliana
  AT3G12750 chr34053202-4056201 REVERSE (source_file=TAIR9_upstream_3000_20090619)
acatatgtcagctaacatgggacaatccatcaatcaaatcgtaaaccagataaaagaatggcgactcaattttctccttacgttcatctttaagctcagaacgcataaatggcagcttttcctgcaacttcccgattgaatctacactgccatcagaaaattgggtcttagtactccacatcaaaagacccttgatcaggtcacaacataaaagaaaactggaaagacccttgatcatttcactcaccctaatgcctgtaatccactgataaattcttgcctggaaaactcacacgctgttgcagcattcatatgccacgaaagaaccaactgaaagaagatgagcaagaaaaccaacgagtttaactcagcatatgtagtttggtagcccaagacgaaagcaagaacacatggacaaagttgtaggaaaagtttgatattgaacataccgtgactatgtcttgcggttccacctgaagaaaaattcaagacagttgttattaatggaagataaaaccacatcattcttaaagcattatgcaatgatataaaatacctcaagatcattacacagaaccgagataccctccgcaagaatcatatcagaatacgggtctgcaagacgtaacatgtcaatactaaagcacaaggtacacacaaatgatcaactcaaaagagtaacaacaagaagcttgcctttatatctattgtagagctcctccaagcgtcttacttcagcagcattgcttcttggttgaggctggctgtaaaacacatcaaacgctgcttcaaggtgccaatcactggctttgagagcctgaagagcattcttctcactgcccacattatttaaacaaatcaaaccaaacacacaatttgagataaatatggacaaaaccaattctagtccctgttttggcctaataacaacaaatctagtgataaggcttgctctacggagatagatccagttacctagctcctgtgatagccacgaactgctgaagtttgtcacggttgcttctgctcaacttatgctacagaaaacaaaaccaatcatatgattaacaaagatgtcgacaatcactgaaatcgcaaaattaaggagccttgttcgttttctactcgacttaaaatgaaactctaagaatcagaaaagcaaagacgccataagaggatccgcaaacctaaagcagagggaaaaaaacaaaaggaggagaaggagaaagcgcgtaccatagtgaagaagaatccagaagccaaaatacaaaaccctaacaaggtaatcgaaggaagatgaaatggaagacgttcgatgtttactatctctctctctctgactcttaatcgcgaagcaaaaaggtgtgagaggagaggagtgactcagggaagactcgagagagtctctcacgattgcttcttcactttcctctctctccgcctctctctcttctatgcaaaatgtttttttcatttctacttttctttcttataaatgttgtttcagttatgcaaatactccggttcgccaaaccggtttgtttagtaaaatgtctccctatacaaaatggaccggaaatttagtccggtatactgttctgtctttaagctttggactagagttaatcagctgggatataaattgtaccttatagtttataaaatcctgatttgatagagaatttgatacattttaattcaattggagctagaatgtggtatgaattttttcgcgggaagaaaaaacggtaaacgatataaaattatcttgaaatggtaaaaaaaatagtagacaataatggtataaaatttaactatatcgtttttggtatcgctttgatatagttattatgattcgtaattattaaatcgttaaatcatatactgcagcgatataaaattaccaaccaaaaataaaaagtgaatgtcttacatataccagagattaattaggtacatatatgtttatatataatctattagatcaattttaggaattaaaaacaaaaaaaaagtgaaatgatagtcaagtgtgatcaaacttggattctttccaatttctttgcttatgaaaattcaaccattgttttctcaaacctctatatcttatttatagttcagagttttcgaaaacaagatataaaaggccaaaatcattaggttcaggtaaccatcacactcaagtggcgatggtgaaatctgaatttgaaacacgtggtttgattgggccagcattacccacccatcccaccttgagaggacgatgagaccatcttacgtttcttgaatccaaaagtagatttaacaaatcaaaccaaaaccactctcctcacggctcacatagtaacatgacgacattctgtcttcacattttactttctagtaaaaaagtcttcaaattttcaattcataatacacatatattttgcttttggtaaagtgaaaagatatgtactaacctaataattacaaggattagcattcacagctaatctcttttccaccctattctacgcccctaaagtttaagaccaataatgttccaaactcacttttagtctctatgatctttccctaatcatctatctttgaattgtcgtctttgttttattattgtttgtatatctcaaaaatgtataaatagataagtaatgattagaagagatcgaaagggcgtcttacttgaaccatccatagactacaatctaagatgcttgcagaatttcttatactaaccaatgaattctttcgcagcctttttccgtaaatgttccatcctattttatctagtcattgttaaagacttaatggtatacaataagagtaataacagaatttaattatttttttgtgtgcgacaaacaaagaaatgtgtatagtggtcaactctacgtaactagagattctgctactaacatttatggaccactcgcttcaaaagtgaggttctctctcgctttctctctataaaaagctcctctttcttcaccataactcttc

SEQ ID NO: 30 - DNA, 3000 bp, Arabidopsis thaliana
  AT3G15170 chr35106883-5109882 FORWARD (source_file=TAIR9_upstream_3000_20090619)
acgcatcttctgtattgtttttctgaagactttccttagaaaatcattagaacaggctaattgacaatgtttattgatttctctcctctagatatgagtgtagcgtccattaatatttcttcagaaatgccaatgctattttgacgttttagtgtatagacctgtatcactgccatattggacgttatcttacagagatattgtttgcgtacacagatccgtaggaggctactggaatttgtactagctgcatttcagaggtctccaaatttcattgccctgttgaaagtaatgtctgcctattttattttatttttttgtgcgagtaatagctttagaatttagataagaagatttcacagtgcattattagatacatagtcttatgattaccgcttattttctcgagtaatttgcaagatatgcattctcaacttcttaaataatacagaagcctattattgaccgacttggggaagcttacgatgatgttgcaaaggtcagtttctaatatttagacggatgctttgtaagataatctcctctaagcattttcttccgtagagagagttagccttacagctatgttgggcaattggagagtatggtggtgggggagaatctcacaaggaagctgctcgagagctttttgaaagtttagagttgcttgtatatgagaatctctcatcaaggtattacaaacttttgtttgtgtatcatcaatctgctttcttggacagaaaacataaatggtaattacaatgttttccctatttgtttccaacgaccaaaaaagccgcctaggtttgaggcaagaatctgggaatggttcaagaagaacaacacagtcaagacttctgtgttttgtagtgacagctatcgcaaagctcgcaacatatcaccgagaattattaccaagagcccgcgtcgccttgggaaaggtacacaggaaataattgaatctattaatgcacttaccaatgattagttgattactaaacatctggaatctgagttccacaattatgtacatgtgtaggtagtaaagtctcggatatctgatgcaagggtttggagacgagcacatgattacttaggactaatgaacgaaccaggaatctgttggtctgtgttaggaccttcacgggtttcagaaaaacggtttccaggcacagttaactggagcgagggtggacaaaaaatggttgcacacattccatttcacattcttagtgaacaaggaggttaagagactcttctcatcactctgttaaaagttaaattcaacaagatgataatgtggttggatttagaaaagatctaacgttgtgtcgttgttttttgcaggtcctccattccatgactttgctttctccgatatcatccccaaaaactaatcaaacaaacaagtctcttaaattgtgttttattgtgaatagtgaaactcaaaggtgtttgtgtttgtatacatcggcatctcttttttttctagcagatgtagttttgtaaccttcttgactatggttcgttcaaacttggcaaaattcttaatcagacccggttggatccggtttaaatttggaagaaatttacaaaagaaaatttagtccgacccaattatgacgaattttctctcgccgtattctttttttgatccacttttctaacaagctccgaatgagaaaatgccaaaagaaaaactgtgggtgggtccggaacttagccgtagactcgaaaaacgacaccgtagggttcagggcggaggtcggcggagatgattctctcatccaccacactactctcactcacacatacgtcactcgacctctgtcgccacctgtgtgtaaaaatctcttgatacgcgagtttttttaactctctctcttttctctgtattaattctctcttggtttttaaaacaacaatccataattattgatcctcagcgtcatttatgaccttctattttaatctttctttctcccaacaaaatcagcagctgacattgatcttttattattgtttcaatgaattttcttgaataaacgtgtgtgttttctcaaaaacaaaaatgaaactcctgagatatttctttcgtctacttaataacatatcgtgtaggtttttaaccaaaaaaaaatcaaaacacatgacgggtcggaccataaattaagttgttaatttttttgcatcaaaagtttttgacctttttaataggaaaaaataataataataggtagagttatttgtgagaccgtagactagggggtgaaaatacagaaggagagaggggaaaaaaatgaaagatgcatgcaaaataaaagaaaaggatgggaatatgatttgatgtaaagcctgtgacaactctcccttttaaatatttttcaacgcgacgttaccttattgcagacatgacgcgtctatatcctatccgtccattttttttcttcagatgctgatggatggatgtgatttcttcttatgatttgatgcatccatgcacctctttctttcttcatatttgtttttttctctctaataagttttggattaaatgtactaatgcgtttagaattatcgttaaaagtatcttgtaaacagacaaaatccaagtagtatccaataagctaaaggtctaaagtgtttcataaagttaaatttatcgtctttttctttaggctgacgtagaatcccctgaatttcacagtcacgcacgcattgcatgctcaaaagacaaatgtcgtccaataatatcatatagatcctaaacaagttttagcaaaattattacaatcctatacacacacatatatattcgttttgtgtgatgtattgtacacatcaacatcaactctatctgtcactagctactgtcaaatatcacatcagttgcttgtagaacaggagaaacacagatcattaaaataaaggaccttttttattttcctcttcttttctcttcctttatctttcaacatttgggaactctagggttttgtt

SEQ ID NO: 31 - DNA, 3000 bp, Arabidopsis thaliana
  AT3G16340 chr35536897-5539896 FORWARD (source_file=TAIR9_upstream_3000_20090619)
tctagtgattgttaaaaatcgttatttacttacttatttaaatgtcacatagagagaaaagagatttgcatacttgtaataatataatcgtgcttgtataagaaaaaaaaaactttatgagacaagaaaaaaaaactcgggtgtacgtacgtcgtaagtaaaagggccaatgcaagcaagctgtttggctctttccttaaagcaaaataaaagaaaaagttatctttttctccttttatttcaacttttttctttaattttcttttccattttttatgcttaatttttcaactccgagatatgaaactctctactttgtgtttagtttgaactcttttagtcataaaacaacaaactctgccgcaatcaacaaaaagcgaaggagtcaaataaaaacttctttttacgagtgaaagtgactattttacttcaatctcttttgattttcaaaactaagaaaaaaaagaatggaaatcaaagttgtgcatgttcttcccttagtcgatatgtaaaagtgatttgatagacattattgtatgtaaaagtaatacaactctgaaaaattttaagaaaaattgctagtatcagctaatccaacaattagttatatgttttggagaatgtgtaacatgaccgattataaagctaacatcactttatttattgaatttataagtaatgcaattatttaaataggtcttctcgaagaacatctagatggtatacgcttttgtgaggtgagctcccttttatgtttgatttaatagaagaaatgtaatgtgtaatgacttgcttcaaatgatttgtatgttcctattggataagggcatcttgattttatttttattttttgtatctataatgtgcagtaaactttttacttttcattttagtatgatagctggagtttggttgtttcaaagataggcaagtattgtgtttaacaactaaaaataaaacaacacatatttttcaatagaattatttacaaattataaatctaaacacagggaaaatgttagatacaaaaacaaaaaaagtatcaaagacaagcagtgtatcagacatatataatatcatattttcaattcattagaaatacaacttgccatatcgcaaattagtccataatcaccaaggtagggaacgattgtctatcgataagttatgattgaaagaaaaggatttcatgaagaaaaaaaaacttaatttttcttccagaatcgtgacttagaatttttgttaacaccgacgaatttagataacaaaagctactccctcattgactaagccaagaaagttgagtagttgactaacaatgggactaatattttagcaaaatcaatacaattttggtaattattttggttcatgaaaatgttcaatttttttggtctgaattgtggaattcacgatttatttagcctttttgcggtatatataaaattaagatatttttatgtaacactttgttttataaaggataagcttgtatagtttatatataacggctagtcatgctcaaagcctttatcatctgtgggcttcacaatcttatttcttcgacccatttgcttcaagcctcaaaccaaaacttggctgagttcttcgaaagctcattatttcttatatagtaattcttttacatagtgttggaaaatgaagtaaaagaatatcgaaacttgaacgcttagatgacgaatctgtgacattatgatttcaagtatcgacgtgttttatcaaagtgaaaagttgttaatgttggtgttgggtccgtgaaagctcgaagccatggagattaatttcaaacacttttgacctttgagccttgcaagtttggtttatttgttcaacgccagacacgtttgcacagatatgagttggtgagtgcaaatcccatcataccaactaatataaagttataccatcaatgatattctatcgtgaaaaaatatgtaaagtctaatatcattaagtatcatttattaaaagaatgaattgaagggaaaaaaaaaaaaaaagataggcaatccactagtgttcataatgaaagatgatggtcgatgtaatatgtatactaatattatatctctagctttgatcctcaatagtcaatatctatagtgtttatagtgttttaacagcttttgtataatgttaactgtgcaaaatacatgacataaaataaatgattttttttggcttttatgttataagtactattagttggttatctgttttaaaggtttttccttgaattagaggatcagatttacatgcccataacagaaccaagctggtcaccaaatccatcttatgtagtggcataacagaaccaatcttggtcaccgaatacagtagtttcacctttctctttctccaaatataataaaaaacttttttattttgtgcaaaataaactttagtgagatatattgagcaaatatgtatatatatataaagatttcttgagacgatatctctttgcctctttgggtcattgttaattccatgtatcgatttttagggttgaagagacttgtttgtaactttgaccaattgtctactctttttagaccacaatgttagtaagtatgtaggtaggacttgtttcgaatgtgatgatttatttaggcccagacaaattatttactagacccattgttgttctattatttactgtttgaaagaagttgtttttgtcgttgattgtttactagtatttcaaagaaaagggtatgcatagtttaaacttcaaggaaatcccttatatatagtaaaattaaaagaaattccacaattagatttcgtaatacaattttctataacaaaacctagttaattagtccacatgtcctagctcttacccactcagcccgcatattcctcacgctttctttttattttccgaaattttttaaacggctaccaaatcaaaatgcataaataaacaaacccttctcttctttaccaattcccaaatccaccaaaaaaaaaaaa

SEQ ID NO: 32 - DNA, 3000 bp, Arabidopsis thaliana
  AT3G27920 chr310363507-10366506 REVERSE (source_file=TAIR9_upstream_3000_20090619)
aaccaaaattgcaatgtcttcaaacggtcttatggtttggattggtttttaatttcggttcagacattgggagtgacgagtctgacacctagaaaaaatcaaaatgagaaaaatgaaaaccgaatttcgtcggcttaatggacagttacctcatcttagcaatgatgcatagtactgattttgcggctttgtagtaccaaatttttttttcaacactctctcggatgcatagtactgattttgcaatgatgcatagtactgattttgcggctttgtagtaccaaaataaaatttcatgattgtgcggcttagcaatgatacatattacatagtacattagtactgattttggacctttattttagtcgagacaattctgaacttacggattccgaagttaacaaagtccttttatagcttttattcagttccgaacatggttactttttatacatgaaacttcctaaaattttacatctacaaacattcgagtttgaaaatttaatggtttgattttcgactcattaccaagaatttctttttgatattattcttcatgatttatcaaaagatgtacgtgtattatatggttatgtcttgagttcatgtcatgtgttcgaatctttataatatagtcttacacggtttaggttatttgttcaattgttaagtcattttacggtccctatttcacattatgttagttttttaaaatatttaaaacaacgtactgtagttttgataaattattgagtttttttattaagttgtgaacgcatgctctcgttgagagatcatttactcgagctaagttatgacttggatggttgtgttgcaggtgaaaggtagctttgcaaaaaaaaaaaaagtttgaggttatgtctgcatgtttccctgtatacaaactcaaccatttgtattttattttttgggtgcaaaaactataatttacgtagtttttgaacactttcataacatcaaatattacccacgtgataagtgaaaacgaaccgaatatgtaatgatcagcgaaggtccctacgtggaatttaatatttgtgtcattctttttaagcaacaccaacaagacactgacatgggttggtatttataaatttgcaacgtaaagtatatgtagtaagattgtatcttctatggttgggactgtatctaaaccaaaccggtttaatagggtatatattttttttgtctttgtctttgaatttgtggggtcataaatcacgggcatcttcatttggaaatgtattcatagtaatgtatataaactactttcgagccgacttggtaatgattacgagaatcctctgcttttggagtaaatttctaatttaatatgtacgtgtatctagatatatatacgacgacatgtcgttacccaaatgcttcgttagaggatagcttgttgagaacttgactatgccaatcatatatgtttgtttggctttatgtagccccaatattaacaacttaatactaaaaaacctaataacaaaaacattgagtaaaaaactacagtacttataagtaaaaagaataatgatagtagctacttcatatacataatttacatttcttagaaataattgtttgtactttctcgagttagtcaaaaattaaagtagtttttttataaccgaaatactatttagctagtggaaactatatttttctgtttttgtgcaagtaaatagtatgcatatttataagggtatatgtataagctagctcgaaaaccaaagtagaagagagtaggaaggtggtaactggtaagtagggagacaagtgaagagcagagcttctattcctccaattatttagttaaatagttgtgagacatattccttacatattcagtttcactattttattctctaccacttaacttgttagttggaactaggttatatggtattcaacatatactcatactttgtacgtagacgctttcttgagtaaataaattaaacctttcatcaaaatatatatatatatatatatatgtctttgtatattaacagtatgcacacacatatatataatttaggtccataatttttctaccccgaaaattagaggagattaatttggtctcttatctactataatgttcgaacttgtgacttgaaaattctcattttgactaactaatcagtatacaggctaaactatctgattagatttaagaaattaaatcgatatgatatgtaaatgaacacataaattttgtggtctatttttaaaactcaattaaagagggtaacctcatgaacttttaaaaagtatgtcgaaaagttgactataatttgagaaacaagacatatggaaaggagaaccatttcattgatccatcaaagtttcttttttcatgttttatcttctgcaaaaaaactgttttagggtgaactctgtggtacttttaaataggttgttccgatcccactttgttgtataaataactaaaaataatatgtagttccaatgaaaaagatcattatcaaatattagtagaatgtaagtgtatacagtgcataaacaaactggttaacgtgtcaaataaattacaaatcagttaaatcatacaatatagaacatttcgcagtaatctgccccatatttagtagatttgtttttgtaaatttcatgtcgcaagtatagtatttagatattgccagaggagaaaaaacaatttttttataaattttttggccatagatacaattaaaccaactgccaaattttagcttttaatacgtaacgtagggtaacaaaggataacgctatagtcgctatctatacattattaggtacatatttatgcttatagccatgattacacaaagatacgtattgatgtgagtgtatatataagcacgtgtcacgaaaacccatcataagttcactactcataatctctttctctctctcacacacacacacagacacacacaaacatagaaggaatcagagaataaaaaaaga

SEQ ID NO: 33 - DNA, 3000 bp, Arabidopsis thaliana
  AT3G30340 chr311953601-11956600 FORWARD (source_file=TAIR9_upstream_3000_20090619)
catgggttacttaaaactaaaccctgaaatatattggctaacaaaggggaaaacacacaaatttcgacctaaatatatataatccaacgatggtgagaatgagaaagtatgcttacgacggtttaaagaaccaaataacccgttatttacgctacttctctatttttaagaggagataacaaaactcaattacaaaaaaaaaaataacaagaaaaacttgagaagtataactagttgtttatatccatcggacataaccgcaaaggatgcgaccagttgtgaggagaaaagtgctttactccctgccatcggacgtctcactcaagaccatttacgtcaacttgcccaaaagacaattactttccaataccactcagcaaaaaattcacaagcaaatttttctgataagaaaagtctttgtttggtttagtttatttataaacaaagagtaaccgttgacgttgagtatttatcacccaattaatctttctatattgaactagtcgtggagtagttcactcacctaattttttcttttatcatccacttttagttaaaaagtaaaaaagccattaatgtgaaacattgtttatacataactgatgtgatacagtttaaaatgaatttttgtatcaaattatctgtttttacatttgcgtatcgaaaaatttaaaataaagagaaatttgtgaactcatccttatcgctctgaaatttttccaaatattttgaaaatgcttgtcatttagttgaaaaacacataatcttcaccacatatgaaaaatctgtaaaaataattactaatcatacatttcactcacctaattttttaataaaaatataataaaatacaattttgactagcaaaataaaaatggtttgataataacacgttcgcaaaatttgatccatgacctaaaaaagagtttgttgttatagtttccacaaaaaaattgggatactattcattttacatggcaagaaacgtaaaggaaccgaatcataataaacaaatgaaagatacttagagctaagcattaagcatcccattttgcgacaggatttgacgcattatgcttacctccattttaaaatttgatatctcttgtcaaatctttatttatatatattatacatgatatgtataaatatttcacgaagaaacaaagtatagatgcttcttattgtctgatcagatctctactttttattgagaatcttgtatcaaatatggttctttttgagccaaacacgccaactttcaataatcttcactttgctgactactacttcatgttgattaacatacttattccaaattacggctctaaagaaagtgaaactaattaaaacgtgcgtgttgatacttgatagtatatatacttcttacgcatcgcctgattagatatttgtttttattaggattccatgagagctatattgcatggttaattggataaatggtcagagagacgattaggattaaagtaaatgacaaggacatttttgattatttctcgattatcatggtgggtgtaatttaacatgctgtttaattgaacgaagatactgtactaatattttacacgccctaagtaagactcactttatcccattttatatagatttttttaagaaataataaacattttatcatgataaaaattcaaccaatagaaaaaatacggcagaattcaaatagtcataaaatatcaaattaatagttatgcatagaaactataaaaaaaaaaaaccttataacaaaccatcaatatattgcgacaaaggcagttataagtaatctcaaggtaatataacatgcttatacatccatattacaaaactcatggataaattataatgggttttctgcaaaagaacctttcaagtcaatctcgtttgcaagtttaccctccaaatccgagttatgcaagtcaaccccatcatctctaaagtcatccgcaatacagccttcatgtatatcgataacgtggcactcaataactctgtttaaaaacataagggttttatggaaaaaaaactctcaatataaatttgttttgcaaaataaaccttcatgtcaaataaatacagatcaaactacaatctaaaaatctcatgaaatataccccttttgtgtatcgatgacatgacagtcgatacccattcagagcatgtgcaataggtatagattttatgagtcgtttatttaagattttctgctatcacaattccattaataataattttatatttttatctattcactattctcgatcctggtttatctaaacatagtaaataatttttcttattttttattcatgataaatcaattacttatttaatttaacgttctataatttatatttcttttaataatttattcaattaatacttaaataataaagggtgtgctggtaaatttcaatataaaaagagtaagcatagttttttcaaatttgcataaatcatttgaactcgttattttcttaattatataaatggtatgtctttcactttgtacggagtatcccaatgtggggtacataaatattaagagaaaaaaagagatcaagcttactagtttaagcgttaataattatactgactgaccaatctacttgttcatgtagctctaatgttattaattaatatatagtcttagattgatctaattaatgtctgtgtatgatgtcactttgtgatgatggaagtcatgagagcttccattacttaactctctctctctctctctatctatttttacggtcatgtatagtgtaatcaacaataacaagaaaaagccaagttcaaagcactatactattagttaaagacaaatgcgataatgatacgattctgagcattacttgagtcacaatatatagtcacaccctcatcaaattctttctatcttaccaacttctgaaaaaaaagaaaaaaaccaaaagacaagtaaatacatatataaatatacactcacacacacacacatat

SEQ ID NO: 34 - DNA, 3000 bp, Arabidopsis thaliana
  AT3G42670 chr314760086-14763085 REVERSE (source_file=TAIR9_upstream_3000_20090619)
cttcttctttcacaagcctgcaaacacagcttaaggaagctaactgcaaaatgaaggaacaagaaactttacaagcagagcgtgaagaagaggctttgcaaattgcagctgagcacttgatgatcgtgaagtacttgagcgcaaccgacccaaacattctcgccttcctcgagtctcaatctattgatcccgctactgctgatgtccaaacataggtaccttttccatccttctctgaatttgaaaacgtgtgttggtttgaataagaatttttgttttgttttgaacatataggttgtttggttttgaattgaacaagaatgtgtggtttgttggatctgttttaaatacacatttcttatgtgttaacaagtctgttgataataaattagaattataatcaatttatcaagaattaattgaaggctaaatcaagaattaaacctctaatcaatcacaaagtagtagcaattcaatcaccaaaacgttcacaaaacaattgtgatgttgtggcaaagatgtcgcataacagtcgctatacttagcgacgacattgcgactaaatagtgaccacttaaatcggttgcacattagtttgttatgcgagtgattattttaacactcgttgtttaggaaatacgtgttttagtcgcaaataagtcgcaagtaggggttttgcgactgattagtgacttcttatgttgtcgccaattagctgtttttttgtcctaccaacaaaaaaaattagttgttttcttgatctagagacttgatcgtagaattcgatggtagtgaggaatgttggtgagatgagagagagagagagagagagagagagagagagagagagagagagagagagagagagagagagagattgttggatagcatgacaaaatttataaatcaatgataatagcatgttttgtccttgaaatacatgcgtttataaaatcatatataatcttatatgtcacactgacgagtgatggagaaaatccggtgagaggagaaacacaaaatatacgagggataagaaagatagcttctaggaagaaagaaagagagaattgatggaatcacatatccaacgagaggaagataggaaaaaaggaatgtggtggtgatgagattcagtaaaagaaaaagatatctagagagagaatgactataatgttgcagacaaacaagaaaaaaaaagaagaagataataaaaggataagatgaaccaaaaacccttaaaaaaacatatagcaaatagtgtccatttagtcaaaggttatttcgttgattcttttcgagtcttaggttagtttctatatattctctaaattttataattttcttttcgattctagtgagagctgttgctaaagctaaaaagagaaaaagatatttaagtaaagttttctattaaacattatatattttactatccatatcaaatcatggaacattcatagccacccaaaacattttgagtgccaccagcaaaaaaggcctaatcatatcaaaaaaaaaacatttttgtaagtgtaagagttgaacctactacctattgttctatcacccaacgattaaccagtatagttataagagatatttggaaaatagatgacggttaattaataactatatcttataccgtaaacacatgcttccctagcttgtgactaaggcagattctggtaacaactcatattttatgcataagataaagctcacatagaccttgttcaaaagcattacatatccaacgatttacaataactctatcatttacatttccaaatgttatatattttagtgtttgtttgatttaatggaaaaaaaaacagttttgcattaactttgtaagggatacaacaattaaaaaaagagtcagaaataaaatagaaaaataatataaacaaaaatagagaaatctactaaagaaaactagatatttgatcttccaacatattaaacagaaaaataaaaaactaaagagaaaaaaaattagaaatggagtaaagaaaatacagcaattcaccgttacccttaactactttttaaaatattaaatgatattttcttttagaatcttgaaataaaaataaaagtttcttttttgatatcaaaaccatattaaaaactcatcttttactaattttagtataataattaaaaaacaaactaagtaaatgtttttgttttgtgcaaactaaatgttaagttcatgaatttgtgtaagatattttccctataaacctatctcaatgtggacccaataggccaccatgcacacaacttggcttttactctttgtgtcagtaaaatctaaagatagtaaagacagaccaaatcgcagccgtttatttcccatataaagaagtacgtacatggaaatgacaataataccctcactccttaaaccctcctcttctctcactttatttttcagtctctctttatttctctctatcactttttatcgattctgcgcaaattcatgcaaaaagggaaaataaaaagttctacactgtccccaagattctgcgtgagtgtggtattgaattcttcgccgtcccttttctatttatcatattttgttccttctcttattttcctcttagtgtcaatcatggactagaatcaactctctttttccctcaaaggtgatatattgattttgtgttctgatcataaagttgtggttttttcaatctgggttttgtttgttgttgatgacgatcatgtttcgtttttcattttgatacgacactgttactctgttttacacctttttagtacaataatgttgctttcgcacattaaccaatatgatcaagttttaaagttcatatatcatatatgttctgggttgtctctagttgcagataaagtttgaatcttttttttatttgtcaagtggtgaatgaatcctaatgaagctatacatgtattggttacaggagatcatatgtggatccacaagtgatcttgaagggaaagaaaaaa

SEQ ID NO: 35 - DNA, 3000 bp, Arabidopsis thaliana
  AT3G44970 chr316429443-16432442 FORWARD (source_file=TAIR9_upstream_3000_20090619)
tatattgttcatgtaatcatgtgatctcgtagataccatactcctcaacttagtgactcgtagatatgtacattacatttcaaaatcctttgttgaggctccaacttcctttcgtacttacagaggaaaaaacggagtcatggggcatgtgactgctgaaacaattatatgtatcattaatatactataaaaaaaactgttaatatcgaaagaatagctatgacaacgttatggctatgtgcgttatccacaaacaagatacaaactttaaaaactaaaaaatgtagctattttgtgaccaaattatgaatagctacaggcaatcatttgtggatatacgtagcaacttaaagataaaaagcgcgacaattttttttgtatctaattagctacgaactatttacgtatatttcgttgcaaatataacaacaacaacaaagtgtagataagtgtgaccaagacaaggaacgagttgtaactatttctagcacattttgtttttagaatagtcatatcaaattatgcagtataagatattttgcctaattaaacaacaaatatacagatatgaccacaaaacaaaatgtggttattttgaggctgaagccacggaattgttttggcgaaatttcttgcagataacaaatttacatgaatatgaaacacttattatacatcatgaattgttgtctgaaaaaaaaaaattattcatcatgagttattatactctctccatttcagattagatgttttcaaaatttcttcatacatgtaaggaaaaaaatcatttgtatccttcattagtatatttgtattattttttcattagataatacactaaaagagtaaagataaaattagaaaatttattaaaactcacattgaaatataaaattacatataatctgaattaaaaaatcaagcatcaaaacaacaacttaatatgaaatggataaagtgttgtcacgatatcacctgtggataggtaatgtattcaattttatataataaaatatggagagaacttttgtgatcattttgtctccacgtcacaattttagtatattatagcgttttaataataatatttattaccatttcttttttgaaagcaataaaataaatataattaaactcgaataaaacaaacataataaatactattcaatacatgtctcctacataataattattaacccgatttattatatttgttttatttgagtttaattttatttattttattgcttttaaaataggaaaaagaagcttgattccacacgtgatattgtgataatacttcttcataatgttaatacttaatagtgattaaaaaaaaatcgatgataaacccatagtgaaagttggtaacatgttgcatattggctaacattagtattagaccatcattattggtagttgcttagtatagttcttagattaatcagattagaaatataatgtttatattgatttatttgttaagaatggctcttatataagagaaaaaataaacttaagagctggacaggtggcgtcttttggatatttcttcttaaattgtttattatatcaatatatgtctctgtgcttagaatctaagtctacgaaaattaggcattaattctcaaatccgtttactatattgattgttctcacaggtcctagtcaagttctcaaaagaaggatcttgttagacgtcaagttctctgtgttaaaggttgcctggcaggatcaaaagctgaaaacctttttgaacaatgcgatatggttctggcagtccataagatgccagttacatgcttcaatgacatagaaactgcttgccaaacattggataagggtagttacagcgatgaaaatctcaatccaaccagtgagttttaccatgtatatattttgacgctgaagatggccggatattagacactaccattgtgatagtactgactatggtacgtacggtgatgacgaaacggccttttatatatattttttaactaattggtatcatcctatggcaggtgttatagttgacaacagtccgaaggctcagtgttacaactaatgataattcgacttcgacagttggatacaacgttagtatttcgaaaacgattcgaatctcatacgtatgaagtttttggggtgttgggaaatagaccaaaactatgactcaaaagcaatcaaaaccgaccaaactcacaaattaatcaagatcgatcaagattacaaatggagatcaaccgggcatgtatatgtttgtaaatatattggcataggaagtttagttttatttttatatattaattttatttgtcatttttatatataattttcatttgtgtttattttagttttaaaaaggaagatgcctgaatttgtgaaataataaaaagaaaactaaaaaattaggtgctatttcgcacgcatcacttgagctaacacatgcatgtgttccttctatttcttgctatatacgaatctctttaagaaaatttaccagccaaaatctacaattctcttttttgtttgttgcatatataagtcaaggtttgaatttcataaaacggacacaataattaatatagataaatggttctagtttctaaataagaacactttattctatgcaacacataaaaaaactgatattgaacaaaatcatattataatctatgtgttcgacttctacattttttaaaaaagggactattattgtaaaccattccccaaaagtgaactttatatccaattcacatttttcattgaaattctgttaatatttttaaaataaataaccatgaaaactaatcttacaattaagaatactccctatcattctaattaatccgagttttagaaaagaatacaacattttaagatggtcaagtttgtcggcagttttttagaaactggcacatttatcatctagtttgttactctaggattcatcatctatataagacccacgagttcatagttccaaggcacaacaac

SEQ ID NO: 36 - DNA, 3000 bp, Arabidopsis thaliana
  AT3G49950 chr318519510-18522509 FORWARD (source_file=TAIR9_upstream_3000_20090619)
tcctattcctaacatccttgtctagctttctcattagttgagtcgggggagtgtgcttatcgccatgtcttctacaatttctttctctccaaattgagtagattacaatctgaaaagaatatcttagtagaaataagcgaaccttatcaagcgtgttgtccgtgagcagccttagaatcacgttcctatcggtagagaattttgcagctaggagtttagaggtgaggctcgaccaaactattttagagtaatggcagtcgaagaacaagtggtttctagtttcttacctagttactttgcttaaaaatcagtaattactacaaatttctaataaatgaataaatagggggggggggtattggatcaaagagtttaattgaattgaattggatacataattacagattttcactcattgcaaaagatttcaaatcttgttttcaattctaacgattacattttcactattttattagaattgaatagaactgtaatagaattaaaaaaagtactaataattcagttttgctaacaaccggacctatttcttctccacagtctccacttgtaaatctctaatcacacgtttcaaaatctgcaatatttcctccttcttcttcgtctcaaaaccactctcaatactactaagtttaacagatcccatagcttctccactttcctcgctaaatcctctgtgatcattgtagatttcgaaacatcttcattgtattcttctcggaattcacgttgataacgagagctatctccgcagctaagggcgtcgatgatgattatgcacaatgacgagagtactctatggtgcaaagaaccgttttctgagaaccagtaacattttaggcttgaaagtttggagagtacttgaagacaagaattaggaacatcaacaataccaacaataggcttattaatcgtgcaaaaggggaaattagcagcttattagcctttccactagatcgaacatgcccttatacgacaatttgaggccccttatatattttcatccaaatcacaacttaaaaggtaagaatttgttttgtgcattttgcactaatatttttattaaattcttgacaaatgacgttgtcatgaaaaattacaaaatattgaataacacttgaatttagtagaattaagaaattgagaaactgaataatactagaatttaaaagaatttataaaatgcctaattgaataacactagaatcaacattctaataaattccgtcaaattcattgctccaatacccccctccccccccccccccccccaagtaattgaaatgggcttgaaaccatggggccggaaattttcactttgtattattttgatgaagcccaatagactttacggaaagaccttaggtttcaagtaggtttttaagttttatgaaacagagaaacatatacgtgttcgtgaaggagaagacgtgaaggtaacttggactataaagtgtatagtgttagcgaaaaggtgaagcagaataaataaagtgttatgcgttatcaagtctacctaatataactagctagggatttctactcttgttttcataatcgatctacggacatttctcggaacgtggtcaagattcatgagtcttctgttttttatgtctctgttcaatttggtttagagattagtatgcttatttgtttatttcatatatggttatgagaggagaggctaatggcatatactctgatgtttgtgatggctgctaatatcgttgaggagttattcacgttgtttcatgcgcaaaaatcaacagaaaaaattctgattatgagccaactctgtgaacccttatagtgcgcccagaggtttgcgaggcaaaatcccgatgaaccagaaggaattttagatctctatcaacaataactatgatggagctcgtttaaattcatcacagcgacaacatcattaggctgcccaacgtctatgtctcctggaggtgatggtacttgatctctcaaccaattttcttgaaaatatcatgccttgtgagcgctttcatattgcgcctaaaatacccaatacgcaatgaacctacttccaaaggcatagaaaaaaaactgataatgataatgagatttgtcactatacttatcctatccctacataggagccgtttgattgtttagtccatgttttcattttgtttagtctaatgctatataacttttctttatcagtctattgttatatgacttatatatatctcaagagataaggccaataaatcttcttcttaattatatctgaagactcaaaacatattttgagtttaataaaataaataacgtccaaatgctacatacaaacggaccaaattcatggaggtataaatttaaattattttttgttccaaagtgtatgcagtgatttattgatgaatgcgatagagcggcgaaagagaataatcgtcacctagaagacaaattgatcggccgtacatatatacataaatacaaacctgccacttcacatgtcacccacctttaagcacccccttcacatacatactttctataacaaaaatatcagcttctagttcatatttatgttacaataactcgagtgaatcatactaaaaaaatgtaatgctttctctaaataggagataaaatgcaccctccgacctaactaaagattccttattttagctatttaagacatattgcacatgtatagagatacataaacacatatgcaatatgcacatcttctatacattgaaaaaagctgatcttgcaaatatttgtcttacacaacacaagcgaccaaagcgatgcgtttcccaatgataaggttacgacatacttacacgactctctctattgtctcgtctctttctttcctcatccctctcctttgtctcctttcactctatttttcacttttcagaatacttttacgtaaaaatcatggacatgtcattgtctccaccctactatactctttttttgttctttttgttt

SEQ ID NO: 37 - DNA, 3000 bp, Arabidopsis thaliana
  AT3G50870 chr318907990-18910989 FORWARD (source_file=TAIR9_upstream_3000_20090619)
taagaggtgggtccaatagcttttcctttgtttcttttcactagttcaagatcaaatttatggggagtattattagttctttgacctttcctttttagagagtccttctttgacccatcaagattcaagagcagaatctctctcttgctaagctaagaaaacatgaaaagtggaataaatatttttaaaagaaagaagagtatagctcatacatgtgtatgcattggttaattgggggtatgttgctttgatgattgtaggaaggtacttcacttgaatagaggggattcttccctttatcttttgggttcttcacaatctaatctattagcttttcttttcatgcagtatttttcttgttactctaaacctcttccaaatcttcttggatattctcatatcattatgattccaaattcgtttctctttattaaaaaccccaataagaattggattctcatggttttcgatacgttattcagccatacgacgactatgagtgattctagtgagactaattgaggattttttttttctatttttacatgagaatactgaagatgtttaatttatctctgattaaatataatccattatacagcatattacgataagtaacaccgcctcgtttgtttttgtttagctagtaaaccgaaattttgatcgaatacattatataaaaattcatgtaaaatactctacaatataatactctcttgtagtctgaatgtaattataaaaaaatgttttgtgtagacattaggtaaaccatctagagtattagaaacaaaataatgtaagtaaacgtaaaactaagacgatttcatgactaaattattttgaaggtcgaaccaaccaatcttaaatacacatatatgaagaaatatatgttttcgaacaaatggcaatcaagaaaatgctttttgtgtgttaatgaaatataaggttgacacatcaatatcgcgttgatattctcattgtagattgttggttatgtagggagactaataatgcgaactagaaaaagatatgtgagtcaactaaggttagtgttgatatagcaactctcttatgtttttaaatttgccataaaataggttgttagtccaaatagccgatataaacatcaatagaagagtaattaatcgttccatcggaacatccgatgaaattggaacgagacctatacagaagaaaacaaaagagcaactaatcatcttgatcacatgcccaatatgcatttaagtaaattggagagaatgatatagaaaaacaagacattatctacaactacaagatatcaaagcactcggttaaccgcgtccctgtgaagtgttagttatcagtacatgaattgtgatttcagcatcttggtccttttttttttggacaaccgtcatctttttcttgatcggtttttataacgtacaaacatatactatttcaacagttttcttttaattttttttttttttgaatatacactttctttcttttatttgtttgattacaaaattttcaaaaaatagtgttcggcaaaaaaaaaaattagatcggccaatgcattatatcccctagcgtaagctctttttcaaccaatcgagagtccatgttttcaataaaaatagggttttattagtaacagtagttaaagcatttaacatacgtaatcccaaaaaaataaaataattctgtatagggacgaagactagtattcgttgacgacaagagtagagtagactctagctagctgcaagttttgaagaaatctaataaccaaatctaagtcattttacttacatcatcatggatcatcattaaccacatgaggctgagcagatatcgaacgtgtgattgcgatgtggagaagtaaaggacgaggccacgcgcaactgagcaggagcgcgtgagattcagggttttagggcagagatacggaaagagacagaggttttatagcattatattgaaagagaacaaataaaaataaaaatatgagcctcgtgatcggagatttttgcagtgatgactgtactcttcttcctcttctctcccccttgtcacgtgggctaaattttgaccacccttcttcaatgaccatcaacatcgcatcacactctctccttcttcttcttctacgttttttattttctgttaaatttcctccacaaaatcgataatttcaattttttcatacaatatcatagttctagtaaagtaaacattcgtgtgtgagtgacataataattgcttcaaaaagaaataacgaattaataagagttgtgtgcttgatggtccaaaagtcgtatgtttttcaccatcacaataagatttaccatatctacttaaaccaaacttcaatctatgatatttggaagctagcaatgaatgattttgttttagcatatgttaccgagcctattcatctttagattgaaagttttgtgtccaacaactagcaaacccttcgctttttaaaccaagatcagtttttctacatagtttcgactttttgttattttacttcccatcatattttgttttatttactactgacattttcataatgtacggtacattttcatatattagcatccttatctaaaagctaatcaactccataacgtacttgaaatagggatattttagttgaattattcaaaacaatcacacagtgacacagactctatgtagaccaagcatgtgttatgtaattagaaaaaataaactgtgattaaaatatcttttaacttgttaacttggctgcctaaagaaacttgatactgtatcatcgttcatcgtattttgtaattaataaaacataaaaaagcaaggaaaacccatttgagtaaatgatatattaatattacttcgtaaaaagctgcgtccccacatattctattgattttaaatactattcgtattaatataaacctgcatataagcgtacatatacatttctgattctatatataggtgtgagaccttcttctccattct

SEQ ID NO: 38 - DNA, 3000 bp, Arabidopsis thaliana
  AT3G54990 chr320376523-20379522 REVERSE (source_file=TAIR9_upstream_3000_20090619)
aaatagagactataaacataagtgcataaagcacaaggttaacaaagaaattgacccatcactagtaatattctgataaaaatttcaaagcagttaaatatcagagatttttgcatatagtacacgttcaagtattttaaccatttcaagaaatttgagtggtagacaatatttcaattttcgattgagtaataaactttgaagtgtgtagatgacttcaaaacacattatcattgtgaattatagggtaatacgttattgtgcaatgcaagttattaataatgtaagaaaaaaggttactcttttacctattggaactagaatgaacagagatatttagtcttaccaaataaggaaaggccgattccttaaaatctaatattttcttgcatatctggataaattgaacggctgggatttgagaaccaacttatcatcaaatctatcataattatttgtaaaaagagagatatagatttggagtgagtgattggtgagtggttgaataatatgacaattgacataaccccactgtccctctgtctccattaaggtttctctctcttcctactctcaatacaatagtagtagttggcatattggacccatcctttttttttggtttaggccccaaacgtgtaaacatcacaccatctctcttattctttattcattgactataacacatttatcattttattgtatagtttatgttgtggtctagattatcatctttcttgttcctccaataatttggaattgaagatccatcccactagtgataaagaagaagaaaaaaaaggcttgaggatgaatgtgttaaagaaggatagttaaaatattttaaatggtttatttaaatttatgacagtccatttgaaactccttttcttcaccaatatttcagtataaagatgaaatgcaaatggtatctccttcttatagattaaaccataacctaaatcaactcttacgaaatctcagttgattgtgatttatgtatattgggatgcgaaagcacaacaactaggtttaatttcttctaaaatgtgtgataaataaaatataatataaaactaacaaaaaaaatctgtcggtctctctaatttttcgttgctatgttttatccatatatcgtaatctttaatttaaatggaaaagggttgtcctttggcaaaataaagaaaagaaaaagaaaaaaagatagagaaaggattgtcctgattcgttaaatgacgcatagagaaacaaaatcacatgtgggtttatttaacacgttgagaagataccattgcgacgcgaagtaccaaaaaatccctgaccgttagattttttttcatatcttgacacgttggaatcacacgagggacctatcaataattgttgtcgctaaatgccaaatttaatgaattttatcgaaaaataatgacctaattttattactaaaatgcagagagtagacgatgttaaccggttaattttgttgttgaatgctttaaaatgtgaaataactatgttgaattgtgtttcatattttcttttttaatcgtataatttactaacatgttagaagaaattacaaattcagatataatataaaaatttatttatccttaccaaaaaaaataaaaaaattatttattggattttacccatctactattcattctcatcattaacttacgttttccgaaaatcaaagatatttaggtcattttatgtaaggaaggataaaaataagtaatgattaagaaataacatttaaaataaaaaaaaattgacaacaacggctcattattcctcaaaattctaaattttgctgagaaattgtgttaccaataacaaacataatcgcatcgataatcgctatataatctgttgctaattaaggataaattgaaaatttgtaaaagtcggcaaattcctaatattgctagatttaggttgttgaaaatttcagatattgactttttagtacttttataattgatggggctatcttattttcaattatattttgtttgtggtcacactaggcctaataacgtacacttgtcaattaagaacaaatttataatcttacagacaattccacattcgagcatgcattaacaaatttcttcggcattaaactatatcaaattagaatgctttggtttgaaaaataaaagtaataacgcttgttttttgatataatgcatagaaattaaaaactacttactcttcaaccaatcacaatataatacgtcctttaaggtttgacttttggttttgatcttaatactaaaacaacattaaaaaaaaattcttaaactctttatgatgttatctattttgttaaacttgtcgatgacttgatcaaaagatgtatcattagataaatcaagtagctgtattttacgagggaaaaataatctatcatattatattcacgaggtctctttaccaagtagaaggctaaagaacataagtaatagtataagctagtatctttatactttactctcctcacctatttgatggcattagttttgtttactattttaaaattacaggtcgacttcacttaaaaggaagagaaaacttttgtttttgttattacatcgtattaacgcggttgagtttacaacatcaaaagtgttaatatataaaaactcatcgtacaaccctatctacggctctcccatatttaattaacaaaaatgaaaactaatatatagtacaaaatatgtttattcgatacatcggcaaggcaagtacaatcatataattatagaaatcaattttatatataaaaaaaaagagttgaaaaggcaaataaagggcaagtgtatccaatgcaaccttcacagtgaacacgtcccacgttcttttttttcttccctcttttagaactctctctctctctcgtttttgacacttatcctctctcttttttctctctccctctctctctcgccggaaaaaagaacaacgtcgtttatagctaaagattcgatc

SEQ ID NO: 39 - DNA, 3000 bp, Arabidopsis thaliana
  AT3G59270 chr321907873-21910872 REVERSE (source_file=TAIR9_upstream_3000_20090619)
aatgtgaactctttaatttttcaacggtcaatattgatcctcggagtagaagatgggcctgatttatttagcagcccaagcccataaataatacgtgttgagtttctattgggtattattaaagaaaacgaccttgggcatcaatcccctgtttcgtgtttggtcatcggtgttccgtgtttctcccaaacttttattacacgaacacaacaaaacaacacaagtgagagaaccctagattcttcttctattcccttcttccttcaagtcctttttatttctctaccaacctcgaaaacgaatcaagagattagcatcttatcgagatttaaaccatcaattaatcggctgccatggtgggtttcctcctcctctcttcgttttttccaaatttttaattgtttcttgccgctttaatctcttgagtgatgactcttttatcatttggtaaatcaattgatgttagcatgatttgagatcatctattagatttatgttgttcaattgtataaattagagaggcagatgggttttatgaattgggtacggattattcaactggttgtttcattttgagataatttcggaaaattttggtgtttattactaacgaaaattttctcgttgtatgattgattgattgattgaaattcatccatattcgtattcgcagtgaatagaacattttccaattgaaaaaaacaaacaaattttttcggttctcatttgttctttctaggcaaagatgtctcaatgttgaagttttaatgtattggcgatagatcgatgattttgagctggcttgaatttggacatgtttctttgtttacaaatcccttcttcatctctacttgtcatgttgcatttattctataacattctcatttcttttctttttacaggctgggagactacttgcaaatttgattgtgatgggttctgggatcattggtcgtgctgtctttcaagcctatcgtcaagcacttgctagtaagtttccaacactttttgttttatattcacccttttcacatcaagtgattgctttatgaatccaatgataaggcaattacatttcttggtaattgttgattctggcagctcctgtttgtgtgtgtttacaactgttagaacggttgatatgtttctgaatcatattgctgctctcttttactcgatacctaagacaaagggtggtcaggtatttgttacgaaggagacctgtcttacctaggagaaaattcttttgcagatgcgtctaaatctggtgttgcgcaggaagcaatgcaaaatggagtacgtcaagcagggaaagccatcactgagcaagaggctaggcagattcttggtgtaaccgagaagacctcttgggaagagatattacaggtcaacatttctcttttaccaatgacgaatgaatgatttacttttggaactctgaatcgaataactaaaaggactggctttatatatgaatgcagaaatatgacaaactgtttgagaataatgcgaaagcagggagcttttaccttcaatctaaagttcatcgagccaaagaatgtctagaagttgtgtacagaagccaaggcaacggtacacctagttaagaccttactctttttgttttccagactcaaaattcttgctcttaatgtaaaatcaagacgagttcatggtttctttgagatgatgtggaacttacaaaacggccatagttttcttcacacattaaattagtaataactaagacaggtttgctctgatcagttgatcagtgtcaaagaacctctgaaaactagaaaggattgttctgtttgtgtatgttttatccttctctcctgagagaagactcttttaaagctatccttcctgtttttgattcagattccaaactgatcatctacactctgtaatgtataatgtataataaaaaacttcgttatttgagtggtttttgtgtaataggtaaaatctagatctctaagtgtgacttgatcatgttttgggtagcccgagtctcatttcgtaagggtggatctatacaaaataaaattctatgcacaatcacagataaataacgacccaattttggcaatccgaattgaagaagctacgatgaaaacatgaaaggagatgagtaggcctgttgaagtagaagcgaagtggttcgtcttgagagagttttatagacgttgagtgaaatgtgacgatcctgatttggttcacttcttaatctcctcatacaacttttctttattccaacagatatcaaagactgctttagttgtatgaattcgagataaggcatgaaaataatcaaagagtttagatgagaagttgtctgtctttagcatgaacaatatggtaatttggttgacgtcatacagacgcaatttcgtctcaaattatattaaaaggttaaaattcaaaaaattggatagagaagacagacgatatgaaactgatattacttgattagactcttgtgaaaaattcctccttggttatcacagagatctccaaaatctcaggacttgacaacacacaaaaagaaaatccaacattggaactaacccctcccgccaaattacaagggctacagttaccaaaaccctaatctacaactttcacttcactttggctataaaacctctgaatccggtttctttgaaagtacattgaaaactcaacagttgtataatgatctctgcttcacaccccaagtaagcttctcctcttctcaagtagtgtctctagtttaaaaactttcgaaaagaagaacatttgcctatgtgtgttgatgttttgattcttagttatcaatatttctctttctgttaagaatctaaatggaaacttaaagattgtgtgaaaggcttagaccttgaaaccggctttgaaattggtaatgttgtatttttggaatccctttgttgtattttcttacttaagctcatttgctctgttcaggctataagtggtgatttgcatttgaaaaag

SEQ ID NO: 40 - DNA, 3000 bp, Arabidopsis thaliana
  AT4G00180 chr475385-78384 REVERSE (source_file=TAIR9_upstream_3000_20090619)
gttaatgaaactaaagagagacatatacacgtgcatatatacactcagatgttgattgttttccaaggtgtttgtgtgagaaatgacatctctctctctcacccattcaggcggtggaaaaattcaataacacttttaaaaaagatttttgttcaataggttttgacctgaaaatacttagaacaaaaaataacacaagagggaaagtgccgtaaaagtaaaaaaagaaaaaaaaaaagagagagatcatgtcgctgacatactatacatgcctagctagctcggtacatttatatataatgtttgtgtgtaaggtagattgagatatatgaatggtcaaatcggaaataaaacagtatatgttcttaaataagcttctcctttgttcaacttttttgctttcatttccgttacatgatttcgcgattaattactttttataattagtcaacttcgacgccttgtgtcgtcctcgattttaatgttctgtctttctctgcattcatagtttatcttatttcatttcatatccttacttcttttttctacaaacaataatttctagacgaattgaaacaaatttccaaaactttatgagtatatataggatatttttagtggcaaaggacgacttaaattgtaacgaaaagaatttgaacttttttccattagtccctacgatgaatatttcatcctatagaacatcatatgaattgattacttaatgaagttaaatttttgaaaactctattagggacaatgtcccacatcggaagttgagtaggatcttaagtggtatatataagatatgagcctctccacctattgccaattagttttacgttggaagcccacagtctgatatggtatcagaccccgatccgcacatactcaacccgatccacatcggtccggcccaatacttggcccgccgatccatgcccaaacatatcgagatcaatggctagaagaaccatcatctcgagggggcgtattagggataatatcccacatcggaagttgaataagatcttaagtggtatatataagatatgggcctcttcacctattgccaattggttttaggttggaaacccacactctaatatggtatcagagcctgatccgcacatactcaacccgatccacatcggtccggcccaatacttggcccgccgatccatgcccaaacatatcgagatcaatggctagaagaaccatcatctcgagggggcgtattagggacaatgtcccacatcgaaagttgagtaggatcttaagtgatatatataagatatgagcctttccatctattgccaattggttttatgttggaagcccacactctaataaactcaatacaaatactaaagttgcaaacaactatactatatttgcttgaagttctattgtgataggattttgacttacaaaaatcatattctatgtctacactatatattgtgaaaccggatagccataatatttacaaaaacttaaactaatttcagatataaataaatttaaatttcactatgacctagtaattatcacgaacaattgggaagttaccacaaaatggtggtcctgtattatggaatttagcgaaaccacacacttgcgggtaaaaataatgatgacaaaatgatatacattggaatgtagagaaagtatatacaaatactaataaatagaaaagggcatggaattgacgaggatcgagggaaagtgtggtgtggcagaaatgttattaagtgtgaaaacaactgatgaatgagcctttgcttaatcacttccatgcacaagtccctttcctcttcctctctctcccacttcatcgaactttgcgtcctttaaagaaaagaaaaaacaaaacatttttcatccaattgaattcaaaccgtaaccagattagatttaacttcgtttattttacattatcttcttcttcttattttgcggagaagacttgtacaatgacctcaatacgctgaagagttggattcactgcgagtgtatatgtgcatatagaaacaaaatatctagctatatgaccacttttaactctaaatatgggtgtaatatgccaaggactttctggagatccaccacaacacacattcaagttcaagttctgctgcttttaggtctatgaaaacaacaccaaccttattttcatattgttctctctttatctcatgcataagtatattttcatattgttccctctttattttcatgcttttagttgatattttttttgttattgtattatcattagtgcgacgacacaaagtcacaaaccatgtaacaaaatgcttaaccaaaatagaaaaccttaacaattccaatatattttctgttattgcattatcaattatcatcaaatttttttccactgttgcattatcatcattgcgacgacacaaggtcacaaaccatgtaacaaaatgcataaccaaaatagaaaaccttaacaattccaactttaacatgtgaattaatattatcttaatttttggatgttcgacattcaaataaactcctaattaaaccctatagatatggttaatcttctatacatgtgtcacgtacgacacttattagccaaattgaagttttgttttttccgcaaatatataagcggtttaaatagttggtaaaaaaaaaaaaaagttagcatgtttgtcgtatgtacatgtttgatgctgcacaaatcttgtcgttataaaactttgaaatctttatatctattattcctcaccgatcggctactaattcaatggtacttgataatgataattagtcccaaagtagagtttttatctgattatatagaaacaaagactagttccggtttgatatatagaagaaagaaaaaggatggataggaataataaagaagataaaccggttcaaaaggtagtaaatgagaatgcaggtatgcagcttatctctacaatggcctcttcatcacatgtcgccaatttgtttaggtac

SEQ ID NO: 41 - DNA, 3000 bp, Arabidopsis thaliana
  AT4G00480 chr4219744-222743 REVERSE (source_file=TAIR9_upstream_3000_20090619)
aatgcacaatttcttcatcaacctaacaaaacgaagagtaaaattgattctggtcacaaacgagtgaatcatccaaatgaaattccacacttacgagctgatcagtagaatcgtcgtcgtcatcaattggaactcgatcttcctcagtactctctgctctcagtttcaccttcgcgagaaccaaaggcctagcaccggaaaatgtagaaacaccacgccaatctccaacgctgaacggtaacgagctccgagccgatactctcgtcggagctccaagcttcacgctcactggaattacactatgattcaacctaatcgccatcactaaaccaaaattatctcaaattcagttaaaattggattttcgtggaatgtgaaatcaaaagccacgtgaaggcttattcatgggatccaagaatcattggttggttcaaatcaattttgaaatcctaaaaaagttttcaggattcactcagatccgttgagtacaaaagggttttgtcttttaccactttttttctaatccacgtggcgtgacctgacacgtcagataagtacgcgcgttgcaactttatcaaggggcttttgtgaatattactatgggcttttcagaatatattattgggcctgtaaagtcacgtcactaaaaaaagtagtttatctacatcaaatgtgtttattatttaataactttagtgtgttgttgtacttgcacgagtgtatatgaatttagaataatactatcttaaacaatcggctaagctatttataatctgtaagattgatactaacgatgtaacattaatgtattaatccgaagacagtcgtgcattgtagtggcaaatgttgtatgtcagatgtgtcgcgtctgtaatcaatgcgctacgtctctctgtcttaacaaaatcattgaagctttaaatttgaatttcattattttacaattgcatttctctcgtaagtcgtaactttcatattcattttttgaagattttttaaggataaaatgtgaattactattaccaaaagaaacattgttatttactttgggacagcttctagatatggaccccaacaaaaaaaaaaaaaaaaaaaactgaaactaatacaagtcaagacccaactatagtaactaggtttgggtgaaccattgggacaacaatcttccacaacctttgcgatattgtcacggatggttatgtcaatctgccacaaaacaatcttaggagagaacttgaagtgttgtgcaatctctcatttctttctctccgaagaaagaaaattgttgcttgtataatgagaattaaagacttcaagttctcactcaactttaaatttatctaacttttctctttgtcgtggttcatgtcgcttctttttcatcctgaatgcaatttgatataggatcccattggttaggattgtatatgttttgtaaaatttagggtaatatatagacttttatactataattttattattatatacatttaaaaaatgtctagggcatctaaaattaaattaattgtttctatgaaaatagaaaatataaacacaaacatatggctataatgagtgtgttgtgccaacagaaataatttataaaattaaacacaaagttttgtcgttatggaaataaatgtgcatgcaaccacaagtggtgcgttactaaattatgtagggcttcgtggatagagatggatcaattattattttcacgagtttcttgccttcatttcagccagctctctcctgatcttgccttcaattttattactatttttttactgccacttgctatatttacttacttcggacaaaatagtgctttgatggtaaacaaataacgatcggtatatgcgatgtctcgcgattgattatcaaacttgaacagtagtgtgtatttgtttaatatataatctattagagtatcatggaattattcgggtgaaatgcgcatttatatctatcatagaaactagaatcttcactatccaaatagtatattccattttcttgcaactacgtataactacattctgtttgtcatatatttaagatcgagtaacaaaaaattaatcatcttcattgatgtgagccagacaaagagttcaatttttttggttgttaaagtatgttgatacttgatacgtttaccaaaacacaaaaaaacaatgtaggtcgatacgttccatttccacacaacaagatagatacaaatatataatgattgtaagacaataagaccccgacatgtcttaacacgaaaaaaaaagtaactaagtttatataaaccaacacaagtttaactaagaatctaagaatctctactttgttgatttcataagtgtattcatctaaattagttataagtacaatgtaatatcaacatgtatttatattgtctcttgttggatattaactatccaaaccaaacctataatatgaaatcatcaaaacattaatcattatggtcaaactatctcaagttgtattctttaaaaagtgtcacgataaactctaacatcatagtggatttaagacaacacaaatccaaaagagttaaaaagatcggtcttaaatttatgattcatatccatgcatgatgttggccaatcacctagtatagtagtaataaccaagcttttgactaaatgaataggaggaaacaaaaaaactatattgaatctttctttttgcaatatttttcagcatcatcactaatttagagaatattttaccttggttttcaccaaatagaatccaacactattaccatatgtattgtataaagataagtcactccaaaaaccaagatcatgacccaagtcaaccactctctctcataattcagacacagtactcaactttaaagacactcttttaagactttccatcaggaccaagctgagaaagagaaagcaataaagaaagaaagaaaaggaaataaagcttcaataccaaacccttctttaataaattttgccttttggttgtctc

SEQ ID NO: 42 - DNA, 3000 bp, Arabidopsis thaliana
  AT4G12450 chr47386811-7389810 REVERSE (source_file=TAIR9_upstream_3000_20090619)
actaaaatcagaacaaagaataactaagagacaacaaactgaaactactaagaacaggaccggcgacggtgctatggaagccaccaatcaccggccaaaacgaggaggaagatggccttgacggctagggcgaacaaggagaggggagagaggtcgggaagaaagaaaaaaaaaaatttgaaaaccataaaaaattactaaataatttccattttgattactaaataattttcattttttgaaaaccataaaaaaattactaaataatttccattttttgaaaaccataaaaaaaaaattgaccagaaaaaaaaattttgaaaaccataaaaacaatttccatttgctatataccataaaaaaattactaaataatttgtttctatggtctataggtcacgtattttaattgatcaatttgatgatgatgctatatactatcaaatttggaacaaattctaggactaaaaggataaataatttgggcaatagacttagaccaaattcatattagttcacaacttcacatcacaaaaagcaatttagaccactttttttttttttgtaaaatgaacaatttacattttacaccacttttacctaagtccttaatgtggtggcccttattctagtttgattaacaaagttggtttcaatgatatttctaatcctttttagtttttatatgttgatattatagggttctttttttgcgaattagatagttaaaaacattaaaaaaatttaaaaaaattataggctatacttttattttagggagagaaataaattgttaagtaaattcggcaatttattttattaagaaattttttaacaagttaaactaaagaatttagcacaaagttaagtgctagttttaacttctcatattgctttaaatcactcatacactatgtaaatgtcagatcttgttatccacccaacccacaatatagacggatacatgttatgggccgagaacagatatcaggtctatttgcttaaagacccatgggtttaggaatgcctatcgctgcccaagaaagatctagaaggggattagtatttgcgcttcgtaacggacgaatcacagagtaaagtgaagagcgatgatggcggaataaggattcgaaacgaacaacattaaatcgagacgtgatcggcgacacgagaaaaacattatttgtgattacgaatctcacaatattaatataaaaggagagaaatgaaattgtaaaggagggtcagattgtaacgacattatacataacttaatacattattcttcaaattcgatcttgttcgattaacaaggcatagagacatcgaaacatccacaaatttctaattgaggtttctgacatctacatactagttagtggtaatcaaactcttttctatattactcacaaattaaatgaaatgaaaaaatcaattttttacttttttcaatgcataattcgatttgtaccaatggataatttgttgttcgataaataagagttgtttttttcagaaaaaaaaagataaataagtgcggtttatgagatatcatcctacctaaaatatgcaaacattttttttgttcttgaaatacgcaattgtaaagatctctaatcaaggttttagagtgaaataaaaatgttgaaatttttctattctcaattttgtttaatttgtagcattcatgataaataaaatcatttttattttctattttcttccatttatgttaaaaaaatagagtagattttcttctactttccataagagtaaaaaaattatactctattttgctattttctcttgatactgtatattctatttctacttgtttactctcactcttttctactattttctctaaataatactaatcaaactctatatttaagctatataattatacactaaataaatatattttcttgaaggagtatatttttttaatccatatatttatacactatatatgtttcccagaatatattttcttaagccatatatttatatattatatataaattatccaatacatacactatacatattttagatgcaataacatgaagccactatatataaaaataccacgaaaatacatatcttttctcaaaattaaattaaagttgtcgcttcatgatattgcagcttcaaatgtgacatttttgtcttattaaagcataaaaaaaaattaatagcgacgcattacctttttcttccttctcatagttctatatatttttatattaaaatttatttagtttgtttcaccaacaaattgtatgcaattagttcatataataattcaaaaaataaactgtagttcgttttggtgaatccgacattccaacatcatacaaagaaagctactatttaaaaacacatactattaaaatttgatattcttttataattattatggaacaaatatttcatttcaatcattcaagtcacaaggttcaaaaaaaccaagaatacaaggatcataaagtttaagacaaaacatacaaaaatattttacaatgctaaaaagggataaataaaatatatccacaaaatatgtataagagcttgaaaacaaaagctaaaaagcaatttggcgcaagtaataacattatttgtcctgaattcatcatccaactatgcattggatagaacaatggtcataatcaatagaccacaaacgatattctcgctgacacaagaaaaactagatcaagaaattcatcggcaaggaaaaagatatgttataactaacttcaacattacaagaagttaactaaaatgagacatgtgttaattcaatcatgattaacaaggtaaaagcattgtacgtggttatgatacataattagtttcttttgtttttacagacaagaagctggaaaattcacgttgacattggtcaggacccaaaataaaaggggtaatatcgtcatttactaccataaagaaacaccaaccgataaaacgtccattctaagacggcgggacaat

SEQ ID NO: 43 - DNA, 3000 bp, Arabidopsis thaliana
  AT4G14819 chr48504263-8507262 FORWARD (source_file=TAIR9_upstream_3000_20090619)
aatttcctcgtagctgttatgcagatatcttcgtgaaatatctgatatcttgaagaatttttcttgaaggctctttgcttagtttgaatattcgttgaccgtggactcggtggttcgagttaagtttcgtcagaagatgttttttcttgtcgtggttttgtttttcggtggtcgtgaagatcgtgaagtgttggctaataatggtttacgtatggtggagcatatcggaatctcagtttctgaataagaacgcaaactttaccatatttatattgcaaaagttggtcatgctctttatcaaatagagaagttagatcatgcagattaagccacatcctgacatccttctttttgttttttttttttatgcgacatatatcagtttctatttatttacgaactcccaaaaagactccaacacaaattaaaccaataaactcaaacaaccatattaaaatgaacaattcacaagtaagtgtttggtgttgcttataattttaatggtttctacaaatttatctacgtatcttattttttttcttgtttttttcattgaaaaggtcagcaatcaaatttcaatcaggtgaaactcaaaaactttatataatcacaaaaaataattcaacaaacatatccttaaaccaaacgtaatcatattctttcacaaacttcaccataaggtgttgcaaaagttggtcatgcaaattgagccacatccttattttcgtctttttctgcggcctcaacttcttatttatttacgtaaccacaaaagaaaagactccaacacaaaccattaagcgatcaaacaactaaattaaaaaaaaatcttgagagattcttggaacacaagaaaaacatgaaacctagaatgtgtttaatcttgtttattgctttaatgagagtaatgtcgattgtttctgctcagtcgagttgcacaaatgttctaatcagcatggcgccgtgtctcagcttcatcactcaaaacacttctctcccgtctcaacaatgttgtaaccagttggctcatgttgtccggtattcttctgagtgtttgtgtcaggttctcgatggtggaggttctcagcttgggatcaatgttaatgagacacaagctcttgctttgcctaaagcttgtcatgttgaaactcctcctgctagtcgctgccatagtaaaataaactcactttccatgcattagattcatatttttgctatcttttttctctcacacatcagaactttattattttcaggtggttcttcggttaattcacattcaggtaaaggactaacttataaatagaacctaatctaatcatatctattttgacattatccttttttgacattatctctatgaacagaacatggaaatggatcaaaaacagtcccaagagaaaagtcatcatctgatggttctattaagttctcattccctcttctcgccatcctattcacagcttcttacattacattaatctacgcaaaatactgacttattttaccaataaagcttttggcctctttgaagaacatattcatttttagtctctattaaatcgcggtgtattgtgctgaagtattttgtgctctaaatccctactttctaggtgatattggtttgtagtccaaagagtgtgattgtatatctttgatttatgcaataaacattattttctagctaggacatatttgaaatacatgatatatatatgtattgttatcactaacatatgtctaaacatcacttaaaatttagagggagtactattgttatcactaacacatgtattgtagaaggagtacgattgctatcacaaacacatgtattgttgccattttagtcacagtttctaaatatgagctttgtctgttaccctaaatgttattgctcaatgttcaccataaacttttgcgcagagtgtcgcgtctctatccactttttcttttgccggtaattttacgaagggaattatcgcctttctcaaaaatttctctatttttaagttgcatacttgtaaatcgttttggtatttctcttttaatattccagaacatatgttgtgaaagctatagacaaacatttatatgctggctgaaatctggaagacagtttctagcaagagtcaaacaaatgtctagagaagtcaaagcaacaatgataatggaaattaactaacacctacttttcttaatcaaactagtgattcactaatttaaatgtgagattgacagaaaaattctgcaaactttattaaatttagtgattcttatagacttgtttagaagacaagtcatcagtttttttatagaatgtagtattgttttgtgataatgaatttgtagtttttgttcgcaagttatactaaatattaattgatatgtgcatgaaatgaaggtcaaataatggtagaaggggacccttgaacaagaccacctttgtctccatttccacaaatcacaacctcatgtccctttagaacatggagccaattattgcaactccttgccctcccatagttttgtttaaatatgtcaaatctttattttcatatacaaaaatcattggtatcctgcaataaatattttcatcaacgtggacttaacacatctaaaaacatatcatatcaaaagtcacaaccctactatttattttggtctaagaataatatgattcaattcatattattttcaaaattcggtttgagattatccttatagcttgaaatcgatgaattatgtggtgcatgcgttaattgcaatagggtcttcattagagattattttttgttttgattataagataatgtactacttttcttgattgtacgttaatgtagttccaagtgtaataatgcatttgctctagcacttctcaatttctctagaatataaaatattaattgtcccaattctatagagccaacatataaaccccaccatgcaacagatgtattattacgtgtatataactaacaattagctttggtattcaac

SEQ ID NO: 44 - DNA, 3000 bp, Arabidopsis thaliana
  AT4G31610 chr415314499-15317498 FORWARD (source_file=TAIR9_upstream_3000_20090619)
ttttcaactcttccaaagtaatgtccatttcttttataaatttgaatattctgagcaaaatcatggaataaatgaatgtactactaatttcacgccaagaaaatgaactctataaatgctatagcctttcgtatgaattatgagtcatttatttatcttcatttatttgaaatctctaaagtccaactaaaaacgtttgcaaattcaaaaaccaaaaatattagttatgatccgtatgcacaaaccgtagatttaaggaaacgaattaagacttaaacagaggtcactgacgttgtaatgacttatgtgctgtctttttctatcacaaaatgtcttaatccaaagtattaattagtagtagtagtagacccatttaaaaaacttttccaccattaaatagaacaaacatttcatgtgttcttgatagttgatagttgataccaacacatgtctaatgtttaatatggttatgctacagccacaacttaacaattatacattagcatgattgcatctacatataaaaataggtattgattttttttttttcttaacctaaacgagaaattaactgatcaatgaagtttcacatcaaaattaaatctcactctaggaccgattttcctggttcaactctcaaatacatcggttcgttctgatcattgatttgagtggcaaagtctataaaccggggtaatgccttcccaaacgaaaccaaaccggaccgaaattctcattttttgtgagcttccaaatcagacattagcttagccggtttagcctttttctgtcggtacttggcatatgagtaccacacaccaccagctgtgttgaccactaaaccggacacgttcaaagcatgtacttcaacaccacccaagaggacaaaaccgagcgtctaggagattaaaaaggaggcaaagattagtaaatgattgcctctatagatttcatctcgaaagagagccaaatgagggtttatacttacagtggaaccaacgcctttgagaacaccaacgatggttgttgttagtgcagagttcacaatggtgcaaagaaacatggtgaagttgaggacaatgcccataaccagtgaaagaatgagaatcaccaagaacggcaaataagaacactgtcaaagaaaaaaaaattgattttgacattttgaagagactagtttctgagaatagccgcaagcttggaatgatgaagtttctaacctttgcaagtaatagtgatagagagtttgggaattcgccggtaactatgatgaggatggacaaaaatgggagagaaaggaagctgttatagaacattatctcgatggaggaaagcccgtcttctgcaccagacttctccaccagcacaaggtacatagtctgcagcaatgatgagaaacaatattcatttactcgtatgggtgagtggagactgatttaagagaaggaaaagaacaaatgaaaccaacctggaaaaagacagatgttaaagccaaaccatacccaaaaagatcaaaagaaaaatcaccaagagctgcaattacacagcctgcagcggtaagtagtacagatagagcaacctgaaaattaaacaaaagattcagagcttgaaacattatacaaattcacaataaaaaatcaacattcttatggtaagtacctgagttgttggtttgcctttaccaaacaaaaccccagaaatcaaaacagcaagtggagtgagtctcttaatggcgatatacattggaatgtttactcctttcaaacttgcaagagcaaatgcaacattagcattgtagaagatagaaaccggaagaagctttttggccgtagccatatcaattcctttagctcttgtgtatcccattcgtctaccaaagtgtataagcaaagaagtagccaattgctaacacaaccaaacaaaagcaaacgggaccgaattcataatcagcatttacaggaagtgaataatctctttagatatctagtacttgagaaatatggatatcagtacctgaagagtaagaacagtcatggaatgtggatactgcataatcacagccttgttgataaaaaccattgccattgaagcaatgccatatgaaacagctgcgactaaactgcatcatagaacaaaccagtaaataaggttacttaaaatgtctatagctttgattctctataactctctagagatccagatctacccagatgaagaaccgcaattcaaagtttcaaaaatcacaactttaagaagttatcgaatcctctgaaccaataattatcacccctactaactaaatcgatcagaaacatccctaatcgaattgaatttgaagaaacagagcactaaagaaggagactacagtacctaatggacgaggtaggctccatttcagcttgaacctccatggggaaagaagagtgatttgagttataaatctttggaaaattcctctcagggtacacgtttcttggcagattcaacgtgatttagacgcatttcgagaagaaagagcgattaaaaattacttctttatcagattttcatttcaatttacagtttttttcccttggattgaggatcttagttcttaagcgaggttacagctttttccagtggattactaaattggacgagagagtaatagtcaacactcaaaaagtctttaaccttctgtcggaacaagaagggcttgctgtccgttgctcatctcatatagtaaggcccaaaaggcccaaaggcttctttttccaattaaaactaatttttgtatagcgaaattatgatgaagaaaatcgtttcatggcacgtcaaaaaactttcgcacaacaatttttttttacaaaagggtcacttttatcagaacctaatctatcatagagttcccatcaacaaaaaacctatcatagagtctagtcctgtgttaacgtaggtgatgggttggtttcatggtttacatggacaattattatggtgtaagaaaaagatgatgggtac

SEQ ID NO: 45 - DNA, 3000 bp, Arabidopsis thaliana
  AT4G31615 chr415322195-15325194 REVERSE (source_file=TAIR9_upstream_3000_20090619)
gattagaaaatggaccaggtactttatgtttgcaaccatggagttaagtatgtcactggataatatttcggaacttttccgtttccacttgtcttggatattaattatcccatagcctttatctcattcacgtctccgtcaagataaagttactgacattgtctttagattcgtatcccagtgattgaactgttgtactgaacgggacatgtctgtcttggtaatgatggtaagatagacttttaatttaaactggatattttcttttttttacgaaccaatctaaacgattcgttcttatagtatgaatcaaacgggtttaaataatgggttccataacattatcattaggtatttggctatgatatttaaataatgctgacaaaaataaaaatatcttggttgaagaattaaatttacaaaattattaacaaaacgtataagactaaacaaaaaaaaagaaaaaaaaaagacaaatttggctataaaaccaaaaatccataaagacctcaagaaataacttaaagagttcaacaatggcgaatcatccactatcttcttctcgatccaaccgaccttttttcgtcagatcacttgctggacacacctcaaatctagtaacctctctctgtctctctctctctctctcaacttaccaagaaactcaaatttagttgtcactgttgttgcagataattcctgatgagtttttcactgctcacttagagggtaaaactggcttaacgaaattgaaactgacttctgatgcttcggatagaatctgggatgtgagactgaacggccggagattcgccggcggctgggatgatttctccgccgcacactgtcttcgagacgacgacgttttggttttcagactcgacgtgaagatggttttccacgtcacaccttccggacgcagtttctcacagatacgtacatcttctagctccggcgactacgacagtgatgatgatgatgatgaagcaggggacgatgattcagattcgaagaacatttcgttgaagaagaaatcaagatttgaagcagagtcttcttcatcggaaaaatattgtcttctcggcctcactgcttccaacctacgcctaaatcgagtggttagtactacttggcatcaaattctttagagttcttgtttatgagttatttatatatatgtgaacattgaaattgtgttttgttcttttttggcagagttttacaaaacatttttcgagagcaaatggtttgacgaagagatgttgtatgattgatctaatgaatctaagtggggaatcatggactctgggtctgcgacacaacaaaagaactggtcaggcttttatccgtggacgctggagaagtttctgccatgcaaatgagctgaaacccggatcgttctaccggtttaagcttgtccggaatgggacaaggcctttgctacaattgtgttttaaagttattccacaaggaaactgttccaattccaaagcaaatggaaaagctaatgtttctgaaaagtatagcagggaagatgggagtgcttcaacgaagcagaacaagtttctgacagtaaccttgaagcattacatgatccagtcagggcaacttgtaagttttcaacgtcttaggtttagggttaatttgattatgcattttcttgtaatacttcacgaatgcttatgcagcgtctccggagatcatttgtgagggagaacgggatcaaggaggctgaagagataattctagtggacaaaaatggagtagagtggccatcctatgtatcttcttccaaacaacgaagagagttttacatggcacatggttggattaggttttgtgaagccaataagttaaagacgggagagaccttcacgttggagtttgttcgaggagaaggcacaactcctatgctcaagttctgctccgaggccaaggtcaaggtgaataaacaccttgtgattcaagtcgccttttttgttataatgaactatatttagactgttaaaatgaattgtattcagatagagcaagaagaggctccagaagagagggggactccgttaccaaagagagctcgagtgtctgcagaagtaggacactctcgtcgcactcaggcaccaaacaaatccagtgacgatccaaaaatcttgcagcgcaagcaaccacttcaaccctgctcattctctgatcaagcgaaaaaggtgaaacagagtattgtaaacattctaactggtataaaacggtttcggtcagagcttgagttaaaggagcggaatttagaagctgcgctgctggaaatcgacgccttaggtatgatctaaacactgatgaacagttgttttcttttactattctgtttctttgtttctaaaattttaatgttgaaaataaacaggggataaagtatcggaaatcaacaaaatcctcaagtaatcatcaagtctagaagggaagtgaagattaggaaatggaacatgaacttttgcaaatcttgttagtatctgccatctggaactcgttcaacaatcaagacaagtagtttctttagtaaatgatatatgatttacgtgtttgtgaaaatcgaaactctgtagtaaagctgatttctaaatagcctttggacgtgagattgtttaaagggatcagtttgattgtctaacgataagatgtgtgcttgtgggatgattggtaggtagtagaaaagtgaattgagaatccaaaaaaaataataattaaggtcagttttatagacctatcaaatttcaaacgtgacagtggtggcccccaccttactgacctatcaaatttcaacgtaaagtctccttgccagatttactgcttctgtctctttcagcgttttcgagaaagaacttgcatgctctttaattaaaacatttctttaattcgaagaagacttggaaaagacaaaattttcaagatcagactcacatatatctgagattattagtagagttgtggagattactttattcaata

SEQ ID NO: 46 - DNA, 3000 bp, Arabidopsis thaliana
  AT4G31620 chr415324682-15327681 REVERSE (source_file=TAIR9_upstream_3000_20090619)
atagtttgatatcagttatgaatgaacaaaaatttgttattgaatagtgttgatttttactatatagatatcaaatagaataaaataaagtttttaatcctgggaaaaaaataatcaaatcaaacgattagatttttagaaattgggttgaaacttattcttctgacttacttttcagtcctacatcgatcaaatcagaagaaaaaaaaaagccttcggttattaatgcgtttatcaagaaatatctttgtcattaaataattttgttcaccaagtcattatttagtttttttagttttttttttaacacagtcattatttagttaatgacaataaatcaggtgtttaatgagaatgaataacggagtttagactttagattataaaccttactataacgccagcgaagaataattcgaagaggcaaattttcagagaataattactttagctgttcgacaatggcggatccactgattccctccccaaccaacaaagctttcttcatcatagatctatctggacaaaaatccaatcctgtacttttccactgtctctatctcatcgtctctttccatgtctctgttacaatgcatcaatcggttctgtttttcttaattgttgtggttgcagatcattcctactgagttcatttggaatcacttcaatggtaagattcagtcaacgaatatgaaactaacttctgacgcttcggacagaaactgggacgtgaaattggacggcgcgagattcgccggcggatggaaagatttctccgtctcccactctgttagagacgacgaccttttgagtttccggcacgacggaggcatggtcttccacgtctcacccttcggacgtagtttctctcagatacagttaatctcttcttctacctctgacgatgatgatgatgaacgcactgtttttgatgatgatgaagacgacgatgtaggagatgatgatgacaattcgatatccgaagatgatttttgttcaaagaaaatttcgtcgaagaagagagcaagaaaagaaacagagtcttcatcagacaaatcttatcttgttgcacacgtcacaccttcaagcctactccgagataatatggtgattactacttcgctggaatctttaatacaattctcattctattcttttgctacaatatatatgaagttgtgattttgcagtgtgttttaagcaaatttgcaaggtcaaatggtctggacaggagagagtgtgagattgatctgagggatgaacatgaaaaatcttggactctgcttttgagacacaacaaaaaaactggtcaggcttttatgcgtggaggatggagaagtttttgtcgtaataacgggatcaaagctggatctatctgtaggtttaagcttgtccaaagcggaataaaacctgtgctacagctgtgtcccaacgcctctagcattccagaaggaaactcttccaaagcaagaaaaaaacgaaatgtttctgaaattgaaggtgatgagatagaatctgagaattgctcagagactattccattgaatcagaacaagattctgacgtttgatcttaaaccttacgtgttcaggtcatgtcaatttgtaagttttcaagacctttgttctaggtgtttttcaagtctttttgtgttgtcattcgcttaaatgtgttgaaaatgtacagtttcttcctgcatcgttcgcaagagagaacgggatcgtggaagcaggagaggtaactgtattgaacaaagatggcatagagtggaagtcgcatctagttaacatcaagggacgcgatcagttttacaatagaggttgtcaagatttctttgtagctaatggcgtaaagaatgttggtgatcccttcacattggaggttattcgaggaggaccatctccaattctcaagatctgttccaaggttaacataaaatcagttacagtctttgttcttggcttttgctattgtgaaatcgttgttttaaaacgaattgtgttcaggtgaaacaagcagcatcatctgatggccataagactgcggataggaaaccccgaatgacggatcaagcaccactggctgaagaacaaactgataatcgggtcgaaaagagagctcaagttactgaggaaggagggccatctcgcagcactagggctgatccgggaaacttgcagcaaaagcaaccttgctcaatatctgatcacgttaaaaaggtgaaacagagtattgtggatactttaaccgatgtaaggcggtttcagtcggagctcaaggtaaaagaacagaatctagaagcttcattgcaagaaattgatgcgttaggtatgatctgaataccagtgaacactacttttaattgtgtgtgtggtttattttggttccagattttttgataatgtgatgttggaaacaaaaacaggggaaaagataatgggaatcagcaaaatcttcaacattagtcaagtttaattcagaaataaagattagaaaatggaccaggtactttatgtttgcaaccatggagttaagtatgtcactggataatatttcggaacttttccgtttccacttgtcttggatattaattatcccatagcctttatctcattcacgtctccgtcaagataaagttactgacattgtctttagattcgtatcccagtgattgaactgttgtactgaacgggacatgtctgtcttggtaatgatggtaagatagacttttaatttaaactggatattttcttttttttacgaaccaatctaaacgattcgttcttatagtatgaatcaaacgggtttaaataatgggttccataacattatcattaggtatttggctatgatatttaaataatgctgacaaaaataaaaatatcttggttgaagaattaaatttacaaaattattaacaaaacgtataagactaaacaaaaaaaaagaaaaaaaaaagacaaatttggctataaaaccaaaaatccataaagacctcaa

SEQ ID NO: 47 - DNA, 3000 bp, Arabidopsis thaliana
  AT4G31805 chr415382280-15385279 FORWARD (source_file=TAIR9_upstream_3000_20090619)
atagctaattcttaatttcttatcatttatttctagttggtagagacttataaattgaacgtttcaagtctttggacggtatttgagatttcttgtaatttcacttgtaattgtaccacctaacagtgactaaagcagaataaattacgattcgtatacaacaaagtattcaaatttctaaatgtggatatattattgattttattttgggaaacatgtttatgataatcacctatatctcctctatagtctactagaaacgatatttgtcaccttcatcgtacaagactataatgtataattctatccataatgaacgatctagtttttaacatgtactatagagatgaatgattacgacagtaaatttaacccatgcatacgaaaatacataccgatagatataacacaattcagaatgttttacacaatcccatatttttagccaatcagtgtgattttaatagacagctttctatgtcttgttgtgctttttcttcttctttttttcccaaagtctttaatgtcttctggactttatagaagacagctttttatatgttatcttcactttcgaacgactttccttcctaacatatcccaaaacctatctctttaatttattcctatcgtacttacagcttttattaaaaaaactttcttattttaatcactaaacaattaataaaacatttatttatgatttttcattttcgttaaagctcttttattaaaaacttaatcacaacaaatttattaatcttaaatttgaccttaggataaaatttatatattaatcttatatctgttatacattaaataaactttaatttctgcgcaacaaactaatttattatttatttcttgtctctgtctttgtagttttagtataaataaagccattttgctccgaggaacttctcattcagtcggaccaacttgtgaccgaagaaagcaaattgagactacgcaccaactagtcctttggtttgtatcttaagataaaggtttcttttatggacggttcttcgtttctcgacatctctctcgatctcaacaccaatcctttctccgcaaaacttccggtaagtccggatttcgtctgatccattttcccgaaatctaagttgatttttgtatcccttcaatttctatgaactgggattttgggaattaatcatgggttcatttcaaattttcgtatttcagaagaaggaggtctcagttttggcttctactcacttaaagaggaaatggttggagcaagacgaggttagttctttcgtcgaacacttggtgtgcactaccttcgacttcatatacttgttcgaacattcagcttttatcaattctttaacttcactttagtactttacgaatcaattaagatgggtctgagtttgaatgtctttgcatcgaaatgaatcggtgatgatgtagttagtataaagtctgaaactttacataatcaatcttgtttggaacaaaaaggaagaatctttttagtaatttgatctttgtggtggtaatgaacagagcgcaagtgagttacgagaggagctaaacagagttaattcagagaacaagaagctaacagagatgttagctagagtctgtgagagctacaacgaactacataatcatttggagaagcttcagagtcgccagagccctgaaatcgagcagaccgatataccgataaagaaaagaaaacaagacccggatgagttcttaggctttcctattggactcagtagtggaaaaactgagaacagctccagcaacgaagatcatcatcatcatcatcagcaacatgagcagaaaaatcagcttctttcatgtaaaagaccagtcactgatagcttcaacaaagcaaaagtttcgactgtctacgtgcctactgaaacatcggacacaagcttggtaagggattttgtctgtgaatcttttgattaaagagtcatatgtttgaaattgcatagagacaaatgactaagcagagtgtaactctgcagacagttaaagatggatttcaatggaggaaatacggacaaaaggttacaagagacaacccgtcacctagagcttactttagatgctcgtttgcaccgtcttgtccagtaaaaaagaaggtaatttacacacatcgagtttattgtatttatagcttcatttgtaattgtttatctctgcttgcttcttcagttgtctaatgatctgtggtattactgttcaggtacaacgcagcgcagaggatccatctttacttgtagcgacatacgaagggacgcataaccacttgggtccaaatgcttctgaaggggatgctacaagccagggtgggtcaagcacagtgactttggatctggttaatggctgtcatagactagcgttggagaaaaacgaaagggataatacgatgcaagaggttctgattcaacaaatggcgtcatcgttaacaaaagattcgaaatttacagctgctcttgctgctgctatatctgggaggttaatggagcaatctagaacatgaacgtttttagtgaatgtattgtttttgtttgtttagaatgattcttcgttttcgaattgtgtctttcgattaggagataaaagatgtatataaatattataagtagatgaagaaatcgtataagtattcgagaaactttaaatgtacgaattctatataaccagttagatgtcgtctgaatactgtatatgaaaattttgaaataaatgatgtggttaaaaaagggtcaccataaatattcaatcacaatagtctctttcatttattggaaatttggagtcatttgagaaagaaataagaattggacatcactaaatagtatcttaagtattctttaataatgtgttacacaagtaaattatgtattaagttggtaatacaatgatcggaaaaagaagcgcaaaaattcaaatgagaaaaatcaaagtttccctccataaactcactctgcaagtctcgtgattctc

SEQ ID NO: 48 - DNA, 3000 bp, Arabidopsis thaliana
  AT4G31877 chr415415874-15418873 REVERSE (source_file=TAIR9_upstream_3000_20090619)
tttttgaaaatgttactgtaccaaaaactttaaaatatggggcaaagtgaaactcgttgtgactgggagttgtgtagtcttgtatactcagagattttcctatcttatctttgtaatttaatactatgatttgtttctagatgaaacaaagacttttcttacgaatatatgtgtggtttacgaaatgctcggagattcctaaaaggatacccaatgtttgcttcccatatttaaggaatattccatttacattagccatacgtaggatttaaaacttttaatttatcaacaagccttgcaatgaaggaccttaggccagtagatctgaggtcttagatggggttttatagtatttttttgcgcataaagttgcaagttatacattatttctttcattttttcaggaaaagaaaaagtaacaaactaatagtaagagatgtttaattaaaacaagatctagagacattaacaaacggaaaagaaacattattgttatgcgtataaaaaaatagatgtaatacattggtgaatgggataacatgtggtccatgatcccatactctctctcatttctcacttttcataggcccagatgaaatatgggtaaaatgtaaacacacgattcgtttattaaaaacctaattagaaaaatgattttttgcgttctgtgaactggttttatccaacataaatatattttaagaactcttttgatttgtattaaggtagactactagactccgagcaactatctttttttttttttttaatatagatgatctgcttcgttttttcggttatgaagataaccagacatttgatgttttgatagatattataatcataatctaaatttgacatgcaatatagtttaatttttaaaacatataaaaaaagcgatcataatatcttgtaatatttacttcaaaaatttatatttcgaatgtgcttgtcaactataaaatgttcatggttgtctaattatcaaaatgttgttttaaaattaaattaaataagcgaccgtcaaaactgttttctagatttttagagtcatatttgtcataaattaactattaggatagatagatagagatagtacatgccacttctacttaacaaatccaaaaggaaacatcttttcataattatatacatgtgatatcttagactatgcaaagagtcggagttatgtttttctctgtcatcgggaagtaatgaatctggacagtcttaggtcaacaaacacacaaaagttcttgaccttttcagccttcacgcgatttctaaaattatgtttttcccttcgcaatttctttaacatcatcacacacacagtgcatgatatacaaacacattaaaaaaccttttttaacactttttaatatatgtgttaattgtattatttcatttatttttctggttggatatgttaaattattgcaaagaaaatcgatattatgaaatctaactcgtaatttttttgattttttttatctttttatctatgtcctaatcttttgccacgaaatatttagtagttgatcacaaaaagtgaaaataaacgacaacgcatcatacttgacgattttttaataataaaaataaataaataaaaatatatatatatatttacatagcaattttgttttcgaaaatagtgcactggaaatgttattaaaaagcagtgccctgaatggattacacatgaggaagttccacattagtggtggtgttacaagatcagcataaggattctctaaccattttcttagaacaaattcttgttgtgacgagtgcatatgttttgggggatctatcttttctttatgcatggtattatttcatttttaaaaatctttgttttatgggtggatgataaacgatactaatatatttaatatattctataaatattcagcttattctcagcattttgcaccaattaaaaacgtttcggcatctcagaaagatatgaaattagcaaccggaaattggattgtttacctggtttcttactcgttaacacgtgtccaattgaattaattcttaaaattgtgaaataattttaaagattaagttacaattgatatttgaggttccaatatgtattcctccctcaaaactttaatatatctgtgttagtttcacctagaccttacttctctttacttctcttactattaattattacttctcttacagctattttcacatatatatagaaagtttaaaacctgtatacacgtttatttaaactagtattgttttaggtgaaaatagcaaaaaggacaaagtatctgggtgaaattggtggtatatgaaaatttcaaaaggtcgacctgacatttttgtattatatgtgtttggtaaaatcacaaaagatggaaaacctatgaaataatatttccaaagaaatgtgtgatctttttacaaagattagaagatgacctataggtggcatcaacatgttatatgatcaattggtaacttttaaaactatcatggttttagtaaatcaattagaaagagttaaacaccaaatagttacaatttggtcaattttatttgttcggttaatttgtgtttgtttgctgtagtagaagtctttgataaagatttattgtatttgtattttaaaattattataatattaaaagagctgattcccaaaaaataatattttaccactcccatcgtgaaagaccaaaattcatttctcaaaattctagatcattttctaggcttgtcgttgccgtttataggttatgagattgaccactataatctgcaatggtactgttgaatccaatccgttccattcaaatgtctctctcttcagacatctgtcccattgcatgtaagagggagagagataagaaacagacaaaagccaaaaaaaaaaaaaacagagataagactcttcttgaagagagtgagagacagagagagataaggttttttgtttccttctttaacagattcttcctctcttctcctctc

SEQ ID NO: 49 - DNA, 3000 bp, Arabidopsis thaliana
  AT4G36060 chr417052321-17055320 FORWARD (source_file=TAIR9_upstream_3000_20090619)
tatcattttgatctcatcactcttttacttgtctctgaattttgatgaagcccatttgaaataaccacagtttgcttctggattagaggaaggtccctgcaagagttgtagatatacttagcatgtagttacaacggttaaaaagacttggttaaaatctttttacctcagctcgagagcagacgtagaatctgcgtccgaatgtgggacctggtttcttgacgaccctagcaacacaagcttctttatgtcctttgcagagaggtatgctgttttgcattaggttctgtattctttgccactccattaaagctgcatcgtttttttgtttagctgaagaacctgtttgatcctgttcctgtgttgaagtagtcggttcactgtcttctttgcctgaaacattcggttctgtgatgctttctacttgggagctgggactggaggaaacataggaagaagaactatcctcgacattgtttacttttgagtttgtagtgaaaaaggacttgagagaaagctgagatgattggattttccttgcttttttcctgtctccatctctagagatattcaaagctctaactgatgatgcacgtattccatcactaaggttatcaatggaacctgttgctgccaccgtttctgcttctgtaacgccggaagtggatttgttctcaaaagaacaagatttctcaagtgaaattcccatcgaaccacaatttctcagtggtcctgtagaaatatccccacaaattgatgaagtattactttgagttgatgatgaacatgacacctcaatggctttggcttcttcattggcccgcctcttcttaaataccgatactgttacagtaaaaaaacaagtgaagcaaattataaaaaaacgatagagacaggaataacttgagaatatgaaccaatacaagacagcggaatatataggaattttcttgtaccaagagtttgttggaaaccgtaaatcatgggaagatatcttgacgctaatggcggcgtgctgtgttccgggatgtctggtaaatcatcaaaactaatgaatacaggtacgtggtctgatcccttaaattttgtaaccaatccacctttccacctgtagtgtgttcagaagttgattcactgtaaatttgagatccaagtaaaatgaaaaaaggaaatctcaagtacttattcttttaaattgaaacacacatgtgatcattcttgatcatctagacttaccttgttggcatattttcattcttaaaacgcttatactctgttaatatgtcacattccttgacatggcaagcaagaaaactatgcccctgcttatcttcgtcctgatgcaagcatgacccagcaactaagatatgatcaatcctcgaaccgtagttaaattgttctgctccactacttgaggaccaacatgtgaatgcatctttcctgttagaaatccagaaagaagcaatcaatgataaacaaaaatttcaaagactgtctccctcaataatagaaacagaagaggttatcacaaaatttcacctttcaggatgttttgatctgaaaacatcagagaatgagcctccacgttcaactagcagagatctaaaccatttcctgaacctgcatttttttaatgacatgaaaatgagatatctagtagagaaaaaattattaagtaaagcaaagcgatttaaacagagcatgagaatcagttacatactcgtttttctcaaaatcaggcccagcttcacatcgatccatagcaaaaggagcaatgttgagatccccaacaacaaataccctccttccttgacgcagaagacactcccatcttctctacaagaaaactaaaaaacatttaatccaagtatcattagaagccgaagcatctcatcgatacattcagaaaccgataaacttacctctaaaacaccatagaaccgatgcttaaactcgatcctatcagcatcatcagctacagctcgcggcccataaacattgaaaacaactgacaaaacacataaagacatacaaactgtaacaaaaagattcatttttacacaaaagaagttacattaaaaaaagaaaaggatttgagtcataccaaagtggccgtgatcagttataacacatcgtccttcttggtcaatcataagaagctcttctttctcatattcctcaagaccttcagctactgtagacgtttcactcttcccacctcgtgaattactgtttacaagaccagtgatgccttcttctgcagtaacaggcaaagcagtttcacagcttgaagatgctgacttcaccctgcaaaacgttgctacacctgcaaaaatttcaataacataaaaactccaactttttaacattttctcaaatttcactctaaagccactgaaggcaaagcttgatgcttaccagaataaccagtacgacctttctcactagtgcgagtgcacgagaaaaacgattcatacccatcagctatcgccaaatctgctgtcaattcttgccttctcagtttcgtctcctagattcaaccagagtaatactcttatcatggattcatcggcgaaaccgaagaaaaggattgaaatttcaaaagtcgagtaaaacctggaagcaaatgatatcggcgtcgaacgaatcgagaagtttgagaagagaatcaaactgcgaaactcgttgcctaaggccattaacgttgtaagtcactatcttcatcttctccttcttcccactgtagttcgttcgacaaagatcattcctttaggggaaattgggtatctctaagattggacggttgagatcttagaagactaaagtcagtagaatcgacggttgacacgtgaatacgtaagtttgtaactaaccgtagcatatgatatcttttcaatggtggcgcgtggggagcacaagttattgatttgcctcgtcggcgtccaccaccattatcgccggatttttatatattcacgtagaagaagaatgagaagacga

SEQ ID NO: 50 - DNA, 3000 bp, Arabidopsis thaliana
  AT4G36470 chr417216531-17219530 REVERSE (source_file=TAIR9_upstream_3000_20090619)
aatcaaacagtaagaaccctttccaagcgagccaatggatgaaccaaagtatctatttagaaaattattgtcttcttaccaattgtaccatagaatccacgaggcccacaagaaccgaccccatatttttctaatgcagaagtacatgattcctggcataagacaaatcttaaataaaactattcaaacagaagagagattgtattgaaaacaaagtagcttcacgttaccagcaacttctcgtggccaatgagcccaagataattagctgaggcaaaattcaccacatcttttccattaactgttgtatgcggtccagcagcactgtatggacccaacaaaatctttaagcataacacagaataaggctatacaacgaactacatagaagtcttctacgttacctttcaagaactggtggctcatgcttcatgtcttcagtaattggcggtataagaggttcaggaacccactcatcacacaactcatctatttcctgagtaataaaaaaccccgaaaactcaatacatttcaactataccacacatcaaagatgtcaccacgaaccaaatcctatgtgatctatcaaaccaatatcaagaaaggatctgagacagaacctgctcggtcaatggtcgcttgggaggtttataactcttccgagtgaggagaatgataatgacaacacccaacaacccttccacaaagaagtgccctggaaaacgaaaacaaaaacaaaaaagttgaaatctttagtaatctcaaataaccaacaaacttggatagacagacaaagttcacttaaagttgtttcttcaaatctatacctcggattgggacaccaaacaagacaactctagctgaaggactctccagaatcatagtaacccaattcaaagcagcattaaacatttccacgagattcgaagccattgctaatttgctttaataaagctctgtacttttttcttctatagtattaatttgctcgacgaatcgagcataagagaagctagctcaactttgggaaaacctacaaaacccaatcttcaattagtattacaaattttcaaaattcacaacaaaaagtggcaattttgaatccaaattcgacccaaaaaaaaactatcaggaaagatctggcatcggatcactgagcaacgagaaatcgaaatctcggagaacagattaagaaagtatatatgattacaaatagatccgacaaataaacgaggaacgttacctgatcccaattggattttgggtggcaaaatggacaaggagcagaaggtatcgtgcccacaagagtgtcaaacacacgaggagtgttgttcaaattcggcgatgcaaaaagattgactttcaacgttttttcgagatttttgataacgcagacggcaaaaaaaaaaaatcaatttcagagagaaaaaaaaaaaagtaaaagaagaagacgagcggatctgtctatgttcgtaatacgtcagtagaatcctgggcccagatctgtgtgttggttcatcgatacgacgccgtttaagtgttagatatgggcctagaggcccaatgtttcaacagcacacactgtgacacagtcactagtaagcattcatcaccaccactaaagtcttaaaggacaatgcaaagagagaaagaaagagtactaaagcctaaaggtaaggaagagacagaaagaggaaaatggtggtcacacacaccaatcatcaccactcattcttgtttagcttttttctaaccttcttgtaatcatattcgaccccaaagttccccctactttttctgtaatattttgtagcatttacattacactatacatatattattcctattctgttcaaccatagtatcatatcatattcatatttacatctcacgaattagttgcttagatacgacgtcattttaatggaacggtcttaggttttaaatatgggctttgttagaggcccaatgactaacttttaagtgattaacttttcaccacacttgaaaaaacaaaacgtaagcaacaagagagagacgatcatggttgaagaagagattatatgaagaaaaaagattgatacgttacggttttgcattaatccctcaagcgacacgcgtgcgtccccctcggccctctctctaaccacaaaaaccttatcataccaagtaccaacacacagtctctctcacctaacagagcatttacactacccttatgacgcaacaagcattcatgaccaccactaaaatcttgaaggcaaggcagagagagaaagaagattactaaaaaagccttacggtaaggagaaagagagaaaaagagaaggaaatggtggtcacacacacacacacaccaatcatcatcactcattcttgtatagctttttctaccttcaaaagtcaatcagtcttcttcctcctatctcccaaagtttcctatacttttttgtatgttttgaaacatttacattacaattaaaaatatcatccatattctattccacgatttttcatataaatcaccaaatagattctagcaagggtaatttttctaatgaaatttagtttatgtttaaacgaataaaagtccttaaacaagaaatggcaatagagtccaccacgcaaagcaagaacagagtctagcactgatagcaagaacagagtaaagaaagtacaaacattacatgaagccatcatgatgttgtttgactaatttatcctttatatttacatgaaacattatcctctactttgtttattcctatttttggtcactttggactacacctgccatttctccttaaactaaattcatccaaccaaagaaaaatttattttcgttgaatcatgaccaatgatgaacgaatcccacatcgtattttcaagaaagaaaatagatggaaaagtaaacacacaaaaaaaaaggtagggtaatggaagaatgaagtgacaggccaaagatttctcctttaagaaaagcaaattctaacaccaaaa

SEQ ID NO: 51 - DNA, 3000 bp, Arabidopsis thaliana
  AT4G36850 chr417355892-17358891 REVERSE (source_file=TAIR9_upstream_3000_20090619)
aatcttagaccagaagtggaagaaggaatatgtcaagttttagcacacatgtggttggaatctgagacttatgctggctctacattagtagacatcgcatcttcttcatcatctgctgtggtgtcagcatcgtccaagaaaggtgagaggtctgattttgagaagaaacttggtgagtttttcaagcatcaaatcgagtcggattcttcatcggcatatggggatgggttcaggcaaggtaaccaagcagttcttaagcatggtctgaggcgaaccctcgaccatattcgcttgaccggtacatttccttaaatggaatttaaactataaaaagagagaaaacaagctttgttatagttttttagagtatagacttgagaagaaaagaaaaagaaaaatgtataggttacaacaatacacacatttaagtgtcataaagtttccactttttaagtaaaaaatcaagagtcaagaaacaagaccaaaaattcttggcttggtttttatctttttttgccaaacgttttgcttgttatttcttagatagcaaattattctgttttcaaaatctaaattactacaatcactagtttgacttcatcatatctggaccagtggataaaagtatgaaagataaggaaaatccgaaaatggatagacttgtatatccacaattccacattatatcataatatgtactgtatattatggctcttctattcgctgagaggacgaaggtcaaatatgtagaatgtagaagtatcctcatattgacctgtctaactagattaatattatagtatgcatgcttgccatctgttaattaaaatacagacattgtgaaaaaatacactccatgctctctatctttctatcgtttgtaaaatgatattttgttgatcaaaactactatggtctacaagatcgtgtaaaacaacattcaagtaacttagtattatgggaagatgagttctctcgatcgagatgttcagtcaattctctagagtataaaaaacgaaaacgtctcaagtcaattgtcaatcattaatttccaaatataaatattagacattatcaacaaatgatgataaagagatacaaaaaacattgtgaagatgtgggatatgaggataaacatagagagataggatgatatattcacacgtgtttcccacttcctcatccaacttgtcgcggcgcctcgtcgccgtttctttcttctctctttggtttttttttctaaaacattttctctatcaagcgtttatgtttcaacaaattttatttaagctaatgttttttcttataaccaaatcaaaattttattcatcgatatagaaattaatggttttttgctaatattgaaatctaattttaaagtatatattaaaaagtttttgttaaaaaaaaatgaggatactcatgttggagagggagagcaaacggattcttgatgtttttacgatcgattaaagtgaagacgttttagtggacatgatatatttctttggtatgacatattttaaaagtcacctctaagaattcaattttttctagatattatcgtcattcgtcaacattatatcgtatgatgttaacaaaattaatggttggtagagtactcgtcatttagaattagggctaatgaattgatcgttcataatgtcttgtcgagggcaaaaacgacgtaacgtgacaaaacgtattaacctttgtttctttttcttttttttttgttatttcagaattcatgaaccagagaatttcattaatccactagctaatcttgaaattattctattgccttttgaaaaagttgtaaagtataatttgaaggttgcaaaattttaattttggtttttagtgtttgagaaacaaatcaaaactgcaacattatatacagaactatcttcccttttttttttaactaacttgaaatttatgcaaaacagttcaccaaggtgtcattaaaagatgtcattaaattcgaaagtcggttcatgaaagatgtcgtcatcatcgtaacatgatgccaccacgcagtcagcactaagcaggctatttggctttggcaccttcattcaaaaataaaataaattttaaattcctggttcagtaaaaatgtggaccgttgttttgggggcagccaatcgcacgaatatggtggctacgccacaaagttacaaatgtacaactttgtcgggcttttccagattcattaacatcaataggcaagtaaactacaaaatgcaagttgcttaatcaataatgttgtaaagcttttacagcatcttttataccataaaatgtcaacatcatggagtttactccaattaagttctcgtacactttgatttgaataatcagtacaaaacataaccaaaccggattagatgtggttttaactattgcatacattttcggccaatggattaggattactgaaagaacattaaaaacatatatgtatgacgacgaaaacaatttcttagggcttaggttatatatatttcgcagaaatgtataccaaaatgttggagaatcgtgaaaaagaatatacctaataaaccgtactaaaaaggctgaacatctgagattcagcagagcatcaaaagataaaagagaatatgctcattaatcataggtcgactgttttctccaccctccaactttaatttttgaaatctcttgtttttatgtgcccttgataattattaatcaccttaacattaacaacttgttaatgcttgttaatccaaaagtaaagcaatcaaagaactatattataagattaataatgtaatgattcgaaacattacaataaagtataacgtaacaagcttagatatggtgggtaaaatatacgaagtaaaataaatggtcccaagtatatgaatgaatacatgtatttacagatatctctctcccctatctccttatgatttgtcattttcctcacccataatcctataaaataatcaagaacctccgagggagaga

SEQ ID NO: 52 - DNA, 3000 bp, Arabidopsis thaliana
  AT4G37970 chr417846648-17849647 FORWARD (source_file=TAIR9_upstream_3000_20090619)
taataccgttgaagttaaaaagggtaaaaaataatttactgacgcaggtagacgataagtacatgtactcggtggaaaacaaagacaacaaagtacacggttggatatcgtcggaccaacgtatcggtttctggatgatcacgcccagcgacgagttccatgcatgtgggccgatcaaacaagatctcacttctcatgtgggccccaccactctttcggtgagtacatacattctttttcaaaaaaaaaaaaaaaaaaaccaatttctagttttaattgtaaaatgttactaaaaatttaaaataacagaaatgtgatatatggtttggatgattgtgggtgtggcatgattgattcgatgtactattgattttattgagtctacattgtgtatttctgtttatctcttattcaattctaagaatattgtgtgttttagatgtttacaagtgtacattacgcgggaaaagatatgaacacaaattataaaagcaaggaaccgtggaagaaggtgtttggtcctgtctttgtttatctcaattctgcttcttctcgtaatcttctctggaccgatgctaaacgacaggtcttttacctatcaatgaccccattctttcattcgctttaatatcatcatcattattgtttacgtaaaactaaaatgttttagaaactatatatatatatgtagatggtttccgaggtccaaagctggccttatgatttcgtgaaatctgtagattaccctcttcatcaccaaagaggaacagtcaagggtcaattatttgtcatagacaggtaaataaatctatcattttcttagaaaggtttacttacaattacatgcaattgtatatcgtcaaaataatgtttttcaatccatatgtgagttattacaaaatgttgcagatacatcaaaaacgtgacgtacttattcgggcaatttgcttttgtgggattagcattacctggtgaagctggttcttggcaaaccgaaaacaaggtagtacactactactatatttgaaatttgtaaatatataccattacattgccatgacaggtacatgcaaaagagagtgatttttgtcatgagatttgtaaaacaaatttactgttactttcatgctacacaatatatatcgtccgtttaggtgatcgaaagtactcatttgctccgatttaaagattgatctatgaattgactatgcggatgaagtaacgatcttttttgccaaatggactaaactaacggcttttaataatcttttacaataaaatctggacaaagggatatcaattttggacacgagcagacaaaatgggaatgttcacaatagcgaatgtgagaccagggacgtatagtctctacgcatgggtttctggatttatcggtgactataaatatgtgcgtgacattaccatcacaccaggtacgtttcttttatttatatcacatcttttagtataaacattacacaacatgaaatgtgtgttccgtgtcccgttgatccaactacagacgcattaatggccattatttggtccatatgtttttatgtaagggttataattgttatgtgccacagctgaagatgtaataaggcccattactaggcctaagtatcgtggtgcatataattatgtatgtgtttggtttggccgataggtagggagatagacgttggacacatagtgtacgtgccaccgagaaacggcccaacactgtgggagatcggacaaccggaccgaaccgcggcagaattctatatcccagatccggacccaacccttttcaccaaactttatctaaattactcaaatcctcaagataggtaacatattaagcatctaattaaatattatttacaatatacttttattttaatatactaaatggtgttttatttaatttatgtaataatacagatttagacagtatggtctatgggatcgttacagtgttttgtatcctcgaaacgatcttgtttttactgctggagtgagtgactataaaaaagactggttctatgcccatgtcaacaggtaataatttttatttttatttttttgatgattcaaagtgttgttgtataaatcactgatctctctcgatacaaaaactatatattttataatatataggaaagctggaaatgggacatataaagcaacgacatggcaaattaagtttaatttgaaagcagtgattcaaactagaatatacacacttcgaatagctttggctgcagcctcaacaattgatctactggtttgggtcaacgaagttgattccaaacctctattcataactgggctaatcggtcgggataacgcgatcgcgaggcatgggattcatgggttgtacaaattgtacaatatcgatgttcatggaaagttacttagggttggtaataatactatttttcttactcatggtcggaactctgattctttttcaggtgtaatgtatgattatcttcgtttggaaggtccttccggagtttgataaactaaatacatatattaatgtgtaagtaataggagggattatatattttaattagtagtcaaaagtctaaaatatatatagaaaaaaattgtttgacaattaaatttgttacttatttggcctattttagtttttcttttgttattaatgtaaaactaatcattcgataagttcatcaagttcttcatactttagagtatttttaattaaaaacaaaaaagtggccaaaactgatttataaagcatatagttatatcaaatagtacaataatcacgatgcatgatatatttgttagtatatgaacattataagtaatatgtttatgttaaatatgttaagaaaaaatacatatatgtaagtcaacttctgattggtatgagagacctaaagtcaaaacgatatttctcaaacgaaacgtcagcgtttagccccatttatgttctcactcttttctatataaaaagaaaggtactctagctcgcttaat

SEQ ID NO: 53 - DNA, 3000 bp, Arabidopsis thaliana
  AT5G03840 chr51025813-1028812 REVERSE (source_file=TAIR9_upstream_3000_20090619)
aactgtgtactgattgtgtatgcatttgattgatgttttaatgtgaattttcatggttttggttgattgaatcagaatggattctcagatcaagcacgctgtggtggttaaggtcatgggtaggactggttcaaggggtcaggttactcaggttcgagtgaagtttaccgactctgatcgttacatcatgaggaatgttaaaggaccagtgagggaaggtgatatcctcaccttgctcgagtctgagagagaagctaggagacttcgttgatcttcttcttcatactccaaaatgcggaaacaagaaaacatttggttatttctcttttgctttctttgattggattagtggattatgcatttggagtcttaattttgttgttgctttaagacaattgctgctggatttataagttgtttggtattcaaaatctcttcatcccttttttatattgttcatcaagaatttgctctattctccatcttgttctacctgaatcatgtgttttgggttatgtttataacttgggtaagcagtggtgtggtcttgccatcgccacagatcttggattcgtagaagaatggttatattggtttggtttttccaattgaaaatcgaagctttaatcgagtccaaaatgtctctgaaatgtatcaaccaaaactgatataggaagctacaaggctaagataaaatttgaaagcaaatatgtttctaccctaatgtttgaaatctctgcttcaaattaaagtagtactccaaccagagcagctaatacctcatcagaatcactttcttctaccctggtccgttctgagtccaaccttgatttcatccgtggtggtaagagtcttaagactgtagcgagtgtagactttagtttattgataacatccctactttgtcaccatagtgaccaggcttctcgtatttgagttctatggctcttacttgccaaaagagcttccttcgtcaaagaggtgttttggggtcgccaaagttaataaccagaggccggggatagagaagattcttttgggggggtttggggtcgtgactcatgtcgtaagttgttgcttgctacatcttttggattaagcttcacctaagctaaatagctagttgatacgttgtctactttttttttttttttgtcgttgatacgttgtctacttgtattgagcacattgatgtttctcttttctttttgaaaattcgatttatctttttttttttttctaaattgttaaagggttgtcttgtttggagaatttgttgtagacaactttatataagtttgatatattttgctttgtgcctttattctgattaattaccaaatcttaataaagcatcaatcaagactttctaatataagaaaataaataaattattatttagtggagaatccataataagttaataatcattatattcaataggacaaaacctttgggtgtgtttatattagtctacggattttctgatttgtataacacttttggtatttcagattaatagagtattatggttgaaatatgcattcgtaaaatcttcggaagttttaaggttttgtaactaataatcatatatagaatcccaatttgaagaatcggttcttcacgttggttttattgattcgataaaaagaagtttttttaagaatcaatgcgacaatgcgttgtcgttccacgagcaaggagtcccaaatctgcgatatgtttctttcaaatagcctctgagctaggcctgggcattcagtttcggttcggttgcgcatggtttcggttatttcggttatagtaatataagaaccatttggatatttgaggaattttggttcggttcggttcggatatctttcggttcggttcggtttggatattaaacttggtaaccataaataaactaaaaccgaaataaccgactaaaaaccgaaacaaccaaaaaatttgaccaataaccgatatagaacgtacgtaaaaaagagaaatattcacaactcacaaagatggtccatactctccataatattaaaacacctgattcacaactcacgaaattcaacttgcaaactaataaagagaaaaacatagttaagtgtcaatacatactgtgatatattatctatcatacatatatgctttggttttcatttggttatcggttattaacctaaccgaaaccgaaaccgaaatctaagacatataatattcaaccggttattttaagctatccaaacctgaaccgaaccatgtttttcggttcgattcggttcggttaatcggttagcggtttttttgcccaggcctactctgagcaataattgtatccggagttgtaatagaatcaaagtacgatgagagtgtttttatgacaaatatcttaatcttggccaattatatgttctactgaaattctttttgaattcatcgaccagtgagacttaaaaatagctttttattcgccgaggtatatatagctaggaattttgtcgaaatttagacgttagtgggttttgttcttcgtgacacaaaagatattctatatattaacgaaatctagcgatcgatatggtatttatataaagtcttggtcatagataggggttgaaacttgaaaccatgcatgatatgccaatgttgctgaagcagtcaatgttgctgaagaagtcaaacgtaattatatagtgaataccaaaaaagtgatatttcttaattcaattaaatataattatagttttaaatcacctaaaataagttacttattaaaaccccccaaatttactttaatatagttggtgtacatgtttgagaaagcaaacaaaaagaaaaagaaaaagaaaaaaaaaagagaaagaggttagtacacataattgggaattaatgtctattgattcttttatctttctctctctctctaagacggaaaacccctataaatagatgtctcggtcgtctctttgtctcccaaatcactacaaatctctcttttcctct

SEQ ID NO: 54 - DNA, 3000 bp, Arabidopsis thaliana
  AT5G12330 chr53989755-3992754 REVERSE (source_file=TAIR9_upstream_3000_20090619)
ctccaacatgtacatttgatattatggttcatccttgaaaattcaactagcataacttgtcacatgtcatttggctttgtttgatagtcatggtctttcaaaaagagaaaataaattaaagaagacaacaaacgacttattcttcattttctacgtaataaccctgctcccacaaagatgttggctactttttcaactttgaaatgttccatatcgccattaaaaaaaaaaaccttatttaatgataaggtgactctaacattggaacgaacgagtctgttgctagagctaaactggtcttaacgtgatctggttgaacacgtgcatgtacatgcacactctaagataagaagaaaaggttgggtaagtaatcttataacacaaatcacacatcctgattcctgaaacaaactcagcagcataattaaacccaaaaaaaaaataaacctagtagcatagttgtgagaaaatgagcagtggataaatagttcaatgaaaatgtcagaacatgttctagtaaaaaatggaacaatgagagattttcaagcaaaaatgcgaagttacgtattggtttggtttggagtagagagcttctcctgaacacgactggcgtctattaagtcaagctggtgcacaatatggcggcggaggtagcaaatgacaccaagtagagtaaagccgtaatggtataacgaatatgacaaaactgtcaccaaaaaaatacattatgcagtttcaagaggaagagtgtaagatgaaatttcagagtaaaggaacgagctatacctctttaaacttcttcacccactcttcaaaagtaatatccctagactactttctggctttctcttcatattgatcagacgggaatcaatatatatgagaagagaagccaataaaagcaaacttgacctagactatatactttctggctttctctcttttttttttaactcgttctcataaaccaaaatcaaaccgttaagccatccatctcgtaataaaccgatgaatcaaaccggtcaatttggggtctggttctttaactataaaaagctgaaattaggcgcgtggggccgacgcatgtcggagggaagtgagacagcacgccacctccgccgattaggcaaagaagcattagtctttgtctcaacaagtctccgacgcctccattattgttttttttttccttttggttttgcccaacattttcttatcttgagccccgtgccttctgctccttttcttttgctttgtcataaatctactggtctggtacatgctttttttccccgaatgttaacaccttctgtatttccataaaatcgttaattttcttacgagttttaatgttaccaccctctccatttggaaaagaatagtcagaatatgtagattttcttcatataattggggatactctccatgttgaaaagaatagtctgaatgtgtagaaaatcggttttatttaaagtgtttagtggaaaaaagcactgcattgatttttgtagcttgtggaactcgatctctaattttaacccacatgacgaatcttgttccgtttgtatagtagattaggccacacactgttgattaaactcaaggattatttaaaacttgggatcattgtctagatatacaagtatcatttcattctagaaaaaggtcttcgaaagatatcaagtaaaacatacgagcataaaacaaagtgattaacgtaaattagcacaatgatcagacgggctcgaaagatctcaaagtaaaatttatcaaaacaaaaaaagacctcaaaagaaaagcaatgattcgacggccgcaaagcgtggggaggggccgacgaacattttactgaaagggttctttcacgtgcaatgctcaaactaattattctcattaaagctttttatataaaacataaaagctactttcttacttaattgcaattataccacgtaaaaatatgcatacaaaaccaattaagctaccccccctaaagacttaatttgaatgattttagtaaaattggttactactaaaccaaacaaacttttcactaaatattaacccagcttacatatgcatatgctttgtctgaaaattaaatccggatattatggaagcaatctttggcgtttgtgtaaatttagaaagattcacaaaggcaaaagtccaagatagatatcaacgtgtgccatattaataacgaagtatgcttcatgtatgttgaatctactcaacatttattgtatcatctataaaccatattttgaattttggacttttcgttggtcttttccttggtctccaaagcatttcacatttatgtggaccatgaaaagtttgtttttcttcttattatttgtaatgtatggccagtaagcaatccaacagtgtcggaaggatgaatatgaaactgctatatcaccacatatgtggtctctgttcggtctttttaatcggtttttgtctgtttttctgtagtaagatagatgacaactattacagagcaaaatgtgtgtgattaagatacgatgacaatgttaatcatgacctcgaaatctgaatttattacttcacatatgttgagtttatataggatgttgacaaaaaaaaatgtggatcaaaggatcttggatatttatgtaggccactagactggtaagtacaaaacgcgtatctaccgaaccatgtacctaaaagaatgaaccggtggagccggttctggagaaaacagaagccaagcgcaagtggagccacgcgcacgagcgggggtgggtgtgaacttcgtcatctgataaagatcaatagaaaatacataaattattataaacttcagctaaaatccaagaaaaggagaagaaggtatcatgtgccatttctttgagctattattataaaataaaataaaactataaaataaaaaataaaattaaaccaacaaaaattcacttgccttattctatctttctgcagttgtccatgatgataattagagagaga

SEQ ID NO: 55 - DNA, 3000 bp, Arabidopsis thaliana
  AT5G14070 chr54538836-4541835 FORWARD (source_file=TAIR9_upstream_3000_20090619)
tagtgaatgaatatggtttacttatggttatagtgaatggggatatgtgtgtgtgtgtgtgtgtgtttcaggtaaacatctcattgatagtgaatgatgaagaggcagagcaatgtgtgagggctctccactccgccttctttgagaccgatccttagtgcaacacagagcacccatctctctccttcaactctgcttctgtgcgtagtttaatgctaagaatgtgtcaagacttggaactatttcaaattcttgtccattgttgttgttgttgttgttgttgttgtgatacaatacatcataatttgagccaaagcctataatcttttaatttaaagagtacatagacaagctcaaaaagaaataaagaaataaccagtgtgttgagtagggattcagatttaaagcttagtttttaggatttaagatttggttttatgacataaggtttacaattattagtcaatggtaattatactattttgttcacaagaattaagatataaattagtttcttcattttgacatcagtatattttcataatttcttcttgtcaacaaaagaagaaaaaagaaaaagaaaaacaaattcagtatagaagagaaagtgagcattgaaggagcaggcttaggccaactccaatgggactctaaaacaccattttagtgtaatttttgcactaaaagctctccaatggaactctaaaattcacaccattttagtgtaatgctacagtgttacatcaaatttggtgtgatactattcacaactctaaacactattcacttattatatcaataaaaattcaattaacaaatataaaattacaaatgattaatagatataaaatataaaaatgaatggatacattaatatattatatctcttgaaatagtttatttagttatgatatttttatattattatgtatgaaaatactcaaaatagttacaattttatgtatttttaaatatatatttcaatatactatgtttataatgttatgtaattctatactagtaataaatactaatcaaaactattaaataataaaagaaatcttttaaatagataatataatagtatatattttttttagtatgatttttggtgttatggtcggaaaaaacacaagttttagtgctaaaaatgatgtaatttgaacaccaaaatggtgttgagccttgaagatgcccttagaaagtaaagaaacactttaaatgataccgaaaatacccttcctgaaaagcataagcaacagttacttgaccagggtcatatccgaccgaccagccggtcaacgagaaatacacatctgatgaaatctgttacgtaaggcttctgtaacggccttccaattttttccatataatatatgataaagtggtccgacaaacgcaatccctcaaagattgttattttatcatcatcttaagaacaaagatttagtcctccaaatcatataaatcagctttaattagtttatacaccaaatcttaaaccaatgaagggacatttaactcttcggactcacttaaactatatatacccatatcttaagctttcttttattttcttaaaaagtaacagaaatgttctcaaatagatcgatgtatagaatatgaataatttagggatattattcatatataatcaatcgatgagaggtgactctatattgcagattgggtacctacaaatttcataacaagttgtttgtgctgacaacgataaatgtttttattttagcatttttcctttttatggaaggggttggtcaaacatgagaaacaagaaggcaaattttgcagataacaaaaggaaggcagaagcatgcatgaaagccaaaaaagcatgtgaccaactttgtaaaattcaaccttttttctcagccttttggtaaaattatctacaaatccaaactatatatatatggaactatacatttttgtctaactgaattcacaacatttaataatcatatataaagaaaaagatggtagcaaaccaagaacacatgccaacttgtaatttccatacatcaagaacactgacattctgatggtaagcaactaaacactgttggcaactacaatctcttttaaaagttcattctcaagaatattgatgactcctttatttctttttatataacattaactagccaattttctatatcccattaggggctaagaataataaaatggttacgaaattcgatcttgatcaccaaaagatattccaacttgtatatatgtttagcgctacatttgtgagatgctaattggattacacaatccaaaatatatattccactccttttgcgatcatattgataatggtattatatgtagcttgtttatagagattgattagagaggatttgagagcataaagtgaatacggaatataatggaaaaccaccggccatatatccaaacattcgacctcatctctgcgttacgacttgctcacataatgttggcagcatcaaatttattattcggcgcgtctctatatacaatatatatatatacacatgaatattggaccatttgtatttatatatatgcatatattggaccatttgtatttgataatattggaccaatttcgcagacaaaaactagctagctagtgtaaatgtgtaagaagatattacagaaaaagccgacaagaaggatagatatatccattaccaacaattcccttattctactaacctatttaactgaataatattctacttttatatggttgtaaaatcttgaaaaaataaataattatacttaaatgggactcaaatgtctcttaaaaatatcatcctatcataacaaaagctagcaaccatgatggtccatgactatcacatcaataactttgttatcatttggcatatcctctctaataatatgccattttaaaataaattaaaacctaccagttaaatttagaacttcaccatgtataaatactctgcatgcctctcactctctccc

SEQ ID NO: 56 - DNA, 3000 bp, Arabidopsis thaliana
  AT5G16410 chr55367386-5370385 REVERSE (source_file=TAIR9_upstream_3000_20090619)
attatgtgattgacttgcctctgaagtctgaaccatttggaatatctctggcccgcttcatttctactagtcttaaggaatgattgctgcagggaaacatcactttatatacactagcgcttgtctctacataacattgttgttaatgtttgattcttttccacattcctctcttcctattaggatattttcttaggtccacagtcccgttagagatacaatgtattgttattagtagcatatcttgtgagttcaaaacataaaccaatcatacattgagctacacttccatgatctaacttggttttgcctatattctctaaattaaagttaacactaacagtatcaaactctctttccacaactatctcaccagaaatcatgattttcactctttaattagcatctatgttacaatcaacattcatatcataagttatcaatttggagatctcttcattctttgcaagcctccatcttcctccatcaatgtcagctgcttgattatctatatagcttgaagcggaacatggatgccgaatcttaagcggcctggagtggctgcttctcttcccttatacgttcatctgccacaagagcctttggtcctatttatattattcataatatgtaaaacaatgcatctaaaggtctcgaacgcataaaaccaaactcaagagtaagatatacatacctccaggacatagataaagccttttgagtattgatcatagtcctggttcttcaagagacagaaatttcatgcaacaattctttaagaggcaagacccctttatctagttcaccagcaaatttcttcaagaaaagttcccaagactctttatcaactttcccattcataacagccatggaaacaatcttcatcgcatcctcttctttccctaacttctgcaacccctcaaacaatatcaggaacgttgttttgttaggaaagcaaccaatctccagcatctcttcaagtaccctgactccttctttaacattcccattcttggacaacccttcaatcaaaacgttatacgtgaaagcattcggcttacatttcctctcatacatatcgtcccataatctacccgcctctgtcaactctcccttctcgcacattccagcaatcaacgtgttatacgtcaagagactagggatcgagcctttctcaaactcatcaaacaatttcctcgcttcagtaactctcccttctttacaaagccaatggatgagagtgctcagaagggcattatcaggcatacaattgttcttcaacatttttctccacagaccacatgcttcatctaccttatggtcttcacacaacgcatcaataactttacaacaaagagaagaatctggcataaaacttctctcaagcatttcatcgaacatgttacgtgcttccccagatttcttctccttacataaagctctgatcataacaccataagttacttcattaggctcaatctcattcttttccatatcatccatcaccgtagctgcctcagagaatctccctaacttacagtaaccatccatgagaacagtgtaagtcgtcgcatccggataccacccacgatccaacatctcttccaaaaccctcttagccgattccatatcacctctagcaacatacccacctagaatcgtagtgtaagtcaccaaatttggcactaatcccatcgaaggaatttcgtcgagcactttatatgcactttcaatatcatttttcttgcagagagctttgactagtagattacaagtgaagatgttcggcgtaattccaaaagactccttactgttcttaaacatggcatgaaccaaatcaaatctctgattctggattaaaacgtttaagagtgtgtttaacgatctaacggagcgtttaacaccgaaatcagggattcgaagaaagatcctcattgaagattcgtaacgacctgcgagaccgtagtttcgaagcaagtcgatgaagagattctcgccgcatttgatcggagggtatgagttccgaagatccgccatgagagactccacgggatcgaaagcacgagcacgggatagtttgaagagaattgagtggtaagtatcgtaattatgagtgaaaccgggatgagatttaccggcgtagagaaatatttggagagcgagatcgatgttctgctgttgggtgatcatggagactaatcgcttcgggaatagcctctgcggccacggtttgatcggtggcttctccgtgcagtattgctgaagagaagcggcggaggcggaggcggagaagagacgacgagaagctacgaatggagggaagcgcgtggggttcacgcgggaaaggaacattgttattgaattttgagctgcggcgactttacgaaggcgacggaagatgaatttaaatttgccggtgtctttattaaccggatacgtgccggtttgagtattcccggtcttctaaatccattatctttcacagccatagttaaccaaaaataaaaagtagtttggttagtaattttggtttagtttactttgtatttaaaagttgtagtgatacgtatacgtagttacgtacatgaaaagttgattttgtctactgcaaaccggttttgtaaacaaacaaaaaaaaggtctggactaatttgcccacccgctgctgcaactattcacatctttttaaacagtggtttgttatgtgtggctcaatgcaattatacaaatcaagcacaagattctgctaagttggaaccttttagattctaaatcacaatacattggcacgtgttgtaatagatagttattttaatactttttgtcgaacaatttgcaaaaatataatttggtcgctatatgatcgctaggttagaggttaccatcactgaacagtgctttggtcgtaaggttaccccatccgcattcgtattattctggataaggttttattttgggctcatttccctttcgtggaaaacaaagaaaaaaaaacaataa

SEQ ID NO: 57 - DNA, 3000 bp, Arabidopsis thaliana
  AT5G20740 chr57026533-7029532 REVERSE (source_file=TAIR9_upstream_3000_20090619)
gaagttttgactttgcgggaaggacgaacacctgacttgaaagaggcagacttcgactctacaagcttctccatatcagcaaataaattagagcgcagcaaatgtaactgttgagagaaccaatccttcaaccccagcaaatcagaatgggtaacaaaagcagcagaacctttctcaccgcgcggatgagagccatcagcaggcgcagcaaaaccactatttttgcttttcctggaaggagggttacaaataggattagacggacctactccacacgacttgctagctgaacgagtaaagggtttgcggggggaggggggggggaaggatgactgctttggttctttagctgacatctcagggataggatgagcttgtgaggtttcagccaggccaggagagggacgccggttaccactagggtgttgttcatcattagtaggttgcacagtagtggcatcaataggccattggttaggttggaatcgatgacctttactaatcaaaccaagcatatgttcaaccttcggatcatcgttttctttagaccaagcaaattcattctcattaagtactacagagggagtcaaagtgtaggcaacagataactgggagaaatggaaacaagtggcttatcatataaatagaagatcaggaaaaaaagaagaaaatgatggaaaaagtaatattacttacattagcatcacactcaacctcaagaacaggagattcacggaggagaatagtcttgcccagcacaacaggagatcggtctatgaaattcctcatatcagtaggatcttttatcttgctacacagggccggaatagagttgaggacttgaagctgaagggcaagagggaagccataacaaaggctggttttctgacataaacgtttcttcagttcaactatagggttcgaatccccaaggtcagggccaaaacgctccattgttgaagagaaagcctcacggccccatgggtaagccatgaaaaactcaggttcatgtaacatttcaacaaaccgagaggttactctcccggctaaccggttgctacatacaaccacaccatcaacaatgactattagagctagacataattttttccaagtcgttagcttcttgttagccggcgcttccaaatcatcttggagcatctgaagcacttcggtgacaaacacaattttcttctccccaaaaagacgattccaaacacacagatatgcagggtcttggtgttcttcaacctcagcttgagtgggaatttgatgacacaaaagaccagtggaaatgtggaactcgcgcagagaaaaacgaaaaggaaacccaccataaaccacccacagttcgttaattttccgggtaatcacttgacgcgacagcattccatggatcaatttcgctgagtgagagcatctttcaacagggagtaagaacaatttcctaaattgagaagacattaatatagcccaggcttcagaaccgtgcagtagcttgacgagagaacccaaatactccggtgtggagtagatgttaagccgagcaccctctggcaaaccgtcaacagcgaagaggcctttaggcaaagtagggattaagtcccaattatcaacgtccgatgaagctccggtggaagacaccgccggatctgcatttcaatttgggattccggtactaagcattcacaaattcacagaatcgaaaacataaaacgaaattagggttaagttgaaaatttacctttagtagaatacatcttacttttactacgattacgaggagattgacgttgattcactccgacggaatcaacgaaaattaattttttttactggatttgatttctctttttttcgtcattctctgtataattttgttttgacttcagacgagacaaaaggggaaaacaaaacgacatgggcaaaatcagaaaggatttacccagtggcagttttgaaaatacagaaattttaaaagggtacacaggatgtttcaatgtgtggggttacgaccaggttactaaattgctaattgtgaatagtatgttgtccaaatcaataattaagttcaaataagtgactagatagcaacttatcccttgaagttattgggatatatcgatcatcacatggttatggtgaagaaatcggtctccgttattgtgaacttgtcgatctttcgcagactaaagcggcctcaaagacgatgatcaaagttatttttcacacaattgacctgactcctgaatctccaaaacaaataaacatacgaagatgtgacattttcttttaagtgggtccttctaggaaagcatcattaaagttattacagaccaaaactttaggatagtttggtgttattttttatttctttgggataaaatagtgtggtgttattttatttagtaaattatacttggttaatcaaatttgctgtttgttttcaaattcaatatcagaatatgaattttgatttaatttttactcgtttttaatagcatgtaaaaatgtaatgtagaattcagtttacccttaaaagttacaagagtggagcaaaagaaatggcagccatttaatggcagataaagattttgtctctgaaacctcttttgtcaattcataacccaacgtggacccaaacttctgaaacctctttgaaaataattgggcactggtgaccagtccgccaataattctacaagttttgttccttttcgtatcgttacttacatatacaaattatttacatttttgttattaatctatgcattgcacagtcttatttctttcgctggattcgctattctctgttctgtaatatgcggtgcatttaattttacagattttcccccataatcaagagatgctacattcaccttcctacccctatccttttacacgtaaaacacgtaaacataggaaatatctgagggtaattaagtaatgttctacggacctacgactttaaattcttacccaccaccactctccttttca

SEQ ID NO: 58 - DNA, 3000 bp, Arabidopsis thaliana
  AT5G27690 chr59800764-9803763 FORWARD (source_file=TAIR9_upstream_3000_20090619)
atatgaatgataaatatatagatgcatgcattatgcataacataactatgcaaaacatgagcaaaacatatgcatatgaaaaaacttttatatgcaaatcacatcacctaaaacatgtttagcattgtgaacatcatggaattcattcattatgtagagaaataaagaaaaaagcttattagagattatacatattttaaggatatcaactagtctattagaacataatattactgccatgaatggatggaagtaaggcatattcactccaaattgctgcatttccctatgtcgctagaatcaaccatatggaaaacttttccacacttttccacatttgttttaacaagcgtgtcaaatgagattctcaactaaaacaaagactgattattcaaactcaccacaaaggtaaagagatgatgtgcttagattgtgattgtaacgaggcagcacaaaattcacacgaacaaggttctcatggccaatgaatgattgaacaaaagttaatgttgtgaaggcgattgccgtcataaccaaaaccttaactacatacacaacaacaattataaaaggatcttttgagctattggtcatactcatattctttgattaaagaaaccgttatataattttccagctgttttaaattacaaatttagtttgttttttgttccgactcatagaaataattttagttattggtttcttaatcacagaacaagttaaacttgtaaattaaaataaaaactacacaagatttctttaaaaaaaaaaaaagatatggaagttttgaagttcacaaggaagaaagaaggtaaatcttttttacaaaaaaaaacatggtttaaagtttaaaatatgttggaatgtattcatggtttcacatagtctaaattaaaatgatacaatatagaggaattgttagaaatgcttcttttatatttctctttttaaaaatacttttttctagctattcttttagtttatatttttcgatgaccaaaggttataaattttacaagtttttctaaaaaaaatagaagtgtaaaagttttgctaagttttgacggtcttaagtgattttagttttgggcttttgtatataaaagagtaaaatataaaaatcaaaaatcaatattatacaattacaatttaattacattagctaactaaactgtataaacattcattatctatacaatagtatctctttttttaaattatatatatatatatatatatattgcttcaatataagtatctgaaactttaatacacattattacataatatcattatgtacttttgttatcatcaatatgttttcaactatacacctcctaaatttaaatcacaacctagagatgttgtggtgggtaaaaaagcccagcccgacctgaacccgaaataaacccgcccaccaaaaacccaactttagaaaactcagatgggttttttatttagtgggtaaaccccgaaaaaacccgattgttccgaaatatattttgcttataaatatattttaaattatatttattttctgtattacatatatatttgatgggaaaaaggttgaaatgttaaattgcgttttttggcggaaaaatgttaaattgtgtttttggcgggaaaaatgtcaaatccttttcttggccggaaatgttgaatgtcatttttggccgaaaaatattgaatatcttttttggtgtgaaaatgttaaattccgtttttgatcggaaaatgtgaaatcttcttggcgggaaattgttgaatgtcttttttggcgggaaaatgttaaattgcgtttttggcgggaaaatatgaaatcatttttggcggaaaatgttgaatcgcgttttggcgggaaaatgttgaattgcgtttttgacgggaaaatgttgaatatcgtttttggcgcgaaaatgttgaatgtcttttttggcgggaaaatgttgaatgttttttttggcggtaaactgttaaatcgcggtttaggggaaaaaatgtgaaatctttttttacaagaaaatgttgattcacttgaaaaaggacttaaaaaaacccgtgggtacccataacccaatgagataaacccatatgagttttatttatttggatacccagtgggttttaacccatcattaacatctctatcacagcctttttttctttttataaaatatattgcactgtgataattttaaaatacgtctgttataaaagatacaaacacacatacaaatacacttcatataaatactaaaaaaaaaaaaatcctcagtgtagcgagagtccgatcctagttacaattattagactactgaaatgctatgatattatattgttaccaaacttcgaatatccactttaataacagttataaaaattgtattatcgtaatgtgttatcaatacttatattttttatagtgtaagaatagtgtaggaaaatacttgtttatattggaagtcaaaattaaataaaaatgacataaacgatactatgagaagaaatcaaactgagtaaaatgggcataatccaactattttgtgagccgatacatatgacaattttcgatcgctgaatcatggtttggaaatatgtttgattactactctctctttctcggtcaaattaacatccgtaagagagagaaaattattcaaacccctaggtttatggtgtacttcgaacctagctagactgggtattttgacaatttcagtgtcctaaaatatcttctaatagtaagaaaatgaaaatgatggcaaagaaaaatcgaaaatttcatgttttgacaaaaaagaaaaaaaaaaaaactctttttaatcctaactaagtaaatattaatgttttgagaactgagcattttataatcttttccaaaaaggcaactattagagaatattatctgctgaagaagtcccacaaaaaaaaaaaagatatagaagccatgacttcgttactatattacacatgaaagttgcatcaattccctct

SEQ ID NO: 59 - DNA, 3000 bp, Arabidopsis thaliana
  AT5G35770 chr513940868-13943867 REVERSE (source_file=TAIR9_upstream_3000_20090619)
tgttgtgacctaacaagtttttagccacctaacttgttctaaagcattttataagctatttatacattggattcatttttctgagttttatcagagatttcaataataccgaagagatattttctaactctttcttcttattgattttgtttatgctttctcattcataaatgttgtttgtttcatccatcatgtatgagtagtctacttgttagggttagggtttctcattagggatttagatgatttagtagattgttcacctgctaggatatatgtaggatttttcatctttggttattcttaatgctagttctagagtaactaattagaacttaatcttaaataataggtatgcccaaaaaggtatatgttaccagaaatactatttgatgagccaagtttatagatgtgaaataagaatcggcctatctatttaggtgaacttatcaaactcgtttatcatgcatgtcttaatatagggatttgccaagcgaccgctgggaatctttagccctaggcatattgagtagcacggcgaaccaccgcttattctgtaattagggtttgagtttaagaacgattcgataacatcctagctatttctatatctcttatcatctatttcctgagaataccctaaacctaacgtttttattatcttgtcttacaacttcacaatccactatctacttacttgctttctttttattttcttatcatctatctagcttgattttgatactttagcctattgtgtgaacaaaagagttttgtggaattttacccttaagtactaacgacgatctcttatttgagggagtaaacttagagcaatttgacctacatcaaattccattgttgtacgtgtacggtttccaccccatctaagcaacgatatttcatccttattgactacgttcctatatcgctctccaaagcatttgactgaatcatatgcccaagcctgtaacatgaaaaccataccactgatggtgtacgactttccaataggcctcaacatctttatagcattaaccaacgagacaccaaatctccttcaaaagtattattaactgtctacttaatagaaaatgtacagtcttacgagaccacacgaattgactattcgataacttagtaattattccaataggtgcctccttcatttgatcccacacatcttatcatactaattctctaaatcccatagaaatatgtcatcttgatattgtgattaatgagtttattctctagattagactctccttctgcgtaaattcttagagggtaatcctttgcttgttcttcagaaataaccttagccatctaataaaccataaatcagccataaccatacattaactcagccacaacataaaacaaatttagccacagatgcattgataaatcataactcagtcaaacatacaataactcatccacaacataaaacaaacctagccacatatgcattgataaatcataaatcagtcataaaacatacagtaaccaagccacaacacaaaacaaactcaaccacatatgcattaataaatcataaatcagtcctaaagcatacaataactcagccatcaaatcgaatagaaataacaaatcagccaccatatgtaaatcgacggcaaaaatcagccgtaataagcgtcgataaaaaccatcaatatcataacagtggttttagaaaatggttttaatcatgggaatcacttgctattgaacttagatgtctgattattgtcggaaaaatcgaaataaagatttgacacatatcgaaacaagttgagttgagacgaaatgtgactcgatgacaacgacgttaaaagtttcgggcggaagagacgagagaaagagagaatagccacgagaaataggttttcttcgttttcggataaacacctagtaaaagagaagacatttttttttcccaagatgaacaatgtctgggaggaaaaccaaaatgttcctcttagtgacgttaataatgtggctttatttttttttctctcttaataacgtgattcatcattatttcgttaattatttttaattaaaaaagaataaaaatgttttaactaaaaaatactaatcaaaaatgttaattgtaattatataaaagcactatatatatgaataattttcagaaaatgtaaatatttctatatttagattgaggaataaacttaaaagcatcgcatgctcatgtgttggtttgtgatgagtttggactaaagttcacgatgaaccgtggttttgaggatcgtgatagctgcaatatgagaaaaagtagggttaagttttgtgatgacagaatatggcgaattctaggtttgaggattgtaatgataagaatatgcttaaccagatttccaaaaattatttacttataatgacaatagtatgttgggaaaaaaaaatatgttaaactatgctttaaaagtttttactactggtatgaaagtcgagatttggtgctacgagtttgatctctctgacaaattttttgcgtatacatattattttatttctcatttttaaaatggatcgatctacaaatatttgtgtttagccaatagtttaccatatacaaaaaatttactaatgttcgcggaaggaaataaacttagcaaatatattgttgaattcttaatgatatataacactattttaaatataaaaaatcgtcaaaatataaatctagtgatttataataatatagctgttatattgccattataattcaaaatctgcatatcaaaagaaagaaattttgaaatatgtcaccatactgaaccaatacttttcctaaaaaatataaaattgaagctatttcacaaacaaatctgcatctattaagaataaacaaaaacaaataataacaaatcttgacaaaagaaaataaaaataggaaaaaaacataccttcacaacctgggtcccccacctcggagaagtatgtctttatctacactttttgtccttt

SEQ ID NO: 60 - DNA, 3000 bp, Arabidopsis thaliana
  AT5G39330 chr515743234-15746233 FORWARD (source_file=TAIR9_upstream_3000_20090619)
gagagatttcaggaaacaaaatggtgaagatctgttgtattggagctggatatgtaggaggaccaacaatggcagtgattgcattgaaatgtccagatattgaagtggcagttgttgatatctctgttcctagaatcaacgcttggaacagtgatcagcttccgatttacgagccaggtcttgacgatatcgttaagcaatgcagaggaaagaatcttttcttcagtactgatgtggagaaacatgttagagaagctgatattgtctttgtctctgttaacacaccgactaaaacgactggtcttggagctgggaaagctgctgatctcacttattgggagagtgctgctcgtatgatcgcggatgtatcggtttctgacaagattgttgttgagaaatcgactgttccggtgaagacagctgaagctattgagaagattttgatgcataacagtaaaggaatcaagtttcagattctttcgaatccggagtttcttgctgaaggaactgctatcgctgatctttttaaccctgaccgtgttttgatcggagggcgagaaacacctgaaggattcaaagctgttcagacacttaaagaggtttatgctaattgggttcctgaaggtcagatcatcacaactaatctctggtctgctgagctttctaagttagctgcaaatgctttcttggctcagaggatttcatcagtcaatgccatgtctgcactttgtgaatccactggtgctgatgttactcaagtgtcttacgctgttggtactgattcaagaatcggttccaaattcttgaacgctagtgttggattcggaggttcttgtttccagaaggacattctgaatctcgtctacatctgtcaatgcaacggacttccagaagtggcggaatactggaaacaagtgatcaagatcaacgattaccaaaagaaccggttcgtgaacagaatcgtgtcctctatgttcaacactgtctccaacaagaaggttgcgattcttggattcgcattcaagaaagacactggtgacacaagggaaacacctgccattgatgtgtgtaaaggtctattaggagacaaagcacagatcagtatctatgatcctcaagtcacagaggaacagattcagagagatctctcgatgaaaaagttcgactgggaccatcctcttcacttgcagccaatgagtccaaccacagtgaaacaagtgagtgtgacttgggacgcatatgaagctacaaaagacgcacacgcggtttgcgttttgactgagtgggacgagtttaagtcgttagattaccagaagatcttcgacaacatgcagaaaccggcttttatcttcgacggaagaaacattatgaatgttaacaagttaagagagattggtttcattgtttactccattggtaagccacttgacccatggctcaaggacatgcctgcctttgtctaatcacaaggcaaaacctggttgattcttgtttcttactttcattgtttgtgcttttttgctttcgctttcatataaattttagtctcaagattcgatttgtgaagtcttgaagagttgtttcttacattttacaatgtgtttcttatgctaagctcttataattttgtgtttcacattttgtcaaaagctcgtgtaattgttgtttccttagaaaaaagcaataaaggtttagtttttaattcaaagtcaattaaaattgaacctgttcatcaaattctagaattgaagtctaaattattgtagattggtgtataataatatagtaataatctctttagatcggatctgaaaatactggaacatttctagcccataacttattgcgttgacaaaaaaaaatcaaataacctattgagttaaccagacttgagttagaagtaaccaaatttcgaagcttttaaaccacaggattggctccgatttatagtaaactttggttgttgttgagaagttaaaacagaaagaaagtaaatagtgttattgttttcttacttgtggtagaattcacaattgtgaataagaagatcttagcctagtgagtgagggacgtccaaaagttagacttgtatagtgagagtgtagtgggtgaacgtgggacagaacatgcgagtccgtgcaagtcgcagagtggttgcttgcacgaaattattgtaactcatatgtaatgtcaatgtcataatgatatatcttttcgttggagatgactaagctgaggttactcatatctattgtcatcattcatctccaacagtaacctcagcttattttgattatattttattatttttcattgtgaacgttaggtgtgagagttacgtgaaatcggtttattcgggtcgggttatttggttttaggaaattcggtttttgaaaaatctctccgaattgaaccgaaattaagttcggttcgagtcggtcgctactcggattattcggttcagttatagggcttaaaattaattccaaaattatatttcggttaatttgaatcaaattacaaaaaataaaaaaaggagaatgagctgaaaacaaagcccaaacatagtatctctcccttttaaaagagtggttagagatacattttcttaggaacctcatgcaaaagatattattggaaaccttatgcaaaaaaggtaacaacagatacaattttttcatcaacaaaaatatacattgatataaggtaataatatttcccacacatattttttggattattgaatcaattcttgatttagatttccttacactactctaaagtcttataaactttaaagcataatgtaataaaactaacgaaacacaatacaaaatgggaaagatatcagtaaaaagatatacacatataaaacatttttttgatcgatacacattaaatttttgtaaaagaaaaacgctatttatagcaatacattaacgtcgacataaaaatctttgattataagaaaaaaagaaaagaaaaaaaaaaactttgatt

SEQ ID NO: 61 - DNA, 3000 bp, Arabidopsis thaliana
  AT5G42655 chr517102603-17105602 REVERSE (source_file=TAIR9_upstream_3000_20090619)
gttgggttgcttgtatttgaatcaacgaggatcggattcaaatctagtgacttgaagaaaccagatttgaatgggaaagagagtcctagtaagaaaaatgaaggaaatttgaatcgattggatcctactaccaaagttattggtggtgttagacaacgtaagcttgattaatttgtgaagttgtacaaaaaggatctgaattttgagatgattatgtacgaattggcttatgtggaaaaagagtgttactttaatttgctattggtttgtgaaaacttgggtattgtttggttttattcgttctctgatgtggaatgataagttgatagttcaagtgtgtgtggattgtggaaaaaggacctgaatgttgagatgattatgtatgaataggccatgaatgtggaaaaagagagtgtctttaatttgctattgattggttaaaacttgggtattgtgtggttttatttgttttgtgtcgtggaatggttattgatagttcaaatgtgtttggattgtggaaagaggatcggaattttgagctgattatgccggaataggccatgaatgtgggaaaagagagttgctttaatttgttattgattggttaaaactttggtattgtttgattttatgtgttttatgttgtggaatggttaagttaatagttcaagtgtgtatggattgtggaaaaaggatctgaatttttgagctgtttttatgtctgaatgtgggaaaaaagagacatctgtgataaaatttgagtattgttgtgttttatgagttgtgtgtgttgtggaaggatatgttgatattgaaagtattgtatggattgtggtaggttgtttgaagcttttaccacctgaggagcttgagcatcttgatatcttagagcgtaaagattcaggctctccggttaagagagttgtgtatctaactgatacggatgtgtcggtgggagaaatgagaggtgttcgtggtaatgggacgcggtttaatttattcacggggaaccagacttttgctgagagagaaaatagttttcaggtcatgatttggtaactctacaaggcttaagaacttgagtttcctataattttaggaattttttatatggatcttgaactgagtattggctttattgcatcaggtaagggaaacagtttctttgcattgtgggtttttcaatgaaaatgggggatttaggatttcggataaggataagaagtttatgacatcatgtgaagtagtggtctcgacgtgtgcgtttggtggtggagataacctttatgaacctattggaatgtctaagacatcaagtcaaaaggttggtcactctgattttctcctgtccactgtttctcttgcatatcgagtacggagtttgaacaaatcgtgattctctctctgctgcatccaatcctgaatgagttttttggatgcaggtttgctatgttgcgttttgggatgaagttactcttacaacacaagaagcagagggtcataagatcgatgagaatgatcacattggaaagtggcgtattgtgattgtaaaggatcttccttttacagaccaaaggcttaatgggaagatcccaaaggtactactcatctacttcagaggcacatgtgatgatttgtactgcacgtagcaatcttattcgctgggtgttaaacgtttctgattatgcgttcatggtactgtgtaaaatgtttccattacaaccaaatttaaccttctttggttttcattttgtgctgtgtaaaatggtatgtgctcgatttgaattagctcactgttgtcttttttttttccttacatcttttaatcttgtttaaatatggttggagtcacaatatttgagtttccgttttgctttcaagcacatgtatgtctgatataagatgcttcataatgatactaatgaatctacatgctgtcagatgttggcccatcgccttttcccagatgccaagtactcgatttgggtagactcaaaatcccagtttagaagagacccacttggtgtactagatgctcttctttggcgaacaaactcggtgcttgcaatttcagaacatggagctcgaagcagtgtatacgacgaggcaaacgctgtcatcaagaaacacaaagctacacccgaagaagttgaagtgcagataaatcaataccgacatgacaaattacccgaagataaaagattcaacgggaaaaaaggtaaatttctttataccataagtgttgtaccaaagctcctaaactttgatgatggaagtaatttttggctgttttactttcttacagctttatctgaagcctctgtgattgtaagggaacacactccattgacaaatctattcatgtgcctctggttcaatgaagttgtccgcttcacttcgcgtgatcaattgagtttcccatacgttctttggcgtttgaaagttctcaagaacatcaacatgttccctgtctgtacacgtaaagatctcgtcaatagcataggccatgtacgcaaggctaaaccgttagttaccctatgaatttactgtaagcttccaattcattggttattttagaatcttttttgtatttagcgtgtagacataaaaacagaactgacatgttttaaactgaaaccgaactaaacctgatcagaataaaaataacagcttgaggacaaaagaaggtagtggtttagttatgttgtggaagtagtagttataccaaaatcactaatctaactttaaaaccaaagaggtttgttgaaagaacagttagaaactcctctttgtctctcttcttcttcttctccgatcatgttaactagaatcatcttcctcatcgctgtcgccactgctttcattattgtccttctactcgctttgttgtcacctgttcctgattacgacccgccgggatcattcttctccttctccctctatgttcaacaaacccacaccccatcttcctcctactcctctcgccggtccagccagcac

SEQ ID NO: 62 - DNA, 3000 bp, Arabidopsis thaliana
  AT5G53210 chr521588942-21591941 REVERSE (source_file=TAIR9_upstream_3000_20090619)
tcatttcttgatctccatttttatctaaagcatcttatacatcaaaacaaaagaagtataatttatgttatttcttaatctatatgttcctagaaaaaacaactatgaatcaaaatagaaggaagtactataaattgtgtgatccaaaaatcgagtgcgagtaattttggaagccatattgagatctacacaggatttaaatcttaggaaccgagcttgagttggtgctacgtttaaaaagagattcatctgtcaaaagcatgtcgtggtctcacgataatcggatatatgtacatcagtacatgctaatagttcataacttcaccaccgtgaccatctagaatccccaactaaaatatccaactataatttaaaaataaatttgaaagtatatcctataaacgttttacaaaaccaaaaccgaaacaagatcatcactgcgataaggagatgttgtgtatatatatgcttatactccatcatacatgcatccatgatttcacatacatatacatgatgcatgtggatgaatgaagaattgtagaatctcgggcacggagaaggcgtgtccggtcattgaaagccgcccaccatctttattatatttgtttccgaagtactgatctttctgctcaacttgcagattttgatttctgcgcaacttgcaactttctcattctcagtttttttctctgaataattgcttcaaaagtcaattattattgttattataaaaaggcaataaatttcagagtagtcatatattcattcttttagtctgtcgagtttttgtttgtttgtttttgattagtaatcatcggaaccaactcatgacacgacttcttaatcgagactgcaggaagaatataatcaacgttttgttatgtcatataaataaaatgaatttatgtcaatctcttttttgttgttgtagtaatgtaacgggcaacagtgttatagaaatgtagattttgatttgcgaaataaatagagcaagacaaaactgtacgaaaattgaaaacatgtcatagtctccataaaagggtatctatatatccgtatatatatgaaattgaataataccttggatccgagtcatatcaaattaatatcctgcatgcatgaataattaaactttagttacattactatgtccgctaaatcaaaatcactgacaaatatcttgtttattttgtacagtgtaataagtttgtccgggttggttaaaatcgaaaattgaattgggattcttaaaatctattttgttttaagatcagtttggtgtttgacaaaaaagaagaagatcagtttggaatgtgtcacataagaacatgtaattaaaagtaattttcatcattatataagcattgcattaagaaaaacatagttttttctaattgctgatgtttcattaatttgggctagaaacaaagcataaaggagaagagacccgttactaaaaagacatggtcaggtaaataatatctgacagccaactcattaacttcaaacctaacgactacctggtttgtcttggctccatcttcttaaattttcgtttattttctaaaacatttgaatggattatcaaaaaagaaaatgtcaagcaatatttatatttatgtacaaatatttcaaaaacttttgaaagggaaagttcaaatgaagggtggctcctcaagatgcaatttgattttgatgagacaaaatgaggcacgtctagtatgacatcaactatttattgccttagatctatcccttgtcttttgttttcttgaaaacaaattcgtttgctccctgtatcaatataaagattaacttacaaaagaggtgatatatatatatatatatacacattatatcccatcttattaataatgcttactaatattgctaaattcatattctattttgggttggttttataagaaattgaattcttatgccatctcatcaggtgtttatgaaatatgttagggatccagttgaactgaactaacattactaaattgaattaattaaattgtatgtatggctcgggaaagaagaaggaacggattttctttcagaattgtggaatcgaattttgttcttctttttgggcttaaatgttggttagattacaccatgacataaacaaaggccaaagtatatgctttaagtgaaaatctgtatttttaaatcgactggcgataacatgtctgaaattggtctatcaatcataaaaagtctctaaatatgtatctttttattttttgaataatgaatgatctataatgtgaatgtacaacacatcattcaattgattgtctcacgattgaggcgctaaaataaatgtgaactcactaatttcaaatgttttttttttgtttgtttttttgtatatatatatacttggaaaccgggattggtcggaccatattcgcaacccgaacatatacgtaaattaaatacaaagaacgtacatgtccgtgtttatgtgtattacatgtttgtgtgtacaaaaaacaattataccgagcttgtgattattcaaaattttacccccataaaaatcttttcctaaggaatttatgtatctttccctttgcaatatgcaacctttatctctgtaatataaagtatacaataagacgtaccatatatatatatgtagatgcatatttatatctaaaacataaatgagacacgaaaaacctagatcctcccccaaatttcatccaacggcacaaaccgcataataaataaccctatcaaaatataataataaaaattgaaatttgatagcgatagtttagtcctcgatgaatgcgagtccctcatatttactccttttggcattcactctctttatttctctttctcaacttgatatgtcttttggcgtctctttctttccctaaatctttcttttattttgctgtttaaaaaaaaaatccaaccataagacaaaacaacgaacgaggaagagagagagagaaggatatatctctaatcacg

SEQ ID NO: 63 - DNA, 3000 bp, Arabidopsis thaliana
  AT5G56530 chr522886226-22889225 FORWARD (source_file=TAIR9_upstream_3000_20090619)
tgaatctcattttgggtgtgtatgaaattatgatgatgatgtgttcttgtcaaagatcaaacatcaaagtttgagttctcctgttgattacttcttggtggtcttcttcctgcaggttggagaattattctggaatccaaattcaagattttgtagtcatttttctaggaggaagtattcgtactgaaatgtaaaagtttaagaggattccgaatcttctgagctgtatcttacagtggaatcaactttcaaaatcttaagaggattcaatatttgtctctaattatataatcctttatttattttcattaatcaaggtgtttttaagaattttattgtgagtttcataattgaatcatgttgatagataacatatataatttgggccagcccgataaagaatggaaaaccccaaaagtaattttaccaaaaaaaaaaatcaaatttgggccagcaggccaggtctctgccaatggggtccaattagtatataccataatctaaaactagatgtgcattcttcagaagttctcatgcttacgaaaaccaaactcatgaaatgatgaaaacacataacaatatctagtataagattccacaatgacgaatcacgaaatattgataaattctcaaaagatccacaaaatctatctgatgtaagaagaacactgcatttatagatatggccagtcatgctacacagtttggactgactgttttttttttttttttttttactttcccttgaaatattattatattagaattactgttctcaggatccacttggacatttctccaaaatagtcatgtaaccgttgatttgggttaattaatcacttggttgtttagtgcacgcacgttataatcttttactagtaatcatacaaattaaaaaagtagaaaagacaagggtggtgattaacgaggatgatggccaaattagttaagacttctatttcttaaaaataataatcagaaagaaacatgaaaaaagctcatcttttctactacctaacctactcaaagctatctaaccaaacaagtattaagatgttaatcacatctcataaatcttattcttcatactattaattttctgttgacttttacaagagaaaatacagtatacatttttagcatttaaattgattaatgttttggttttaaactttgaaagataaataaaaagaattaaaattgcatattaccaagtaaaaaactctttcttgtgggaatctcataatgatgctctctgctacacacagtttgactgactcattggattccccaccgttcgttagatcctttcttccgtctttacctgaccttcttcattgggaacatagacgctccgcaccgtacacgtgtcatcgatcaaaatgaattaaacgccatcaaaactataaacggcaataccatttcacttgttaacggcgagcaaaccgaaacagaagaaggaaagaccaacagcttcatcgggagacagaatcttgtgtctgcttccccatcataatgctactattttacttactactactccattgattgaaactatttgtaaccaaactaaagacaaacattatcattacgaggcaaccaactctcgtatcaacaacaatatctctttgtaatttctcatataacaacaaaatatgattccatttttttgtaaatttgtaatctagtatcattttttaccactgcaaattaaatctgttggggtagaaaaatgtgttaaaaagaactttgattcttaacctagactcaataaaaacgatgatcattaatactccttttatgacccaaccacactattaatgatcaaataacttttacgtataagctgccttgaattatatttattgatttctttagtattttggaacatccatcttaactatagcctataggtgataaacaaaagctaagcattagtttttttttaataatctgagtagctaacaaaaagcaataagctattcgaccagtagtatgatgggtcaaaacccaaaacaatattttttacaaaagactcttctgaaaaagttgatactttttgcagagaactttgttcttgtttctttatcttatctttaatttaataacatcgttcgatacgatgtttcagaatttattcgtttcaagattataaatttaacagaaagaaacactgatttaaagaggaatatatgcttgaaaagtacgcaaaacatttaaattttgaaaacataatgtttctccatcgttagtaataaggtccactaaattaccaaaattgagtgatattattagtttgatgcaattaaatgcaaggaccatttcatataaaggggtaaatttggaatcttacgtattttggttagtcgctcttcggagcggaggctcgtgaattttttcacaagaagttgcatcggcattctcgccatgactcatgggctccactcgcatcttcttttagattatgttttttttgggtcagaatttggattttgttatttcaactatttagtaagtttaattataagaaaattatgttctaatgcatttttttagttaagttaagctattttcttccttaatgattcaaatgtatctaccaatttttgatgtattttgttcaaaaaaatatcgtagtattattatgaaataattcatctgaatacccattatctaaaaaaatcattattaatcaagccaaggctgacactttttttagaaaaaggggtttcgaataatcattttatgtagcaaaaaaattattttcaacataaataaaaaagttgatctttaattgtcttttaattacctagaaaaggtttatttagtgataattaaaagtagagagagaatatttgacaaataaaagagagagagagtgacaaaaagtggagacctttcatcatcaaaggagagacccacttcaaatgtcactgcagaatcactggtgttagtggtggtgggttacatagtaagagctctctgctactagtagaagaa

SEQ ID NO: 64 - DNA, 3000 bp, Arabidopsis thaliana
  AT5G58780 chr523737336-23740335 REVERSE (source_file=TAIR9_upstream_3000_20090619)
gtttgatagaagaggtagaggaagctacaaagagttacgaagggaagaatctcattatagcaatagactatagtggaagatatgatatcttacaagcatgcaaaagtcttgcaaataaagtgaaagacgggctgattcaagtggaggacattaacgagaaggcgatggagaaagagttgttgacaaagtgtagcgaattcccaaaccctgacctgttgatcagaacaagcggagagcaaaggatcagtaacttctttttatggcaatctgcttacactgagctttactttccaactgttttgtggcctgattttggcgaagctgagtatcttgaggccttgacttggtatcagcaaaggcagaggcgattcggtcgacgagtttaaaacttagaatttgcatgtctcatagttgatgcaatagtacgtagtgatacggttgtttaatttcatgagacctaaattgttgtctttgttttaaaaatgctatttatttactttcaataaattcactatagaaactatcataattagatcgtctcttaccaatttatataatacaacaaaatttatgcatttaaagattttagcggctggctcaatcaacacaaaatagtttgaatggtagataacacaatttataaccatactgactagttcattgattgagcatcatctacttaacagagagattattaatctttttacttactctgtttcaccgaataggcaaaataaaaatgtcaatgcttaactcgtgtttgtttagtgaaaaagtaactaagttaatttttggcaaggtttccaaaactgcgagagaaacaaccagagccagtcaataaaaactcaattatgaattttatgatccatgcaaatcacttgctagtactcttcacactctctacaatgttgtctatgctctggttccttctatctcttctctctcttcttctacttccttgtctccgaccttgctttcctgctaaaggatctctgaaaaacaaaaagaagatgtacaaaatcttgcttttttctggttaaactcgatgaaagttcccgctaaaattttcatgacttagctttttcttaaaaaatgtttttgtagtgacaagggaacttatgtggtcggagaggaggagacacctaaagagcttcagagagagctgatgccgagacacgtggcagtgataatggacggaaaccggagatgggctaaacagaccggattgttgacgtcacaaggctacgaggctggagctaaacggcttctcgagttcgctgacctctgcttcaaactagggataaatactgtctcagcttttgctttctccactgagaattggggaagacacaaggtgtggcagtttcatgcgaattgttttagttatccttaaatatttcgccattttgtatctctaataacttaattactcagattgaagtcaaatgcttgatgtatttgttccaacgttacctcaagtcaaagatccaatttttccagaggtatatataaatgagtgagcttattcgatatagtagagcttctcaatagctcagtttgttaccaataatcttgtttgtttcagcaaggaaatccgagtttctgttatcgggaatcttgcgaagatccctgagtctctcctccgaacagttcacgagttagaggaagctacaaagagttacaagaagaagcatctcatcttggccatagattacagcgggagattcgacatcttgggtgcttgcaagaacattgtgaagaaatcagaacaaggtttgatccgagaggaagacgtggacgagacgttgttcgagagagagcttcagactcggtgtaccgagttcccaagtcctgatctcttgattaggactagtggggaacagaggattagtaacttcttcttgtggcagctcgcttataccgagtttttcttttcgcctgtactttggcctgatttcgataagcagaagtttatagaggccctggtttcgtatcagcgcagggatagacgatttggctcccggctttgatccacatttgatcctatggtctgtcatgtttcctctgttttgttctgtttctctctgtgtctttatgttcatctgacttttgcagaaataattatactgtctagtctctcgtagatcaatgtggtatttccaatgaagtaacatattttatgtgttgcgttttttcttctctactacacgtacgtatgcatcagctgttattaaaatatcttgtgacacaagaaacgtgggttctttgagctggacaaagataatccctaggttagattaaacgaatcactcaaatgttttacctaatttaataaaggattctcgtaaaagtattaaacatataccacttcaatttttcacctactttttttaagttttagatcaacaataataattaaactttagacatctaataagttctagtaaaagtattaaaaatggtattaaaagtgagatgcaaaattttcaactcgtacatatatttaataattattaaatgtaacaacagacagaaaatatgttattttaaggaatttgtgggtgtcattgttgttgaccaaacgaattaagctacaaacactagaaacaatttagtgcagtggagttgccgacttagtttaacaagttatttatttattagtttaaccaattaaagaaaaaaatttggatttgttcgattacatcagtactcttggaagggtctggtaataaactaatacggatcaatagctgatgtattaaggaatctttgatcaaaaattaaacataacatgtcatcgtattggtgacatacgaataaaaaatactagtgatctactaattagtgattaaacattgaaaatgatttagtataagtttctcaacgtacaagtttaatacgtacggcctgttttacgtagaaaaagagaaagaaggtttgatcgatcaggtgatgtctttggattctatatatgagaaactttcaatgagtcgaacaaat

SEQ ID NO: 65 - DNA, 3000 bp, Arabidopsis thaliana
  AT5G61070 chr524574373-24577372 REVERSE (source_file=TAIR9_upstream_3000_20090619)
gtaccaaaacgtgtcccgccgagtcgaactcctgaagcaccaagatctgtcccggcttgtccaattcctgaaataccaagacctgtcccagctcgtccaacccctgaaacaccaagacctgtcacagctcgtccaacccctgaaataccaagacctgtcccagctcgtccaatttctgaagtacaaacacttgtcccgactcgtcctacttctacagcacccagccctgtctcggctcatccaacttctgtagtaccaagccctgtctcggttcatccaacttctgtagcaccaagccctgtctcggcttgtctaacttctgaagcatcaaaacctgtgttggctaatcctgcagcaggtattagaagagaactcgtttgcagtcttcgagctttgagtatgactactacaccagtgagacagagaaccactccaagtctccttaaatctaagaaagttgagcctccaagtccacgtgcgttgcaaattcgttcaagagttaagcaacaagcagcaaatgcaacaaaaattcaacaaggtgttatcttttgtttctcttttctttctgaaagccaaagatcctaaacttgttttgatcggatcgctaaaaaaactattgatttttcttggtttctctaggggcgatagtggaagttggggataaagctaaagatcatggttccaacgagatacgtcgatcagtctctgaggaagaattatcggaattacttcaatatctaccagctcagaatcctacttggaagtgagtttcttaacacttcataatcttttttgagctaaacatgtgttttgttagaagcgtcaatcagatcacgctatatgctaaattgttgccatcactctttcccagttttacaacttagggaatgttgtgtgactacctttttcatgtagctcttagccttatttgcatttttttcaaggaaatctagtggctcttacttgccagaatgtaaaacagtccctaatctctttcacagtttaggcatatgctacattgttgaaactggagatgtgcaaatatagaaacgtgctgggatttcgagagcatgaccataataagattcactgatgccaatattgcacttttctccttttttgattcctttgatcttttgaccttcacaagttatatgatgaaattttccttttaactactgaaccagcggacgcattatggattctgcctcgccgcctgagtttgactgcgagttttggtctaaaccggcatccaatatcaggaggaaggccctcagacgttcaaaggcaatgcctgcgttactcaaggtcgaactgcttcctacatgccacattctaaatgatgtgtctggcagaagcccaactctattgaatgtcgaaatgtaccttttcccagatgagaagaaaacagaaaggtactcaaaacccctttctatgaaggacaagcttcttattcttgttgtatctctgttatcagaaaacttatcatctcagggcatgtacactcattcttttaggtttacaggggaacatgctactctgttcaaggcaatggtaactcgcaaagtcatggccaaagctaatataaacggcactgaattgttaatattctcctcaaaactattggacaagacctcccaatgtatgtatgcattctgaatttctgcagagcttttagtttgtgcatagattttcgatggataacatccgttttgtgttcagcagttctcatcaatacgcagaagaaaacagaaaacttcctttggggtttttttctccagaccaagaactcactaggacttgttccagaaactgcttctcaaattgataatgattttgatgatgggggtgttgttgacatggacttagacaatgaatgcctgaccccgcgtgtggctctgaagcttattacagaatcacaaaaaactccctcaagatcaccggagaaggtgaagggagaagcaagtgtacctcctggtttcgagaagatatggacaccacccttggtgaaacttaatatacaaggaacatcaaaccctctgtctgatctaaccagatcggctggaatagtgcgtgaccaatcagggaagtgggtgtttgggtacatcaggtgccataagagcatccctgaagtcgtagctgggcttttggctatataccaaggcctcaaatatctctgggacagcggtttccgaagaatccacttggagacaacaagctttgagatcatcaatgctctaacaacaaaatcgtccctgttttgtaaaagtaaaacgcttctaggagcgtgcaaggacatgatattgaaggagtgggagtgtgacatttaccatatctccaaagaacagaactcatgtgcagaatggttagcaaagagatcagaagaacagcctcaagaattagttttctttgaataccctcctcgaggccttatggattttctggagaaagaccgccttgcagctatgtagagcttcttcaggtttgaggtacagtcccagtgagcagcttagaggtacatatctcgattaagtagtctgaccttttgaaaatactagtagaagaagagatgtttggagtgttaatgtgacatccttgcgtagtcgtaagtacagtggagtctcattgtagccacagttcaagaacacagacgtttttgtgaagtgaagaatcgaaataactgtagattttccctccagattcagtgttgctcctcttcatctttcgtttaggcacagtcttaacaattcacaaaaaaaaaaaaaaaaaaaattactattaaacgataaattcgccgtttgcagaaaattcggttcaccaattttaaattggtttatgaggttcaaccggtcattggttctaaatgagtttgaggtgtccgcctttgattggttgagatgctaggtttttcattttccctccaaagaagctctttaatggtccaacaaatatatatttaatcgtaaataagataaagaaaaatatattttgaaaagaaaa

SEQ ID NO: 66 - DNA, 3000 bp, Arabidopsis thaliana
  AT5G64910 chr525937711-25940710 FORWARD (source_file=TAIR9_upstream_3000_20090619)
atcaggtaattatattttggttttaaaagtcattagcaatgttttaggaatattatataacatgaatccaacagatatgtacatgtgtttataattgcagaaggaggaaaaagaagacgttaccatggaaaaggaggaggtcgttgtgacgagtagagccacaaaggtcaaggcaaagcaaagggggaaggagaaagttagctcaggccgtcctggccaacataattaggcactttaagttacattgtttagtctaattatttgcagtcgaaatgtgttaatttaatatcactgttttacttttttattatatcaacaatctacagacaaacaaaatttcattaagttcttgttcactatacgagttttttcctctttatttcatgcattttttttttcccgtatttgatgtgatatttgggttgaacaaaagtaaatatgttcgaccatcataagttcataaccagtatcaatattaattgaacgcatttaatatccatccatatcgaacgtatctgtcaaatgttaaacaacattaaagacgaacctaattgatatttttggatttattttaacagtcaacagctatttggttctacctagctagctcacattaattataatttaatgtcttcactcatagaatggtcatgtaaaattactaaagaattaagttttgcattcaccatacttttgtttatagatcatcataccaataatttataaagttagatgttataaaataaaataaacaaaaaccttaaacgcagtgataatcatcctatcacaattcacaacgtgcattatgaatggtaatattatatacaaagttgacgagtgctctctatgagtaacgcatgatcgctctatgatttaataaaatatactccacgaggagcagaagcaacaccaacttcgaatataaaacacatttaacaatcttcactgtgactttagacatcatgatttttttggtgttctttttttaaaaaactcataatctggtgataagtctcgtagttacctcatcgtagaaccacaatttagtatttacacaagaaggtatggtctgattaaagtttgggggtctttgtaattaggcctacaataatcattcacaaatttgaaatattacaatttatgagggaccacaacacctaaaaaggcataaatgcattatgcccagtggcgacgaaaaaagtgcattaaaccaaaggaatatatgtttgatttttattttaatgtgggaatgggatatgcaatattacaattatgatattatctgaaattttatatctttacaaaatttgattttcaaacttaaattctatagtatgatctacatgaacactttatattcttatgcaagaaaaacaagtcctttgtccaatcattgacctcttaattgaatttgaaatatttaacaaagtacccaatagataattcttttgttcttgttaaattagattttcaaaatactaagctttagcattaatttgaataatcaaagatcttcctttgaattacctattcaacatttgtttaataacttaattcaaacaaaaaccaaagaagagcgtgaattgactttgaccaaaaccaaattctctcaagagttgctgccatattttgaccgcgcgccacgtagattagagtcaaaattgttattttattcacttcaacataaaaccaaataagcattatcggttttcaacataccggcaccaacttttccaaagtctgtatgtacctaacaaaaccggtttatcatagaaacggtcaacacaccaaaaatagttgaccaacaactacccaagtgatatccctttaaaaggagtcgcatatgtgttaccaagttccatcatcaacctaataacacacaacactaaatctctttcccaaaaaaagattaagaagtcaacgatggagaatctcagaaatggagaagataacggttctttgatcccatttacgttctttgatcaatcttcagtgacgattcctctcttgaagtgttccggtctcgaaagttcatcatcatcatcttcttcttgcgatctttcgtcatcacacagcgaggaagatgagagtatcgatataaaggaggaggaagaagaagaagaagaagatggcatgaccattgaaatcaaagcgagagggaagaacaagactaagcctacgccaagttcaggaaaaggaggcaaacacaattagagttcattcatataccgaggaaattaaacaaataaatgcatttgtataaaatacttagagctataatacagtggagtttttttatagtcatttgtttcgaatatgaattggattaataaagatcgagttttattttattgtttcgttacagaataggagaatttaattgcagaatcatgatttatcatcgcttagttcatctcgcaagataccttcttcacaggatcttgtcgagtcatgcaactgaatttgaacaaaaaatacttgctaaatgcagcttaagcctctatgggcctaacctatttagcccattaatgatttgtaaagaccagattgtcgacccataaattgtctttaaacttaagtaaatggtgacgactgacgactgacgactgacgacatccttgaagagttaattgtttatctattagttaatgctattattctccggttttggagatattaattactattatccagcctgtcattttcattggtgcaaattaaaaaagatttctatttttccatattcatcatacacgctgaaagataacatttagccgttggatgtatgacacgtcagcgataaacatacgtaatgaaggtttaaatgagtaaataattacgatattaccccttaagtccccgcgcttaatttattttttgagggaaaataaaagaaaggaaaaaaagaaaacaaaaactaacaaattcttatttacatctactgatggatgggacgttaaaatcacgcaagaaaggacgactgaaaagagagagaggtac

SEQ ID NO: 67 - DNA, 832 bp, Arabidopsis thaliana
  G481
gagcgtttcgtagaaaaattcgatttctctaaagccctaaaactaaaacgactatccccaattccaagttctagggtttccatcttccccaatctagtataaatggcggatacgccttcgagcccagctggagatggcggagaaagcggcggttccgttagggagcaggatcgataccttcctatagctaatatcagcaggatcatgaagaaagcgttgcctcctaatggtaagattggaaaagatgctaaggatacagttcaggaatgcgtctctgagttcatcagcttcatcactagcgaggccagtgataagtgtcaaaaagagaaaaggaaaactgtgaatggtgatgatttgttgtgggcaatggcaacattaggatttgaggattacctggaacctctaaagatatacctagcgaggtacagggagttggagggtgataataagggatcaggaaagagtggagatggatcaaatagagatgctggtggcggtgtttctggtgaagaaatgccgagctggtaaaagaagttgcaagtagtgattaagaacaatcgccaaatgatcaagggaaattagagatcagtgagttgtttatagttgagctgatcgacaactatttcgggtttactctcaatttcggttatgttagtttgaacgtttggtttattgtttccggtttagttggttgtatttaaagatttctctgttagatgttgagaacacttgaatgaaggaaaaatttgtccacatcctgttgttattttcgattcactttcggaatttcatagctaatttattctcatttaataccaaatccttaaattaa

SEQ ID NO: 68 - protein, 141 aa, Arabidopsis thaliana
  G481 polypeptide
MADTPSSPAGDGGESGGSVREQDRYLPIANISRIMKKALPPNGKIGKDAKDTVQECVSEFISFITSEASDKCQKEKRKTVNGDDLLWAMATLGFEDYLEPLKIYLARYRELEGDNKGSGKSGDGSNRDAGGGVSGEEMPSW

SEQ ID NO: 69 - DNA, 828 bp, Arabidopsis thaliana
  G1543
atgataaaactactatttacgtacatatgcacatacacatataaactatatgctctatatcatatggattacgcatgcgtgtgtatgtataaatataaaggcatcgtcacgcttcaagtttgtctcttttatattaaactgagagttttcctctcaaactttaccttttcttcttcgatcctagctcttaagaaccctaataattcattgatcaaaataatggcgattttgccggaaaactcttcaaacttggatcttactatctccgttccaggcttctcttcatcccctctctccgatgaaggaagtggcggaggaagagaccagctaaggctagacatgaatcggttaccgtcgtctgaagacggagacgatgaagaattcagtcacgatgatggctctgctcctccgcgaaagaaactccgtctaaccagagaacagtcacgtcttcttgaagatagtttcagacagaatcatacccttaatcccaaacaaaaggaagtacttgccaagcatttgatgctacggccaagacaaattgaagtttggtttcaaaaccgtagagcaaggagcaaattgaagcaaaccgagatggaatgcgagtatctcaaaaggtggtttggttcattaacggaagaaaaccacaggctccatagagaagtagaagagcttagagccataaaggttggcccaacaacggtgaactctgcctcgagccttactatgtgtcctcgctgcgagcgagttacccctgccgcgagcccttcgagggcggtggtgccggttccggctaagaaaacgtttccgccgcaagagcgtgatcgttga

SEQ ID NO: 70 - protein, 275 aa, Arabidopsis thaliana
  G1543 polypeptide
MIKLLFTYICTYTYKLYALYHMDYACVCMYKYKGIVTLQVCLFYIKLRVFLSNFTFSSSILALKNPNNSLIKIMAILPENSSNLDLTISVPGFSSSPLSDEGSGGGRDQLRLDMNRLPSSEDGDDEEFSHDDGSAPPRKKLRLTREQSRLLEDSFRQNHTLNPKQKEVLAKHLMLRPRQIEVWFQNRRARSKLKQTEMECEYLKRWFGSLTEENHRLHREVEELRAIKVGPTTVNSASSLTMCPRCERVTPAASPSRAVVPVPAKKTFPPQERDR

SEQ ID NO: 71 - DNA, 906 bp, Arabidopsis thaliana
  G1988
tgctactctcatcaaccatgaaccataaaaactccaccgctctttctctccctcaatcatttacatctcttccttaaatctctcttcccaccatcatcattccaaaccaattctctctcacttctttctggtgatcagagagatcgactcaatggtgagcttttgcgagctttgtggtgccgaagctgatctccattgtgccgcggactctgccttcctctgccgttcttgtgacgctaagttccatgcctcaaattttctcttcgctcgtcatttccggcgtgtcatctgcccaaattgcaaatctcttactcaaaatttcgtttctggtcctcttcttccttggcctccacgaacaacatgttgttcagaatcgtcgtcttcttcttgctgctcgtctcttgactgtgtctcaagctccgagctatcgtcaacgacgcgtgacgtaaacagagcgcgagggagggaaaacagagtgaatgccaaggccgttgcggttacggtggcggatggcatttttgtaaattggtgtggtaagttaggactaaacagggatttaacaaacgctgtcgtttcatatgcgtctttggctttggctgtggagacgaggccaagagcgacgaagagagtgttcttagcggcggcgttttggttcggcgttaagaacacgacgacgtggcagaatttaaagaaagtagaagatgtgactggagtttcagctgggatgattcgagcggttgaaagcaaattggcgcgtgcaatgacgcagcagcttagacggtggcgcgtggattcggaggaaggatgggctgaaaacgacaacgtttgagaaatattattgacatgggtcccgcattatgcaaattaggacatttagtgtttagtgcattaattatagtttgtgtc

SEQ ID NO: 72 - protein, 225 aa, Arabidopsis thaliana
  G1988 polypeptide
MVSFCELCGAEADLHCAADSAFLCRSCDAKFHASNFLFARHFRRVICPNCKSLTQNFVSGPLLPWPPRTTCCSESSSSSCCSSLDCVSSSELSSTTRDVNRARGRENRVNAKAVAVTVADGIFVNWCGKLGLNRDLTNAVVSYASLALAVETRPRATKRVFLAAAFWFGVKNTTTWQNLKKVEDVTGVSAGMIRAVESKLARAMTQQLRRWRVDSEEGWAENDNV

SEQ ID NO: 73 - DNA, 867 bp, artificial sequence
  P21103 example base vector for the creation of RNAi constructs, poly linker and Pdk intron
ggtaccgtcgacgaggaattcggtagcccaattggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcaattttatatagtattatttttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagttgggaaattgggttcgaaatcgataatcttgcggccgctctagacaggcctcgtaccggatcc